(19) United States
(12) Patent Application Publication
(10) Pub. No.: US 2025/0152740 A1
(43) Pub. Date: May 15, 2025

(54) MATERIALS AND METHODS FOR THE TREATMENT OF LIMB GIRDLE MUSCULAR DYSTROPHY

(71) Applicant: RESEARCH INSTITUTE AT NATIONWIDE CHILDREN'S HOSPITAL, Columbus, OH (US)

(72) Inventors: Kevin Flanigan, Columbus, OH (US); Liubov V. Gushchina, Columbus, OH (US)

(21) Appl. No.: 18/720,972

(22) PCT Filed: Dec. 21, 2022
(86) PCT No.: PCT/US22/82143

§ 371 Date: Jun. 17, 2024

Related U.S. Application Data
(60) Provisional application No. 63/319,176, filed on Mar. 11, 2022.
(60) Provisional application No. 63/292,291, filed on Dec. 21, 2021.

(51) Int. Cl. A61K 48/00; A61K 9/00; A61K 31/453; A61K 31/56; C07K 16/28; C12N 15/113; C12N 15/864

(52) U.S. Cl. CPC A61K 48/0058; A61K 9/0019; A61K 31/453; A61K 31/56; C07K 16/2887; C12N 15/113; C12N 15/8645; C12N 2750/14143

(57) ABSTRACT

Provided are gene therapy vectors, such as adeno-associated virus (AAV), designed for treatment of mutations in the TCAP gene. Such mutations are also known as telethoninopathies and are associated with such disorders as autosomal recessive limb girdle muscular dystrophy type 2G (LGMD2G), autosomal dominant dilated cardiomyopathy (DCM), hypertrophic cardiomyopathy (HCM) or idiopathic cardiomyopathy (ICM). The disclosed gene therapy vectors provide a TCAP cDNA to a subject in need which results in expression of a wild type or functional TCAP or telethonin protein.

This application contains, as a separate part of the disclosure, a Sequence Listing in computer-readable form which is incorporated by reference in its entirety and identified as follows: 56598_Seqlisting.XML; Size: 237,879 bytes; Created: Dec. 5, 2022.

### FIELD OF THE INVENTION

The disclosure provides gene therapy vectors, such as adeno-associated virus (AAV), designed for treatment of mutations in the gene encoding the 19 kDa titin-cap protein (“TCAP” or “telethonin”). Such mutations in the TCAP gene are associated with autosomal recessive limb girdle muscular dystrophy type R7 (LGMDR7, also known as limb girdle muscular dystrophy 2G (LGMD2G), autosomal dominant dilated cardiomyopathy (DCM), hypertrophic cardiomyopathy (HCM), or idiopathic cardiomyopathy (ICM). The disclosed gene therapy vectors provide a TCAP cDNA or a wild type TCAP cDNA to a subject in need which results in expression of a functional or wild type TCAP or telethonin protein.

### BACKGROUND

Telethoninopathies are the rarest forms of limb girdle muscular dystrophies (LGMDs) resulting from a mutation in the gene that encodes the titin-cap protein (“TCAP” or “telethonin”) or loss-of-function of TCAP, a small structural protein found in cardiac and skeletal muscles. Mutations in the TCAP gene disturb interactions in the titin Z1Z2/TCAP complex leading to destabilization of the complex, which results in an impaired ability to withstand physiological stresses during muscle contractions. The most common telethoninopathy is the autosomal recessive limb-girdle muscular dystrophy R7 (LGMDR7 or LGMD2G), which is marked by progressive limb girdle muscular atrophy and weakness, cardiomyopathy, and respiratory dysfunction. To date, no treatment that could halt or slow progression of LGMDR7/2G exists. Thus, patients with the disease are in urgent need of new therapeutic options.

The TCAP gene consists of only two exons, leading to an inability to use preclinically/clinically approved antisense oligonucleotide (AON) or small nuclear U7snRNA therapies, which are currently in development to restore mRNAs open reading frames in muscular dystrophies, including LGMD2B (dysferlin deficiency)¹⁻³ or DMD/BMD (dystrophin deficiency)⁴⁻⁷.

The TCAP protein, also known as telethonin, plays an essential role in regulating sarcomere assembly by serving as an anchor for titin in titin Z1Z2/TCAP complex to increase the mechanical resistance of the sarcomere⁸⁻¹¹. The terms “the titin-cap protein (TCAP)” or “telethonin” are used interchangeably herein.

Because patients with a mutation in their TCAP gene suffer from or are risk of suffering from limb girdle muscular atrophy and weakness, cardiomyopathy, and respiratory dysfunction, there is an urgent need for new therapeutic options. The disclosure provides a replacement TCAP nucleic acid and TCAP gene replacement as a feasible therapeutic strategy to treat a mutation(s) in the TCAP gene and treat, prevent, or ameliorate a telethoninopathy resulting from such mutation(s). The disclosure thus provides nucleic acids, nanoparticles, extracellular vesicles, exosomes, vectors, recombinant AAV particles, and compositions for treating TCAP mutations. The disclosed products, methods and uses provide a feasible approach for robust and long-term expression of the TCAP protein, or a functional TCAP protein, in human striated muscles.

### SUMMARY

Provided herein are products, methods, and uses for treating mutations in the gene encoding “the titin-cap protein (TCAP)” or “telethonin” and in treating, ameliorating, delaying the progression of, and/or preventing diseases resulting from mutations in the TCAP gene.

The disclosure provides a nucleic acid comprising a polynucleotide comprising (a) one or more regulatory control element(s); and (b) a titin-cap protein (TCAP) cDNA sequence. In some aspects, the TCAP cDNA comprises (a) a nucleotide sequence having at least 90% sequence identity to any one of SEQ ID NOs: 1-4; (b) the nucleotide sequence set forth in any one of SEQ ID NOs: 1-4; or (c) a nucleotide sequence encoding TCAP comprising the amino acid sequence set forth in SEQ ID NO: 5. In some aspects, the one or more regulatory control element(s) is an H1 promoter, an EF1-alpha promoter, a minimal EF1-alpha promoter, an unc45b promoter, a CK1 promoter, a CK6 promoter, a CK7 promoter, a CK8e promoter, a cardiac troponin C (cTnC) promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter, or a native TCAP promoter. In some aspects, the one or more regulatory control element(s) is a CMV promoter, an MHCK7 promoter, a native TCAP promoter, or a fragment of any of the CMV promoter, the MHCK7 promoter, or the native TCAP promoter. In some aspects, the regulatory control element comprises (a) a nucleotide sequence having at least 90% sequence identity to SEQ ID NO: 6, 7 or 8; or (b) the nucleotide sequence set forth in SEQ ID NO: 6, 7, or 8. In some aspects, the nucleic acid comprises (a) a nucleotide sequence having at least 90% sequence identity to any one of SEQ ID NOs: 29-38; or (b) the nucleotide sequence set forth in any one of SEQ ID NOs: 29-38. In some aspects, the nucleic acid further comprises an inverted terminal repeat sequence. In some aspects, the nucleic acid comprises (a) a nucleotide sequence having at least 90% sequence identity to any one of SEQ ID NOs: 49-58; or (b) the nucleotide sequence set forth in any one of SEQ ID NOs: 49-58. In some aspects, the nucleic acid comprises (a) a nucleotide sequence having at least 90% sequence identity to any one of SEQ ID NOs: 39-48; or (b) the nucleotide sequence set forth in any one of SEQ ID NOs: 39-48.

The disclosure provides a nanoparticle, extracellular vesicle, exosome, or vector comprising any of the nucleic acids as described herein or a combination of any two or more thereof. In some aspects, vector is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV), a self-complementary recombinant AAV (scAAV), or a single-stranded recombinant AAV (ssAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAVMYO, or any of their derivatives. In some aspects, the AAV is AAV9.

The disclosure provides an rAAV particle comprising any of the AAV as described herein or a combination of any two or more thereof.

The disclosure provides a composition comprising any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; or (d) an rAAV particle as described herein or a combination of any two or more thereof; and a pharmaceutically acceptable carrier. In some aspects, the composition is formulated for intramuscular or intravenous delivery.

The disclosure provides a method of increasing the expression of a TCAP gene or TCAP protein in a cell comprising contacting the cell with any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; or (d) an rAAV particle as described herein or a combination of any two or more thereof; or a composition as described herein or a combination of any two or more thereof. In some aspects, the cell is a muscle cell. In some aspects, the cell is a human cell. In some aspects, the cell is in a human subject.

The disclosure provides a method of treating a subject comprising a TCAP gene mutation or a telethoninopathy comprising administering to the subject an effective amount of any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; or (d) an rAAV particle as described herein or a combination of any two or more thereof; or (e) a composition as described herein or a combination of any two or more thereof. In some aspects, the subject is a human subject. In some aspects, the telethoninopathy is autosomal recessive limb girdle muscular dystrophy type 2G (LGMD2G), autosomal dominant dilated cardiomyopathy (DCM), hypertrophic cardiomyopathy (HCM) or idiopathic cardiomyopathy (ICM). In some aspects, the method further comprises administering any one or more of a corticosteroid, rituximab, and rapamycin to the subject. In some aspects, the nucleic acid, nanoparticle, extracellular vesicle, exosome, vector, rAAV particle, or composition is administered by intravenous or intramuscular delivery.

The disclosure provides a use of any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; (d) an rAAV particle as described herein or a combination of any two or more thereof; or (e) a composition as described herein or a combination of any two or more thereof for the preparation of a medicament for increasing expression of the TCAP gene or protein in a cell. In some aspects, the cell is in a human subject. In some aspects, the medicament is administered with any one or more of a corticosteroid, rituximab, and rapamycin. In some aspects, the medicament is formulated for intravenous or intramuscular delivery.

The disclosure provides a use of any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; (d) an rAAV particle as described herein or a combination of any two or more thereof; or (e) a composition as described herein or a combination of any two or more thereof in treating a subject comprising a mutant TCAP gene. In some aspects, the subject is a human subject. In some aspects, the subject suffers from a TCAP mutation or a telethoninopathy. In some aspects, the telethoninopathy is autosomal recessive limb girdle muscular dystrophy type 2G (LGMD2G), autosomal dominant dilated cardiomyopathy (DCM), hypertrophic cardiomyopathy (HCM) or idiopathic cardiomyopathy (ICM). In some aspects, the TCAP mutation results in an intestinal obstruction. In some aspects, the medicament is administered with any one or more of a corticosteroid, rituximab, and rapamycin. In some aspects, the medicament is formulated for intravenous or intramuscular delivery.

The disclosure provides a composition for treating a TCAP gene mutation or a telethoninopathy in a subject, wherein the composition comprises any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; (d) an rAAV particle as described herein or a combination of any two or more thereof; or (e) a composition as described herein or a combination of any two or more thereof. In some aspects, the subject is a human subject. In some aspects, the telethoninopathy is autosomal recessive limb girdle muscular dystrophy type 2G (LGMD2G), autosomal dominant dilated cardiomyopathy (DCM), hypertrophic cardiomyopathy (HCM) or idiopathic cardiomyopathy (ICM). In some aspects, the composition further comprises any one or more of a corticosteroid, rituximab, and rapamycin. In some aspects, the composition is formulated for intravenous or intramuscular delivery.

The disclosure provides any of (a) a nucleic acid as described herein or a combination of any two or more thereof; (b) a nanoparticle, extracellular vesicle, exosome, or vector as described herein or a combination of any two or more thereof; (c) a viral vector as described herein or a combination of any two or more thereof; (d) an rAAV particle as described herein or a combination of any two or more thereof; (e) a composition as described herein or a combination of any two or more thereof; (f) a method as described herein; or (g) a use as described herein, wherein the nucleic acid, nanoparticle, extracellular vesicle, exosome, vector, viral vector, composition, or medicament is formulated for intramuscular injection, oral administration, subcutaneous, intradermal, or transdermal transport, intravenous injection into the blood stream, or for aerosol administration.

Other features and advantages of the disclosure will become apparent from the following description of the drawings and the detailed description. It should be understood, however, that the drawings, detailed description, and the examples, while indicating embodiments of the disclosed subject matter, are given by way of illustration only, because various changes and modifications within the spirit and scope of the disclosure will become apparent from the drawing, detailed description, and the examples.

Other features and advantages of the present disclosure will become apparent from the following detailed description. It should be understood, however, that the detailed description and the specific examples, while indicating preferred embodiments of the disclosure, are given by way of illustration only, because various changes and modifications within the spirit and scope of the disclosure will become apparent to those skilled in the art from this detailed description

### BRIEF DESCRIPTION OF THE DRAWINGS

FIG. 1 provides a schematic diagram of the rAAV.TCAP vectors designed to test the restoration of TCAP expression. Vectors/constructs 1 and 2 represent constructs with a human TCAP native promoter (NP, orange), the mouse analog of which has been shown to be involved in circadian TCAP regulation in skeletal muscles^{12,13}; vectors/constructs 3-6 represent constructs with a MHCK7 promoter (lavender), which show high level of expression in fast and slow skeletal and cardiac muscle; and vectors/constructs 7-10 represent constructs with a human cytomegalovirus (CMV) promoter (purple), which show high level of expression in all tissue and organs. The human TCAP gene is represented as exon 1 (light green), intron 1 (gold, NI, native intron) and exon 2 (dark green). A chimeric simian virus 40 (SV40) intron (SI, synthetic intron) is represented in light gold.

FIG. 2 provides photographs of immunofluorescent staining showing TCAP protein expression in differentiated C2C12 myotubes (Day 9 differentiation), which were electroporated/transfected with rAAV.TCAP.3×FLAG (Amp+) plasmids. C2C12 transfected cells showed TCAP expression in rAAV.TCAP.3×FLAG (Amp+) treated cells after nine days of differentiation.

FIG. 3A-B shows Western blot images of the co-expression of endogenous mouse TCAP (mTCAP) and exogenous human TCAP.FLAG (hTCAP) proteins in C57Bl/6 (C57) mice tibialis anterior (TA) muscles which were intramuscularly (IM) injected with scAAV9. TCAP.3×FLAG (Amp+) vectors. FIG. 3A shows individual channels showing FLAG (red) and TCAP (green) signals in the animals injected with diluent (C57, Group 5), SCAAV9.MHCK7.TCAP.NI3×FLAG (Amp+) (Group 1), scAAV9.MHCK7.TCAP.SI3×FLAG (Amp+) (Group 2), scAAV9.CMV.TCAP.NI3×FLAG (Amp+) (Group 3), or scAAV9.CMV.TCAP.SI3×FLAG (Amp+) (Group 4). FIG. 3B shows their merged images showing the co-expression (yellow) of both proteins in TA muscles. The transduction of scAAV9.TCAP.3×FLAG (Amp+) resulted in human TCAP protein expression in TA muscles of C57Bl/6 mice 4 weeks post vector administration at a dose of 1.5E11 vg per muscle.

FIG. 4A-D provides representative Western blot images showing the restoration of TCAP protein in TCAP-null cells (FM193ML) treated with scAAV1.MHCK7.TCAP.SI3×FLAG (Amp+). FIG. 4A shows the total protein level was detected by using Total Protein Stain (REVERT) from LiCOR. FIG. 4B shows the merged images with anti-TCAP and anti-FLAG antibodies; FIG. 4C shows individual images with membrane that was probed with anti-TCAP; and FIG. 4D shows individual images with membrane that was probed with anti-FLAG antibodies, each followed by secondary IRDye 800CW or IRDye 680RD conjugated antibody and imaged. WB Lanes: (1) FM193ML (TCAP-null) Fibroblasts; (2) FM193ML (TCAP-null) Myoblasts, Day 3; (3) FM193ML (TCAP-null) myotubes treated with scAAV1.MHCK7.TCAP.SI3×FLAG (Amp+), Day 14 differentiation; (4) FM193ML (TCAP-null) Myotubes untreated, Day 14 differentiation; (5) FM021PK Myotubes, Day 14 differentiation (normal female cell line); and (6) FM190KL Myotubes, Day 15 differentiation (normal male cell line). The scAAV1.MHCK7.TCAP.SI3×FLAG (Amp+) treatment was effective in expressing TCAP protein in the TCAP-null cells. TCAP protein was detected by both anti-TCAP and anti-FLAG antibodies in human TCAP-null cells after 14 days of differentiation in transduced cells. Human control cell lines (both male and female) did not show any visible TCAP protein expression at similar conditions presumably due to low level of TCAP protein. Thus, new cell lines are being tested to determine the level of endogenous TCAP in controls in order to quantify the TCAP.3×FLAG level restored in treated FM193ML cells.

FIG. 5 provides representative immunofluorescent (IF) images showing TCAP (magenta channel) and FLAG (green channel) signals in TCAP-null cells (FM193ML) treated with scAAV1.MHCK7.TCAP.SI3×FLAG (Amp+) vector (top panel) and untreated (bottom panel) after 14 days of differentiation. All images contain 4 small squares, each of them represents either merged channels (left top corner) or DAPI (nuclei staining, right top corner), FLAG (left bottom corner) and TCAP (right bottom corner). The images were taken on a Nikon Ti2E microscope with a 100× oil objective. Untreated TCAP-null cells (FM193ML) stained with secondary antibody only (no primary) were used as a negative control to determine non-specific background of images. The fluorescent microscopy revealed the expression TCAP protein by detecting both TCAP and FLAG signals in TCAP-null cells treated with test articles and confirmed their colocalization in the cytoplasm with a more pronounced signal located on the plasma membrane. Some non-specific signal for both TCAP and FLAG antibodies was present in untreated FM193ML cells; however, the fluorescent brightness of these signals is less intense in untreated cells even though the exposure time was almost twice the exposure time used for scAAV1 treated cells.

FIG. 6A-C shows results of experiments measuring membrane damage in human myotubes after laser injury by measuring fluorescence intensity encompassing the site of damage. FIG. 6A provides representative images of laser injury in cells after 5 days of differentiation (Day 5 Diff). White arrows indicate the wounding site at 0 sec, 3.1 sec and 52.8 sec after laser injury in the presence of calcium ions (2 mM Ca²⁺). FIG. 6B shows time-dependent accumulation of FM4-64 dye in FM000KF (normal male cells, WT cells) and FM193ML (TCAP-null) untreated cells and treated with scAAV1.MHCK7.TCAP.SI3×FLAG (Amp+) test articles, the analysis of which is represented as the Area Under the Curve (AUC). AUC is the definitive integral of a dye accumulation curve between 0 sec and 53.04 sec. FIG. 6C shows AUC of FM4-64 fluorescence traces displays the membrane-resealing process in normal human myotubes (FM000KF, Day 5 Diff), TCAP-deficient (FM193ML, Day 5 Diff) or TCAP-treated (FM193ML treated with scAAV1.MHCK7.TCAP.SI3×FLAG, Day 5 Diff) cells. The effect of the absence of calcium ions (0 mM Ca²⁺) is recorded for scAAV1 treated FM193ML cells. Data represented as means±SEM (n=9-12). The Tukey-ANOVA was utilized for statistical analysis. Human myotubes deficient for TCAP protein displayed compromised membrane repair. Restoration of TCAP function, i.e., the transduction of TCAP cDNA into the myotubes, led to an increase in the membrane repair capacity up to the level detected in normal human myotubes. Results also show that calcium ions play an essential role in this process, because the absence of extracellular Ca²⁺ resulted in significant dye entry following laser injury despite TCAP expression in transduced/treated cells suggesting that calcium is important in membrane repair.

FIG. 7 provides representative IF images showing TCAP protein (magenta channel) and titin protein (green channel) co-localization. 10 week-old hTCAPKI males were intramuscularly injected with scAAV9.MHCK7.TCAP.NI3×FLAG (Amp+) or SCAAV9.MHCK7.TCAP.SI3×FLAG (Amp+) vectors and TA muscles were collected at four weeks post-vectors administration. The images were taken on a Nikon Ti2E microscope with a 100× oil objective. Scale bar is 10 μm. Tissue sections stained with secondary antibody only (no primary) were used as a negative control to determine non-specific background of images. Scale bar=10 μm. The fluorescent microscopy revealed significant expression of human TCAP protein in TA muscles from hTCAPKI-null males four weeks post AAV9.TCAP.3×FLAG (Amp+) vector administration. The expression of titin protein as well as its proper co-localization with TCAP was also confirmed. The significant titin protein expression increase was observed in hTCAPKI treated mice. hTCAPKI controls stained with TCAP antibody showed some propensity for nonspecific binding leading to a background signal seen in hTCAPKI diluent group.

FIG. 8A-E shows that systemic delivery (tail vein) of scAAV9.TCAP.3×FLAG vectors results in significant TCAP protein expression in triceps muscle at four weeks post-injection. Representative images of immunoblots showed an increase in TCAP expression in triceps post-administration with scAAV9.TCAP.MHCK7.NI3×FLAG (FIG. 8A), scAAV9.TCAP.MHCK7.SI3×FLAG (FIG. 8B), scAAV9.TCAP.CMV.NI3×FLAG (FIG. 8C), and scAAV9.TCAP.CMV.SI3×FLAG (FIG. 8D). FIG. 8E shows densitometric analysis of the western blots presented in FIG. 8A-D, which was carried out using Image Lab software (Bio Rad, version 6.0.0 build 25). The intensity of TCAP bands in treated groups was normalized to the GAPDH band intensity of the respective sample. Data provided are the means±SEM (n=3-4 for each group). All systemically delivered scAAV9.TCAP.FLAG vectors showed a significant level of TCAP protein expression in triceps muscles. Samples from C57Bl6 (Bl6) animals revealed that endogenously expressed mouse TCAP (mTCAP) protein is smaller in size than exogenously expressed human TCAP (hTCAP) protein, as expected due to the 3×Flag sequence extension.

FIG. 9 provides an annotated sequence of rAAV.NP.TCAP plasmid sequence (5627 bp) (SEQ ID NO: 19).

FIG. 10 provides an annotated sequence of rAAV.MHCK7.TCAP.NI plasmid sequence (5506 bp) (SEQ ID NO: 20).

FIG. 11 provides an annotated sequence of rAAV.MHCK7.TCAP.SI plasmid sequence (5412 bp) (SEQ ID NO: 21)).

FIG. 12 provides an annotated sequence of rAAV.CMV.TCAP.NI plasmid sequence (5351 bp) (SEQ ID NO: 22).

FIG. 13 provides an annotated sequence of rAAV.CMV.TCAP.SI plasmid sequence (5252 bp) (SEQ ID NO: 23).

FIG. 14 provides an annotated sequence of rAAV.NP.TCAP.3×FLAG plasmid sequence (5693 bp) (SEQ ID NO: 24).

FIG. 15 provides an annotated sequence of rAAV.MHCK7.TCAP.NI3×FLAG plasmid sequence (5575 bp) (SEQ ID NO: 25).

FIG. 16 provides an annotated sequence of rAAV.MHCK7.TCAP.SI3×FLAG plasmid sequence (5476 bp) (SEQ ID NO: 26).

FIG. 17 provides an annotated sequence of rAAV.CMV.TCAP.NI3×FLAG plasmid sequence (5417 bp) (SEQ ID NO: 27).

FIG. 18 provides an annotated sequence of rAAV.CMV.TCAP.SI3×FLAG plasmid sequence (5140 bp) (SEQ ID NO: 28).

FIG. 19 provides an annotated sequence of rAAV.NP.TCAP/SD plasmid sequence (5749 bp) (SEQ ID NO: 39).

FIG. 20 provides an annotated sequence of rAAV.MHCK7.TCAP.NI/SD plasmid sequence (5628 bp) (SEQ ID NO: 40).

FIG. 21 provides an annotated sequence of rAAV.MHCK7.TCAP.SI/SD plasmid sequence (5534 bp) (SEQ ID NO: 41).

FIG. 22 provides an annotated sequence of rAAV.CMV.TCAP.NI/SD plasmid sequence (5440 bp) (SEQ ID NO: 42).

FIG. 23 provides an annotated sequence of rAAV.CMV.TCAP.SI/SD plasmid sequence (5341 bp) (SEQ ID NO: 43).

FIG. 24 provides an annotated sequence of rAAV.NP.TCAP.3×FLAG/SD plasmid sequence (5815 bp) (SEQ ID NO: 44).

FIG. 25 provides an annotated sequence of rAAV.MHCK7.TCAP.NI3×FLAG/SD plasmid sequence (5694 bp) (SEQ ID NO: 45).

FIG. 26 provides an annotated sequence of rAAV.MHCK7.TCAP.SI3×FLAG/SD plasmid sequence (5600 bp) (SEQ ID NO: 46).

FIG. 27 provides an annotated sequence of rAAV.CMV.TCAP.NI3×FLAG/SD plasmid sequence (5506 bp) (SEQ ID NO: 47).

FIG. 28 provides an annotated sequence of rAAV.CMV.TCAP.SI3×FLAG/SD plasmid sequence (5407 bp) (SEQ ID NO: 48).

FIG. 29A-E shows that systemic delivery (tail vein) of scAAV9.TCAP.3×FLAG vectors results in significant TCAP protein expression in diaphragm muscle at four weeks post-injection. Representative images of immunoblots showed an increase in TCAP expression in diaphragms post-administration with scAAV9.TCAP.MHCK7.NI3×FLAG (FIG. 29A), scAAV9.TCAP.MHCK7.SI3×FLAG (FIG. 29B), scAAV9.TCAP.CMV.NI3×FLAG (FIG. 29C), and scAAV9.TCAP.CMV.SI3×FLAG (FIG. 29D). FIG. 29E shows densitometric analysis of the western blots presented in FIG. 29A-D, which was carried out using Image Lab software (Bio Rad, version 6.0.0 build 25). The intensity of TCAP bands in treated groups was normalized to the GAPDH band intensity of the respective sample. Data provided are the means±SEM (n=3-4 for each group). All systemically delivered scAAV9.TCAP.FLAG vectors showed a significant level of TCAP protein expression in diaphragm muscles. Samples from C57Bl6 (Bl6) animals revealed that endogenously expressed mouse TCAP (mTCAP) protein is smaller in size than exogenously expressed human TCAP (hTCAP) protein, as expected due to the 3×Flag sequence extension.

FIG. 30 provides representative images of RT-PCR analysis of TCAP transcripts in cardiac and skeletal muscles (triceps brachii and diaphragm) from hTCAPKI mice (n=3-4), which were intravenously treated for 4 weeks with rAAV.TCAP vectors: SCAAV9.TCAP.MHCK7.NI3×FLAG, scAAV9.TCAP.MHCK7.SI3×FLAG, scAAV9.TCAP.CMV.NI3×FLAG, and scAAV9.TCAP.CMV.SI3×FLAG or saline as a control. C57Bl6 (Bl6) mice were also utilized as an untreated control. Bands represent either mouse TCAP (mTCAP) or human hTCAP (hTCAP) mRNA transcripts. RT(-) and PCR(-) are negative controls, in which water was used as a template for RT and PCR steps to ensure that the tested samples were free of DNA contamination and non-specific amplification. Ladder, 1 kb Plus DNA ladder. The stars represent what appears to be a possible insufficient amplification of expected bands or an issue with RNA quality.

FIG. 31A-C provides representative IF images showing restoration of TCAP protein in triceps brachii (FIG. 31A) and heart muscles (FIG. 31B-C) in hTCAPKI-null males, which were systemically (tail vein) injected with diluent or the following vectors: scAAV9.TCAP.MHCK7.NI3×FLAG, scAAV9.TCAP.MHCK7.SI3×FLAG, scAAV9.TCAP.CMV.NI3×FLAG, and scAAV9.TCAP.CMV.SI3×FLAG. Age-matched untreated C57Bl6 (Bl6) and diluent treated hTCAPKI-null mice were used as positive and negative controls. Animals were sacrificed 4 weeks post-vector administration. Tissues were frozen in liquid nitrogen cooled isopentane and kept at −80 C until use. 10 μm-thick longitudinally oriented tissue sections were immunolabelled with TCAP and Titin antibodies in triceps (FIG. 31A) and TCAP and sarcomeric alpha-actinin antibodies in hearts (FIG. 31B). The line charts (FIG. 31C) show the intensity plots of the TCAP and alpha-actinin signals over several sarcomeres in one representative cardiomyocyte-across the regions shown by a yellow line on the merged images. The images were taken on a Nikon Ti2E-inverted microscope with a 100× oil objective. Scale bar=100 um.

FIG. 32 shows the biodistribution of rAAV.TCAP vectors in hTCAPKI mice after systemic injection (tail vein) with diluent or the following vectors: SCAAV9.TCAP.MHCK7.NI3×FLAG (MHCK7-NI), scAAV9.TCAP.MHCK7.SI3×FLAG (MHCK7-SI), scAAV9.TCAP.CMV.NI3×FLAG (CMV-NI), and scAAV9.TCAP.CMV.SI3×FLAG (CMV-SI). Age-matched untreated C57Bl6 (Bl6) and diluent treated hTCAPKI-null (hTCAPKI) mice were used as controls. Animals were sacrificed 4 weeks post-vector administration. Data represented as vector copies per diploid genome (vc/dg) in right side of triceps (R. Tri), diaphragm, heart and liver. Data reposted as mean±SEM with individual points. Undet.=undetected; LLOQ=lower limit of quantification.

FIG. 33A-E shows that systemic delivery (tail vein) of scAAV9.TCAP.3×FLAG vectors at the dose of 3E13 vg/kg results in significant TCAP protein expression in left side of quadriceps (L. Quad) muscle at four weeks post-injection. Representative images of immunoblots showed an increase in TCAP expression in L. Quad post-administration with scAAV9.TCAP.MHCK7.NI3×FLAG (FIG. 33A), scAAV9.TCAP.MHCK7.SI3×FLAG (FIG. 33B), scAAV9.TCAP.CMV.NI3×FLAG (FIG. 33C), and scAAV9.TCAP.CMV.SI3×FLAG (FIG. 33D).

FIG. 33E shows densitometric analysis of the western blots presented in FIGS. 33A-D, which was carried out using Image Lab software (Bio Rad, version 6.0.0 build 25). The intensity of TCAP bands in treated groups was normalized to the GAPDH band intensity of the respective sample. Data provided are the means±SEM (n=3-4 for each group). Samples from C57Bl6 (Bl6) animals revealed that endogenously expressed mouse TCAP (mTCAP) protein is smaller in size than exogenously expressed human TCAP (hTCAP) protein, as expected due to the 3×Flag sequence extension.

FIG. 34A-E shows that systemic delivery (tail vein) of scAAV9.TCAP.3×FLAG vectors at the dose of 3E13 vg/kg results in significant TCAP protein expression in left side of gastrocnemius (L. Gast) muscle at four weeks post-injection. Representative images of immunoblots showed an increase in TCAP expression in L. Gast post-administration with scAAV9.TCAP.MHCK7.NI3×FLAG (FIG. 34A), scAAV9.TCAP.MHCK7.SI3×FLAG (FIG. 34B), scAAV9.TCAP.CMV.NI3×FLAG (FIG. 34C), and scAAV9.TCAP.CMV.SI3×FLAG (FIG. 34D). FIG. 34E shows densitometric analysis of the western blots presented in FIGS. 34A-D, which was carried out using Image Lab software (Bio Rad, version 6.0.0 build 25). The intensity of TCAP bands in treated groups was normalized to the GAPDH band intensity of the respective sample. Data provided are the means±SEM (n=3-4 for each group). Samples from C57Bl6 (Bl6) animals revealed that endogenously expressed mouse TCAP (mTCAP) protein is smaller in size than exogenously expressed human TCAP (hTCAP) protein, as expected due to the 3×Flag sequence extension.

FIG. 35A-B shows restoration of TCAP protein in heart and triceps. FIG. 35A shows representative IF images showing restoration of TCAP protein in the heart from hTCAPKI-null males systemically treated with diluent or the following vectors: scAAV9.TCAP.MHCK7.NI3×FLAG, scAAV9.TCAP.MHCK7.SI3×FLAG, scAAV9.TCAP.CMV.NI3×FLAG, and scAAV9.TCAP.CMV.SI3×FLAG. Age-matched untreated C57Bl6 (Bl6) and diluent treated hTCAPKI-null mice were used as controls. Animals were sacrificed 4 weeks post vector administration. 7 μm tissue sections were immunolabelled with TCAP and sarcomeric alpha-actinin antibodies. Four ROI mages per heart were captured as Z-stacks at 90× magnification and a final resolution of 27 nm/pixel, and the Z-stacks were reduced to a single 2D image per ROI. Thresholds for positive actinin and TCAP signal were calculated as 50% of the mean whole-image intensity for each channel in the Bl6 control images, and were applied identically and automatically to all images. The proportion of actinin-positive pixels also having TCAP-positive signal, and the mean TCAP signal intensity of all actinin-positive regions, were then measured to determine TCAP positivity and intensity. The same approach was used on Triceps. FIG. 35B shows TCAP quantification analysis in the heart and triceps tissues. The images were taken on a Nikon Ti2E-inverted microscope. Scale bars are as labeled. The dashed line indicates 100% TCAP-positive sarcomere area or TCAP intensity. Data reported as mean±SEM for each group with individual points reflecting individual samples, n=3-4. Statistical significance was analyzed by one-way ANOVA with the Bonferroni's multiple comparison test; *p<0.05; ***p <0.001; ****p<0.0001.

FIG. 36A-B provides images and measurement of sarcomere distance measured in the heart of control C57Bl6 (Bl6) and hTCAPKI-null males systemically treated with either diluent or scAAV9.TCAP.3×FLAG vectors. FIG. 36A shows sarcomere distance in reduced Z-stacks as measured by manually drawing straight lines along the axis perpendicular to sarcomere direction, followed by automated unbiased detection of skeletonized sarcomere Z-discs. The perpendicular axis line was broken at each Z-disc intersection, and the resulting fragment lengths were measured to calculate sarcomere distance. FIG. 36B provides quantification analysis of images represented in FIG. 36A. Data are reported as mean±SEM for each group with individual points reflecting individual samples, n=3-4. Statistical significance was analyzed by one-way ANOVA with the Bonferroni's multiple comparison test; *p<0.05.

### DETAILED DESCRIPTION

The disclosure provides titin-cap protein (TCAP) gene replacement as a feasible therapeutic strategy to treat a mutation(s) in the gene that encodes TCAP and, as a result, treat, ameliorate, delay the progression of, or prevent a telethoninopathy resulting from such mutation(s). The disclosed products, methods and uses provide a feasible approach for robust and long-term expression of the TCAP gene in human striated muscles in the treatment of telethoninopathies. Such telethoninopathies include, but are not limited to, limb-girdle muscular dystrophy, hypertrophic cardiomyopathy, dilated cardiomyopathy and idiopathic cardiomyopathy.

Telethoninopathies are the rarest forms of limb girdle muscular dystrophies (LGMDs) resulting from a mutation in the gene that encodes telethonin, also known as the titin-cap protein (TCAP), or loss-of-function of TCAP. The TCAP protein (also referred to herein as “TCAP” or “telethonin”) is a small structural protein found in cardiac and skeletal muscles. TCAP plays an essential role in regulating sarcomere assembly by serving as an anchor for titin in the titin Z1Z2/TCAP complex to increase the mechanical resistance of the sarcomere⁸⁻¹¹.

The TCAP gene encodes a protein found in striated and cardiac muscle that binds to the titin Z1-Z2 domains and is a substrate of titin kinase, interactions thought to be critical to sarcomere assembly since sarcomere assembly is regulated by the muscle protein titin. The TCAP gene consists of two exons leading to an inability to use preclinically/clinically approved antisense oligonucleotide (AON) or small nuclear U7snRNA therapies, which are currently in development to restore mRNAs open reading frames in muscular dystrophies, including LGMD2B (dysferlin deficiency)¹⁻³ or DMD/BMD (dystrophin deficiency)⁴⁻⁷. TCAP is expressed in cardiac and skeletal muscle at Z-discs and functions to regulate sarcomere assembly, T-tubule function and apoptosis. TCAP is a protein that in humans is encoded by the TCAP gene^{14,15} (TCAP titin-cap [Homo sapiens (human)] Gene ID: 8557, updated on 8 Jul. 2021; Ensembl: ENSG00000173991 MIM: 604488).

Mutations in the TCAP gene disturb interactions in the titin Z1Z2/TCAP complex leading to destabilization of the complex, which results in an impaired ability to withstand physiological stresses during muscle contractions. The most common telethoninopathy is the autosomal recessive limb-girdle muscular dystrophy R7 (LGMDR7) or autosomal recessive limb-girdle muscular dystrophy type 2G (LGMD2G) (MedGen UID: 400895), which is marked by progressive limb girdle muscular atrophy and weakness, cardiomyopathy, and respiratory dysfunction. In some aspects, a TCAP mutation has been identified to result in an intestinal obstruction or pseudo-obstruction.⁵⁵ To date, no treatment that could halt or slow progression of LGMDR7/2G exists. Thus, patients with the disease are in urgent need of new therapeutic options. The TCAP gene is associated with autosomal recessive limb-girdle muscular dystrophy type 2G (LGMD2G) (MedGen UID: 400895). Mutations in the TCAP gene also are associated with autosomal dominant dilated cardiomyopathy (DCM) (MedGen UID: 2880). Additionally, the TCAP gene has preliminary evidence supporting a correlation with hypertrophic cardiomyopathy (HCM) (MedGen UID: 183649).

The disclosure focuses on providing a TCAP replacement gene or “transgene” in order to express normal or functionally active TCAP protein. To accomplish this, specifically designed TCAP replacement genes or “transgenes” are provided.

In some aspects, the nucleic acid of the TCAP replacement gene comprises the nucleotide sequence set forth in any one of SEQ ID NOs: 1-4. In various aspects, the nucleic acid is an isoform or variant of the nucleotide sequence nucleotide sequence set forth in any one of SEQ ID NOs: 1-4. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 1-4.

In some aspects, the polypeptide is a TCAP polypeptide comprising the amino acid sequence set forth in SEQ ID NO: 5. In various aspects, the polypeptide is an isoform or variant of the TCAP polypeptide comprising the amino acid sequence set forth in SEQ ID NO: 5. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence set forth in SEQ ID NO: 5.

In some aspects, the transgene polynucleotide sequence is operatively linked to transcriptional control elements (including, but not limited to, promoters, enhancers and/or polyadenylation signal sequences) that are functional in target cells. In some aspects, the promoter is any of an H1 promoter, an EF1-alpha promoter, a minimal EF1-alpha promoter, an unc45b promoter, a CK1 promoter, a CK6 promoter, a CK7 promoter, a CK8e promoter, a cardiac troponin C (cTnC) promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter, the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter, or a native TCAP promoter (including, but not limited to, a native human TCAP promoter).

Inducible promoters are also included. Non-limiting examples of inducible promoters include, but are not limited to a metallothionine promoter, a glucocorticoid promoter, a progesterone promoter, and a tetracycline-regulated promoter. The gene cassette comprising the transgene, in some aspects, also includes intron sequences to facilitate processing of a transgene RNA transcript when expressed in mammalian cells. One example of such an intron is the SV40 intron.

In some aspects, the promoter is a CMV promoter, an MHCK7 promoter, or a native TCAP promoter. The CMV promoter consists of two elements, the CMV enhancer and CMV promoter itself, and is selected as a stable, constitutive, and ubiquitous promoter for transgene expression. The MHCK7 promoter is highly specific for expression in skeletal muscles, including the diaphragm and heart tissues, and includes the following elements—αMHC enhancer, del63 MCK enhancer and MCK promoter—to minimize off-target effects in other tissues. The murine native TCAP promoter is found to be involved in circadian TCAP regulation in skeletal muscles, where its human analog may play a similar role.

In some aspects, the CMV promoter comprises the nucleotide sequence set forth in SEQ ID NO: 6. In some aspects, the MHCK7 promoter comprises the nucleotide sequence set forth in SEQ ID NO: 7. In some aspects, the native TCAP promoter comprises the nucleotide sequence set forth in SEQ ID NO: 8. Thus, in some exemplary aspects, the nucleic acid comprises a promoter comprising the nucleotide sequence set forth in any one of SEQ ID NOs: 6-8. In various aspects, the nucleic acid is an isoform or variant of a nucleic acid comprising the nucleotide sequence set forth in in any one of SEQ ID NOs: 6-8. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 6-8.

TABLE 1
Sequences of the human TCAP gene, TCAP protein, and promoters.

| Type of | | SEQ ID |
|---|---|---|
| Sequence | Sequence (5′-3′) | NO: |
| hTCAP gene-01 | ATGGCTACCTCAGAGCTGAGCTGCGAGGTGTCGGAGGAGAACTGT | 1 |
| | GAGCGCCGGGAGGCCTTCTGGGCAGAATGGAAGGATCTGACACTG | |
| | TCCACACGGCCCGAGGAGGGCTGCTCCCTGCATGAGGAGGACACC | |
| | CAGAGACATGAGACCTACCACCAGCAGGGGCAGTGCCAGGTGCTG | |
| | GTGCAGCGCTCGCCCTGGCTGATGATGCGGATGGGCATCCTCGGC | |
| | CGTGGGCTGCAGGAGTACCAGCTGCCCTACCAGCGGGTACTGCCG | |
| | CTGCCCATCTTCACCCCTGCCAAGATGGGCGCCACCAAGGAGGAG | |
| | CGTGAGGACACCCCCATCCAGCTTCAGGAGCTGCTGGCGCTGGAG | |
| | ACAGCCCTGGGTGGCCAGTGTGTGGACCGCCAGGAGGTGGCTGAG | |
| | ATCACAAAGCAGCTGCCCCCTGTGGTGCCTGTCAGCAAGCCCGGT | |
| | GCACTTCGTCGCTCCCTGTCCCGCTCCATGTCCCAGGAAGCACAG | |
| | AGAGGCTGA | |
| hTCAP gene-02 | ATGGCGACCTCCGAACTGAGCTGCGAAGTGTCTGAAGAAAACTGC | 2 |
| (codon | GAACGCCGTGAAGCGTTTTGGGCGGAATGGAAGGATCTGACCCTG | |
| optimized) | AGCACGCGCCCGGAAGAGGGTTGCTCTCTGCACGAAGAGGATACC | |
| | CAGCGTCATGAAACCTATCACCAGCAGGGTCAGTGCCAGGTTCTG | |
| | GTGCAGCGTTCCCCGTGGCTGATGATGCGTATGGGTATTCTCGGT | |
| | CGCGGGCTGCAGGAATACCAGCTGCCGTATCAGCGTGTTCTCCCG | |
| | CTGCCGATTTTTACCCCGGCGAAAATGGGCGCAACCAAAGAAGAA | |
| | CGCGAAGATACCCCGATCCAGCTGCAGGAACTGCTGGCGCTGGAG | |
| | ACTGCGCTGGGCGGTCAGTGCGTGGACCGTCAGGAGGTGGCGGAG | |
| | ATTACCAAACAGCTGCCGCCGGTGGTGCCGGTGAGCAAGCCGGGC | |
| | GCACTGCGCCGCTCCCTGAGCCGCAGCATGTCCCAGGAGGCGCAG | |
| | CGTGGCTGA | |
| hTCAP gene-03 | ATGGCCACCTCTGAGCTGAGCTGCGAGGTTTCCGAGGAGAACTGT | 3 |
| (codon | GAGCGTCGCGAGGCCTTCTGGGCCGAATGGAAAGATCTGACGCTG | |
| optimized) | TCCACCCGTCCCGAGGAGGGCTGCTCCCTGCATGAGGAAGACACC | |
| | CAGCGCCATGAGACCTACCACCAGCAGGGCCAGTGCCAGGTCCTG | |
| | GTGCAGCGCTCTCCCTGGCTGATGATGCGCATGGGCATCCTCGGC | |
| | CGTGGCCTGCAGGAGTATCAGCTGCCCTACCAGCGCGTGCTCCCG | |
| | CTGCCCATCTTCACCCCTGCCAAGATGGGTGCCACCAAGGAAGAA | |
| | CGTGAGGACACCCCCATCCAGCTGCAGGAGCTGCTGGCGCTGGAA | |
| | ACCGCCCTGGGTGGCCAGTGTGTGGACCGCCAGGAAGTTGCCGAA | |
| | ATCACTAAGCAGCTGCCCCCTGTTGTTCCTGTTAGCAAGCCCGGT | |
| | GCACTTCGTCGTAGCCTGTCCCGTTCCATGAGCCAGGAAGCACAG | |
| | CGCGGCTGA | |
| hTCAP gene-04 | ATGGCTACCTCTGAACTGAGCTGCGAAGTGTCTGAGGAAAACTGT | 4 |
| (codon | GAACGCCGGGAAGCGTTCTGGGCCGAATGGAAAGATCTGACCCTG | |
| optimized) | TCCACTCGTCCAGAAGAAGGCTGCTCCCTGCATGAAGAAGATACC | |
| | CAGAGACATGAAACCTATCACCAGCAGGGTCAGTGCCAGGTTCTG | |
| | GTTCAGCGTTCGCCCTGGCTGATGATGCGTATGGGCATTCTGGGC | |
| | CGTGGTCTGCAGGAATACCAGCTGCCCTACCAGCGTGTGCTGCCG | |
| | CTGCCCATTTTCACCCCGGCGAAAATGGGTGCGACCAAAGAAGAG | |
| | CGTGAAGATACCCCGATTCAGCTGCAGGAACTGCTGGCGCTGGAA | |
| | ACCGCCCTGGGTGGTCAGTGCGTGGACCGCCAGGAGGTGGCGGAA | |
| | ATTACAAAACAGCTGCCCCCTGTGGTTCCGGTTAGCAAACCGGGC | |
| | GCACTGCGTCGTAGCCTGTCCCGTAGCATGTCCCAGGAAGCGCAG | |
| | AGAGGTTGA | |
| hTCAP protein | MATSELSCEVSEENCERREAFWAEWKDLTLSTRPEEGCSLHEEDT | 5 |
| | QRHETYHQQGQCQVLVQRSPWLMMRMGILGRGLQEYQLPYQRVLP | |
| | LPIFTPAKMGATKEEREDTPIQLQELLALETALGGQCVDRQEVAE | |
| | ITKQLPPVVPVSKPGALRRSLSRSMSQEAQRG | |
| CMV promoter | CGTTACATAACTTACGGTAAATGGCCCGCCTGGCTGACCGCCCAA | 6 |
| | CGACCCCCGCCCATTGACGTCAATAATGACGTATGTTCCCATAGT | |
| | AACGCCAATAGGGACTTTCCATTGACGTCAATGGGTGGAGTATTT | |
| | ACGGTAAACTGCCCACTTGGCAGTACATCAAGTGTATCATATGCC | |
| | AAGTACGCCCCCTATTGACGTCAATGACGGTAAATGGCCCGCCTG | |
| | GCATTATGCCCAGTACATGACCTTATGGGACTTTCCTACTTGGCA | |
| | GTACATCTACGTATTAGTCATCGCTATTACCATGGTGATGCGGTT | |
| | TTGGCAGTACATCAATGGGCGTGGATAGCGGTTTGACTCACGGGG | |
| | ATTTCCAAGTCTCCACCCCATTGACGTCAATGGGAGTTTGTTTTG | |
| | GCACCAAAATCAACGGGACTTTCCAAAATGTCGTAACAACTCCGC | |
| | CCCATTGACGCAAATGGGCGGTAGGCGTGTACGGTGGGAGGTCTA | |
| | TATAAGCAGAGCT | |
| MHCK7 | GCATGTCTAAGCTAGACCCTTCAGATTAAAAATAACTGAGGTAAG | 7 |
| promoter | GGCCTGGGTAGGGGAGGTGGTGTGAGACGCTCCTGTCTCTCCTCT | |
| | ATCTGCCCATCGGCCCTTTGGGGAGGAGGAATGTGCCCAAGGACT | |
| | AAAAAAAGGCCATGGAGCCAGAGGGGCGAGGGCAACAGACCTTTC | |
| | ATGGGCAAACCTTGGGGCCCTGCTGTCTAGCATGCCCCACTACGG | |
| | GTCTAGGCTGCCCATGTAAGGAGGCAAGGCCTGGGGACACCCGAG | |
| | ATGCCTGGTTATAATTAACCCAGACATGTGGCTGCCCCCCCCCCC | |
| | CCAACACCTGCTGCCTCTAAAAATAACCCTGTCCCTGGTGGATCC | |
| | CCTGCATGCGAAGATCTTCGAACAAGGCTGTGGGGGACTGAGGGC | |
| | AGGCTGTAACAGGCTTGGGGGCCAGGGCTTATACGTGCCTGGGAC | |
| | TCCCAAAGTATTACTGTTCCATGTTCCCGGCGAAGGGCCAGCTGT | |
| | CCCCCGCCAGCTAGACTCAGCACTTAGTTTAGGAACCAGTGAGCA | |
| | AGTCAGCCCTTGGGGCAGCCCATACAAGGCCATGGGGCTGGGCAA | |
| | GCTGCACGCCTGGGTCCGGGGTGGGCACGGTGCCCGGGCAACGAG | |
| | CTGAAAGCTCATCTGCTCTCAGGGGCCCCTCCCTGGGGACAGCCC | |
| | CTCCTGGCTAGTCACACCCTGTAGGCTCCTCTATATAACCCAGGG | |
| | GCACAGGGGCTGCCCTCATTCTACCACCACCTCCACAGCACAGAC | |
| | AGACACTCAGGAGCAGCCAGCGG | |
| Native TCAP | CCCCATGAAGAAGAGCCAGTCCCCTCCCAGGTAGCACATCAGAGC | 8 |
| promoter | GGGCAGGCTCCAGAGCTCTTCTGGTCCTCTTGCAGTGGCCAGAAA | |
| | GTGGGGAAAGGGGCCCAGAGAGGGGCAGGGACTGACCCATGGTCA | |
| | CACAGCCTATTTATGGCTGATCTGGAGCCAGGCCTTCTGACTATC | |
| | ACCCATCAGAGCCCCTGGATTCCCATTTCACCCAACACAGGTGTT | |
| | GGGGTAGAACTGGGGGCCCTCCACCCCTACTAGCCTGACTGTCTC | |
| | CTGAGACCTGTCTCCTAAGACCACTTACCTGACCACTGGCCATGG | |
| | GGAAGGAAAACACCTGGCCAAGGCTCATGCCCCAGGTTCCCAGGC | |
| | ATGAAGTTGACTGTCTCCGCCCCTGCTGGTGTGGTGAGGGTGACT | |
| | GGGGACTAGGCACTAGGCCTTTGGTGCAGGCGCCTGAGGACGTGG | |
| | TTGCACTCTCCCTTCTGGGGATATGCCCTTGAGCCCAGGCAGAGG | |
| | AGAGCACAGCCAGGGCAGGACCTGGCAGCCCTGGTACAGAGCCCA | |
| | GAGGGGGCATCAGTTCCTGCTGGTCCTGCTCTGTTTACAGACAAG | |
| | CTGCTGTCCTCCCTGCAAAGGGGAGTGGGTGGGGCAGAGGGCAAG | |
| | TGCCAGGGGGGCACAAGGCTGGGCATGTGGCTGGCATGAGACGGT | |
| | GTCTGAGTAATGTCAGGCACCTGGAGGCATTGACCCCAGGACCTT | |
| | GGACCCCAGACCTCTGACCGGGGGGCAGCCAGCGTCCAGGTACCC | |
| | CAACCCCTGCCCTGGGTCCGGCGTCCCCCCATTAGTGAGTCTTGG | |
| | CTCTACTTATAGCATCTGACACCAGAGGGGCCGAAAATAGCCCCT | |
| | GGAGAAGGGGGAGGAGGGGGCTATTTAAAGGGCCTGGGAGGGGAG | |
| | AGAG | |

In some aspects, a nucleic acid of the disclosure comprises the nucleotide sequence set forth in any one of SEQ ID NOs: 9-18 and 29-38 (Table 2). In some aspects, a nucleic acid of the disclosure comprises the nucleotide sequence set forth in any one of SEQ ID NOs: 9-18 and 29-38. In some aspects, the nucleic acid of the TCAP replacement gene comprises the nucleotide sequence set forth in any one of SEQ ID NOs: 9-18 and 29-38. In various aspects, the nucleic acid is a variant of the nucleotide sequence set forth in any one of SEQ ID NOs: 9-18 and 29-38. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 9-18 and 29-38.

The sequences in Table 2 are the nucleotide sequences of the TCAP transgene sequences without the 5′ and 3′ ITR sequences because the transgene sequences, in various aspects, are used in self-complementary and/or single-stranded AAV viral vectors.

In some aspects, a nucleic acid of the disclosure comprises the nucleotide sequence set forth in any one of SEQ ID NOs: 49-58 (Table 4B). The sequences in Table 4B are the nucleotide sequences of the TCAP transgene sequences with the 5′ and 3′ ITR sequences. In various aspects, the nucleic acid is a variant of the nucleotide sequence set forth in any one of SEQ ID NOs: 49-58. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 49-58.

TABLE 2
Sequences of the TCAP gene including the promoter and
additional elements without the ITRs
(SEQ ID NOs: 9-18 and 29-38).

| | | | SEQ | |
|---|---|---|---|---|
| | | Transgene | ID | |
| No. | Name | size (bp) | NO: | Sequence (5′-3′) |
| 1 | NP.TCAP | 1725 | 9 | CCCCATGAAGAAGAGCCAGTCCCCTCCCAGGTAGCACATCAGAGCGGGCAGGCTCCAGAGCTC |
| | | | | TTCTGGTCCTCTTGCAGTGGCCAGAAAGTGGGGAAAGGGGCCCAGAGAGGGGCAGGGACTGAC |
| | | | | CCATGGTCACACAGCCTATTTATGGCTGATCTGGAGCCAGGCCTTCTGACTATCACCCATCAG |
| | | | | AGCCCCTGGATTCCCATTTCACCCAACACAGGTGTTGGGGTAGAACTGGGGGCCCTCCACCCC |
| | | | | TACTAGCCTGACTGTCTCCTGAGACCTGTCTCCTAAGACCACTTACCTGACCACTGGCCATGG |
| | | | | GGAAGGAAAACACCTGGCCAAGGCTCATGCCCCAGGTTCCCAGGCATGAAGTTGACTGTCTCC |
| | | | | GCCCCTGCTGGTGTGGTGAGGGTGACTGGGGACTAGGCACTAGGCCTTTGGTGCAGGCGCCTG |
| | | | | AGGACGTGGTTGCACTCTCCCTTCTGGGGATATGCCCTTGAGCCCAGGCAGAGGAGAGCACAG |
| | | | | CCAGGGCAGGACCTGGCAGCCCTGGTACAGAGCCCAGAGGGGGCATCAGTTCCTGCTGGTCCT |
| | | | | GCTCTGTTTACAGACAAGCTGCTGTCCTCCCTGCAAAGGGGAGTGGGTGGGGCAGAGGGCAAG |
| | | | | TGCCAGGGGGGCACAAGGCTGGGCATGTGGCTGGCATGAGACGGTGTCTGAGTAATGTCAGGC |
| | | | | ACCTGGAGGCATTGACCCCAGGACCTTGGACCCCAGACCTCTGACCGGGGGGCAGCCAGCGTC |
| | | | | CAGGTACCCCAACCCCTGCCCTGGGTCCGGCGTCCCCCCATTAGTGAGTCTTGGCTCTACTTA |
| | | | | TAGCATCTGACACCAGAGGGGCCGAAAATAGCCCCTGGAGAAGGGGGAGGAGGGGGCTATTTA |
| | | | | AAGGGCCTGGGAGGGGAGAGAGaatgaggagtgatcatggctacctcagagctgagctgcgag |
| | | | | gtgtcggaggagaactgtgagcgccgggaggccttctgggcagaatggaaggatctgacactg |
| | | | | tccacacggcccgaggaggggtgagtgtgggtctgctagagccctgcctctgctcccccagag |
| | | | | caccctcactgagccatgaggccagagcatgaagccctggagaaatttctgggggtgggggca |
| | | | | ggaagaatgccccatggggagagcaaaggggaaccacccttcctgcccccaggtcccagcagc |
| | | | | ccaggggagccccccacccagcctgtgcccagagagcaacagctcccaggagctcactgcccc |
| | | | | tcccctctccccagCTGCTCCCTGCATGAGGAGGACACCCAGAGACATGAGACCTACCACCAG |
| | | | | CAGGGGCAGTGCCAGGTGCTGGTGCAGCGCTCGCCCTGGCTGATGATGCGGATGGGCATCCTC |
| | | | | GGCCGTGGGCTGCAGGAGTACCAGCTGCCCTACCAGCGGGTACTGCCGCTGCCCATCTTCACC |
| | | | | CCTGCCAAGATGGGCGCCACCAAGGAGGAGCGTGAGGACACCCCCATCCAGCTTCAGGAGCTG |
| | | | | CTGGCGCTGGAGACAGCCCTGGGTGGCCAGTGTGTGGACCGCCAGGAGGTGGCTGAGATCACA |
| | | | | AAGCAGCTGCCCCCTGTGGTGCCTGTCAGCAAGCCCGGTGCACTTCGTCGCTCCCTGTCCCGC |
| | | | | TCCATGTCCCAGGAAGCACAGAGAGGCTGAGCGGCCGCaataaaagatctttattttcattag |
| | | | | atctgtgtgttggttttttgtgtg |
| 2 | MHCK7. | 1609 | 10 | GCATGTCTAAGCTAGACCCTTCAGATTAAAAATAACTGAGGTAAGGGCCTGGGTAGGGGAGGT |
| | TCAP.NI | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCAACACCTGCTGCCTCTAAAAATAACCCTGTCCCTGGTGGATCCCCTGCATGCGAAGATCTT |
| | | | | CGAACAAGGCTGTGGGGGACTGAGGGCAGGCTGTAACAGGCTTGGGGGCCAGGGCTTATACGT |
| | | | | GCCTGGGACTCCCAAAGTATTACTGTTCCATGTTCCCGGCGAAGGGCCAGCTGTCCCCCGCCA |
| | | | | GCTAGACTCAGCACTTAGTTTAGGAACCAGTGAGCAAGTCAGCCCTTGGGGCAGCCCATACAA |
| | | | | GGCCATGGGGCTGGGCAAGCTGCACGCCTGGGTCCGGGGTGGGCACGGTGCCCGGGCAACGAG |
| | | | | CTGAAAGCTCATCTGCTCTCAGGGGCCCCTCCCTGGGGACAGCCCCTCCTGGCTAGTCACACC |
| | | | | CTGTAGGCTCCTCTATATAACCCAGGGGCACAGGGGCTGCCCTCATTCTACCACCACCTCCAC |
| | | | | AGCACAGACAGACACTCAGGAGCAGCCAGCGGaatgaggagtgatcatggctacctcagagct |
| | | | | gagctgcgaggtgtcggaggagaactgtgagcgccgggaggccttctgggcagaatggaagga |
| | | | | tctgacactgtccacacggcccgaggaggggtgagtgtgggtctgctagagccctgcctctgc |
| | | | | tcccccagagcaccctcactgagccatgaggccagagcatgaagccctggagaaatttctggg |
| | | | | ggtgggggcaggaagaatgccccatggggagagcaaaggggaaccacccttcctgcccccagg |
| | | | | tcccagcagcccaggggagccccccacccagcctgtgcccagagagcaacagctcccaggagc |
| | | | | tcactgcccctcccctctccccagCTGCTCCCTGCATGAGGAGGACACCCAGAGACATGAGAC |
| | | | | CTACCACCAGCAGGGGCAGTGCCAGGTGCTGGTGCAGCGCTCGCCCTGGCTGATGATGCGGAT |
| | | | | GGGCATCCTCGGCCGTGGGCTGCAGGAGTACCAGCTGCCCTACCAGCGGGTACTGCCGCTGCC |
| | | | | CATCTTCACCCCTGCCAAGATGGGCGCCACCAAGGAGGAGCGTGAGGACACCCCCATCCAGCT |
| | | | | TCAGGAGCTGCTGGCGCTGGAGACAGCCCTGGGTGGCCAGTGTGTGGACCGCCAGGAGGTGGC |
| | | | | TGAGATCACAAAGCAGCTGCCCCCTGTGGTGCCTGTCAGCAAGCCCGGTGCACTTCGTCGCTC |
| | | | | CCTGTCCCGCTCCATGTCCCAGGAAGCACAGAGAGGCtgaGCGGCCGCaataaaagatcttta |
| | | | | ttttcattagatctgtgtgttggttttttgtgtg |
| 3 | MHCK7. | 1515 | 11 | GCATGTCTAAGCTAGACCCTTCAGATTAAAAATAACTGAGGTAAGGGCCTGGGTAGGGGAGGT |
| | TCAP.SI | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCAACACCTGCTGCCTCTAAAAATAACCCTGTCCCTGGTGGATCCCCTGCATGCGAAGATCTT |
| | | | | CGAACAAGGCTGTGGGGGACTGAGGGCAGGCTGTAACAGGCTTGGGGGCCAGGGCTTATACGT |
| | | | | GCCTGGGACTCCCAAAGTATTACTGTTCCATGTTCCCGGCGAAGGGCCAGCTGTCCCCCGCCA |
| | | | | GCTAGACTCAGCACTTAGTTTAGGAACCAGTGAGCAAGTCAGCCCTTGGGGCAGCCCATACAA |
| | | | | GGCCATGGGGCTGGGCAAGCTGCACGCCTGGGTCCGGGGTGGGCACGGTGCCCGGGCAACGAG |
| | | | | CTGAAAGCTCATCTGCTCTCAGGGGCCCCTCCCTGGGGACAGCCCCTCCTGGCTAGTCACACC |
| | | | | CTGTAGGCTCCTCTATATAACCCAGGGGCACAGGGGCTGCCCTCATTCTACCACCACCTCCAC |
| | | | | AGCACAGACAGACACTCAGGAGCAGCCAGCGGccaggtaagtttagtctttttgtcttttatt |
| | | | | tcaggtcccggatccggtggtggtgcaaatcaaagaactgctcctcagtggatgttgccttta |
| | | | | cttctaggcctgtacggaagtgttacttctgctctaaaagctgcggaattgtacccgcaatga |
| | | | | ggagtgatcatggctacctcagagctgagctgcgaggtgtcggaggagaactgtgagcgccgg |
| | | | | gaggccttctgggcagaatggaaggatctgacactgtccacacggcccgaggagggCTGCTCC |
| | | | | CTGCATGAGGAGGACACCCAGAGACATGAGACCTACCACCAGCAGGGGCAGTGCCAGGTGCTG |
| | | | | GTGCAGCGCTCGCCCTGGCTGATGATGCGGATGGGCATCCTCGGCCGTGGGCTGCAGGAGTAC |
| | | | | CAGCTGCCCTACCAGCGGGTACTGCCGCTGCCCATCTTCACCCCTGCCAAGATGGGCGCCACC |
| | | | | AAGGAGGAGCGTGAGGACACCCCCATCCAGCTTCAGGAGCTGCTGGCGCTGGAGACAGCCCTG |
| | | | | GGTGGCCAGTGTGTGGACCGCCAGGAGGTGGCTGAGATCACAAAGCAGCTGCCCCCTGTGGTG |
| | | | | CCTGTCAGCAAGCCCGGTGCACTTCGTCGCTCCCTGTCCCGCTCCATGTCCCAGGAAGCACAG |
| | | | | AGAGGCtgaGCGGCCGCaataaaagatctttattttcattagatctgtgtgttggttttttgt |
| | | | | gtg |
| 4 | CMV. | 1447 | 12 | CGTTACATAACTTACGGTAAATGGCCCGCCTGGCTGACCGCCCAACGACCCCCGCCCATTGAC |
| | TCAP.NI | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCaGGACCGATCCAGCCTCCGGACTCTAGAGGATCCGGTACTCGAGGAACCTGaatg |
| | | | | aggagtgatcatggctacctcagagctgagctgcgaggtgtcggaggagaactgtgagcgccg |
| | | | | ggaggccttctgggcagaatggaaggatctgacactgtccacacggcccgaggaggggtgagt |
| | | | | gtgggtctgctagagccctgcctctgctcccccagagcaccctcactgagccatgaggccaga |
| | | | | gcatgaagccctggagaaatttctgggggtgggggcaggaagaatgccccatggggagagcaa |
| | | | | aggggaaccacccttcctgcccccaggtcccagcagcccaggggagccccccacccagcctgt |
| | | | | gcccagagagcaacagctcccaggagctcactgcccctcccctctccccagCTGCTCCCTGCA |
| | | | | TGAGGAGGACACCCAGAGACATGAGACCTACCACCAGCAGGGGCAGTGCCAGGTGCTGGTGCA |
| | | | | GCGCTCGCCCTGGCTGATGATGCGGATGGGCATCCTCGGCCGTGGGCTGCAGGAGTACCAGCT |
| | | | | GCCCTACCAGCGGGTACTGCCGCTGCCCATCTTCACCCCTGCCAAGATGGGCGCCACCAAGGA |
| | | | | GGAGCGTGAGGACACCCCCATCCAGCTTCAGGAGCTGCTGGCGCTGGAGACAGCCCTGGGTGG |
| | | | | CCAGTGTGTGGACCGCCAGGAGGTGGCTGAGATCACAAAGCAGCTGCCCCCTGTGGTGCCTGT |
| | | | | CAGCAAGCCCGGTGCACTTCGTCGCTCCCTGTCCCGCTCCATGTCCCAGGAAGCACAGAGAGG |
| | | | | CtgaGCGGCCGCaataaaagatctttattttcattagatctgtgtgttggttttttgtgtg |
| 5 | CMV.TCAP. | 1348 | 13 | CGTTACATAACTTACGGTAAATGGCCCGCCTGGCTGACCGCCCAACGACCCCCGCCCATTGAC |
| | SI | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACaggtaag |
| | | | | tttagtctttttgtcttttatttcaggtcccggatccggtggtggtgcaaatcaaagaactgc |
| | | | | tcctcagtggatgttgcctttacttctaggcctgtacggaagtgttacttctgctctaaaagc |
| | | | | tgcggaattgtacccgcaatgaggagtgatcatggctacctcagagctgagctgcgaggtgtc |
| | | | | ggaggagaactgtgagcgccgggaggccttctgggcagaatggaaggatctgacactgtccac |
| | | | | acggcccgaggagggCTGCTCCCTGCATGAGGAGGACACCCAGAGACATGAGACCTACCACCA |
| | | | | GCAGGGGCAGTGCCAGGTGCTGGTGCAGCGCTCGCCCTGGCTGATGATGCGGATGGGCATCCT |
| | | | | CGGCCGTGGGCTGCAGGAGTACCAGCTGCCCTACCAGCGGGTACTGCCGCTGCCCATCTTCAC |
| | | | | CCCTGCCAAGATGGGCGCCACCAAGGAGGAGCGTGAGGACACCCCCATCCAGCTTCAGGAGCT |
| | | | | GCTGGCGCTGGAGACAGCCCTGGGTGGCCAGTGTGTGGACCGCCAGGAGGTGGCTGAGATCAC |
| | | | | AAAGCAGCTGCCCCCTGTGGTGCCTGTCAGCAAGCCCGGTGCACTTCGTCGCTCCCTGTCCCG |
| | | | | CTCCATGTCCCAGGAAGCACAGAGAGGCtgaGCGGCCGCaataaaagatctttattttcatta |
| | | | | gatctgtgtgttggttttttgtgtg |
| 6 | NP.TCAP. | 1791 | 14 | CCCCATGAAGAAGAGCCAGTCCCCTCCCAGGTAGCACATCAGAGCGGGCAGGCTCCAGAGCTC |
| | 3xFLAG | | | TTCTGGTCCTCTTGCAGTGGCCAGAAAGTGGGGAAAGGGGCCCAGAGAGGGGCAGGGACTGAC |
| | | | | CCATGGTCACACAGCCTATTTATGGCTGATCTGGAGCCAGGCCTTCTGACTATCACCCATCAG |
| | | | | AGCCCCTGGATTCCCATTTCACCCAACACAGGTGTTGGGGTAGAACTGGGGGCCCTCCACCCC |
| | | | | TACTAGCCTGACTGTCTCCTGAGACCTGTCTCCTAAGACCACTTACCTGACCACTGGCCATGG |
| | | | | GGAAGGAAAACACCTGGCCAAGGCTCATGCCCCAGGTTCCCAGGCATGAAGTTGACTGTCTCC |
| | | | | GCCCCTGCTGGTGTGGTGAGGGTGACTGGGGACTAGGCACTAGGCCTTTGGTGCAGGCGCCTG |
| | | | | AGGACGTGGTTGCACTCTCCCTTCTGGGGATATGCCCTTGAGCCCAGGCAGAGGAGAGCACAG |
| | | | | CCAGGGCAGGACCTGGCAGCCCTGGTACAGAGCCCAGAGGGGGCATCAGTTCCTGCTGGTCCT |
| | | | | GCTCTGTTTACAGACAAGCTGCTGTCCTCCCTGCAAAGGGGAGTGGGTGGGGCAGAGGGCAAG |
| | | | | TGCCAGGGGGGCACAAGGCTGGGCATGTGGCTGGCATGAGACGGTGTCTGAGTAATGTCAGGC |
| | | | | ACCTGGAGGCATTGACCCCAGGACCTTGGACCCCAGACCTCTGACCGGGGGGCAGCCAGCGTC |
| | | | | CAGGTACCCCAACCCCTGCCCTGGGTCCGGCGTCCCCCCATTAGTGAGTCTTGGCTCTACTTA |
| | | | | TAGCATCTGACACCAGAGGGGCCGAAAATAGCCCCTGGAGAAGGGGGAGGAGGGGGCTATTTA |
| | | | | AAGGGCCTGGGAGGGGAGAGAGaatgaggagtgatcatggctacctcagagctgagctgcgag |
| | | | | gtgtcggaggagaactgtgagcgccgggaggccttctgggcagaatggaaggatctgacactg |
| | | | | tccacacggcccgaggaggggtgagtgtgggtctgctagagccctgcctctgctcccccagag |
| | | | | caccctcactgagccatgaggccagagcatgaagccctggagaaatttctgggggtgggggca |
| | | | | ggaagaatgccccatggggagagcaaaggggaaccacccttcctgcccccaggtcccagcagc |
| | | | | ccaggggagcccccacccagcctgtgcccagagagcaacagctcccaggagctcactgcccc |
| | | | | tcccctctccccagCTGCTCCCTGCATGAGGAGGACACCCAGAGACATGAGACCTACCACCAG |
| | | | | CAGGGGCAGTGCCAGGTGCTGGTGCAGCGCTCGCCCTGGCTGATGATGCGGATGGGCATCCTC |
| | | | | GGCCGTGGGCTGCAGGAGTACCAGCTGCCCTACCAGCGGGTACTGCCGCTGCCCATCTTCACC |
| | | | | CCTGCCAAGATGGGCGCCACCAAGGAGGAGCGTGAGGACACCCCCATCCAGCTTCAGGAGCTG |
| | | | | CTGGCGCTGGAGACAGCCCTGGGTGGCCAGTGTGTGGACCGCCAGGAGGTGGCTGAGATCACA |
| | | | | AAGCAGCTGCCCCCTGTGGTGCCTGTCAGCAAGCCCGGTGCACTTCGTCGCTCCCTGTCCCGC |
| | | | | TCCATGTCCCAGGAAGCACAGAGAGGCGACTACAAAGACCATGACGGTGATTATAAAGATCAT |
| | | | | GACATCGATTACAAGGATGACGATGACAAGtgaGCGGCCGCaataaaagatctttattttcat |
| | | | | tagatctgtgtgttggttttttgtgtg |
| 7 | MHCK7.TCAP. | 1678^{α} | 15 | GCATGTCTAAGCTAGACCCTTCAGATTAAAAATAACTGAGGTAAGGGCCTGGGTAGGGGAGGT |
| | NI3xFLAG | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCCCCAACACCTGCTGCCTCTAAAAATAACCCTGTCCCTGGTGGATCCCCTGCATGCGAAGAT |
| | | | | CTTCGAACAAGGCTGTGGGGGACTGAGGGCAGGCTGTAACAGGCTTGGGGGCCAGGGCTTATA |
| | | | | CGTGCCTGGGACTCCCAAAGTATTACTGTTCCATGTTCCCGGCGAAGGGCCAGCTGTCCCCCG |
| | | | | CCAGCTAGACTCAGCACTTAGTTTAGGAACCAGTGAGCAAGTCAGCCCTTGGGGCAGCCCATA |
| | | | | CAAGGCCATGGGGCTGGGCAAGCTGCACGCCTGGGTCCGGGGTGGGCACGGTGCCCGGGCAAC |
| | | | | GAGCTGAAAGCTCATCTGCTCTCAGGGGCCCCTCCCTGGGGACAGCCCCTCCTGGCTAGTCAC |
| | | | | ACCCTGTAGGCTCCTCTATATAACCCAGGGGCACAGGGGCTGCCCTCATTCTACCACCACCTC |
| | | | | CACAGCACAGACAGACACTCAGGAGCAGCCAGCGGaatgaggagtgatcatggctacctcaga |
| | | | | gctgagctgcgaggtgtcggaggagaactgtgagcgccgggaggccttctgggcagaatggaa |
| | | | | ggatctgacactgtccacacggcccgaggaggggtgagtgtgggtctgctagagccctgcctc |
| | | | | tgctcccccagagcaccctcactgagccatgaggccagagcatgaagccctggagaaatttct |
| | | | | gggggtgggggcaggaagaatgccccatggggagagcaaaggggaaccacccttcctgccccc |
| | | | | aggtcccagcagcccaggggagccccccacccagcctgtgcccagagagcaacagctcccagg |
| | | | | agctcactgcccctcccctctccccagCTGCTCCCTGCATGAGGAGGACACCCAGAGACATGA |
| | | | | GACCTACCACCAGCAGGGGCAGTGCCAGGTGCTGGTGCAGCGCTCGCCCTGGCTGATGATGCG |
| | | | | GATGGGCATCCTCGGCCGTGGGCTGCAGGAGTACCAGCTGCCCTACCAGCGGGTACTGCCGCT |
| | | | | GCCCATCTTCACCCCTGCCAAGATGGGCGCCACCAAGGAGGAGCGTGAGGACACCCCCATCCA |
| | | | | GCTTCAGGAGCTGCTGGCGCTGGAGACAGCCCTGGGTGGCCAGTGTGTGGACCGCCAGGAGGT |
| | | | | GGCTGAGATCACAAAGCAGCTGCCCCCTGTGGTGCCTGTCAGCAAGCCCGGTGCACTTCGTCG |
| | | | | CTCCCTGTCCCGCTCCATGTCCCAGGAAGCACAGAGAGGCGACTACAAAGACCATGACGGTGA |
| | | | | TTATAAAGATCATGACATCGATTACAAGGATGACGATGACAAGtgaGCGGCCGCaataaaaga |
| | | | | tctttattttcattagatctgtgtgttggttttttgtgtg |
| 8 | MHCK7.TCAP. | 1579^{β} | 16 | GCATGTCTAAGCTAGACCCTTCAGATTAAAAATAACTGAGGTAAGGGCCTGGGTAGGGGAGGT |
| | SI3xFLAG | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | AACACCTGCTGCCTCTAAAAATAACCCTGTCCCTGGTGGATCCCCTGCATGCGAAGATCTTCG |
| | | | | AACAAGGCTGTGGGGGACTGAGGGCAGGCTGTAACAGGCTTGGGGGCCAGGGCTTATACGTGC |
| | | | | CTGGGACTCCCAAAGTATTACTGTTCCATGTTCCCGGCGAAGGGCCAGCTGTCCCCCGCCAGC |
| | | | | TAGACTCAGCACTTAGTTTAGGAACCAGTGAGCAAGTCAGCCCTTGGGGCAGCCCATACAAGG |
| | | | | CCATGGGGCTGGGCAAGCTGCACGCCTGGGTCCGGGGTGGGCACGGTGCCCGGGCAACGAGCT |
| | | | | GAAAGCTCATCTGCTCTCAGGGGCCCCTCCCTGGGGACAGCCCCTCCTGGCTAGTCACACCCT |
| | | | | GTAGGCTCCTCTATATAACCCAGGGGCACAGGGGCTGCCCTCATTCTACCACCACCTCCACAG |
| | | | | CACAGACAGACACTCAGGAGCAGCCAGCGGccaggtaagtttagtctttttgtcttttatttc |
| | | | | aggtcccggatccggtggtggtgcaaatcaaagaactgctcctcagtggatgttgcctttact |
| | | | | tctaggcctgtacggaagtgttacttctgctctaaaagctgcggaattgtacccgcaatgagg |
| | | | | agtgatcatggctacctcagagctgagctgcgaggtgtcggaggagaactgtgagcgccggga |
| | | | | ggccttctgggcagaatggaaggatctgacactgtccacacggcccgaggagggCTGCTCCCT |
| | | | | GCATGAGGAGGACACCCAGAGACATGAGACCTACCACCAGCAGGGGCAGTGCCAGGTGCTGGT |
| | | | | GCAGCGCTCGCCCTGGCTGATGATGCGGATGGGCATCCTCGGCCGTGGGCTGCAGGAGTACCA |
| | | | | GCTGCCCTACCAGCGGGTACTGCCGCTGCCCATCTTCACCCCTGCCAAGATGGGCGCCACCAA |
| | | | | GGAGGAGCGTGAGGACACCCCCATCCAGCTTCAGGAGCTGCTGGCGCTGGAGACAGCCCTGGG |
| | | | | TGGCCAGTGTGTGGACCGCCAGGAGGTGGCTGAGATCACAAAGCAGCTGCCCCCTGTGGTGCC |
| | | | | TGTCAGCAAGCCCGGTGCACTTCGTCGCTCCCTGTCCCGCTCCATGTCCCAGGAAGCACAGAG |
| | | | | AGGCGACTACAAAGACCATGACGGTGATTATAAAGATCATGACATCGATTACAAGGATGACGA |
| | | | | TGACAAGtgaGCGGCCGCaataaaagatctttattttcattagatctgtgtgttggttttttg |
| | | | | tgtg |
| 9 | CMV.TCAP. | 1513 | 17 | CGTTACATAACTTACGGTAAATGGCCCGCCTGGCTGACCGCCCAACGACCCCCGCCCATTGAC |
| | NI3xFLAG | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACCTGaatg |
| | | | | aggagtgatcatggctacctcagagctgagctgcgaggtgtcggaggagaactgtgagcgccg |
| | | | | ggaggccttctgggcagaatggaaggatctgacactgtccacacggcccgaggaggggtgagt |
| | | | | gtgggtctgctagagccctgcctctgctcccccagagcaccctcactgagccatgaggccaga |
| | | | | gcatgaagccctggagaaatttctgggggtgggggcaggaagaatgccccatggggagagcaa |
| | | | | aggggaaccacccttcctgcccccaggtcccagcagcccaggggagccccccacccagcctgt |
| | | | | gcccagagagcaacagctcccaggagctcactgcccctcccctctccccagCTGCTCCCTGCA |
| | | | | TGAGGAGGACACCCAGAGACATGAGACCTACCACCAGCAGGGGCAGTGCCAGGTGCTGGTGCA |
| | | | | GCGCTCGCCCTGGCTGATGATGCGGATGGGCATCCTCGGCCGTGGGCTGCAGGAGTACCAGCT |
| | | | | GCCCTACCAGCGGGTACTGCCGCTGCCCATCTTCACCCCTGCCAAGATGGGCGCCACCAAGGA |
| | | | | GGAGCGTGAGGACACCCCCATCCAGCTTCAGGAGCTGCTGGCGCTGGAGACAGCCCTGGGTGG |
| | | | | CCAGTGTGTGGACCGCCAGGAGGTGGCTGAGATCACAAAGCAGCTGCCCCCTGTGGTGCCTGT |
| | | | | CAGCAAGCCCGGTGCACTTCGTCGCTCCCTGTCCCGCTCCATGTCCCAGGAAGCACAGAGAGG |
| | | | | CGACTACAAAGACCATGACGGTGATTATAAAGATCATGACATCGATTACAAGGATGACGATGA |
| | | | | CAAGtgaGCGGCCGCaataaaagatctttattttcattagatctgtgtgttggttttttgtgt |
| | | | | g |
| 10 | CMV.TCAP. | 1414 | 18 | CGTTACATAACTTACGGTAAATGGCCCGCCTGGCTGACCGCCCAACGACCCCCGCCCATTGAC |
| | SI3xFLAG | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACaggtaag |
| | | | | tttagtctttttgtcttttatttcaggtcccggatccggtggtggtgcaaatcaaagaactgc |
| | | | | tcctcagtggatgttgcctttacttctaggcctgtacggaagtgttacttctgctctaaaagc |
| | | | | tgcggaattgtacccgcaatgaggagtgatcatggctacctcagagctgagctgcgaggtgtc |
| | | | | ggaggagaactgtgagcgccgggaggccttctgggcagaatggaaggatctgacactgtccac |
| | | | | acggcccgaggagggCTGCTCCCTGCATGAGGAGGACACCCAGAGACATGAGACCTACCACCA |
| | | | | GCAGGGGCAGTGCCAGGTGCTGGTGCAGCGCTCGCCCTGGCTGATGATGCGGATGGGCATCCT |
| | | | | CGGCCGTGGGCTGCAGGAGTACCAGCTGCCCTACCAGCGGGTACTGCCGCTGCCCATCTTCAC |
| | | | | CCCTGCCAAGATGGGCGCCACCAAGGAGGAGCGTGAGGACACCCCCATCCAGCTTCAGGAGCT |
| | | | | GCTGGCGCTGGAGACAGCCCTGGGTGGCCAGTGTGTGGACCGCCAGGAGGTGGCTGAGATCAC |
| | | | | AAAGCAGCTGCCCCCTGTGGTGCCTGTCAGCAAGCCCGGTGCACTTCGTCGCTCCCTGTCCCG |
| | | | | CTCCATGTCCCAGGAAGCACAGAGAGGCGACTACAAAGACCATGACGGTGATTATAAAGATCA |
| | | | | TGACATCGATTACAAGGATGACGATGACAAGtgaGCGGCCGCaataaaagatctttattttca |
| | | | | ttagatctgtgtgttggttttttgtgtg |
| 11 | NP.TCAP/SD | 1725 | 29 | CCCCATGAAGAAGAGCCAGTCCCCTCCCAGGTAGCACATCAGAGCGGGCAGGCTCCAGAGCTC |
| | | | | TTCTGGTCCTCTTGCAGTGGCCAGAAAGTGGGGAAAGGGGCCCAGAGAGGGGCAGGGACTGAC |
| | | | | CCATGGTCACACAGCCTATTTATGGCTGATCTGGAGCCAGGCCTTCTGACTATCACCCATCAG |
| | | | | AGCCCCTGGATTCCCATTTCACCCAACACAGGTGTTGGGGTAGAACTGGGGGCCCTCCACCCC |
| | | | | TACTAGCCTGACTGTCTCCTGAGACCTGTCTCCTAAGACCACTTACCTGACCACTGGCCATGG |
| | | | | GGAAGGAAAACACCTGGCCAAGGCTCATGCCCCAGGTTCCCAGGCATGAAGTTGACTGTCTCC |
| | | | | GCCCCTGCTGGTGTGGTGAGGGTGACTGGGGACTAGGCACTAGGCCTTTGGTGCAGGCGCCTG |
| | | | | AGGACGTGGTTGCACTCTCCCTTCTGGGGATATGCCCTTGAGCCCAGGCAGAGGAGAGCACAG |
| | | | | CCAGGGCAGGACCTGGCAGCCCTGGTACAGAGCCCAGAGGGGGCATCAGTTCCTGCTGGTCCT |
| | | | | GCTCTGTTTACAGACAAGCTGCTGTCCTCCCTGCAAAGGGGAGTGGGTGGGGCAGAGGGCAAG |
| | | | | TGCCAGGGGGGCACAAGGCTGGGCATGTGGCTGGCATGAGACGGTGTCTGAGTAATGTCAGGC |
| | | | | ACCTGGAGGCATTGACCCCAGGACCTTGGACCCCAGACCTCTGACCGGGGGGCAGCCAGCGTC |
| | | | | CAGGTACCCCAACCCCTGCCCTGGGTCCGGCGTCCCCCCATTAGTGAGTCTTGGCTCTACTTA |
| | | | | TAGCATCTGACACCAGAGGGGCCGAAAATAGCCCCTGGAGAAGGGGGAGGAGGGGGCTATTTA |
| | | | | AAGGGCCTGGGAGGGGAGAGAGaatgaggagtgatcatggctacctcagagctgagctgcgag |
| | | | | gtgtcggaggagaactgtgagcgccgggaggccttctgggcagaatggaaggatctgacactg |
| | | | | tccacacggcccgaggaggggtgagtgtgggtctgctagagccctgcctctgctcccccagag |
| | | | | caccctcactgagccatgaggccagagcatgaagccctggagaaatttctgggggtgggggca |
| | | | | ggaagaatgccccatggggagagcaaaggggaaccacccttcctgcccccaggtcccagcagc |
| | | | | ccaggggagccccccacccagcctgtgcccagagagcaacagctcccaggagctcactgcccc |
| | | | | tcccctctccccagCTGCTCCCTGCATGAGGAGGACACCCAGAGACATGAGACCTACCACCAG |
| | | | | CAGGGGCAGTGCCAGGTGCTGGTGCAGCGCTCGCCCTGGCTGATGATGCGGATGGGCATCCTC |
| | | | | GGCCGTGGGCTGCAGGAGTACCAGCTGCCCTACCAGCGGGTACTGCCGCTGCCCATCTTCACC |
| | | | | CCTGCCAAGATGGGCGCCACCAAGGAGGAGCGTGAGGACACCCCCATCCAGCTTCAGGAGCTG |
| | | | | CTGGCGCTGGAGACAGCCCTGGGTGGCCAGTGTGTGGACCGCCAGGAGGTGGCTGAGATCACA |
| | | | | AAGCAGCTGCCCCCTGTGGTGCCTGTCAGCAAGCCCGGTGCACTTCGTCGCTCCCTGTCCCGC |
| | | | | TCCATGTCCCAGGAAGCACAGAGAGGCtgaGCGGCCGCaataaaagatctttattttcattag |
| | | | | atctgtgtgttggttttttgtgtg |
| 12 | MHCK7.TCAP. | 1609 | 30 | GCATGTCTAAGCTAGACCCTTCAGATTAAAAATAACTGAGGTAAGGGCCTGGGTAGGGGAGGT |
| | NI/SD | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCAACACCTGCTGCCTCTAAAAATAACCCTGTCCCTGGTGGATCCCCTGCATGCGAAGATCTT |
| | | | | CGAACAAGGCTGTGGGGGACTGAGGGCAGGCTGTAACAGGCTTGGGGGCCAGGGCTTATACGT |
| | | | | GCCTGGGACTCCCAAAGTATTACTGTTCCATGTTCCCGGCGAAGGGCCAGCTGTCCCCCGCCA |
| | | | | GCTAGACTCAGCACTTAGTTTAGGAACCAGTGAGCAAGTCAGCCCTTGGGGCAGCCCATACAA |
| | | | | GGCCATGGGGCTGGGCAAGCTGCACGCCTGGGTCCGGGGTGGGCACGGTGCCCGGGCAACGAG |
| | | | | CTGAAAGCTCATCTGCTCTCAGGGGCCCCTCCCTGGGGACAGCCCCTCCTGGCTAGTCACACC |
| | | | | CTGTAGGCTCCTCTATATAACCCAGGGGCACAGGGGCTGCCCTCATTCTACCACCACCTCCAC |
| | | | | AGCACAGACAGACACTCAGGAGCAGCCAGCGGaatgaggagtgatcatggctacctcagagct |
| | | | | gagctgcgaggtgtcggaggagaactgtgagcgccgggaggccttctgggcagaatggaagga |
| | | | | tctgacactgtccacacggcccgaggaggggtgagtgtgggtctgctagagccctgcctctgc |
| | | | | tcccccagagcaccctcactgagccatgaggccagagcatgaagccctggagaaatttctggg |
| | | | | ggtgggggcaggaagaatgccccatggggagagcaaaggggaaccacccttcctgcccccagg |
| | | | | tcccagcagcccaggggagccccccacccagcctgtgcccagagagcaacagctcccaggagc |
| | | | | tcactgcccctcccctctccccagCTGCTCCCTGCATGAGGAGGACACCCAGAGACATGAGAC |
| | | | | CTACCACCAGCAGGGGCAGTGCCAGGTGCTGGTGCAGCGCTCGCCCTGGCTGATGATGCGGAT |
| | | | | GGGCATCCTCGGCCGTGGGCTGCAGGAGTACCAGCTGCCCTACCAGCGGGTACTGCCGCTGCC |
| | | | | CATCTTCACCCCTGCCAAGATGGGCGCCACCAAGGAGGAGCGTGAGGACACCCCCATCCAGCT |
| | | | | TCAGGAGCTGCTGGCGCTGGAGACAGCCCTGGGTGGCCAGTGTGTGGACCGCCAGGAGGTGGC |
| | | | | TGAGATCACAAAGCAGCTGCCCCCTGTGGTGCCTGTCAGCAAGCCCGGTGCACTTCGTCGCTC |
| | | | | CCTGTCCCGCTCCATGTCCCAGGAAGCACAGAGAGGCtgaGCGGCCGCaataaaagatcttta |
| | | | | ttttcattagatctgtgtgttggttttttgtgtg |
| 13 | MHCK7.TCAP. | 1515 | 31 | GCATGTCTAAGCTAGACCCTTCAGATTAAAAATAACTGAGGTAAGGGCCTGGGTAGGGGAGGT |
| | SI/SD | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCAACACCTGCTGCCTCTAAAAATAACCCTGTCCCTGGTGGATCCCCTGCATGCGAAGATCTT |
| | | | | CGAACAAGGCTGTGGGGGACTGAGGGCAGGCTGTAACAGGCTTGGGGGCCAGGGCTTATACGT |
| | | | | GCCTGGGACTCCCAAAGTATTACTGTTCCATGTTCCCGGCGAAGGGCCAGCTGTCCCCCGCCA |
| | | | | GCTAGACTCAGCACTTAGTTTAGGAACCAGTGAGCAAGTCAGCCCTTGGGGCAGCCCATACAA |
| | | | | GGCCATGGGGCTGGGCAAGCTGCACGCCTGGGTCCGGGGTGGGCACGGTGCCCGGGCAACGAG |
| | | | | CTGAAAGCTCATCTGCTCTCAGGGGCCCCTCCCTGGGGACAGCCCCTCCTGGCTAGTCACACC |
| | | | | CTGTAGGCTCCTCTATATAACCCAGGGGCACAGGGGCTGCCCTCATTCTACCACCACCTCCAC |
| | | | | AGCACAGACAGACACTCAGGAGCAGCCAGCGGccaggtaagtttagtctttttgtcttttatt |
| | | | | tcaggtcccggatccggtggtggtgcaaatcaaagaactgctcctcagtggatgttgccttta |
| | | | | cttctaggcctgtacggaagtgttacttctgctctaaaagctgcggaattgtacccgcaatga |
| | | | | ggagtgatcatggctacctcagagctgagctgcgaggtgtcggaggagaactgtgagcgccgg |
| | | | | gaggccttctgggcagaatggaaggatctgacactgtccacacggcccgaggagggCTGCTCC |
| | | | | CTGCATGAGGAGGACACCCAGAGACATGAGACCTACCACCAGCAGGGGCAGTGCCAGGTGCTG |
| | | | | GTGCAGCGCTCGCCCTGGCTGATGATGCGGATGGGCATCCTCGGCCGTGGGCTGCAGGAGTAC |
| | | | | CAGCTGCCCTACCAGCGGGTACTGCCGCTGCCCATCTTCACCCCTGCCAAGATGGGCGCCACC |
| | | | | AAGGAGGAGCGTGAGGACACCCCCATCCAGCTTCAGGAGCTGCTGGCGCTGGAGACAGCCCTG |
| | | | | GGTGGCCAGTGTGTGGACCGCCAGGAGGTGGCTGAGATCACAAAGCAGCTGCCCCCTGTGGTG |
| | | | | CCTGTCAGCAAGCCCGGTGCACTTCGTCGCTCCCTGTCCCGCTCCATGTCCCAGGAAGCACAG |
| | | | | AGAGGCtgaGCGGCCGCaataaaagatctttattttcattagatctgtgtgttggttttttgt |
| | | | | gtg |
| 14 | CMV.TCAP. | 1447 | 32 | CGTTACATAACTTACGGTAAATGGCCCGCCTGGCTGACCGCCCAACGACCCCCGCCCATTGAC |
| | NI/SD | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACCTGaatg |
| | | | | aggagtgatcatggctacctcagagctgagctgcgaggtgtcggaggagaactgtgagcgccg |
| | | | | ggaggccttctgggcagaatggaaggatctgacactgtccacacggcccgaggaggggtgagt |
| | | | | gtgggtctgctagagccctgcctctgctcccccagagcaccctcactgagccatgaggccaga |
| | | | | gcatgaagccctggagaaatttctgggggtgggggcaggaagaatgccccatggggagagcaa |
| | | | | aggggaaccacccttcctgcccccaggtcccagcagcccaggggagccccccacccagcctgt |
| | | | | gcccagagagcaacagctcccaggagctcactgcccctcccctctccccagCTGCTCCCTGCA |
| | | | | TGAGGAGGACACCCAGAGACATGAGACCTACCACCAGCAGGGGCAGTGCCAGGTGCTGGTGCA |
| | | | | GCGCTCGCCCTGGCTGATGATGCGGATGGGCATCCTCGGCCGTGGGCTGCAGGAGTACCAGCT |
| | | | | GCCCTACCAGCGGGTACTGCCGCTGCCCATCTTCACCCCTGCCAAGATGGGCGCCACCAAGGA |
| | | | | GGAGCGTGAGGACACCCCCATCCAGCTTCAGGAGCTGCTGGCGCTGGAGACAGCCCTGGGTGG |
| | | | | CCAGTGTGTGGACCGCCAGGAGGTGGCTGAGATCACAAAGCAGCTGCCCCCTGTGGTGCCTGT |
| | | | | CAGCAAGCCCGGTGCACTTCGTCGCTCCCTGTCCCGCTCCATGTCCCAGGAAGCACAGAGAGG |
| | | | | CtgaGCGGCCGCaataaaagatctttattttcattagatctgtgtgttggttttttgtgtg |
| 15 | CMV.TCAP. | 1348 | 33 | CGTTACATAACTTACGGTAAATGGCCCGCCTGGCTGACCGCCCAACGACCCCCGCCCATTGAC |
| | SI/SD | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACaggtaag |
| | | | | tttagtctttttgtcttttatttcaggtcccggatccggtggtggtgcaaatcaaagaactgc |
| | | | | tcctcagtggatgttgcctttacttctaggcctgtacggaagtgttacttctgctctaaaagc |
| | | | | tgcggaattgtacccgcaatgaggagtgatcatggctacctcagagctgagctgcgaggtgtc |
| | | | | ggaggagaactgtgagcgccgggaggccttctgggcagaatggaaggatctgacactgtccac |
| | | | | acggcccgaggagggCTGCTCCCTGCATGAGGAGGACACCCAGAGACATGAGACCTACCACCA |
| | | | | GCAGGGGCAGTGCCAGGTGCTGGTGCAGCGCTCGCCCTGGCTGATGATGCGGATGGGCATCCT |
| | | | | CGGCCGTGGGCTGCAGGAGTACCAGCTGCCCTACCAGCGGGTACTGCCGCTGCCCATCTTCAC |
| | | | | CCCTGCCAAGATGGGCGCCACCAAGGAGGAGCGTGAGGACACCCCCATCCAGCTTCAGGAGCT |
| | | | | GCTGGCGCTGGAGACAGCCCTGGGTGGCCAGTGTGTGGACCGCCAGGAGGTGGCTGAGATCAC |
| | | | | AAAGCAGCTGCCCCCTGTGGTGCCTGTCAGCAAGCCCGGTGCACTTCGTCGCTCCCTGTCCCG |
| | | | | CTCCATGTCCCAGGAAGCACAGAGAGGCtgaGCGGCCGCaataaaagatctttattttcatta |
| | | | | gatctgtgtgttggttttttgtgtg |
| 16 | NP.TCAP. | 1791 | 34 | CCCCATGAAGAAGAGCCAGTCCCCTCCCAGGTAGCACATCAGAGCGGGCAGGCTCCAGAGCTC |
| | 3xFLAG/SD | | | TTCTGGTCCTCTTGCAGTGGCCAGAAAGTGGGGAAAGGGGCCCAGAGAGGGGCAGGGACTGAC |
| | | | | CCATGGTCACACAGCCTATTTATGGCTGATCTGGAGCCAGGCCTTCTGACTATCACCCATCAG |
| | | | | AGCCCCTGGATTCCCATTTCACCCAACACAGGTGTTGGGGTAGAACTGGGGGCCCTCCACCCC |
| | | | | TACTAGCCTGACTGTCTCCTGAGACCTGTCTCCTAAGACCACTTACCTGACCACTGGCCATGG |
| | | | | GGAAGGAAAACACCTGGCCAAGGCTCATGCCCCAGGTTCCCAGGCATGAAGTTGACTGTCTCC |
| | | | | GCCCCTGCTGGTGTGGTGAGGGTGACTGGGGACTAGGCACTAGGCCTTTGGTGCAGGCGCCTG |
| | | | | AGGACGTGGTTGCACTCTCCCTTCTGGGGATATGCCCTTGAGCCCAGGCAGAGGAGAGCACAG |
| | | | | CCAGGGCAGGACCTGGCAGCCCTGGTACAGAGCCCAGAGGGGGCATCAGTTCCTGCTGGTCCT |
| | | | | GCTCTGTTTACAGACAAGCTGCTGTCCTCCCTGCAAAGGGGAGTGGGTGGGGCAGAGGGCAAG |
| | | | | TGCCAGGGGGGCACAAGGCTGGGCATGTGGCTGGCATGAGACGGTGTCTGAGTAATGTCAGGC |
| | | | | ACCTGGAGGCATTGACCCCAGGACCTTGGACCCCAGACCTCTGACCGGGGGGCAGCCAGCGTC |
| | | | | CAGGTACCCCAACCCCTGCCCTGGGTCCGGCGTCCCCCCATTAGTGAGTCTTGGCTCTACTTA |
| | | | | TAGCATCTGACACCAGAGGGGCCGAAAATAGCCCCTGGAGAAGGGGGAGGAGGGGGCTATTTA |
| | | | | AAGGGCCTGGGAGGGGAGAGAGaatgaggagtgatcatggctacctcagagctgagctgcgag |
| | | | | gtgtcggaggagaactgtgagcgccgggaggccttctgggcagaatggaaggatctgacactg |
| | | | | tccacacggcccgaggaggggtgagtgtgggtctgctagagccctgcctctgctcccccagag |
| | | | | caccctcactgagccatgaggccagagcatgaagccctggagaaatttctgggggtgggggca |
| | | | | ggaagaatgccccatggggagagcaaaggggaaccacccttcctgcccccaggtcccagcagc |
| | | | | ccaggggagccccccacccagcctgtgcccagagagcaacagctcccaggagctcactgcccc |
| | | | | tcccctctccccagCTGCTCCCTGCATGAGGAGGACACCCAGAGACATGAGACCTACCACCAG |
| | | | | CAGGGGCAGTGCCAGGTGCTGGTGCAGCGCTCGCCCTGGCTGATGATGCGGATGGGCATCCTC |
| | | | | GGCCGTGGGCTGCAGGAGTACCAGCTGCCCTACCAGCGGGTACTGCCGCTGCCCATCTTCACC |
| | | | | CCTGCCAAGATGGGCGCCACCAAGGAGGAGCGTGAGGACACCCCCATCCAGCTTCAGGAGCTG |
| | | | | CTGGCGCTGGAGACAGCCCTGGGTGGCCAGTGTGTGGACCGCCAGGAGGTGGCTGAGATCACA |
| | | | | AAGCAGCTGCCCCCTGTGGTGCCTGTCAGCAAGCCCGGTGCACTTCGTCGCTCCCTGTCCCGC |
| | | | | TCCATGTCCCAGGAAGCACAGAGAGGCGACTACAAAGACCATGACGGTGATTATAAAGATCAT |
| | | | | GACATCGATTACAAGGATGACGATGACAAGtgaGCGGCCGCaataaaagatctttattttcat |
| | | | | tagatctgtgtgttggttttttgtgtg |
| 17 | MHCK7.TCAP. | 1675 | 35 | GCATGTCTAAGCTAGACCCTTCAGATTAAAAATAACTGAGGTAAGGGCCTGGGTAGGGGAGGT |
| | NI3xFLAG/SD | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCAACACCTGCTGCCTCTAAAAATAACCCTGTCCCTGGTGGATCCCCTGCATGCGAAGATCTT |
| | | | | CGAACAAGGCTGTGGGGGACTGAGGGCAGGCTGTAACAGGCTTGGGGGCCAGGGCTTATACGT |
| | | | | GCCTGGGACTCCCAAAGTATTACTGTTCCATGTTCCCGGCGAAGGGCCAGCTGTCCCCCGCCA |
| | | | | GCTAGACTCAGCACTTAGTTTAGGAACCAGTGAGCAAGTCAGCCCTTGGGGCAGCCCATACAA |
| | | | | GGCCATGGGGCTGGGCAAGCTGCACGCCTGGGTCCGGGGTGGGCACGGTGCCCGGGCAACGAG |
| | | | | CTGAAAGCTCATCTGCTCTCAGGGGCCCCTCCCTGGGGACAGCCCCTCCTGGCTAGTCACACC |
| | | | | CTGTAGGCTCCTCTATATAACCCAGGGGCACAGGGGCTGCCCTCATTCTACCACCACCTCCAC |
| | | | | AGCACAGACAGACACTCAGGAGCAGCCAGCGGaatgaggagtgatcatggctacctcagagct |
| | | | | gagctgcgaggtgtcggaggagaactgtgagcgccgggaggccttctgggcagaatggaagga |
| | | | | tctgacactgtccacacggcccgaggaggggtgagtgtgggtctgctagagccctgcctctgc |
| | | | | tcccccagagcaccctcactgagccatgaggccagagcatgaagccctggagaaatttctggg |
| | | | | ggtgggggcaggaagaatgccccatggggagagcaaaggggaaccacccttcctgcccccagg |
| | | | | tcccagcagcccaggggagccccccacccagcctgtgcccagagagcaacagctcccaggagc |
| | | | | tcactgcccctcccctctccccagCTGCTCCCTGCATGAGGAGGACACCCAGAGACATGAGAC |
| | | | | CTACCACCAGCAGGGGCAGTGCCAGGTGCTGGTGCAGCGCTCGCCCTGGCTGATGATGCGGAT |
| | | | | GGGCATCCTCGGCCGTGGGCTGCAGGAGTACCAGCTGCCCTACCAGCGGGTACTGCCGCTGCC |
| | | | | CATCTTCACCCCTGCCAAGATGGGCGCCACCAAGGAGGAGCGTGAGGACACCCCCATCCAGCT |
| | | | | TCAGGAGCTGCTGGCGCTGGAGACAGCCCTGGGTGGCCAGTGTGTGGACCGCCAGGAGGTGGC |
| | | | | TGAGATCACAAAGCAGCTGCCCCCTGTGGTGCCTGTCAGCAAGCCCGGTGCACTTCGTCGCTC |
| | | | | CCTGTCCCGCTCCATGTCCCAGGAAGCACAGAGAGGCGACTACAAAGACCATGACGGTGATTA |
| | | | | TAAAGATCATGACATCGATTACAAGGATGACGATGACAAGtgaGCGGCCGCaataaaagatct |
| | | | | ttattttcattagatctgtgtgttggttttttgtgtg |
| 18 | MHCK7.TCAP. | 1581 | 36 | GCATGTCTAAGCTAGACCCTTCAGATTAAAAATAACTGAGGTAAGGGCCTGGGTAGGGGAGGT |
| | SI3xFLAG/SD | | | GGTGTGAGACGCTCCTGTCTCTCCTCTATCTGCCCATCGGCCCTTTGGGGAGGAGGAATGTGC |
| | | | | CCAAGGACTAAAAAAAGGCCATGGAGCCAGAGGGGCGAGGGCAACAGACCTTTCATGGGCAAA |
| | | | | CCTTGGGGCCCTGCTGTCTAGCATGCCCCACTACGGGTCTAGGCTGCCCATGTAAGGAGGCAA |
| | | | | GGCCTGGGGACACCCGAGATGCCTGGTTATAATTAACCCAGACATGTGGCTGCCCCCCCCCCC |
| | | | | CCAACACCTGCTGCCTCTAAAAATAACCCTGTCCCTGGTGGATCCCCTGCATGCGAAGATCTT |
| | | | | CGAACAAGGCTGTGGGGGACTGAGGGCAGGCTGTAACAGGCTTGGGGGCCAGGGCTTATACGT |
| | | | | GCCTGGGACTCCCAAAGTATTACTGTTCCATGTTCCCGGCGAAGGGCCAGCTGTCCCCCGCCA |
| | | | | GCTAGACTCAGCACTTAGTTTAGGAACCAGTGAGCAAGTCAGCCCTTGGGGCAGCCCATACAA |
| | | | | GGCCATGGGGCTGGGCAAGCTGCACGCCTGGGTCCGGGGTGGGCACGGTGCCCGGGCAACGAG |
| | | | | CTGAAAGCTCATCTGCTCTCAGGGGCCCCTCCCTGGGGACAGCCCCTCCTGGCTAGTCACACC |
| | | | | CTGTAGGCTCCTCTATATAACCCAGGGGCACAGGGGCTGCCCTCATTCTACCACCACCTCCAC |
| | | | | AGCACAGACAGACACTCAGGAGCAGCCAGCGGccaggtaagtttagtctttttgtcttttatt |
| | | | | tcaggtcccggatccggtggtggtgcaaatcaaagaactgctcctcagtggatgttgccttta |
| | | | | cttctaggcctgtacggaagtgttacttctgctctaaaagctgcggaattgtacccgcaatga |
| | | | | ggagtgatcatggctacctcagagctgagctgcgaggtgtcggaggagaactgtgagcgccgg |
| | | | | gaggccttctgggcagaatggaaggatctgacactgtccacacggcccgaggagggCTGCTCC |
| | | | | CTGCATGAGGAGGACACCCAGAGACATGAGACCTACCACCAGCAGGGGCAGTGCCAGGTGCTG |
| | | | | GTGCAGCGCTCGCCCTGGCTGATGATGCGGATGGGCATCCTCGGCCGTGGGCTGCAGGAGTAC |
| | | | | CAGCTGCCCTACCAGCGGGTACTGCCGCTGCCCATCTTCACCCCTGCCAAGATGGGCGCCACC |
| | | | | AAGGAGGAGCGTGAGGACACCCCCATCCAGCTTCAGGAGCTGCTGGCGCTGGAGACAGCCCTG |
| | | | | GGTGGCCAGTGTGTGGACCGCCAGGAGGTGGCTGAGATCACAAAGCAGCTGCCCCCTGTGGTG |
| | | | | CCTGTCAGCAAGCCCGGTGCACTTCGTCGCTCCCTGTCCCGCTCCATGTCCCAGGAAGCACAG |
| | | | | AGAGGCGACTACAAAGACCATGACGGTGATTATAAAGATCATGACATCGATTACAAGGATGAC |
| | | | | GATGACAAGtgaGCGGCCGCaataaaagatctttattttcattagatctgtgtgttggttttt |
| | | | | tgtgtg |
| 19 | CMV.TCAP. | 1513 | 37 | CGTTACATAACTTACGGTAAATGGCCCGCCTGGCTGACCGCCCAACGACCCCCGCCCATTGAC |
| | NI3xFLAG/SD | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACCTGaatg |
| | | | | aggagtgatcatggctacctcagagctgagctgcgaggtgtcggaggagaactgtgagcgccg |
| | | | | ggaggccttctgggcagaatggaaggatctgacactgtccacacggcccgaggaggggtgagt |
| | | | | gtgggtctgctagagccctgcctctgctcccccagagcaccctcactgagccatgaggccaga |
| | | | | gcatgaagccctggagaaatttctgggggtgggggcaggaagaatgccccatggggagagcaa |
| | | | | aggggaaccacccttcctgcccccaggtcccagcagcccaggggagccccccacccagcctgt |
| | | | | gcccagagagcaacagctcccaggagctcactgcccctcccctctccccagCTGCTCCCTGCA |
| | | | | TGAGGAGGACACCCAGAGACATGAGACCTACCACCAGCAGGGGCAGTGCCAGGTGCTGGTGCA |
| | | | | GCGCTCGCCCTGGCTGATGATGCGGATGGGCATCCTCGGCCGTGGGCTGCAGGAGTACCAGCT |
| | | | | GCCCTACCAGCGGGTACTGCCGCTGCCCATCTTCACCCCTGCCAAGATGGGCGCCACCAAGGA |
| | | | | GGAGCGTGAGGACACCCCCATCCAGCTTCAGGAGCTGCTGGCGCTGGAGACAGCCCTGGGTGG |
| | | | | CCAGTGTGTGGACCGCCAGGAGGTGGCTGAGATCACAAAGCAGCTGCCCCCTGTGGTGCCTGT |
| | | | | CAGCAAGCCCGGTGCACTTCGTCGCTCCCTGTCCCGCTCCATGTCCCAGGAAGCACAGAGAGG |
| | | | | CGACTACAAAGACCATGACGGTGATTATAAAGATCATGACATCGATTACAAGGATGACGATGA |
| | | | | CAAGtgaGCGGCCGCaataaaagatctttattttcattagatctgtgtgttggttttttgtgt |
| | | | | g |
| 20 | CMV.TCAP. | 1414 | 38 | CGTTACATAACTTACGGTAAATGGCCCGCCTGGCTGACCGCCCAACGACCCCCGCCCATTGAC |
| | SI3xFLAG/SD | | | GTCAATAATGACGTATGTTCCCATAGTAACGCCAATAGGGACTTTCCATTGACGTCAATGGGT |
| | | | | GGAGTATTTACGGTAAACTGCCCACTTGGCAGTACATCAAGTGTATCATATGCCAAGTACGCC |
| | | | | CCCTATTGACGTCAATGACGGTAAATGGCCCGCCTGGCATTATGCCCAGTACATGACCTTATG |
| | | | | GGACTTTCCTACTTGGCAGTACATCTACGTATTAGTCATCGCTATTACCATGGTGATGCGGTT |
| | | | | TTGGCAGTACATCAATGGGCGTGGATAGCGGTTTGACTCACGGGGATTTCCAAGTCTCCACCC |
| | | | | CATTGACGTCAATGGGAGTTTGTTTTGGCACCAAAATCAACGGGACTTTCCAAAATGTCGTAA |
| | | | | CAACTCCGCCCCATTGACGCAAATGGGCGGTAGGCGTGTACGGTGGGAGGTCTATATAAGCAG |
| | | | | AGCTCGTTTAGTGAACCGTCAGATCGCCTGGAGACGCCATCCACGCTGTTTTGACCTCCATAG |
| | | | | AAGACACCGGGACCGATCCAGCCTCCGGACTCTAGAGGATCCGGTACTCGAGGAACaggtaag |
| | | | | tttagtctttttgtcttttatttcaggtcccggatccggtggtggtgcaaatcaaagaactgc |
| | | | | tcctcagtggatgttgcctttacttctaggcctgtacggaagtgttacttctgctctaaaagc |
| | | | | tgcggaattgtacccgcaatgaggagtgatcatggctacctcagagctgagctgcgaggtgtc |
| | | | | ggaggagaactgtgagcgccgggaggccttctgggcagaatggaaggatctgacactgtccac |
| | | | | acggcccgaggagggCTGCTCCCTGCATGAGGAGGACACCCAGAGACATGAGACCTACCACCA |
| | | | | GCAGGGGCAGTGCCAGGTGCTGGTGCAGCGCTCGCCCTGGCTGATGATGCGGATGGGCATCCT |
| | | | | CGGCCGTGGGCTGCAGGAGTACCAGCTGCCCTACCAGCGGGTACTGCCGCTGCCCATCTTCAC |
| | | | | CCCTGCCAAGATGGGCGCCACCAAGGAGGAGCGTGAGGACACCCCCATCCAGCTTCAGGAGCT |
| | | | | GCTGGCGCTGGAGACAGCCCTGGGTGGCCAGTGTGTGGACCGCCAGGAGGTGGCTGAGATCAC |
| | | | | AAAGCAGCTGCCCCCTGTGGTGCCTGTCAGCAAGCCCGGTGCACTTCGTCGCTCCCTGTCCCG |
| | | | | CTCCATGTCCCAGGAAGCACAGAGAGGCGACTACAAAGACCATGACGGTGATTATAAAGATCA |
| | | | | TGACATCGATTACAAGGATGACGATGACAAGtgaGCGGCCGCaataaaagatctttattttca |
| | | | | ttagatctgtgtgttggttttttgtgtg |

^{α}MHCK7 promoter shows a three nucleotide insertion in poly(C) region. The transgene expected size is 1675 bp.
^{β}MHCK7 promoter shows a two nucleotide deletion in poly(C) region. The transgene expected size is 1581 bp.

In some aspects, the disclosure includes a nanoparticle, extracellular vesicle, exosome, or vector comprising any of the nucleic acids of the disclosure or a combination of any one or more thereof for providing TCAP gene replacement. In some aspects, one or more copies of these sequences are combined into a single nanoparticle, extracellular vesicle, exosome, or vector.

The disclosure therefore includes vectors comprising a nucleic acid of the disclosure or a combination of nucleic acids of the disclosure. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein.

The disclosure provides a recombinant (r) AAV vector comprising the nucleic acid comprising a polynucleotide encoding the TCAP protein for use in treating a subject comprising a mutation in the TCAP gene. AAV is unique in its safety profile, as the viral genome, once transduced into its carrier cell, remains stably expressed as an episomal DNA and only very rarely ever integrates into the host genome^{16,17}.

In some aspects, therefore, the disclosure utilizes AAV to deliver the TCAP transgenes, such as DNA encoding the TCAP protein. As used herein, the term “AAV” is a standard abbreviation for adeno-associated virus. An “AAV vector” as used herein refers to a vector comprising one or more polynucleotides of interest (or transgenes) that are flanked by AAV terminal repeat sequences (ITRs).

In some aspects, therefore, a nucleic acid of the disclosure comprises an AAV vector comprising the nucleotide sequence set forth in any one of SEQ ID NOs: 19-28 and 39-48 (Table 3). In some aspects, a nucleic acid of the disclosure comprises the nucleotide sequence set forth in any one of SEQ ID NOs: 19-28 and 39-48. In some aspects, the nucleic acid of the AAV vector comprising the TCAP replacement gene comprises the nucleotide sequence set forth in any one of SEQ ID NOs: 19-28 and 39-48. In various aspects, the nucleic acid is a variant of the nucleotide sequence set forth in any one of SEQ ID NOs: 19-28 and 39-48. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 19-28 and 39-48.

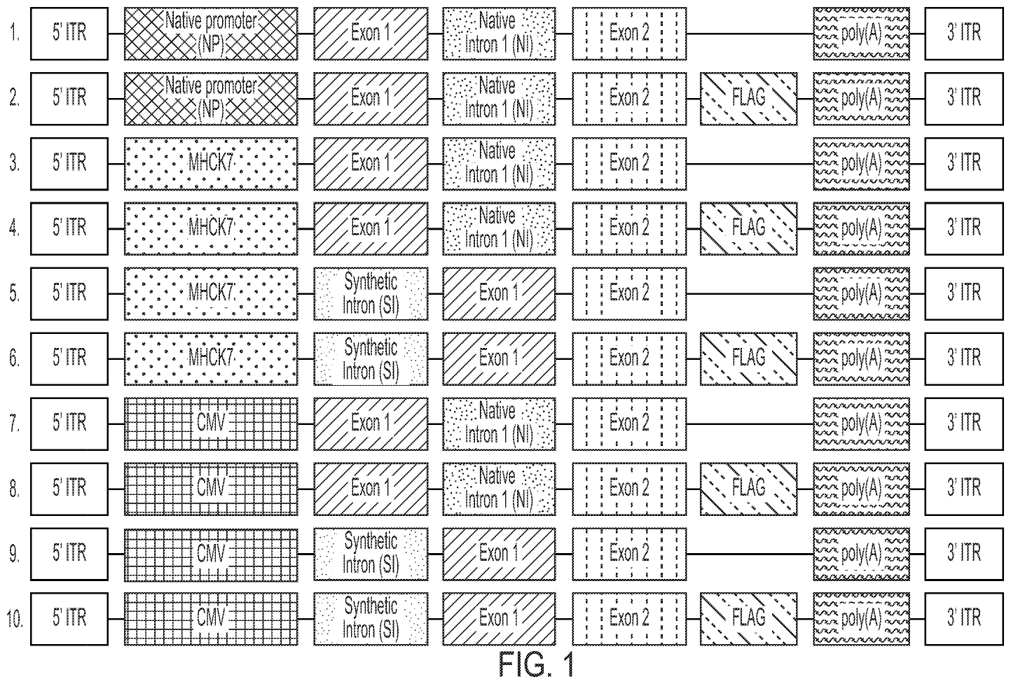

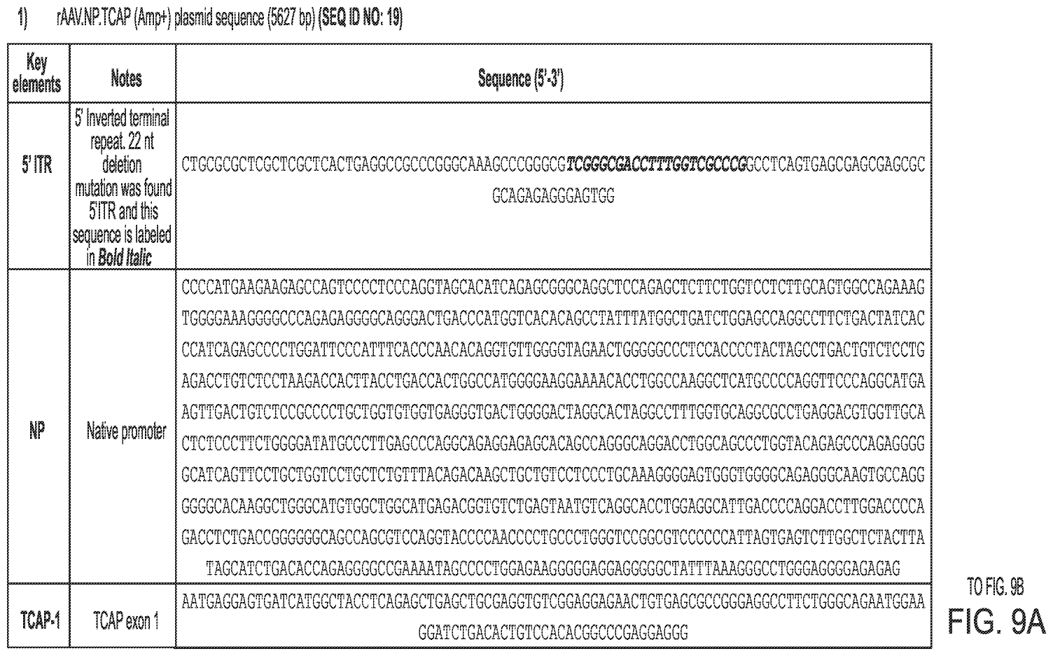

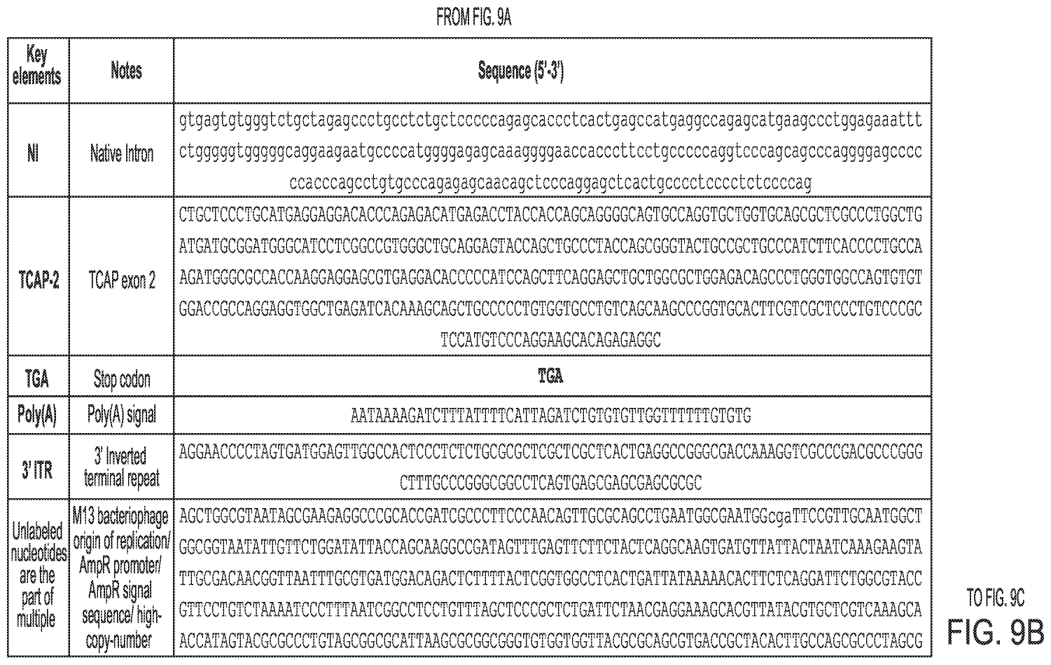

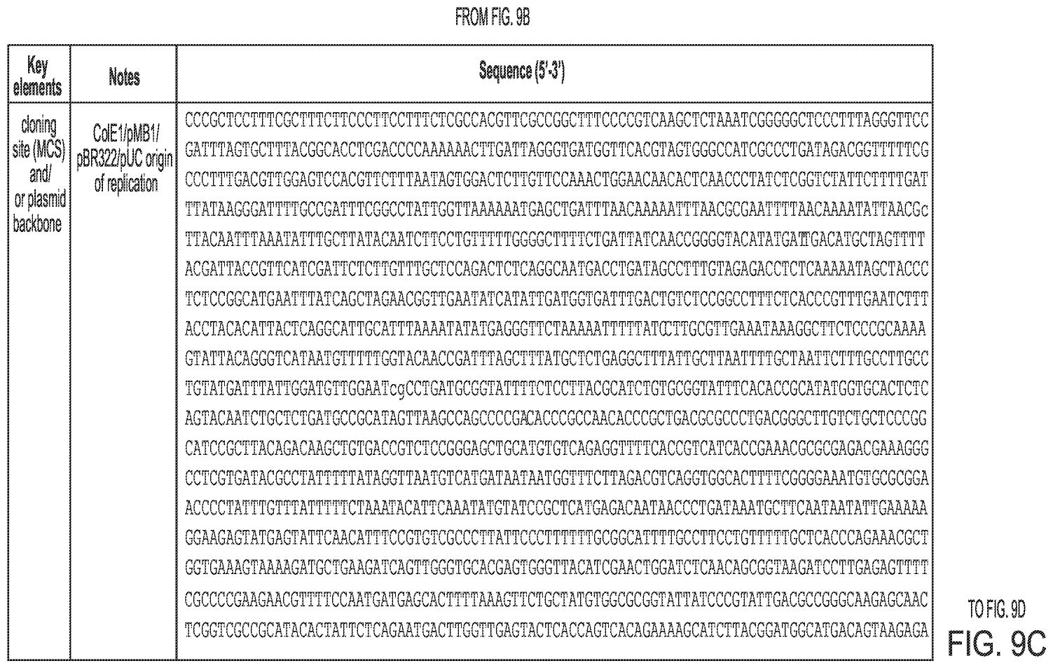

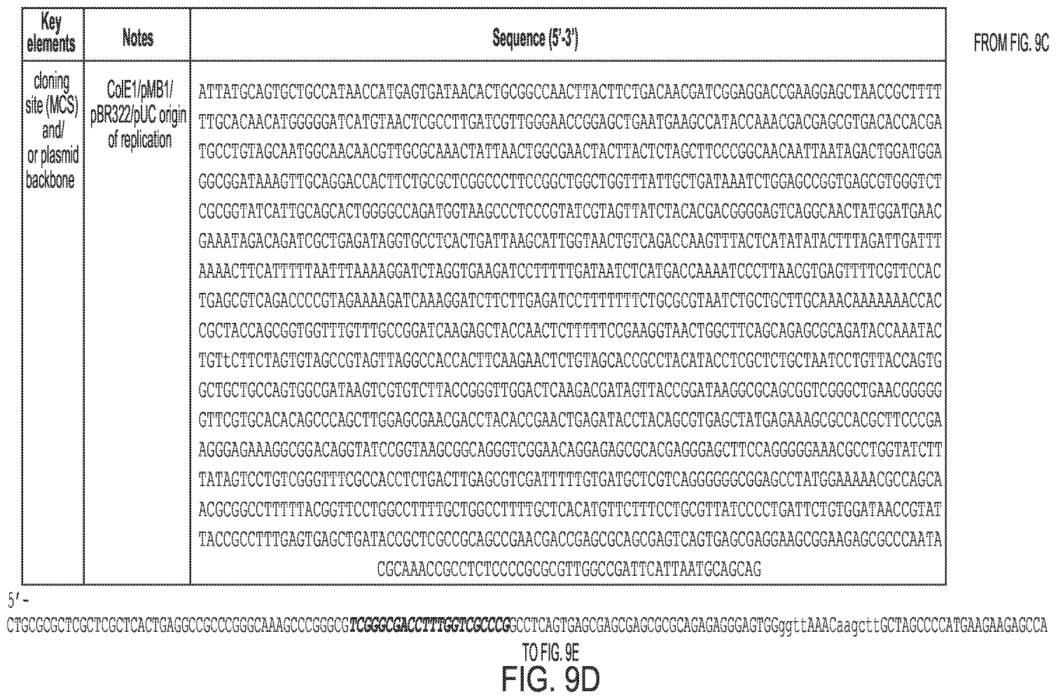

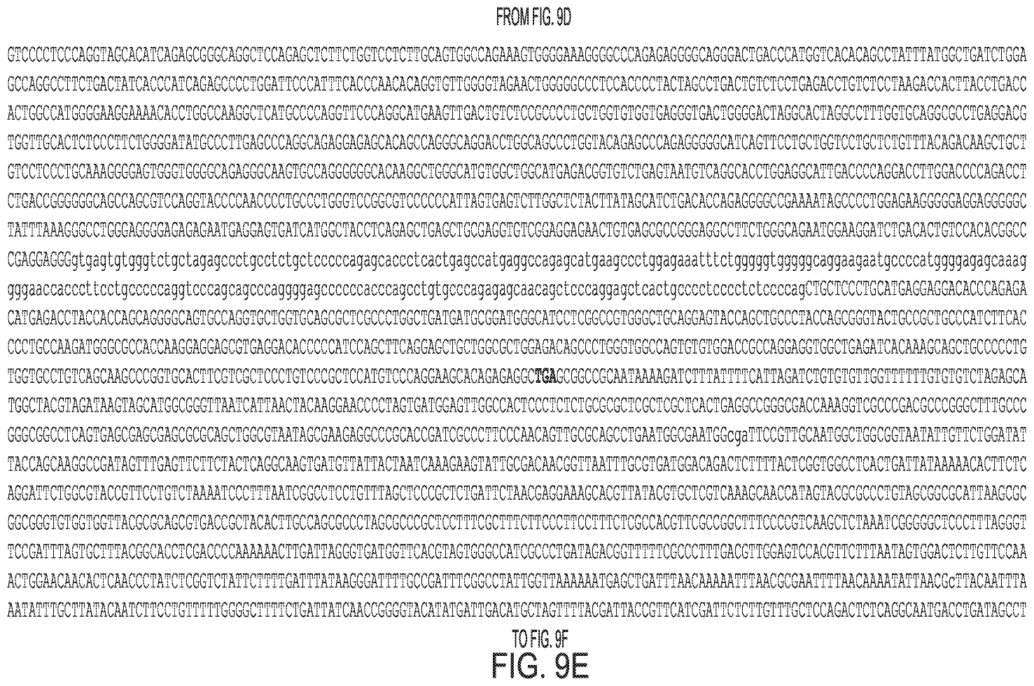

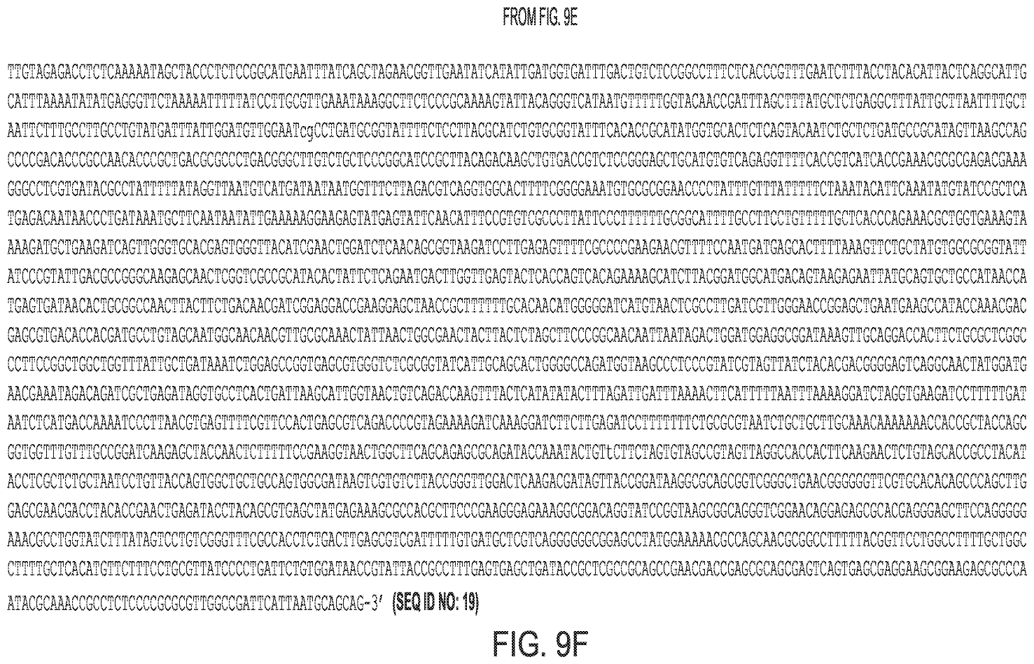

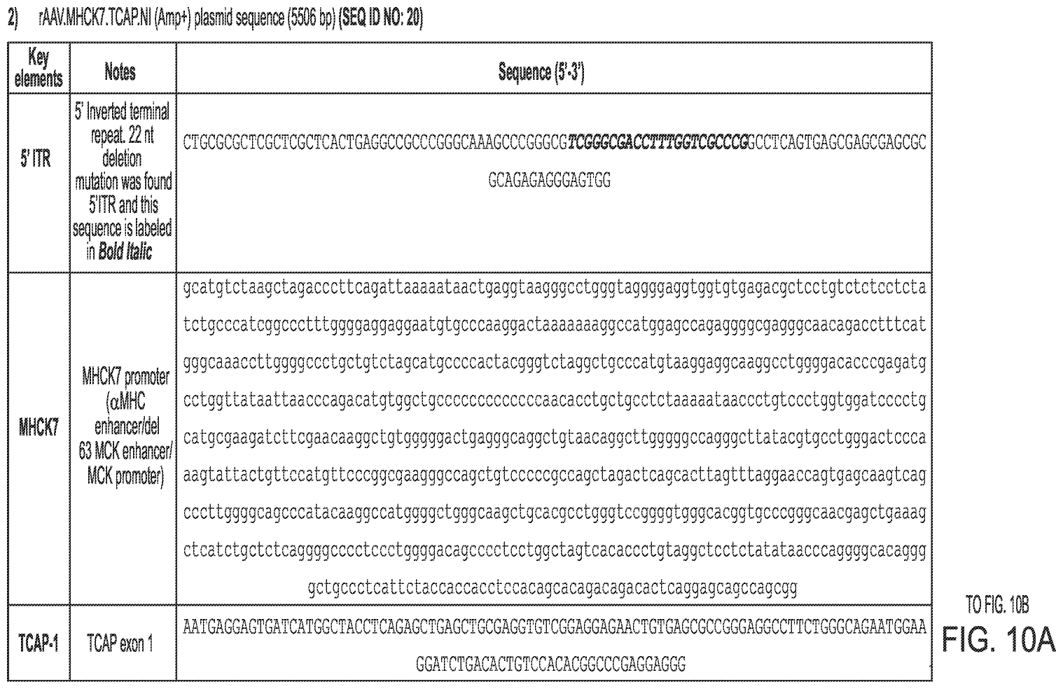

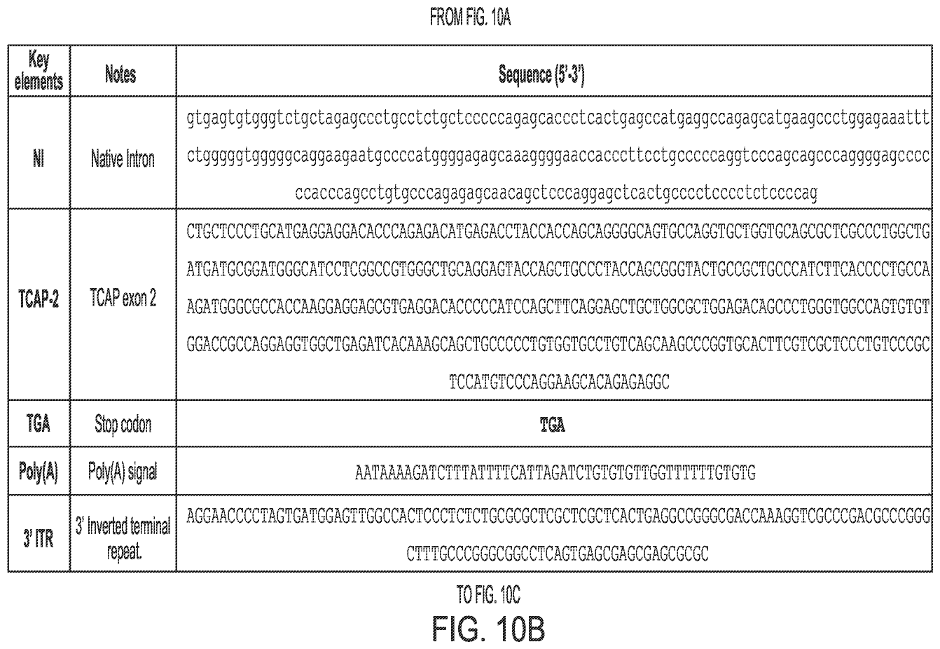

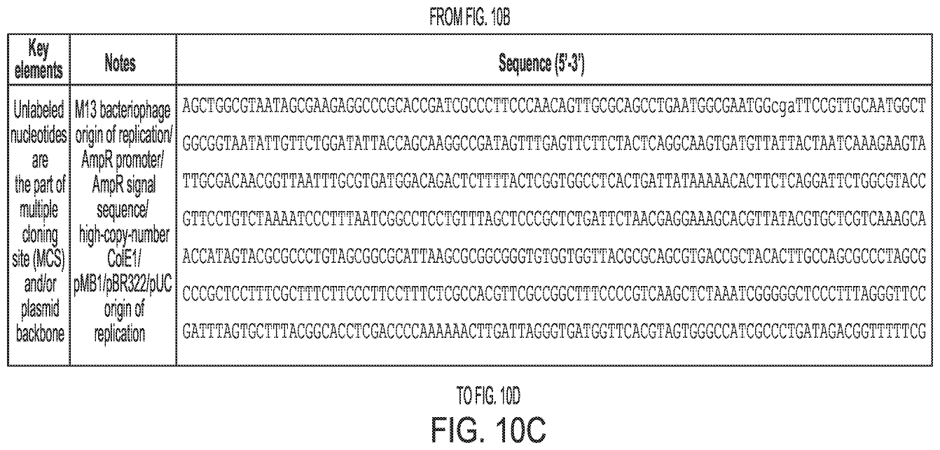

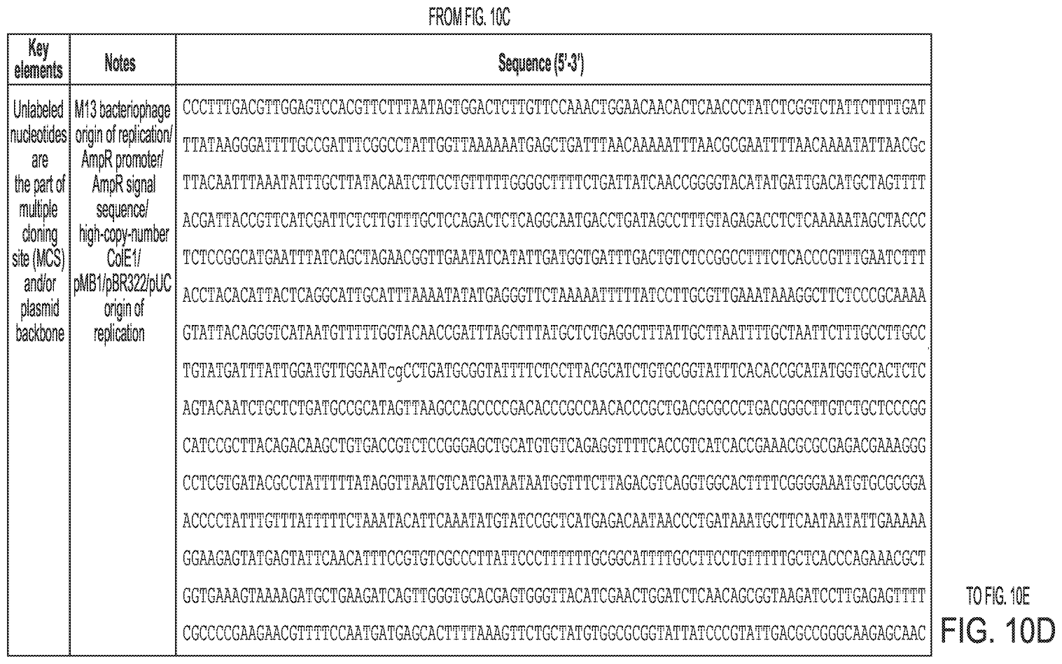

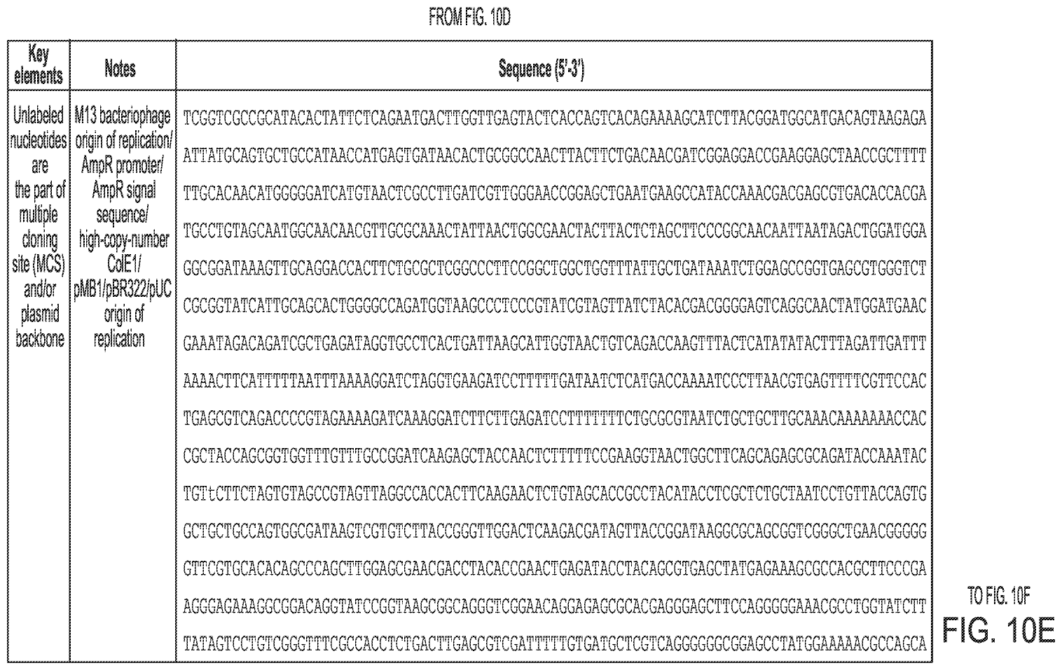

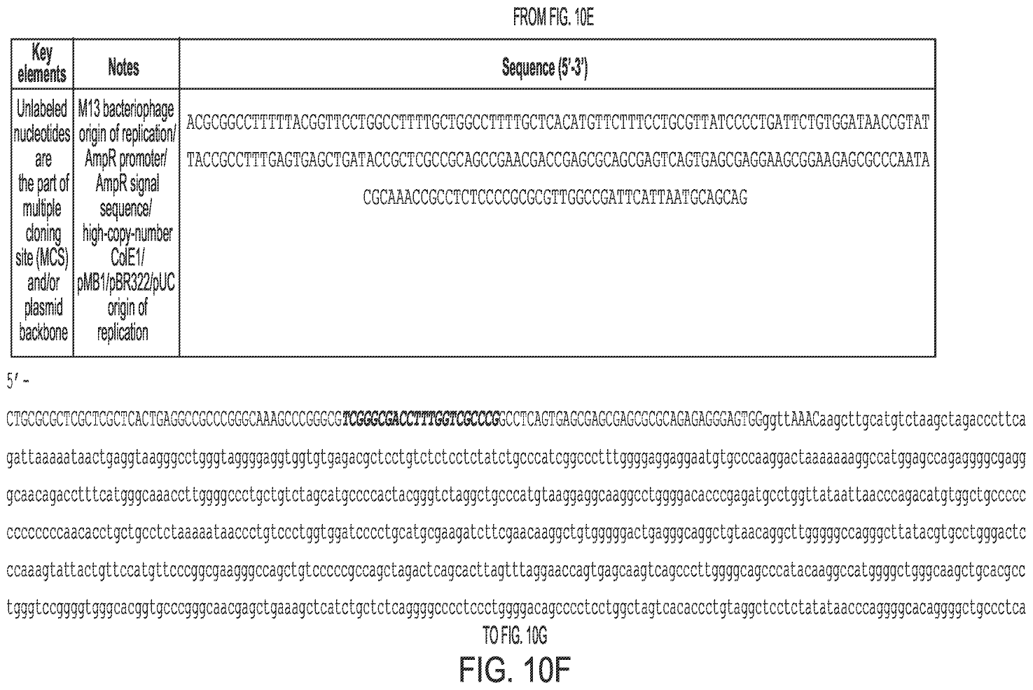

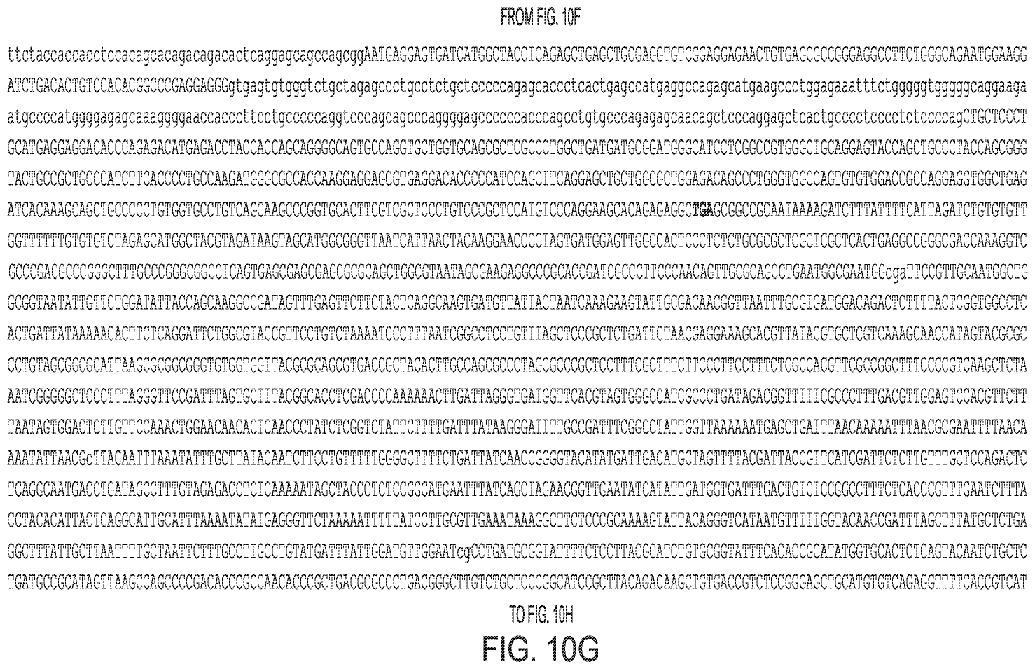

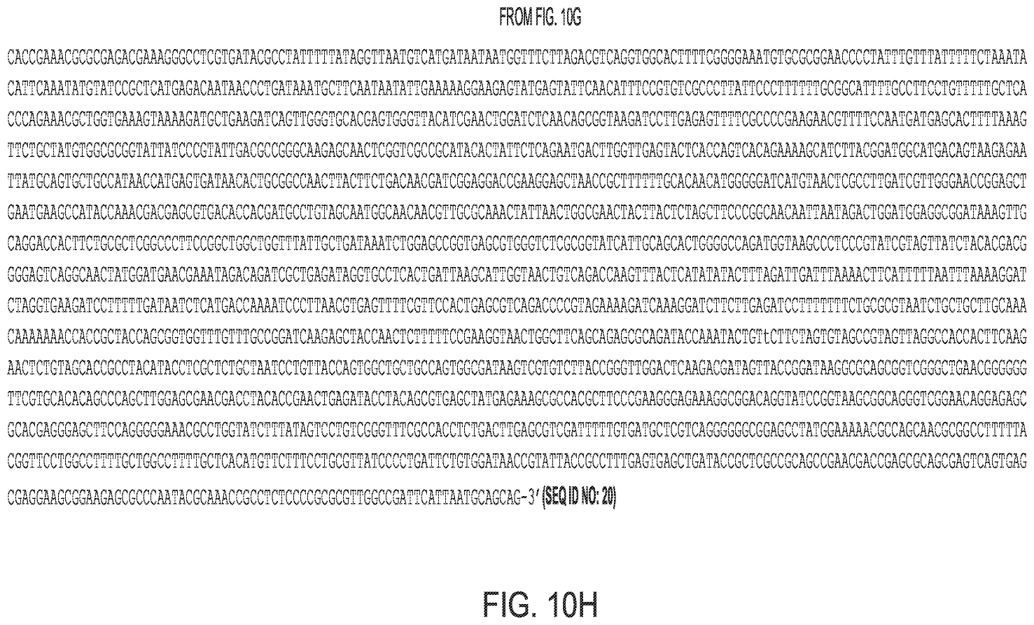

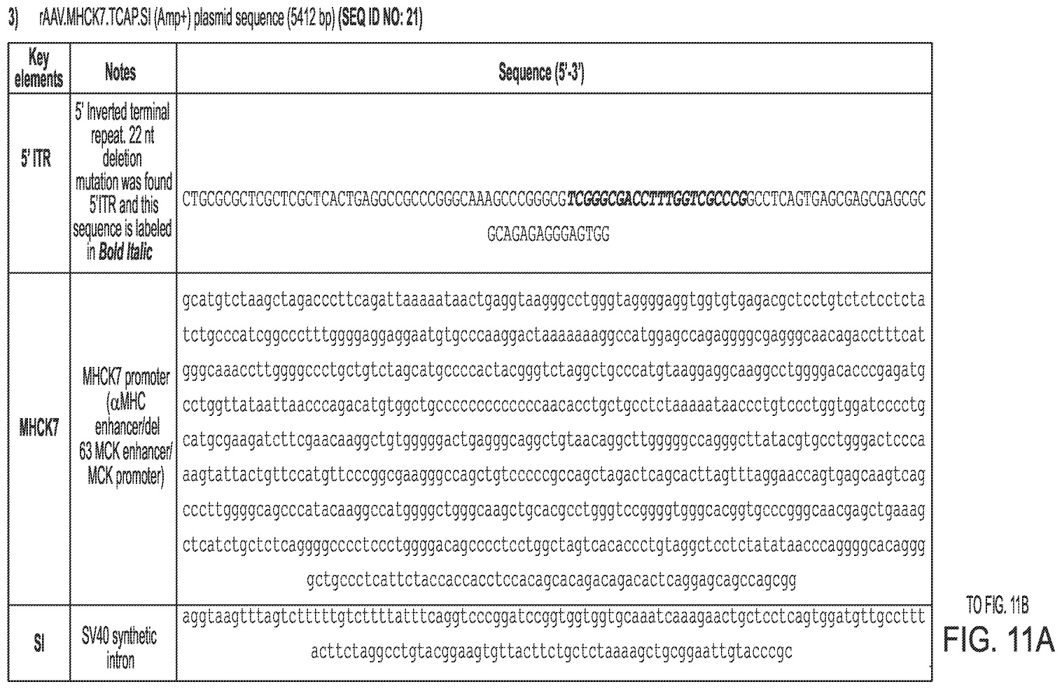

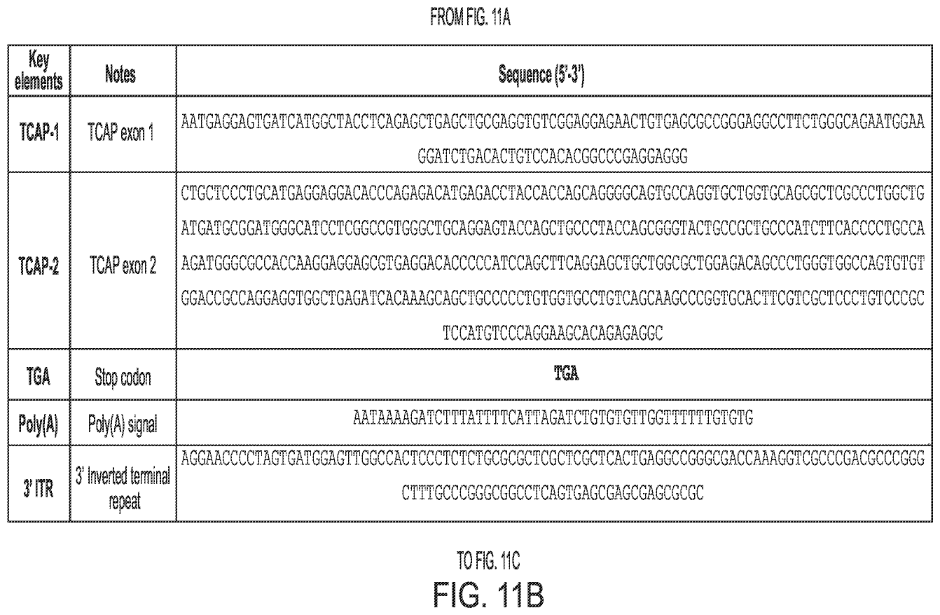

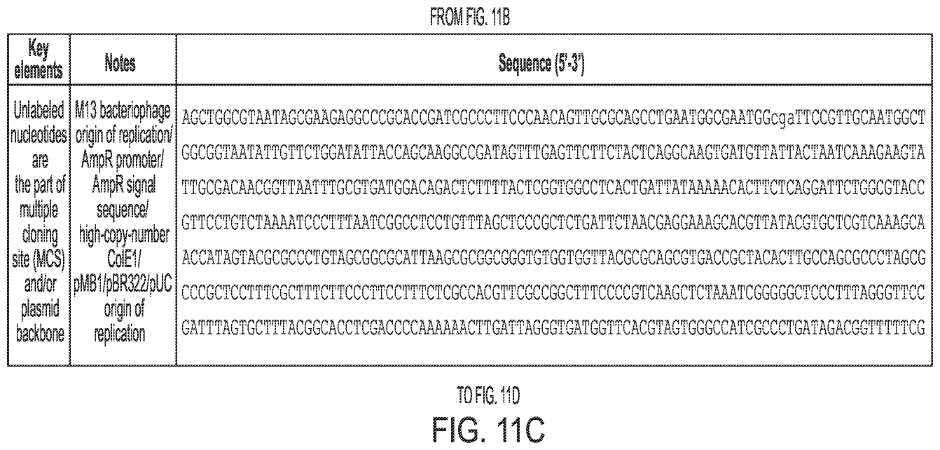

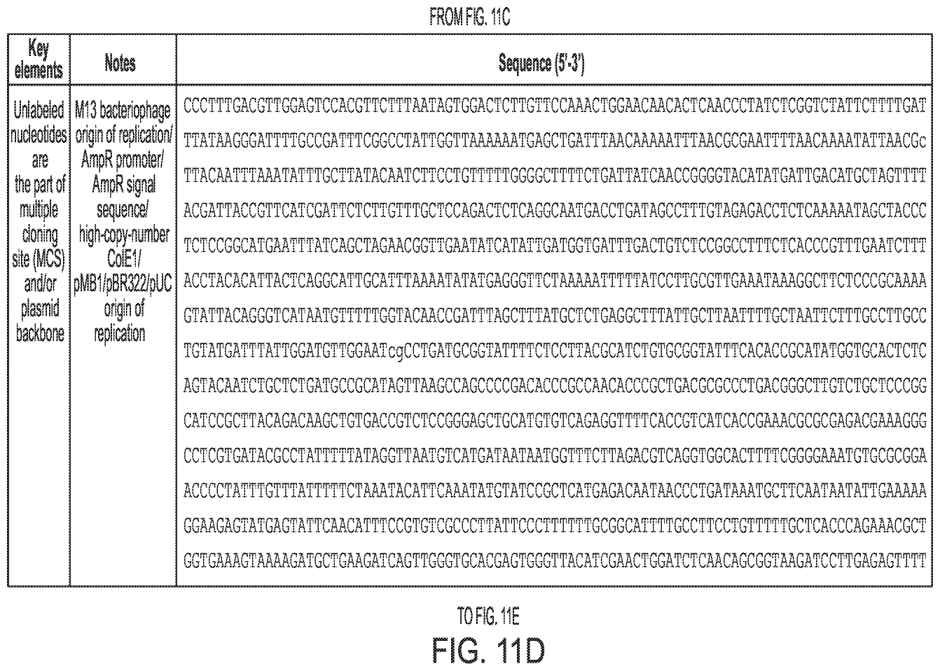

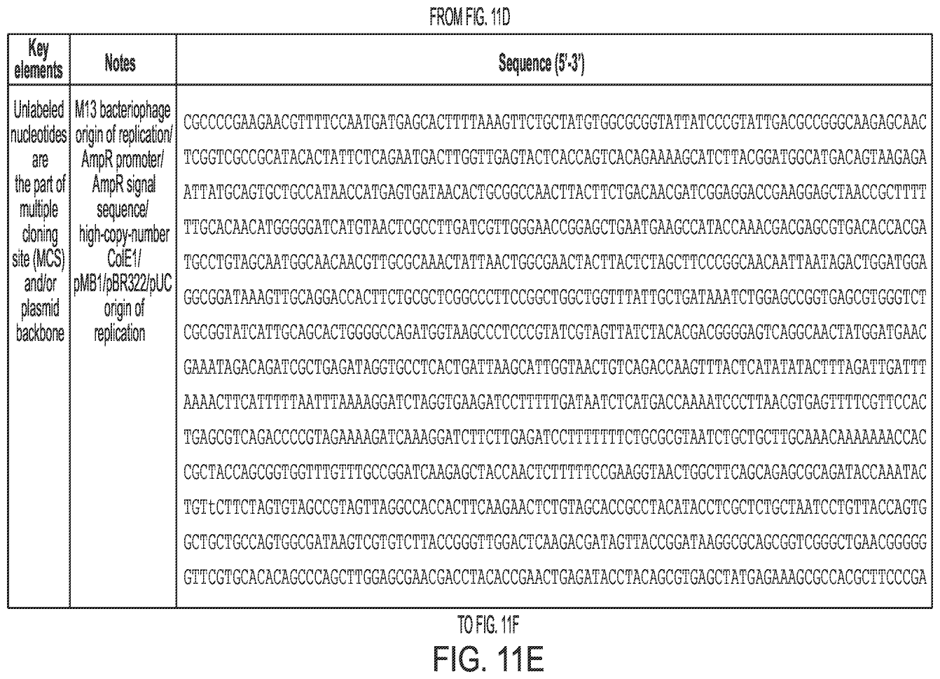

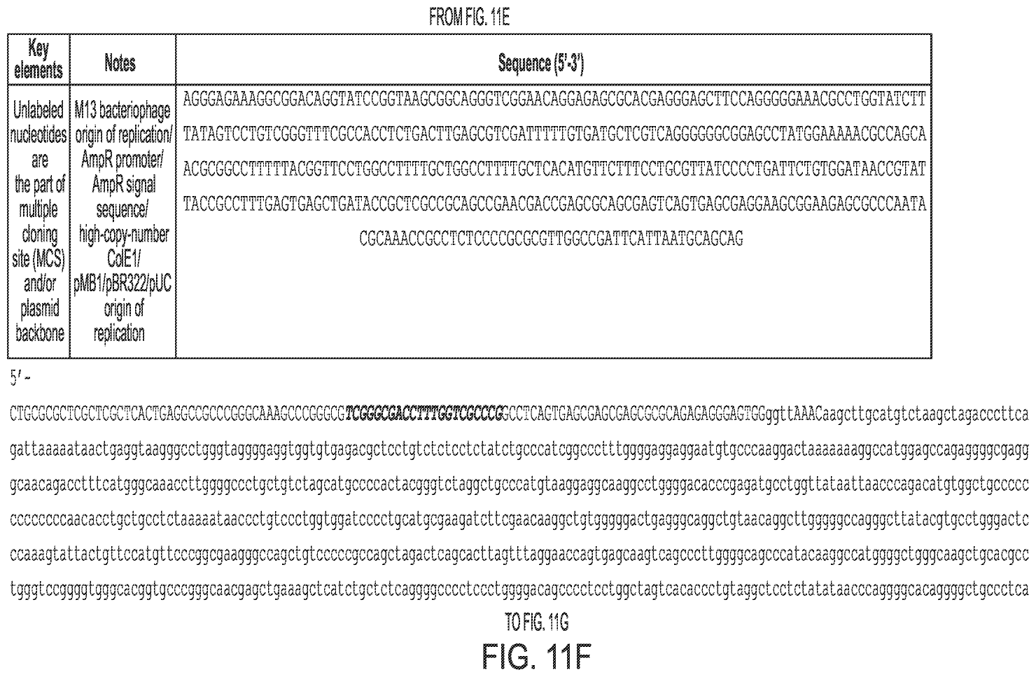

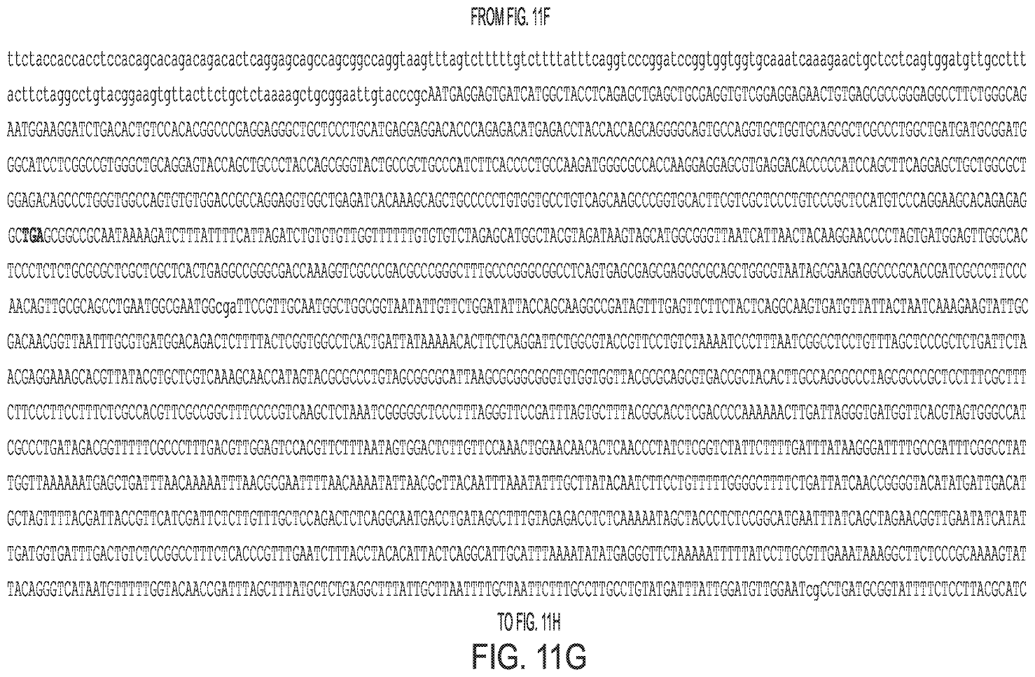

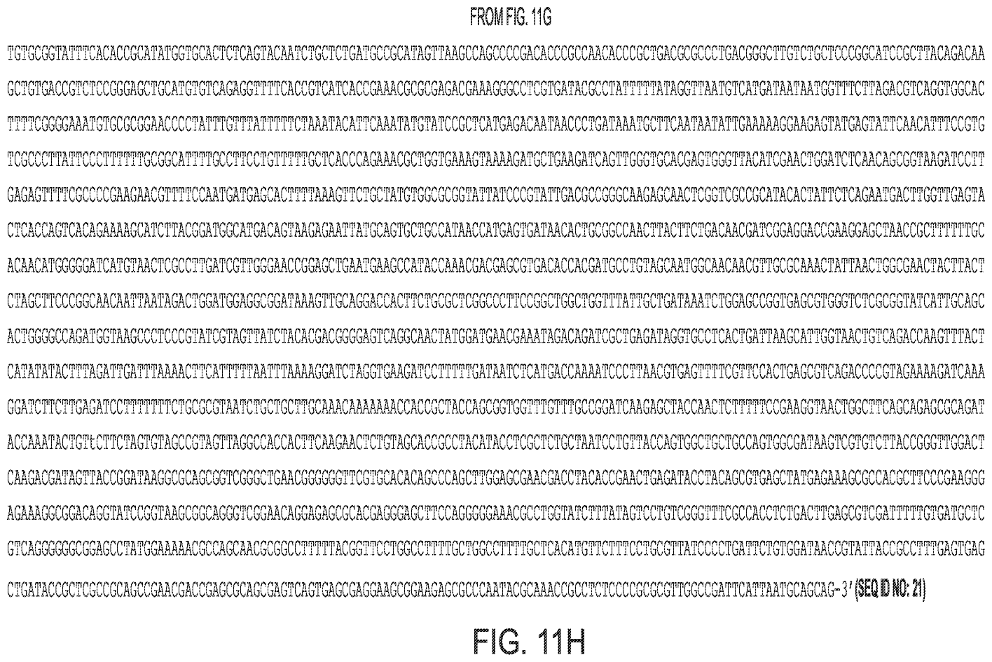

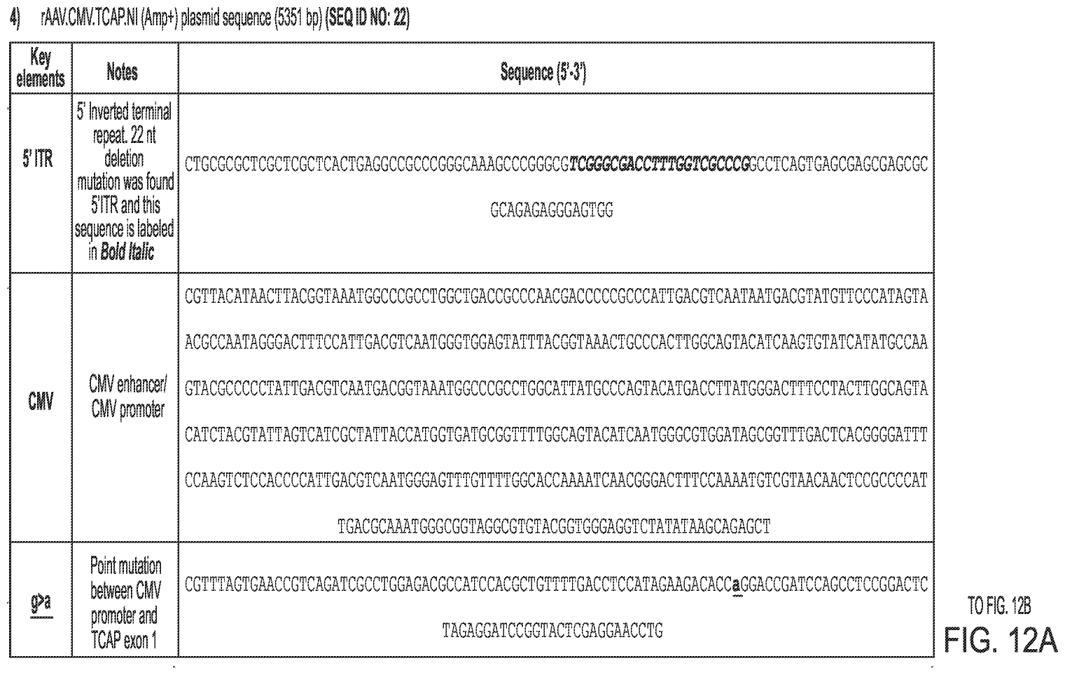

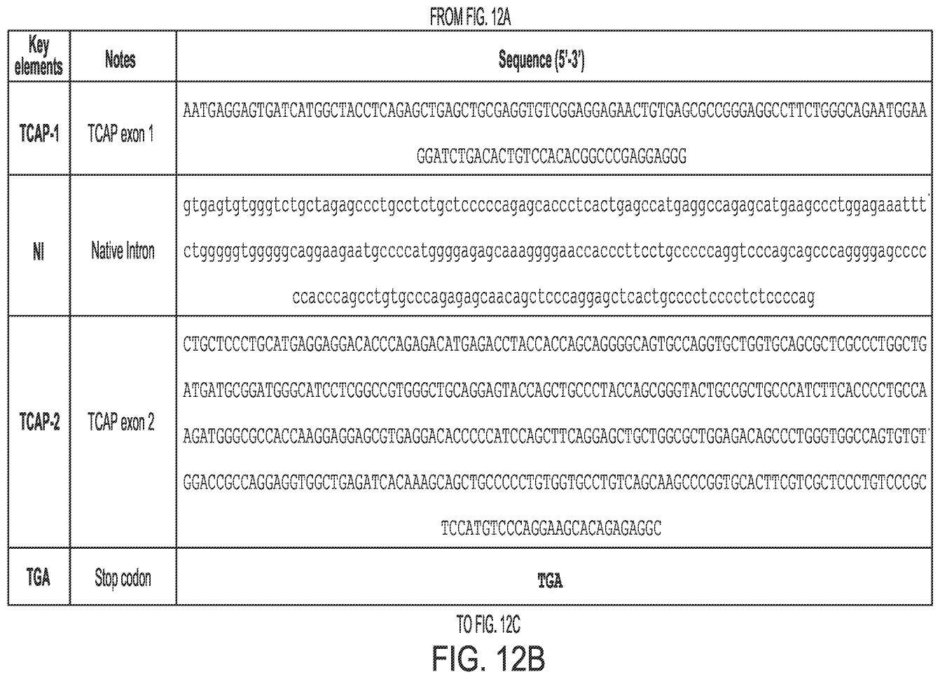

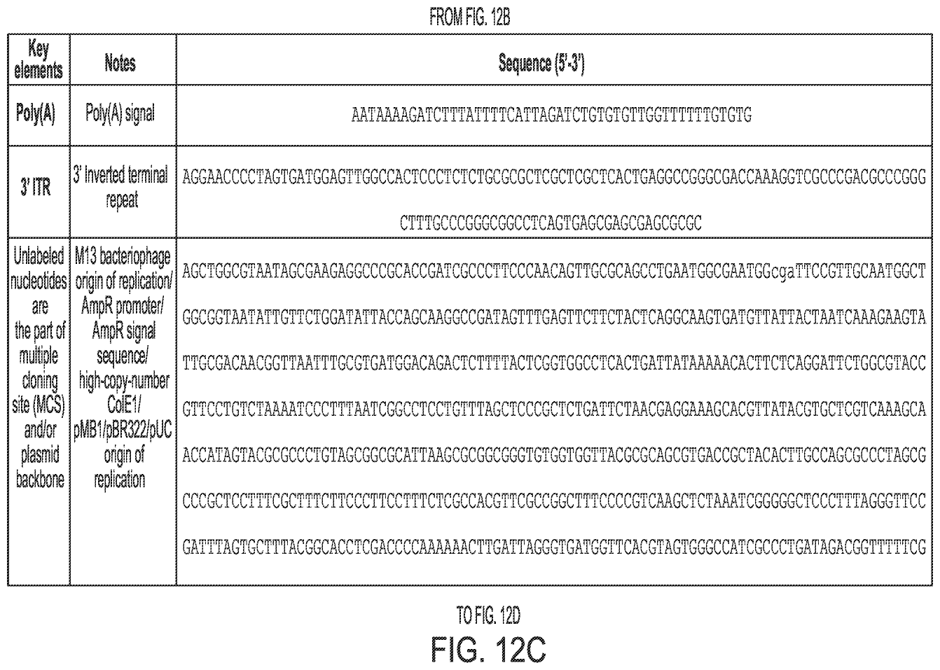

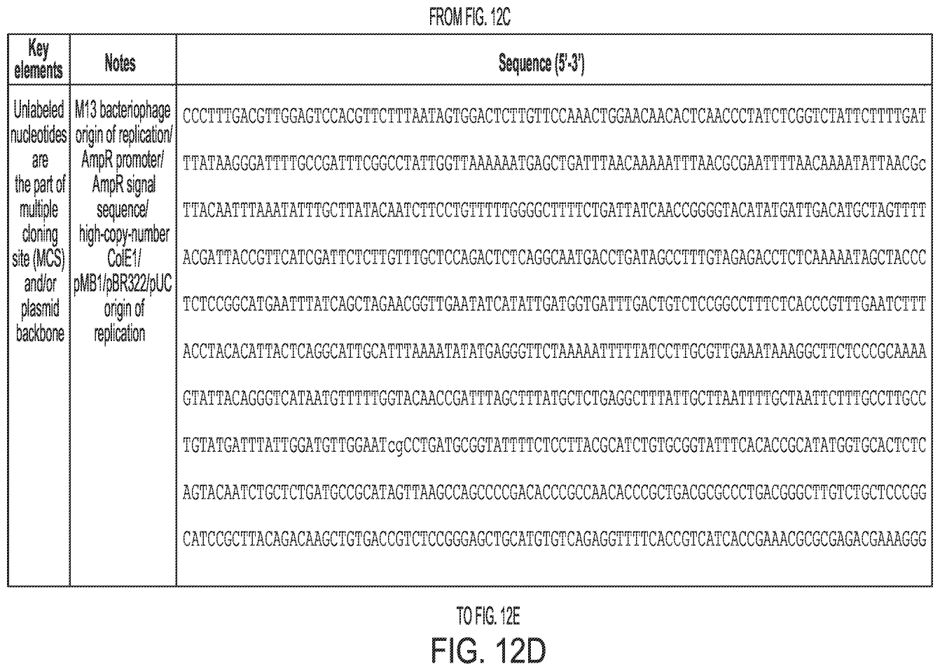

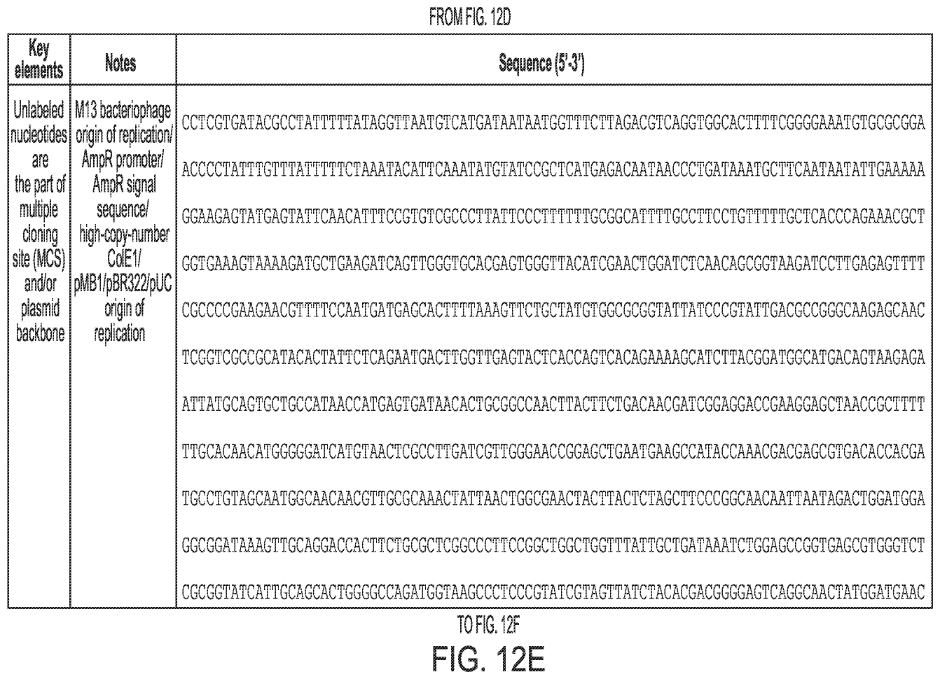

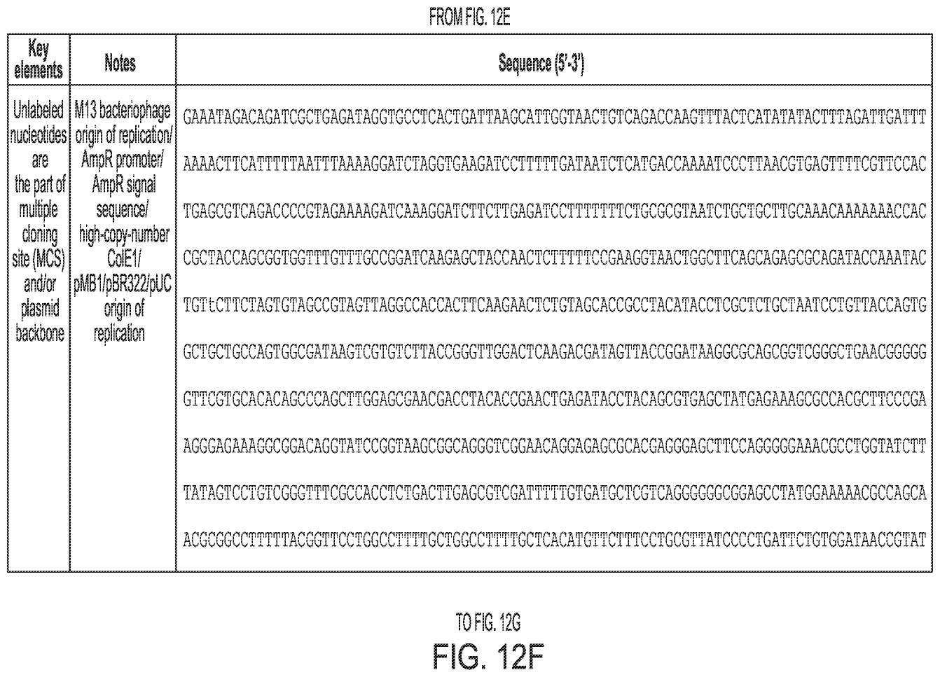

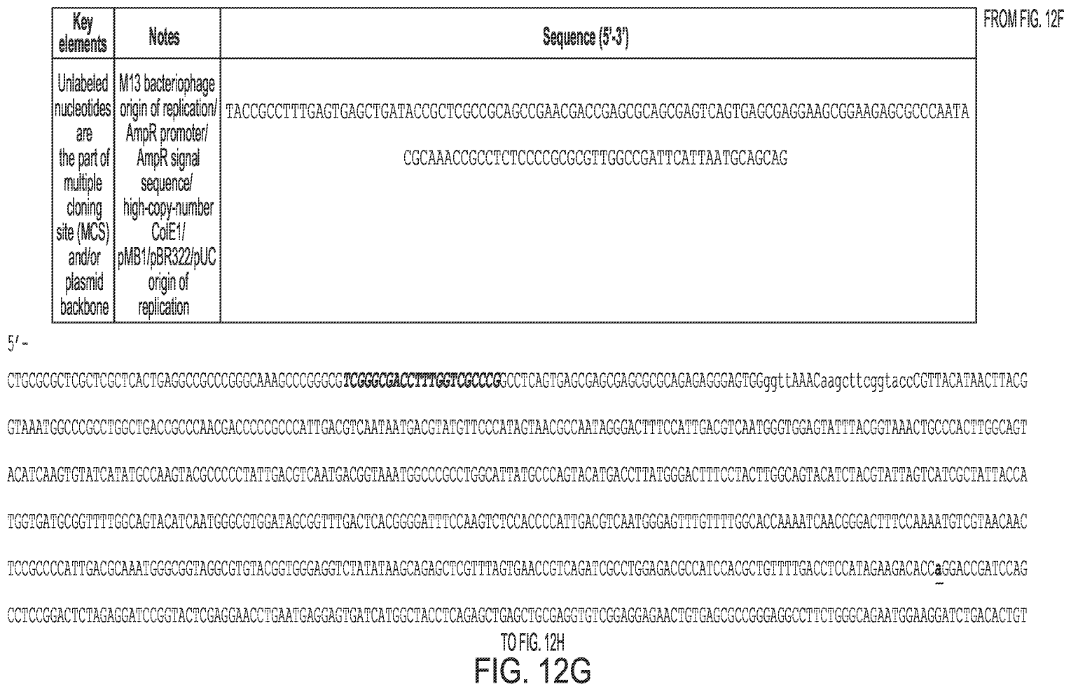

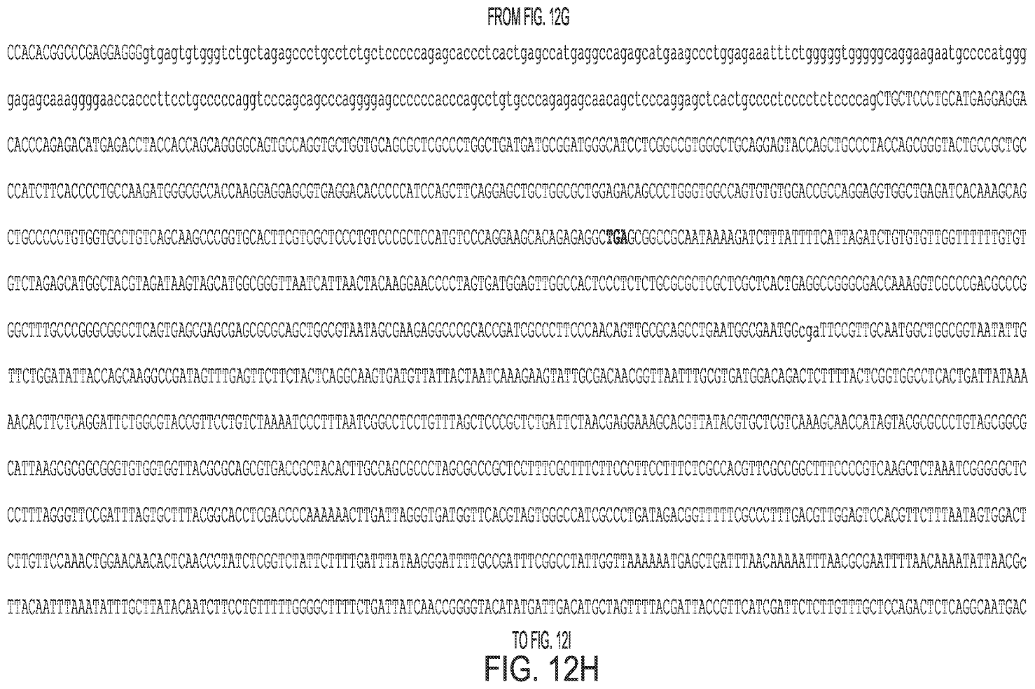

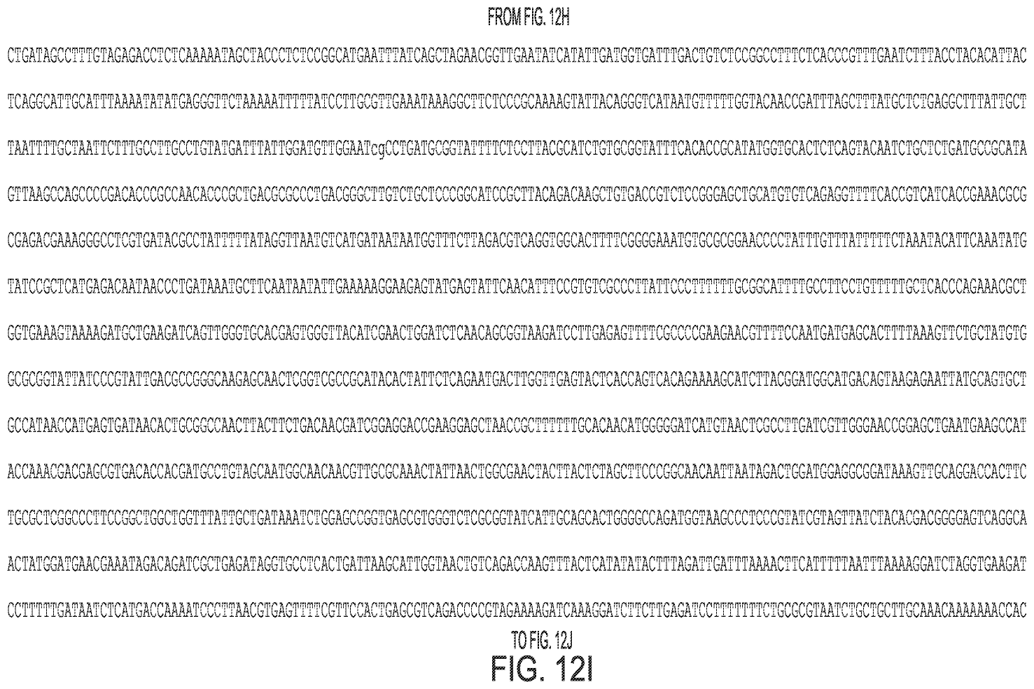

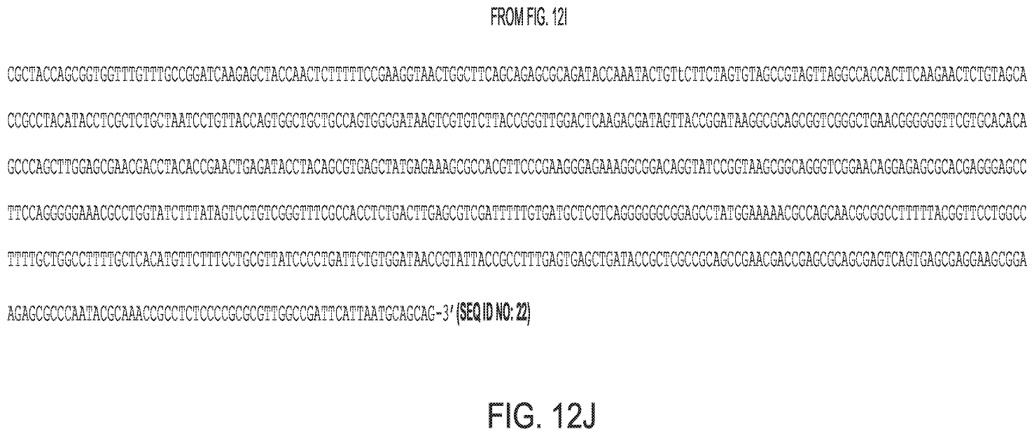

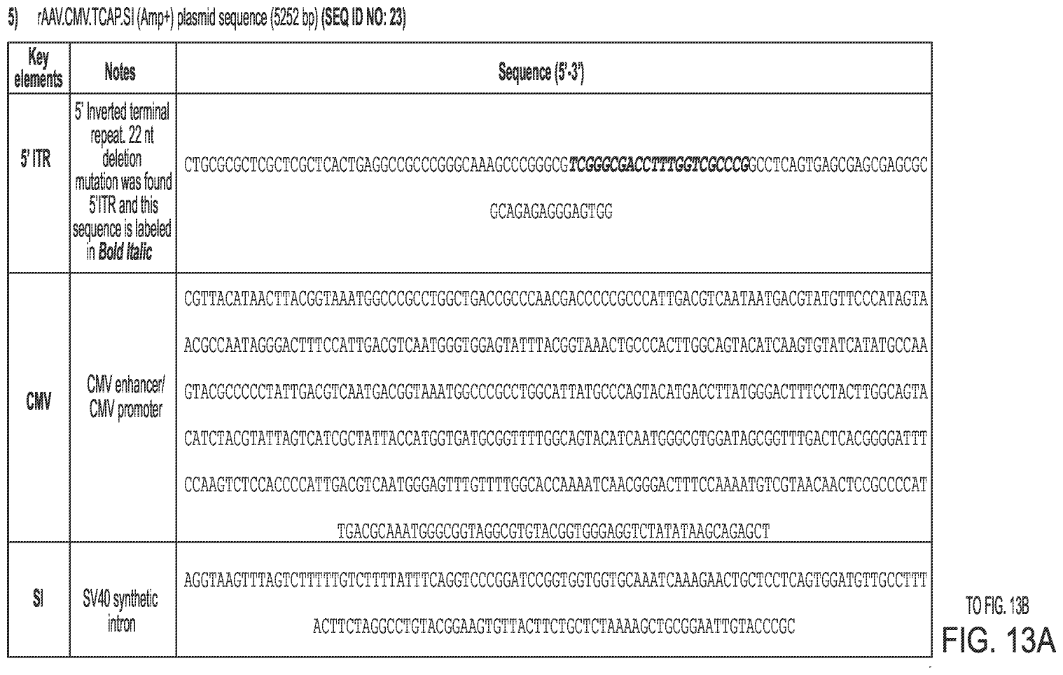

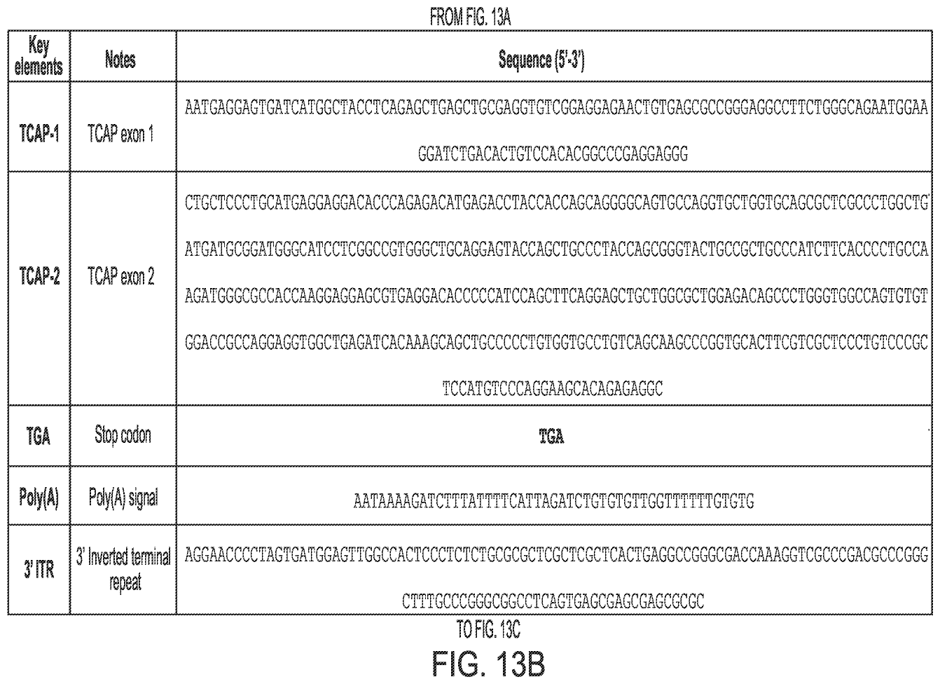

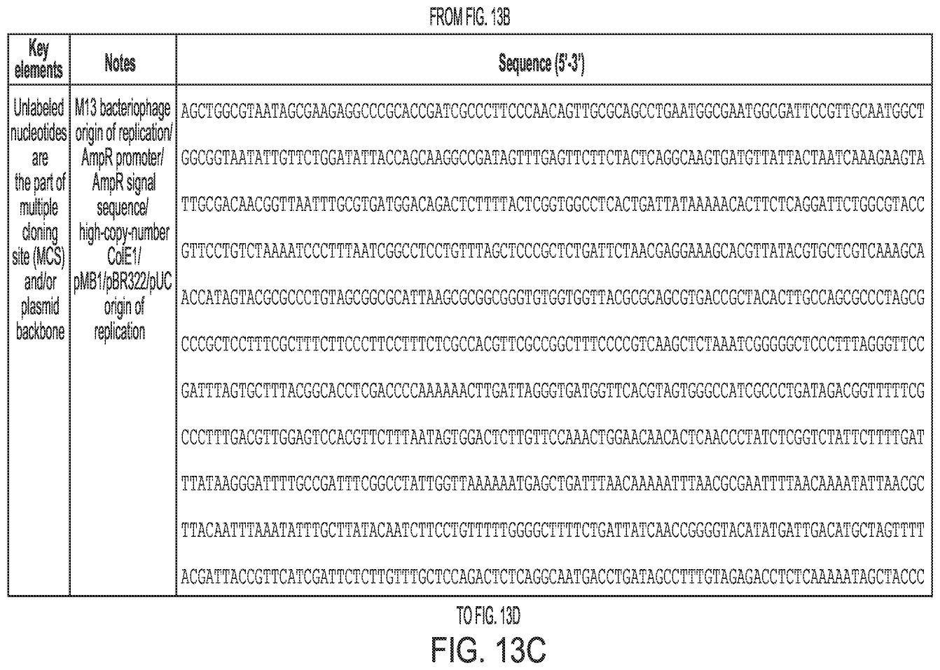

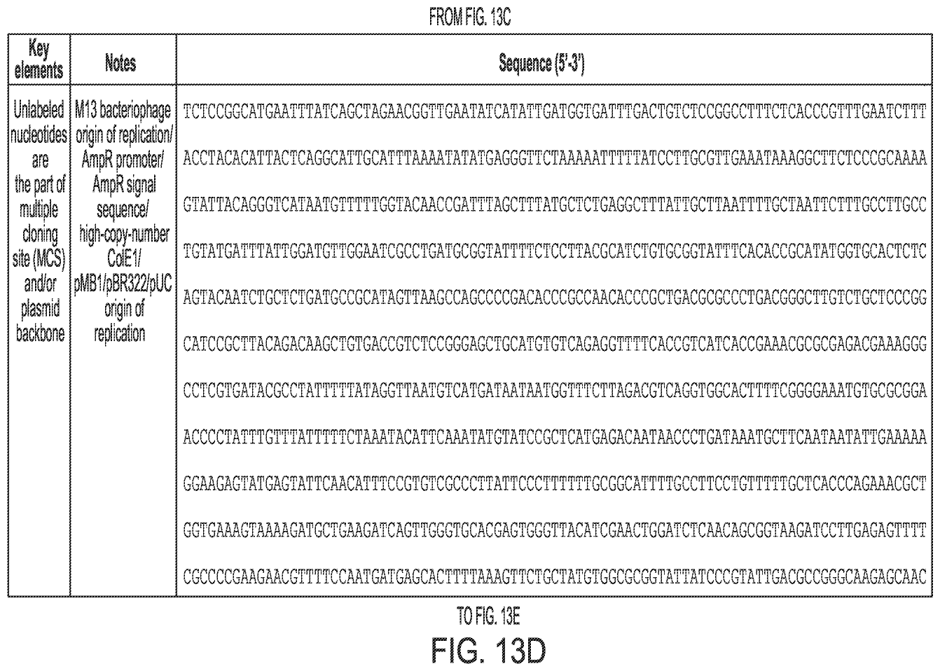

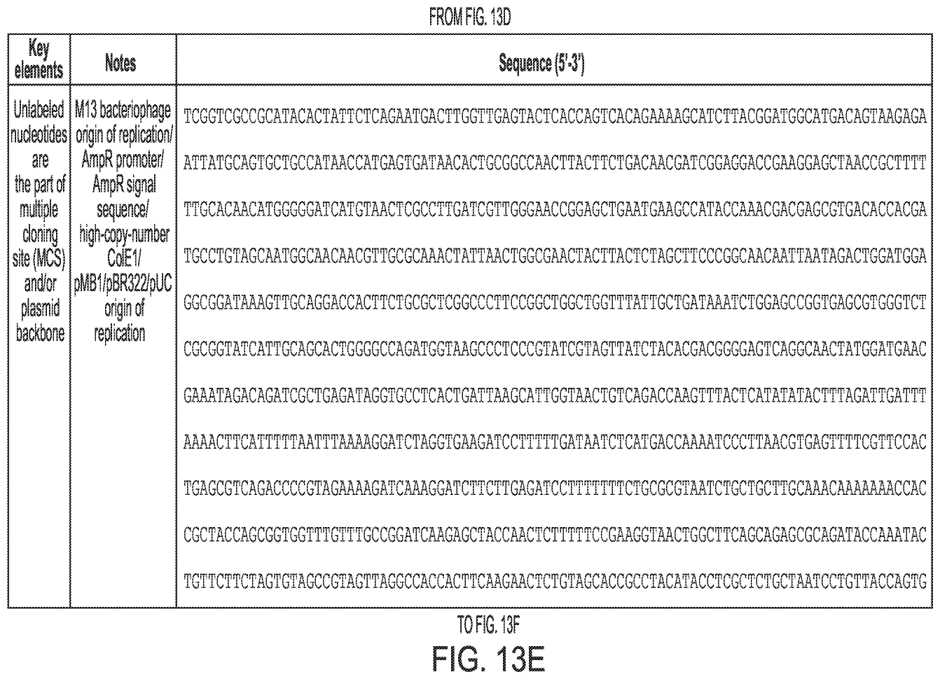

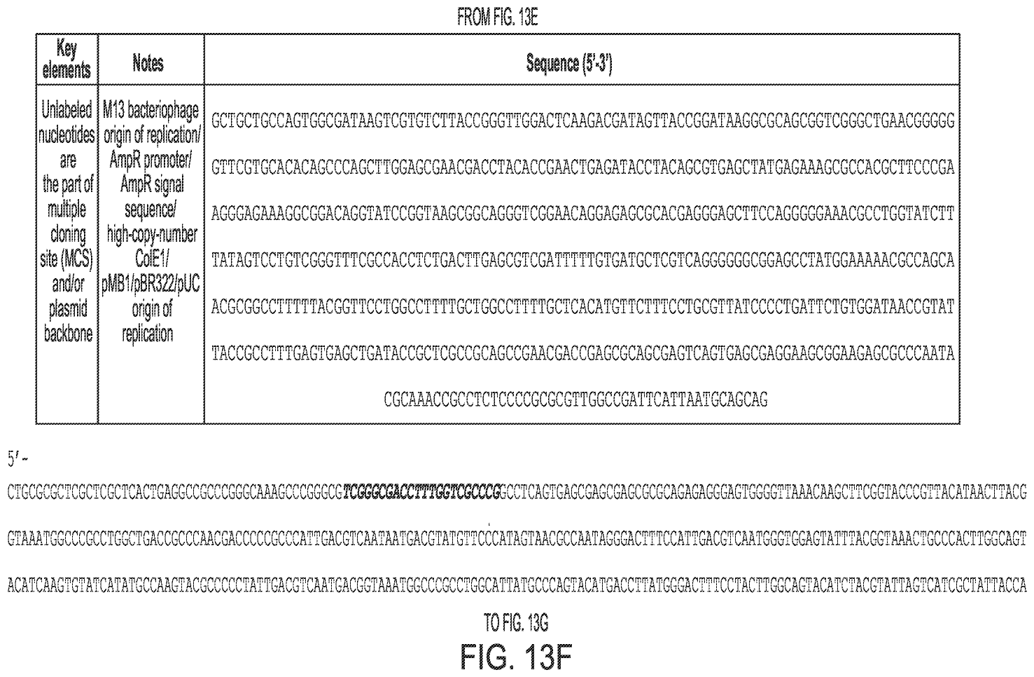

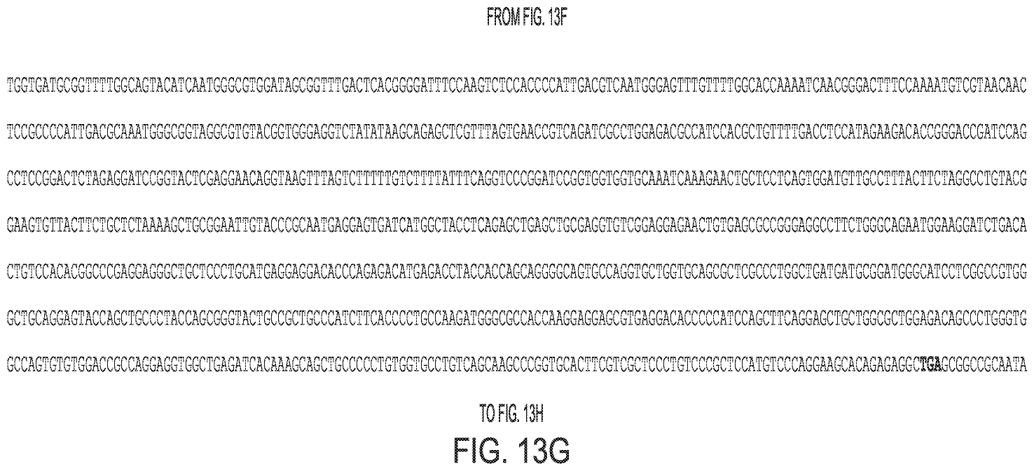

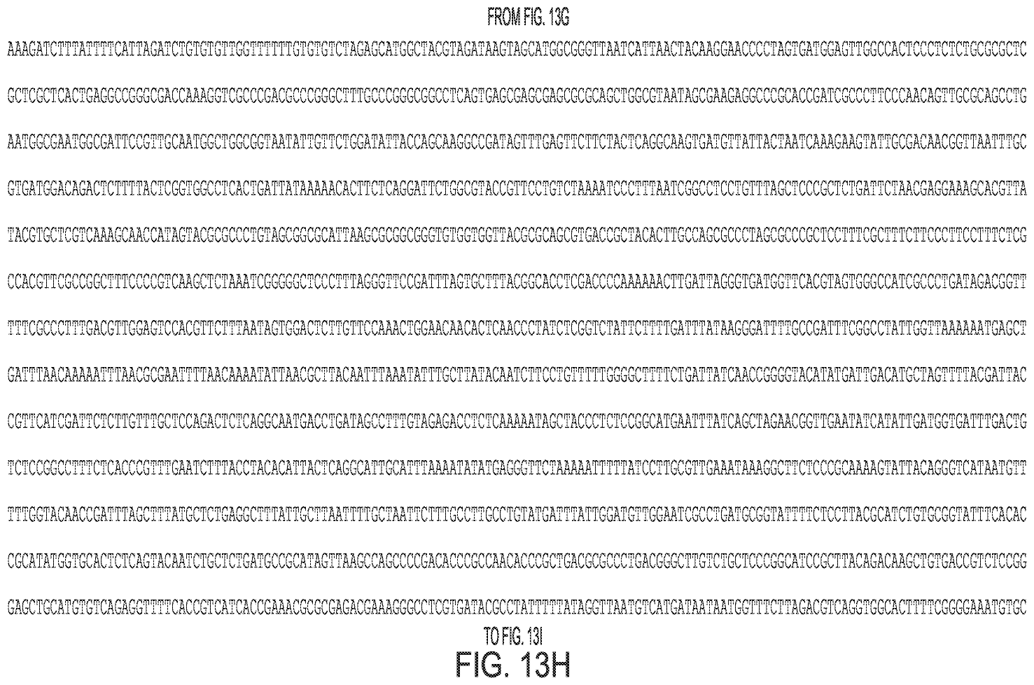

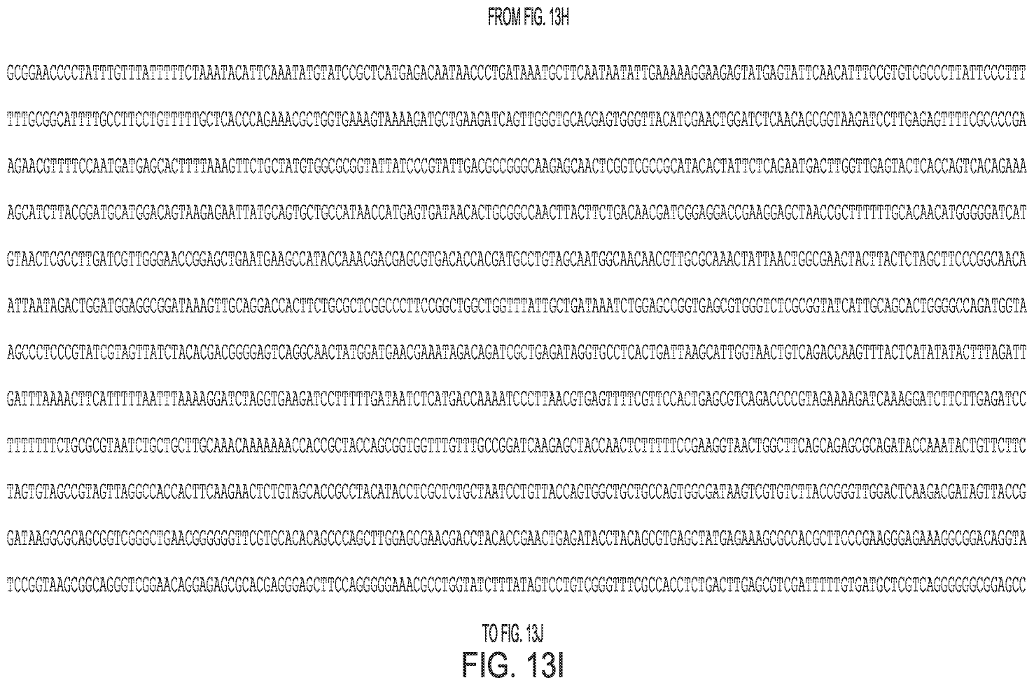

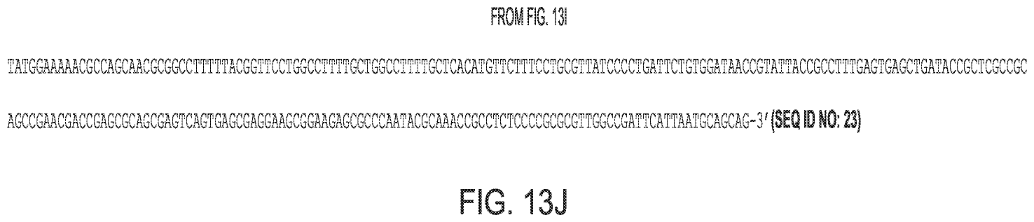

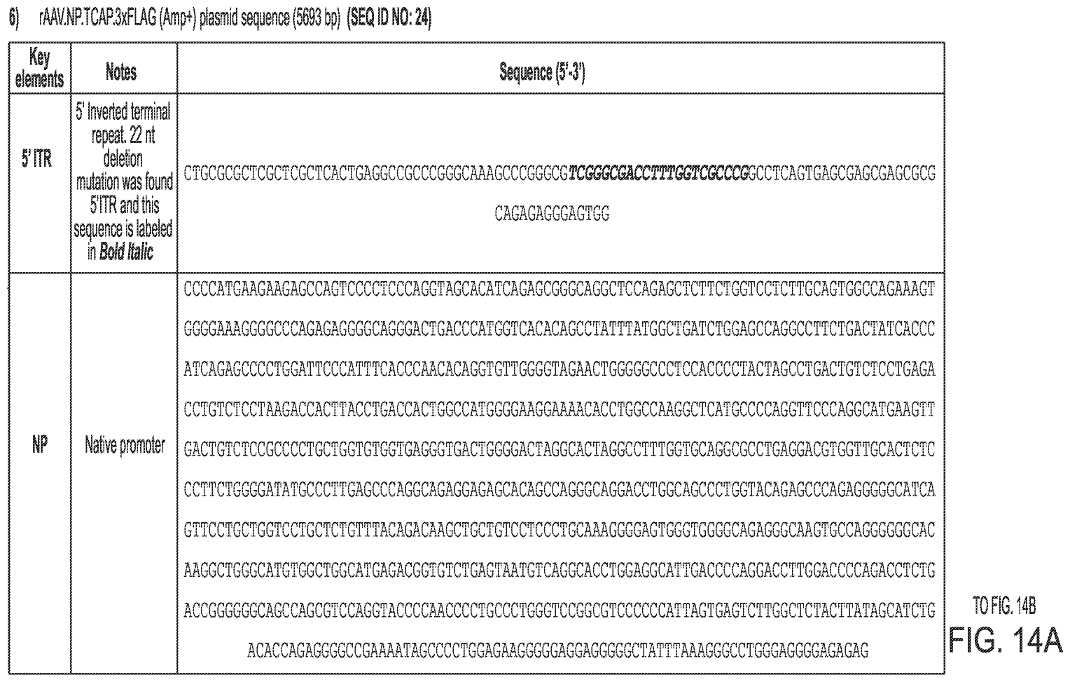

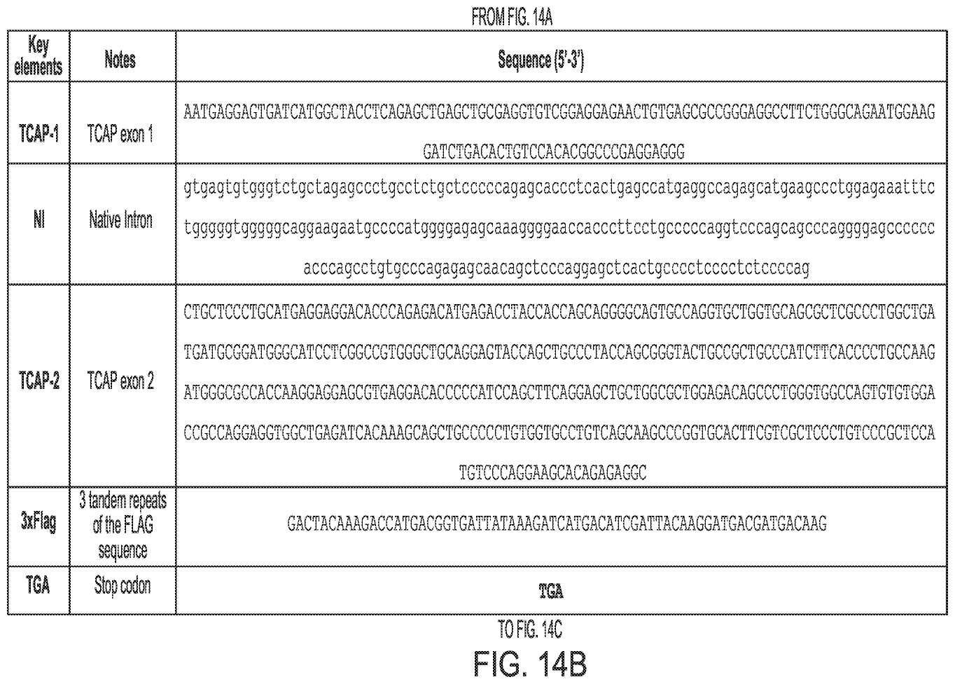

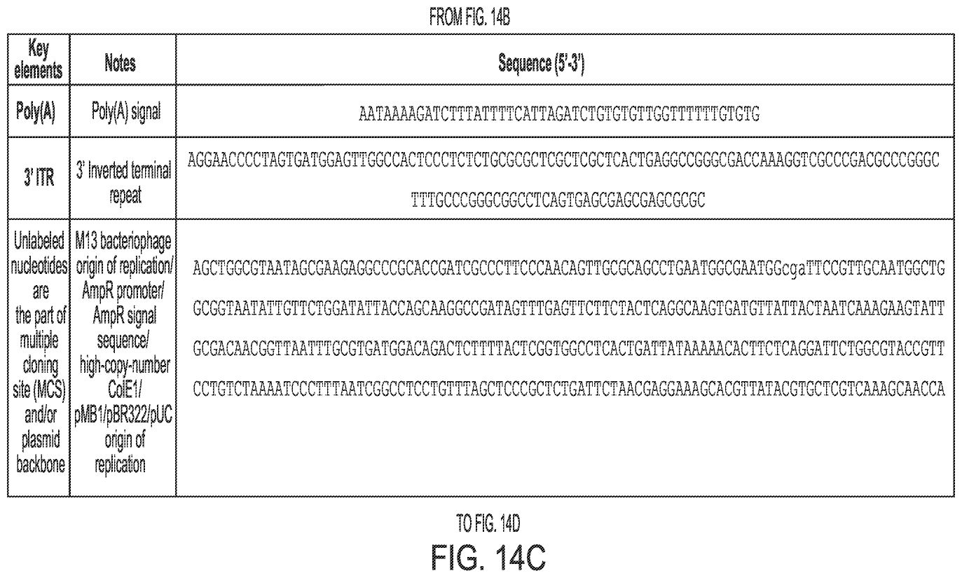

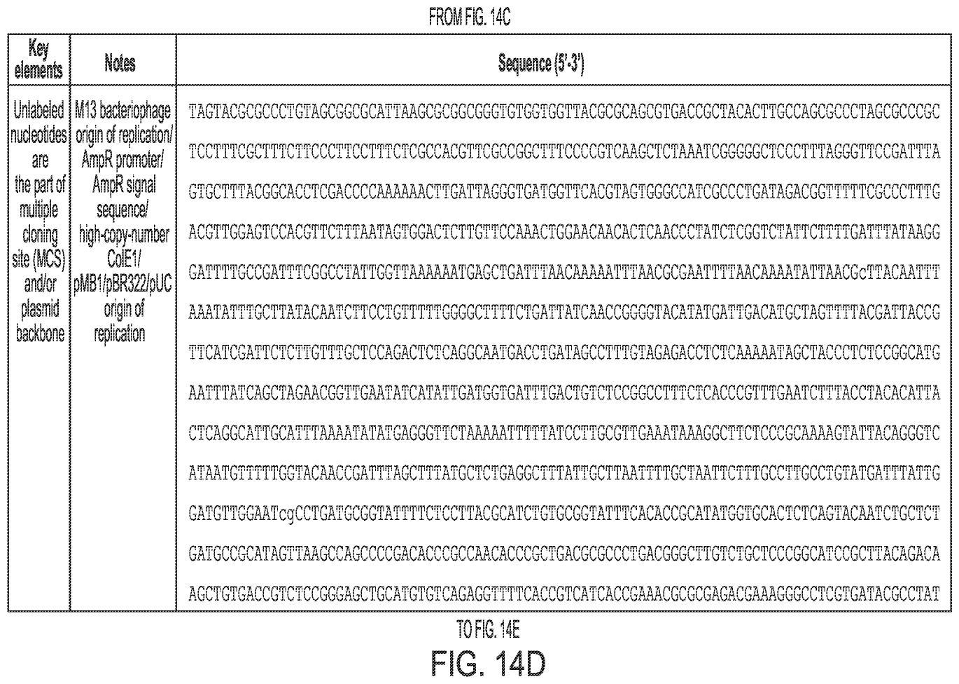

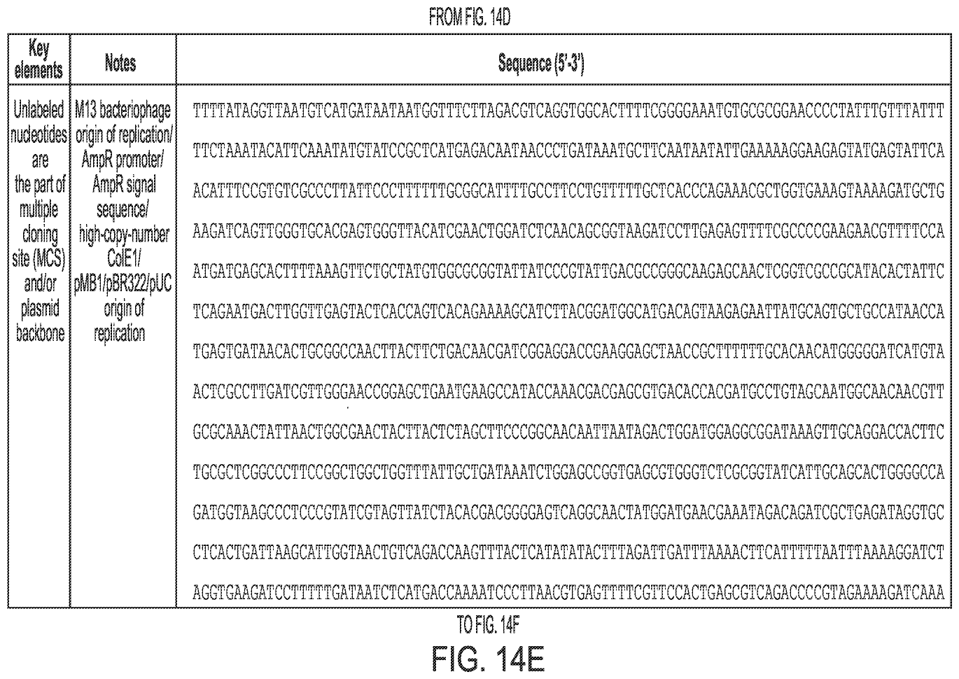

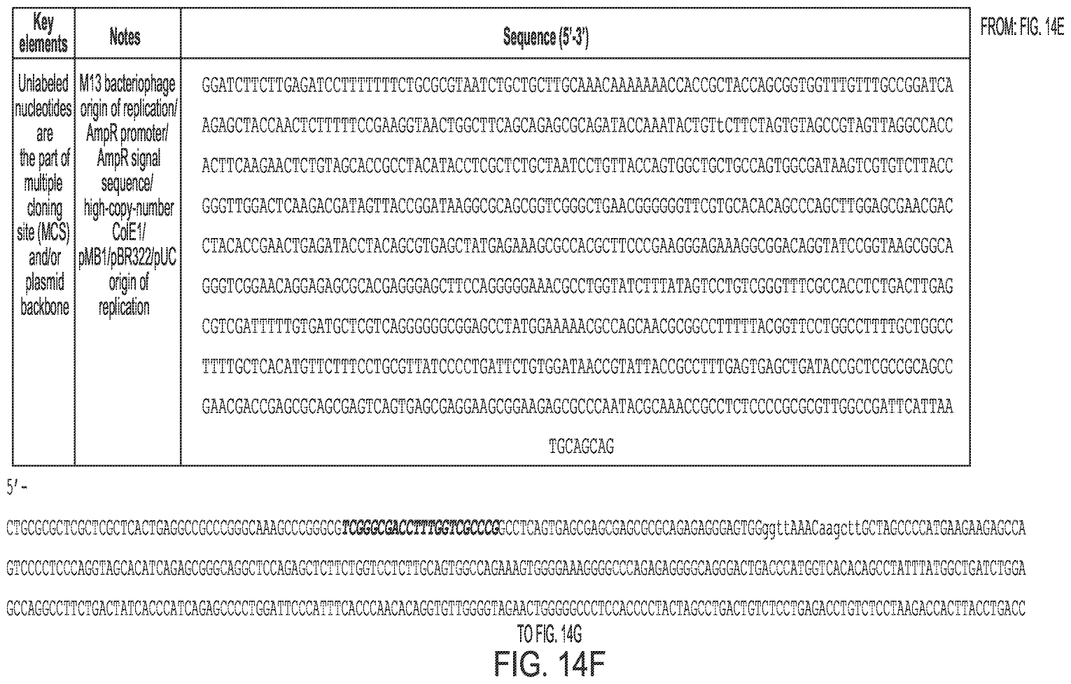

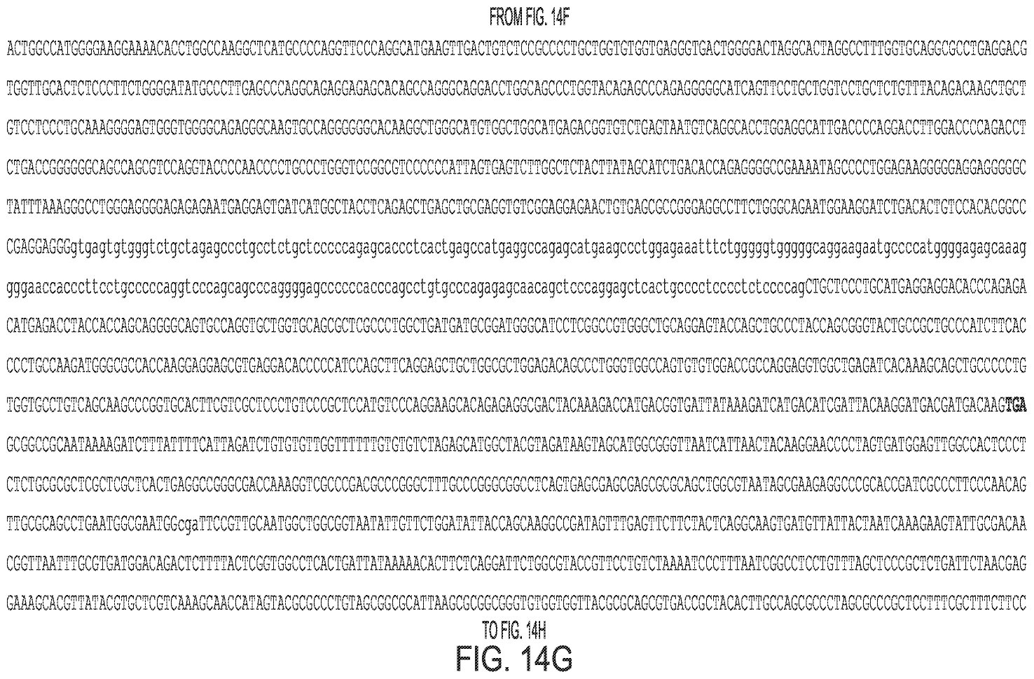

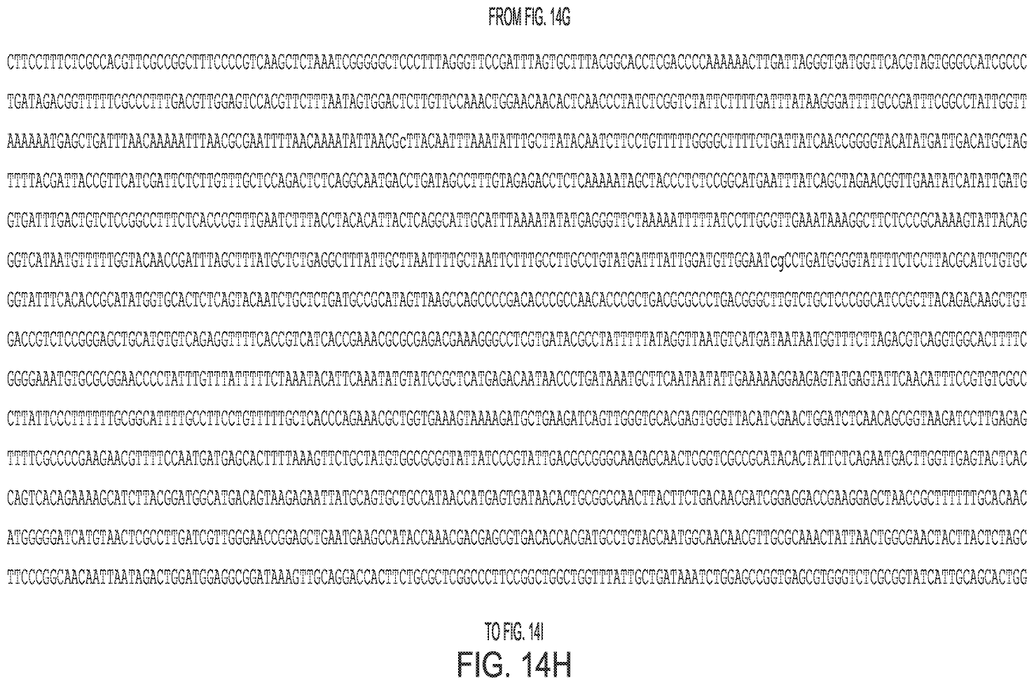

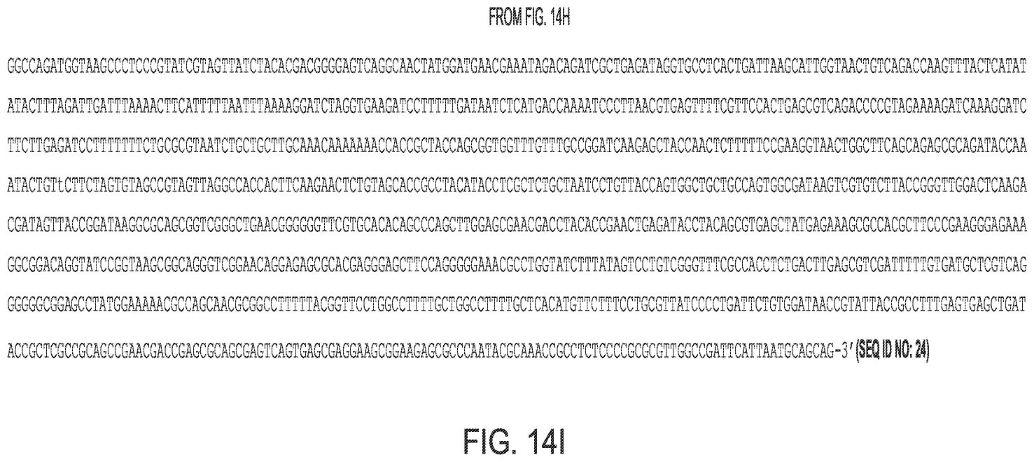

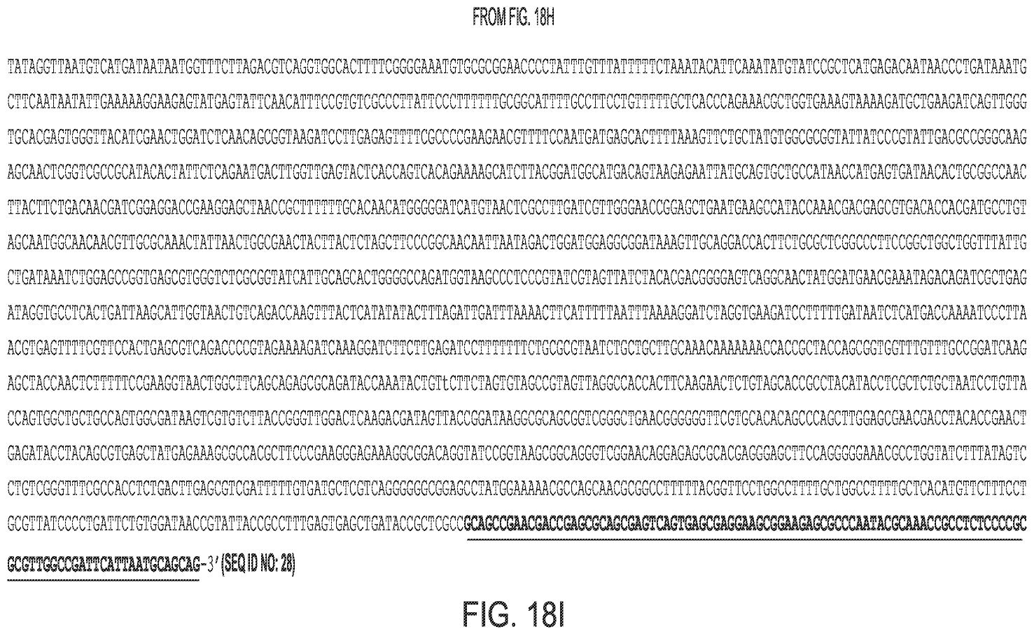

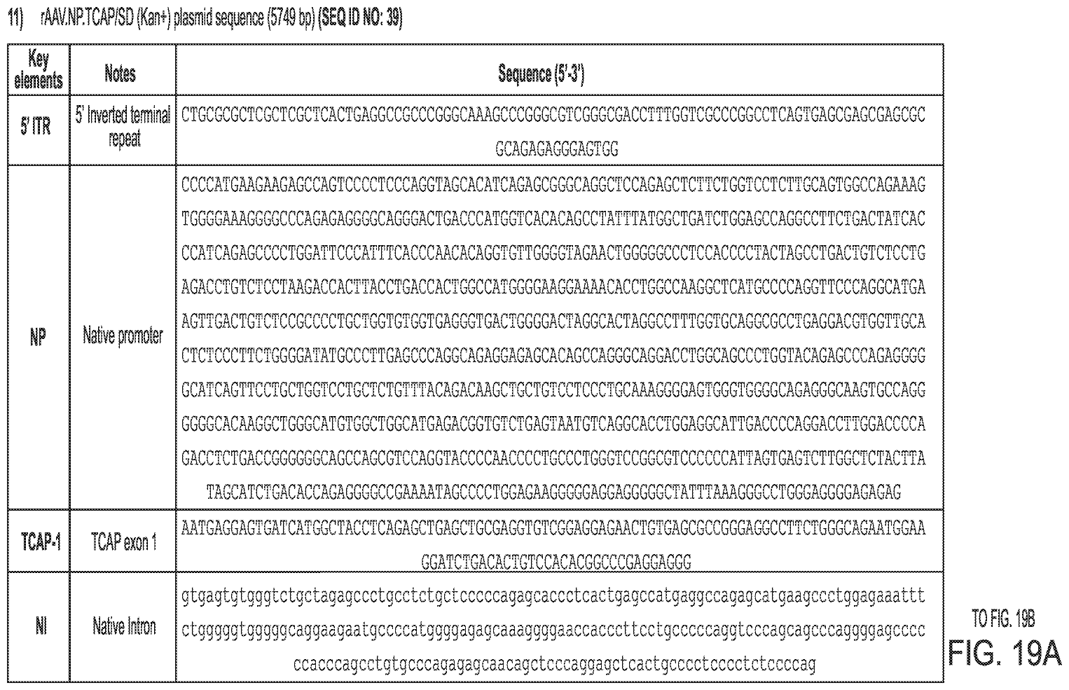

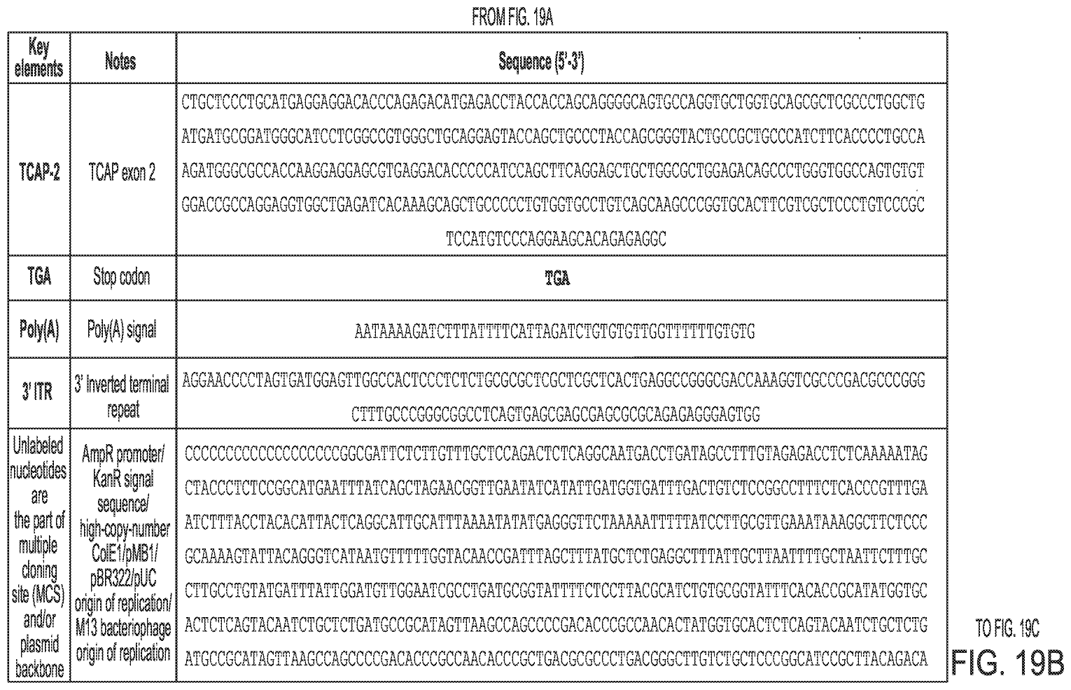

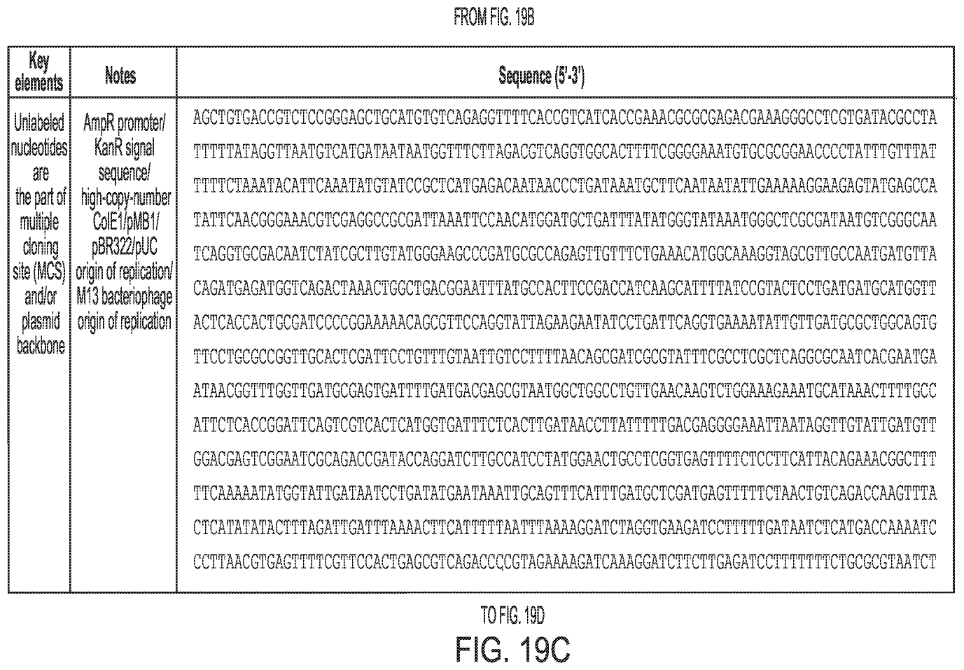

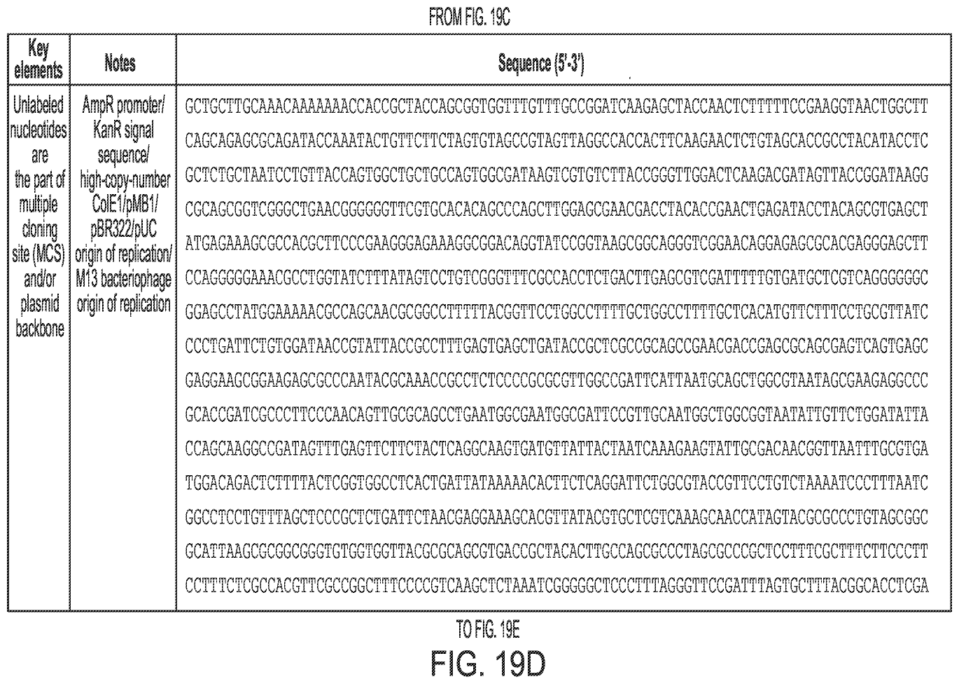

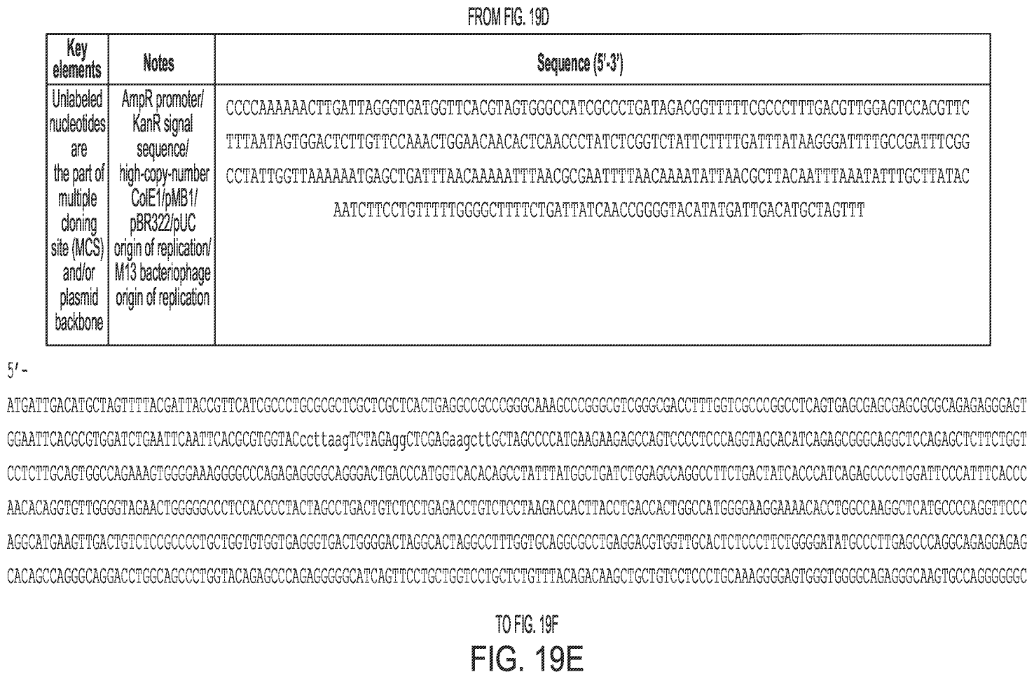

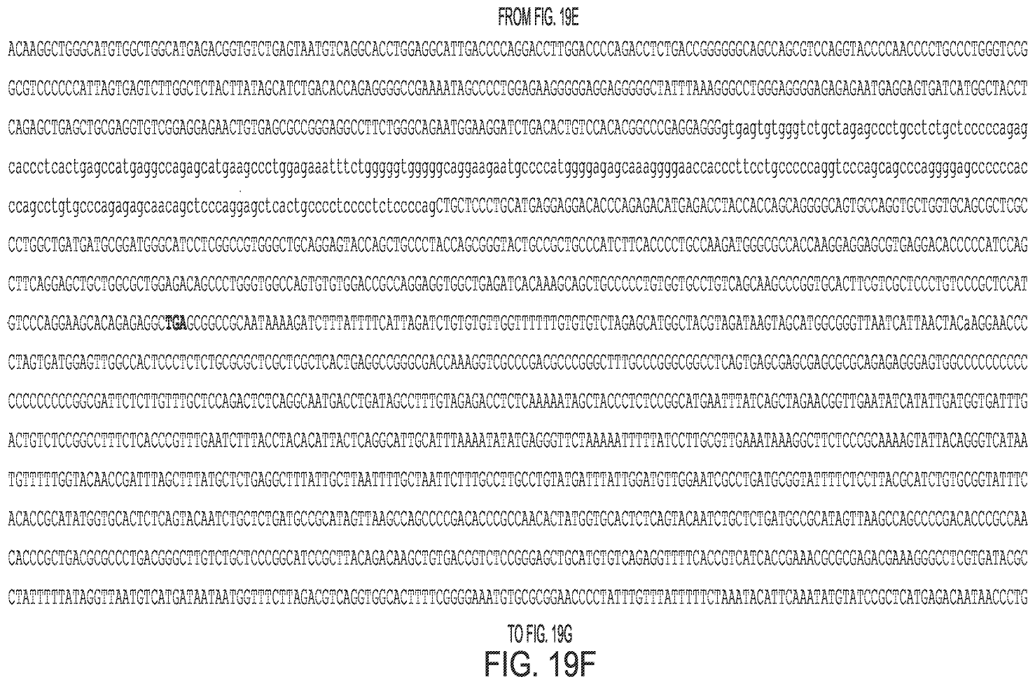

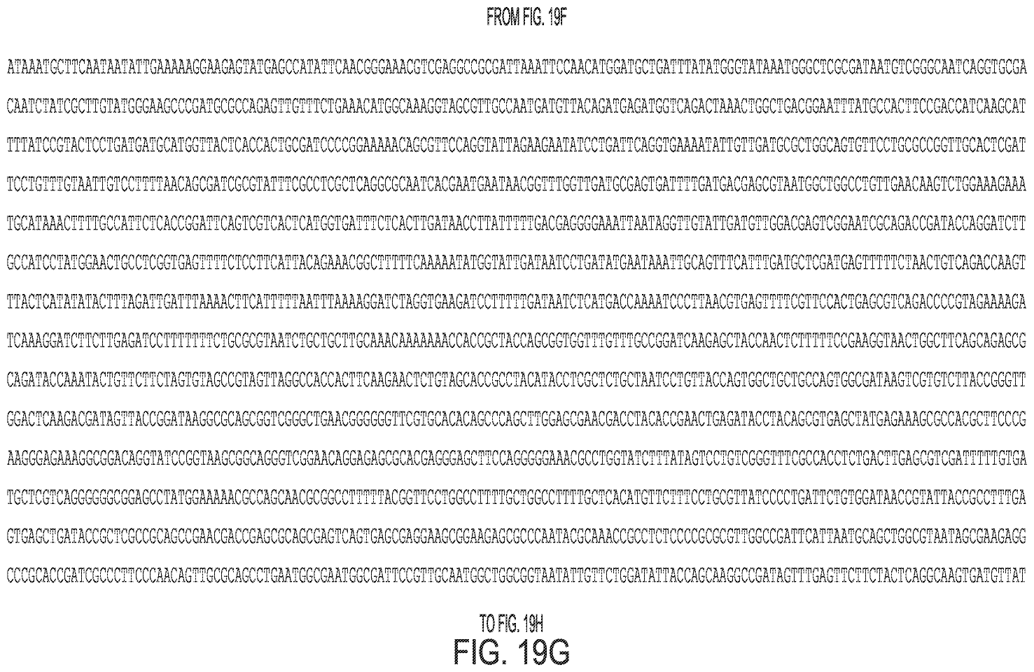

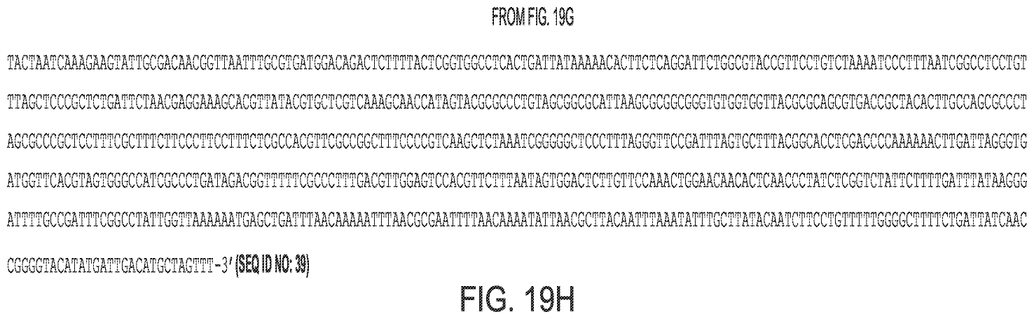

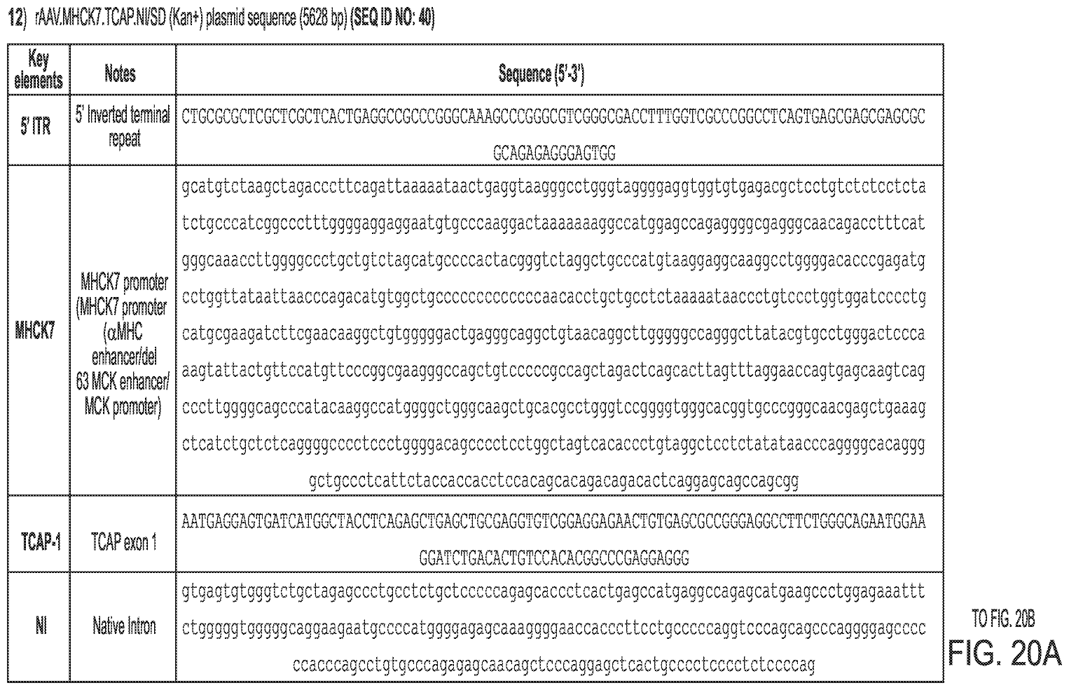

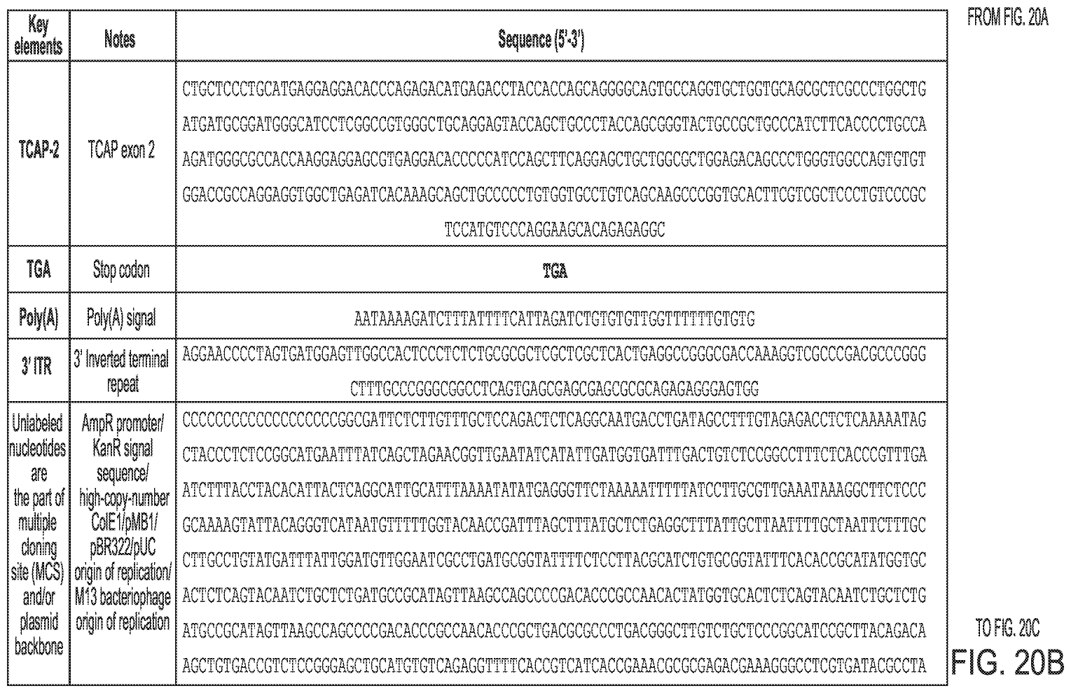

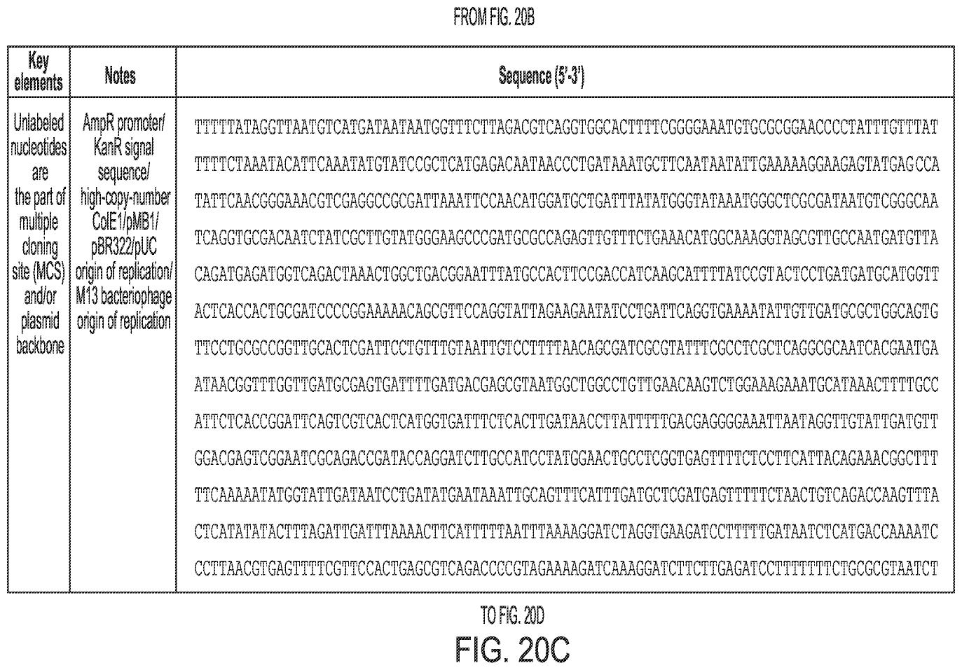

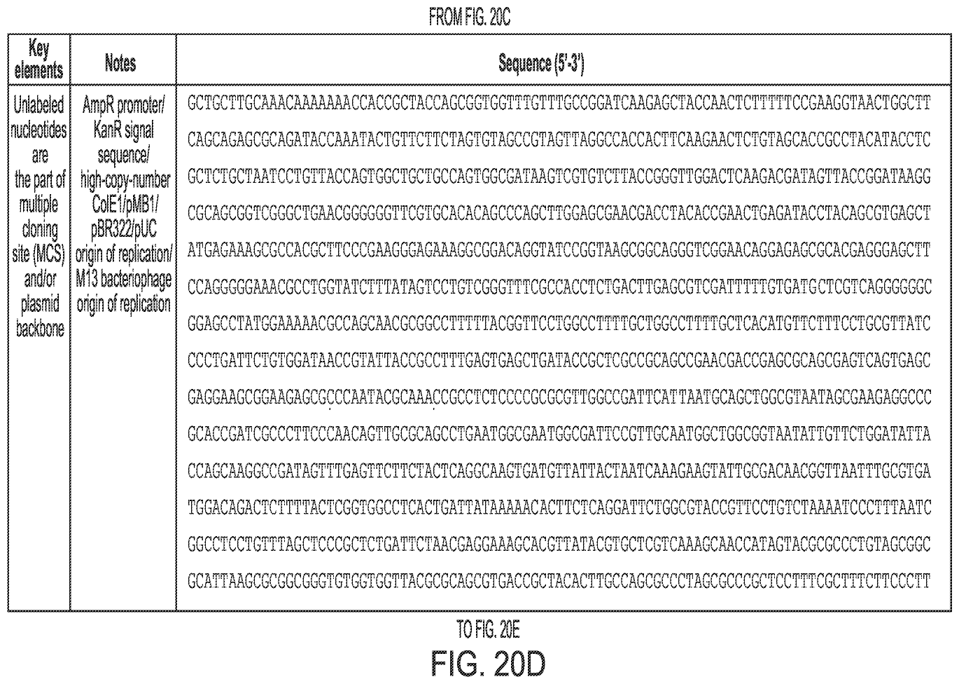

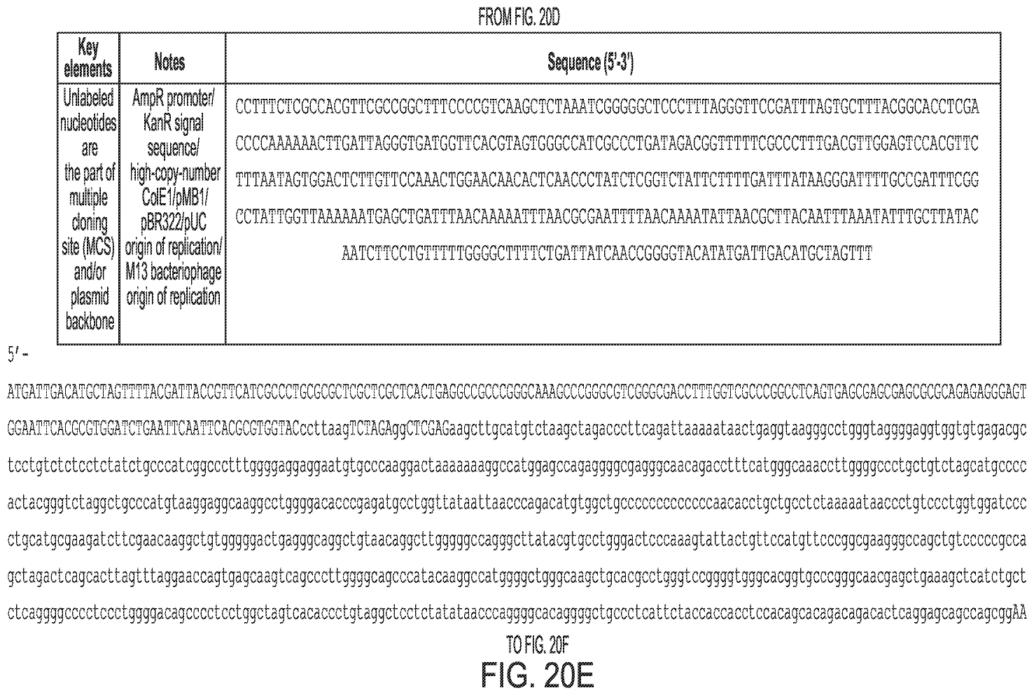

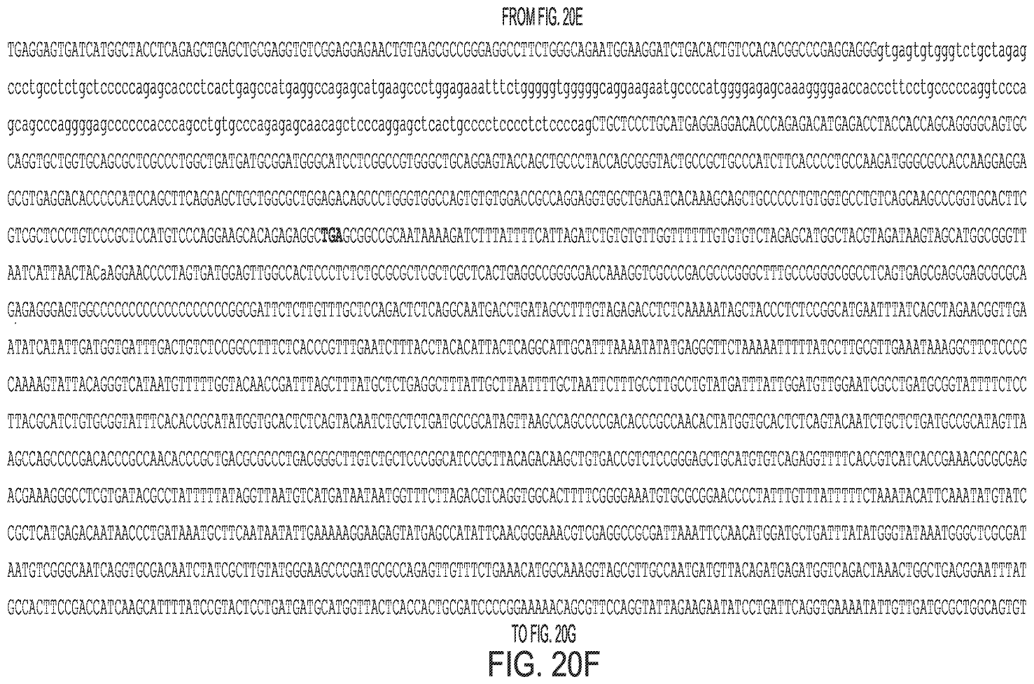

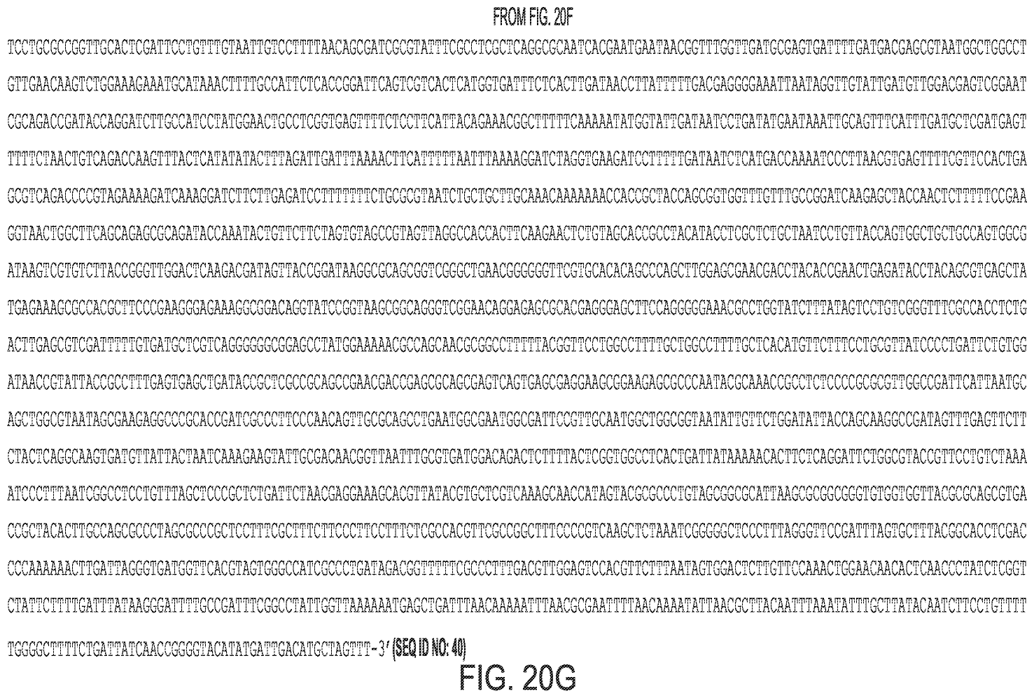

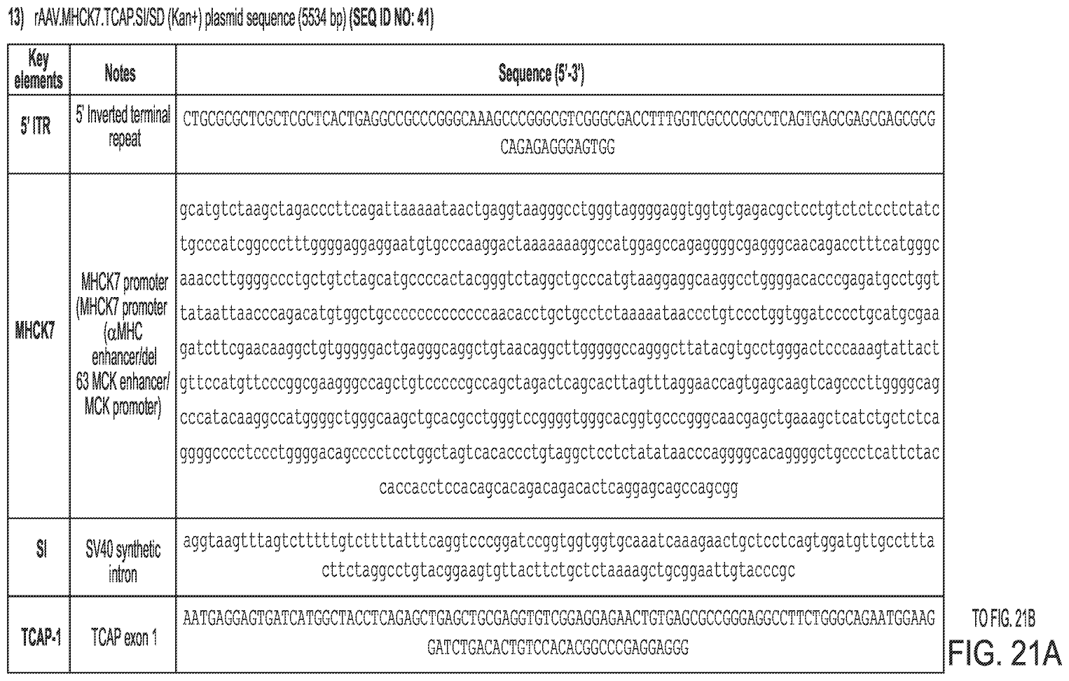

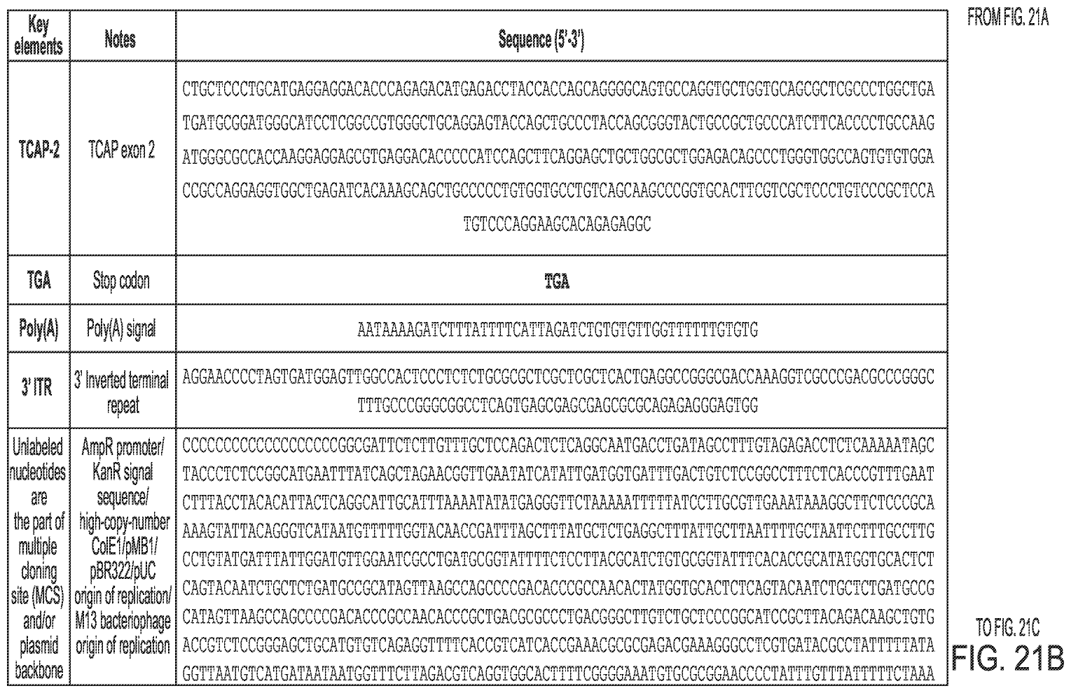

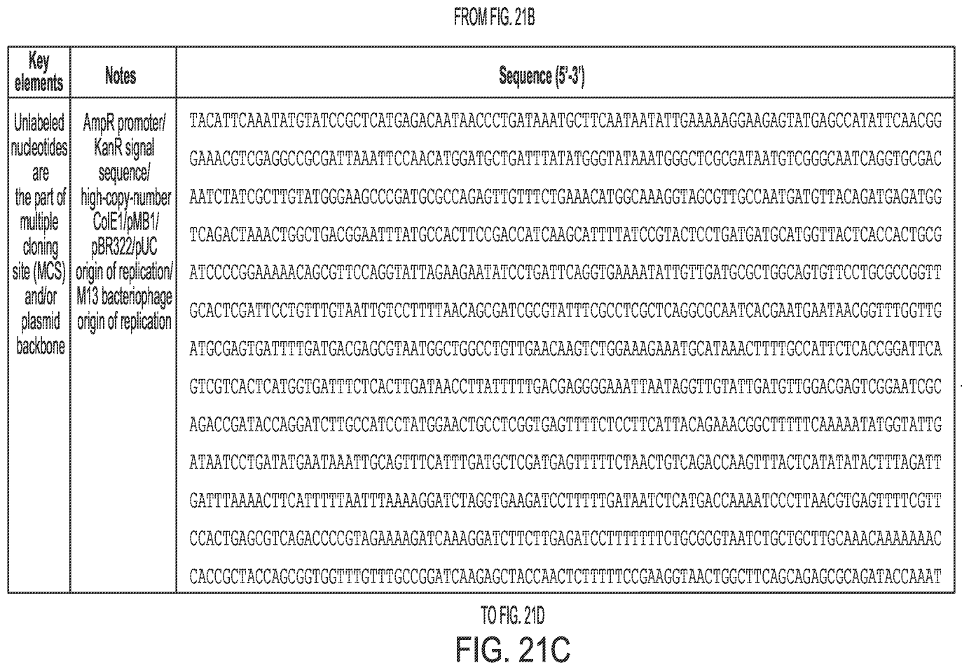

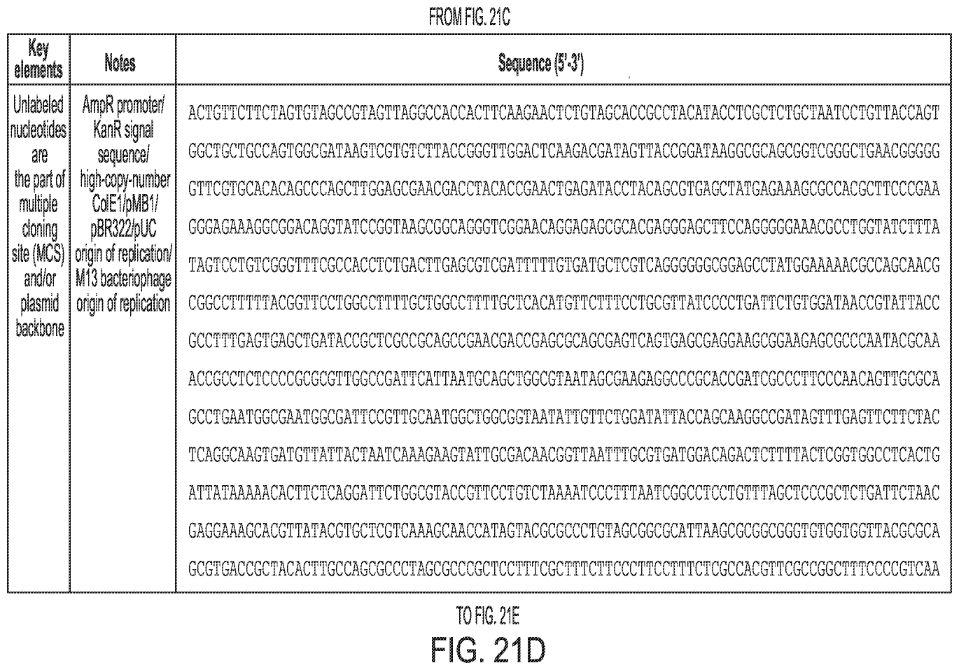

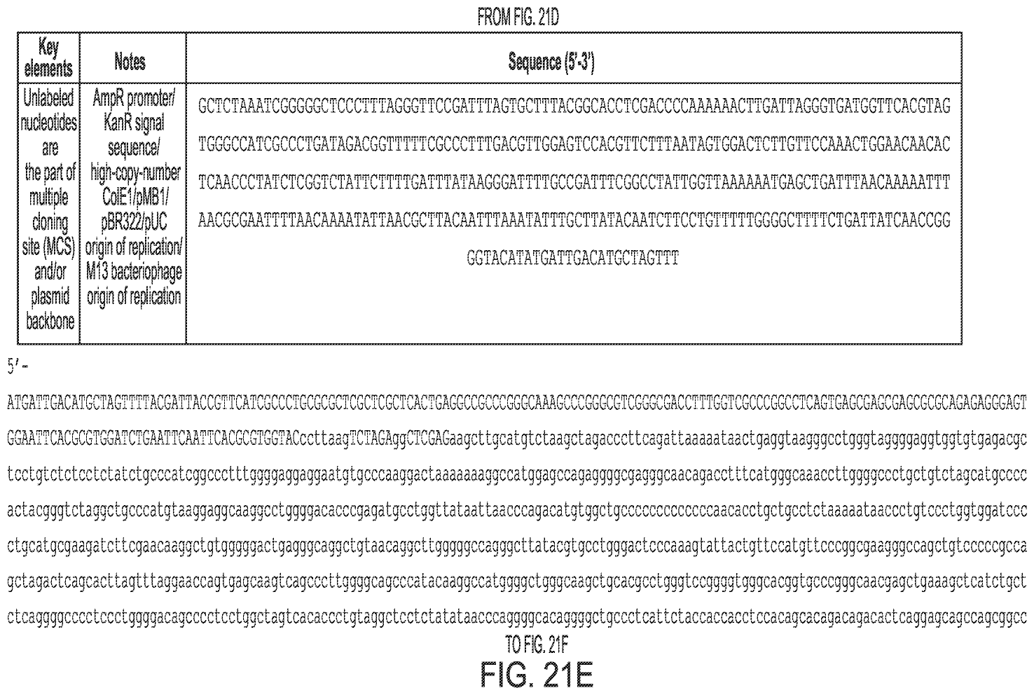

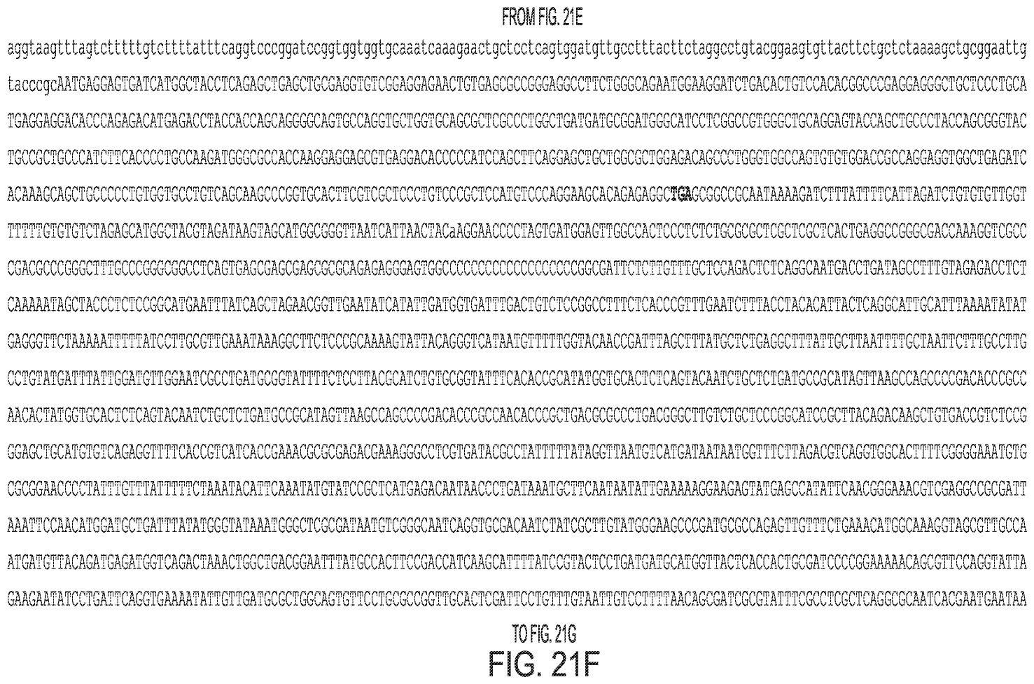

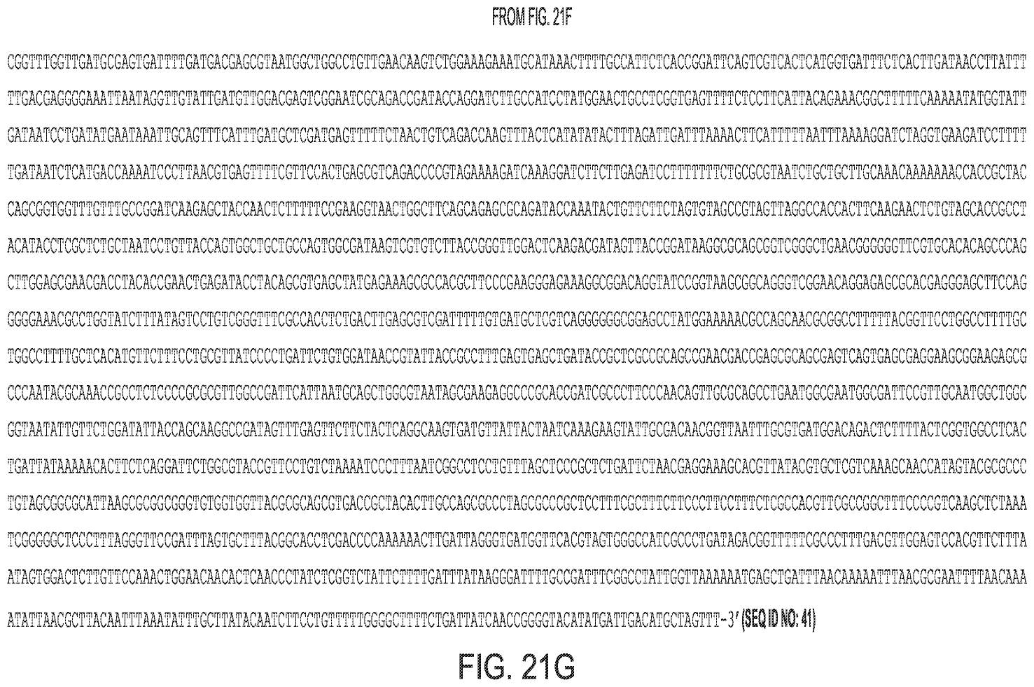

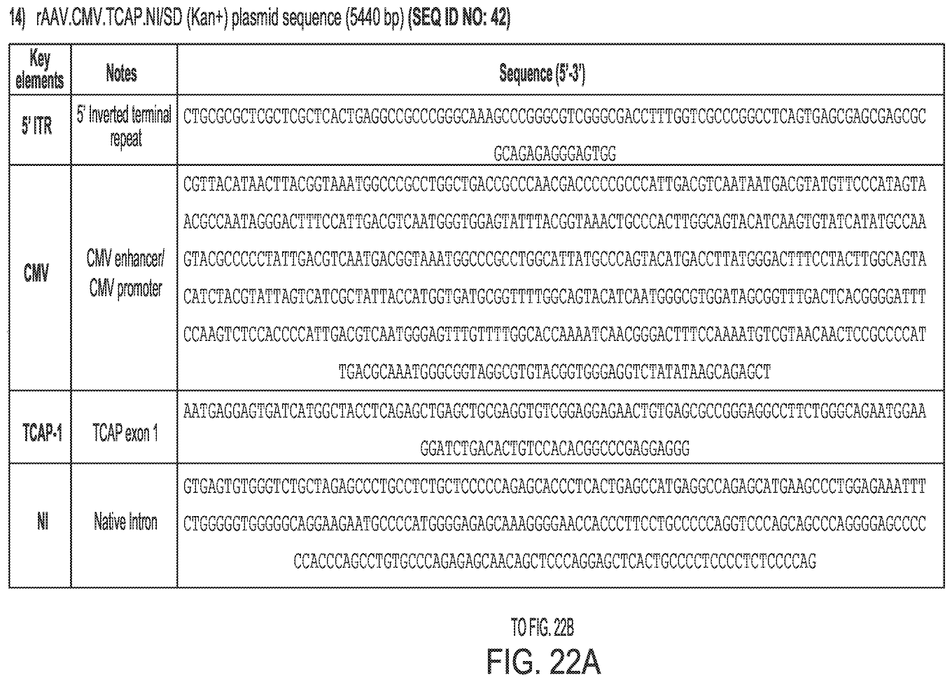

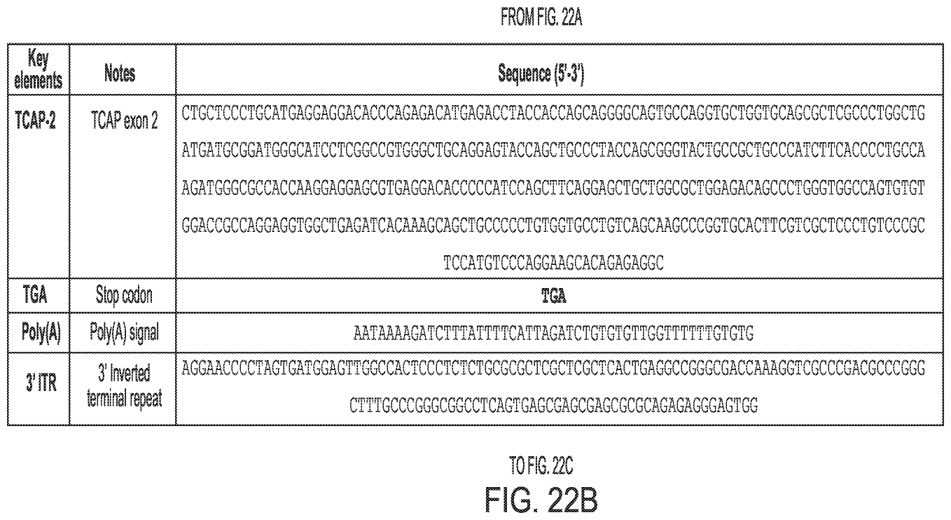

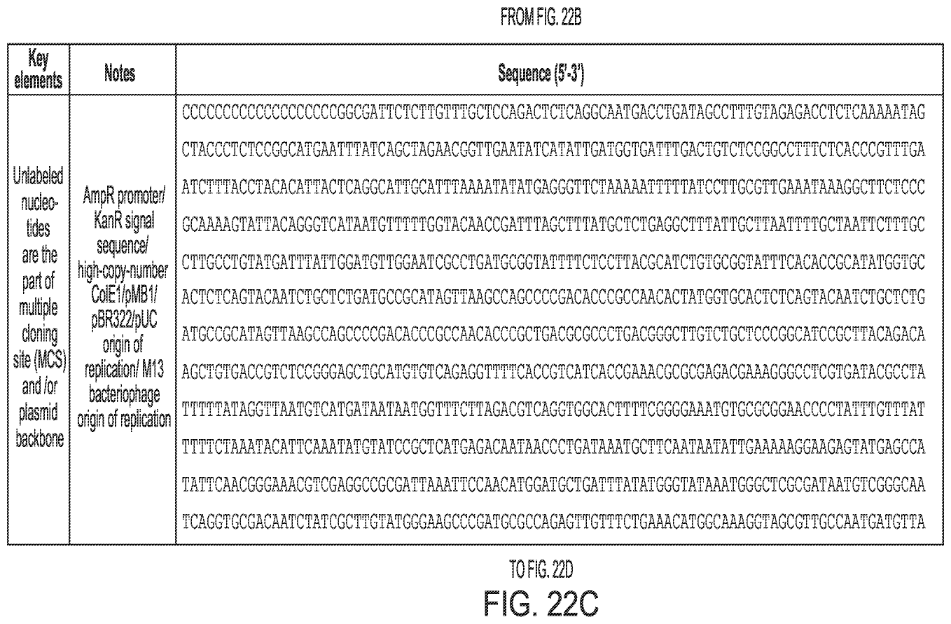

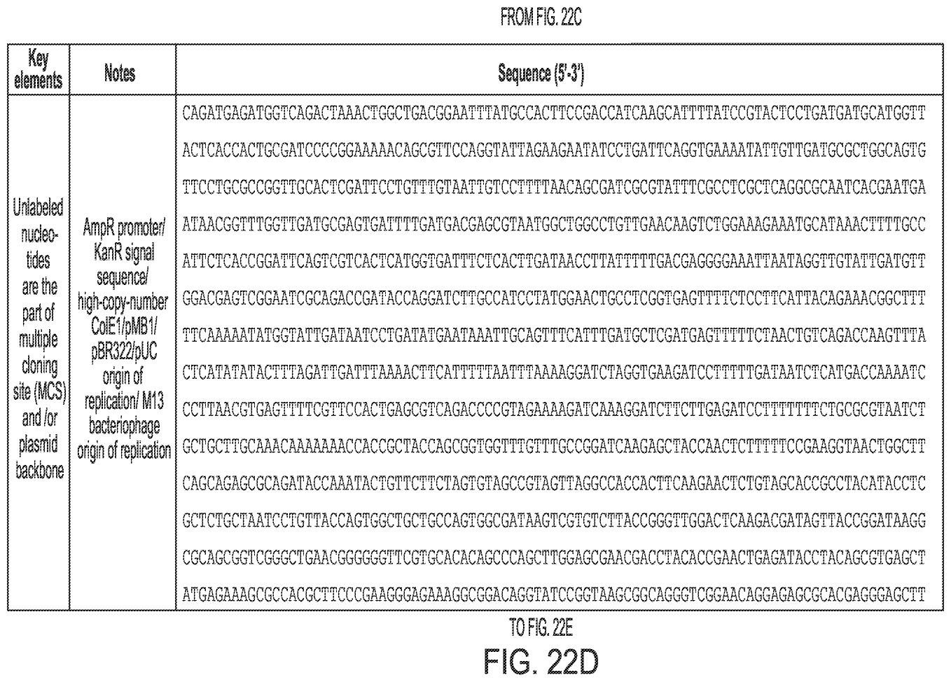

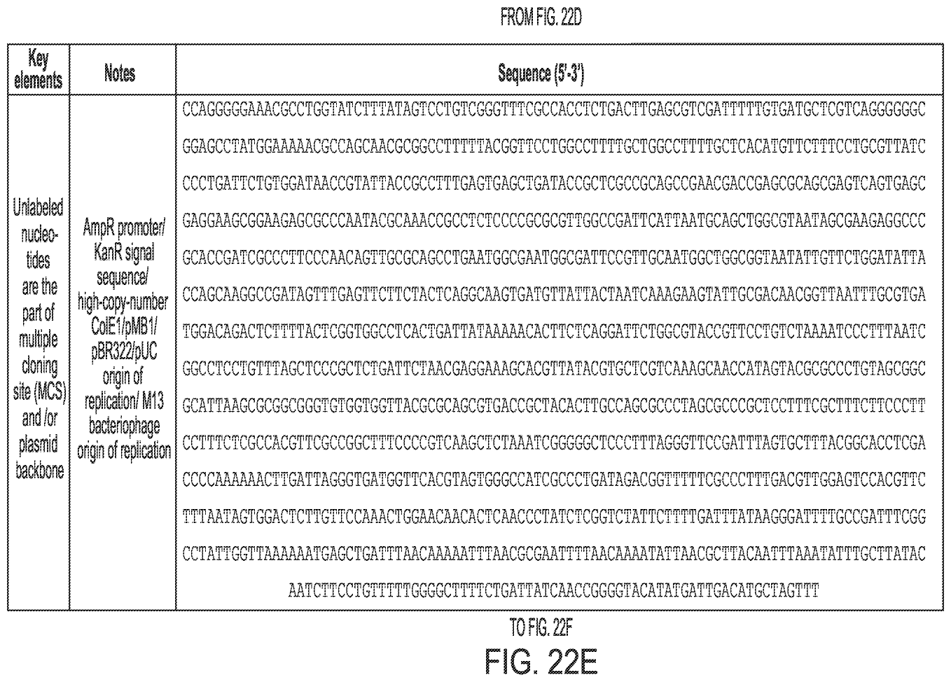

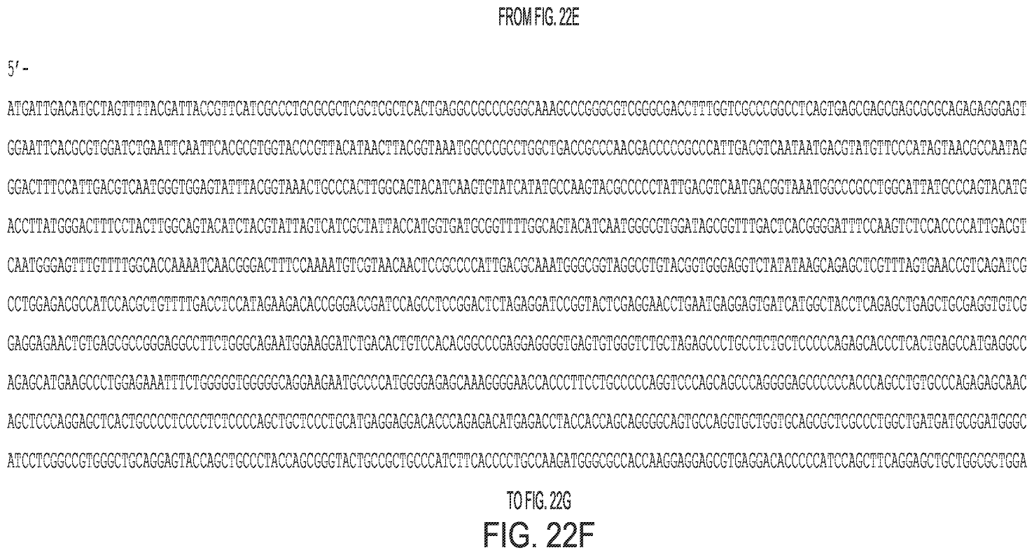

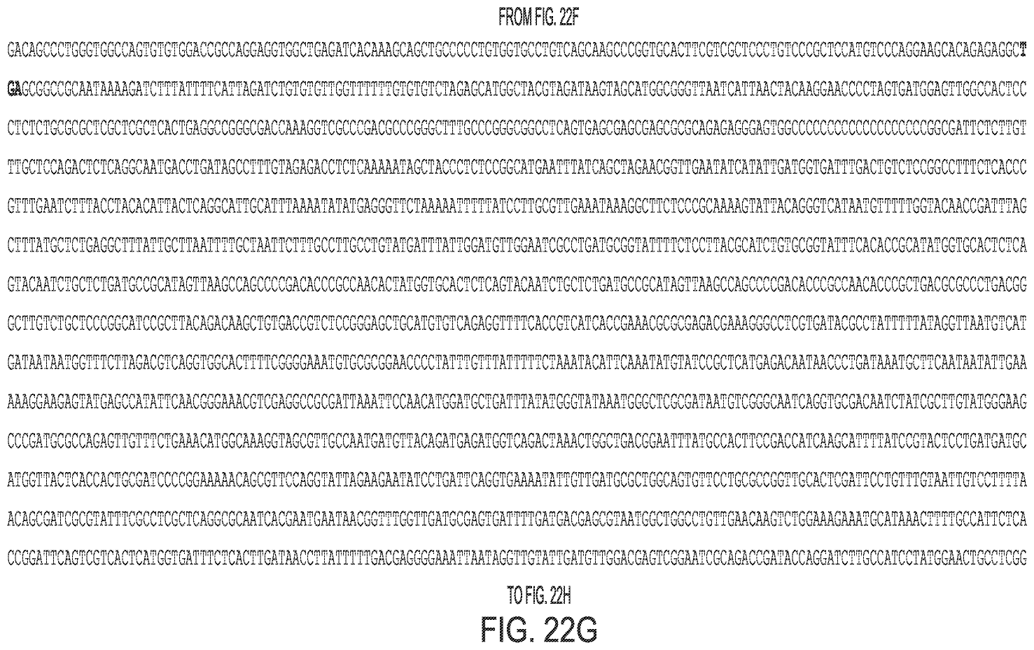

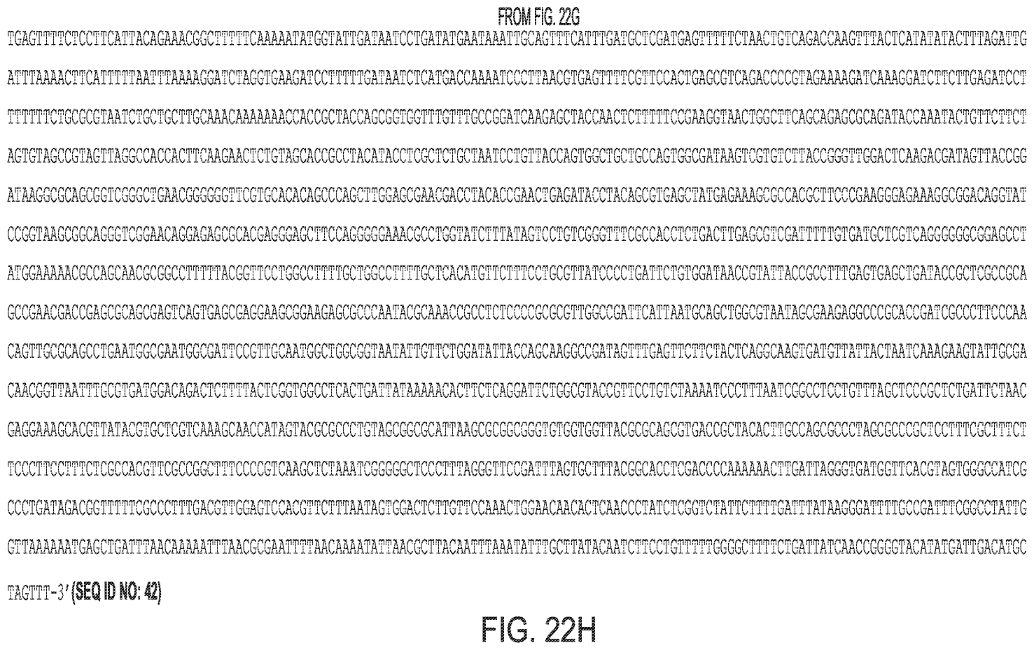

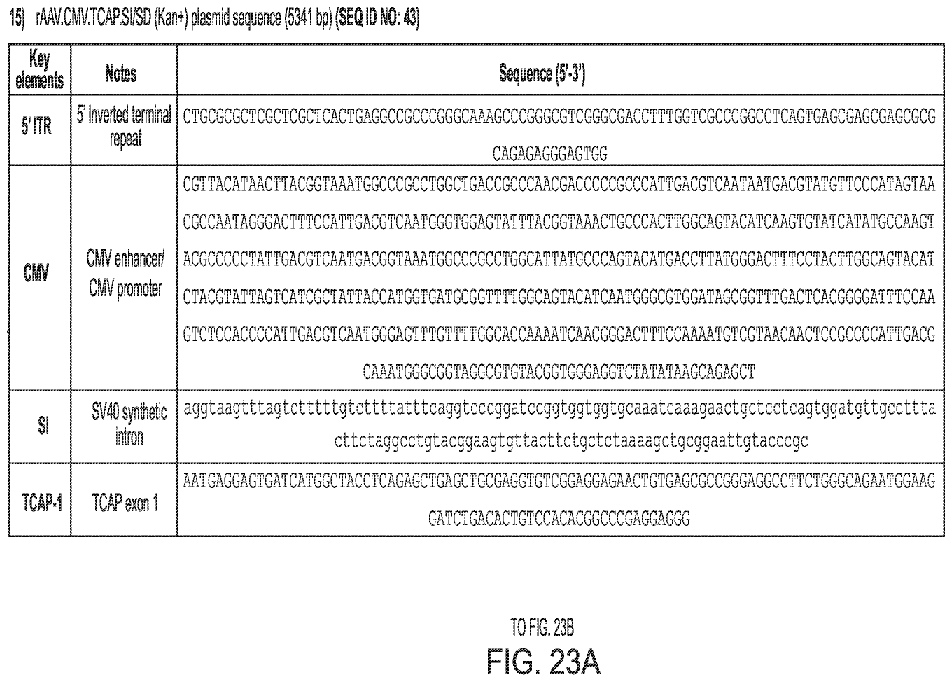

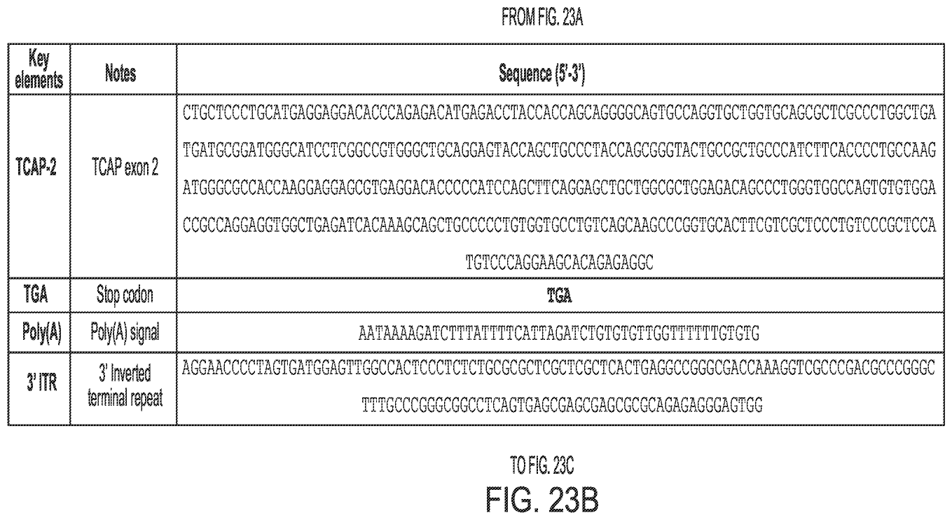

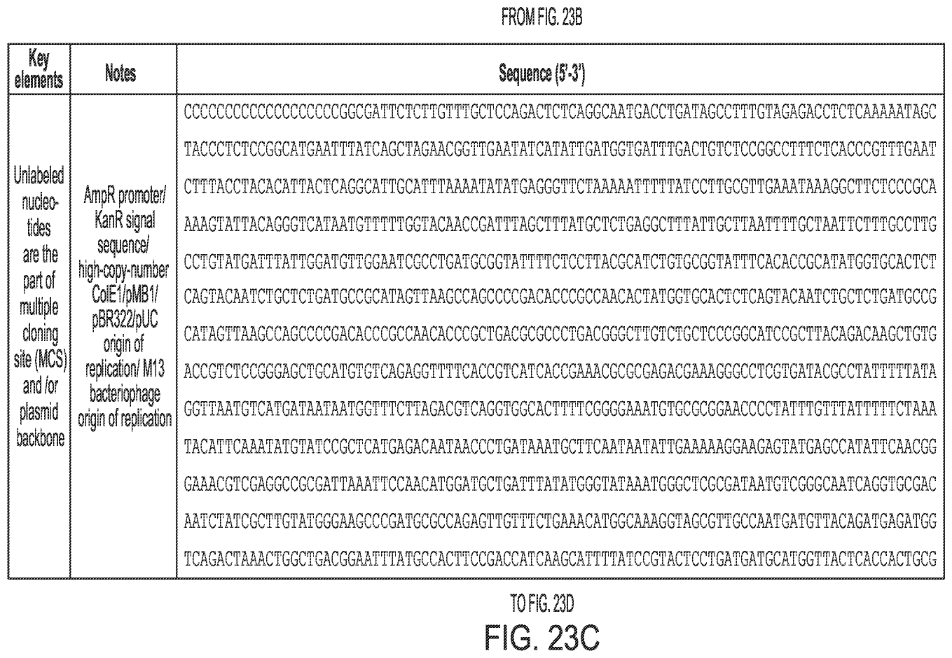

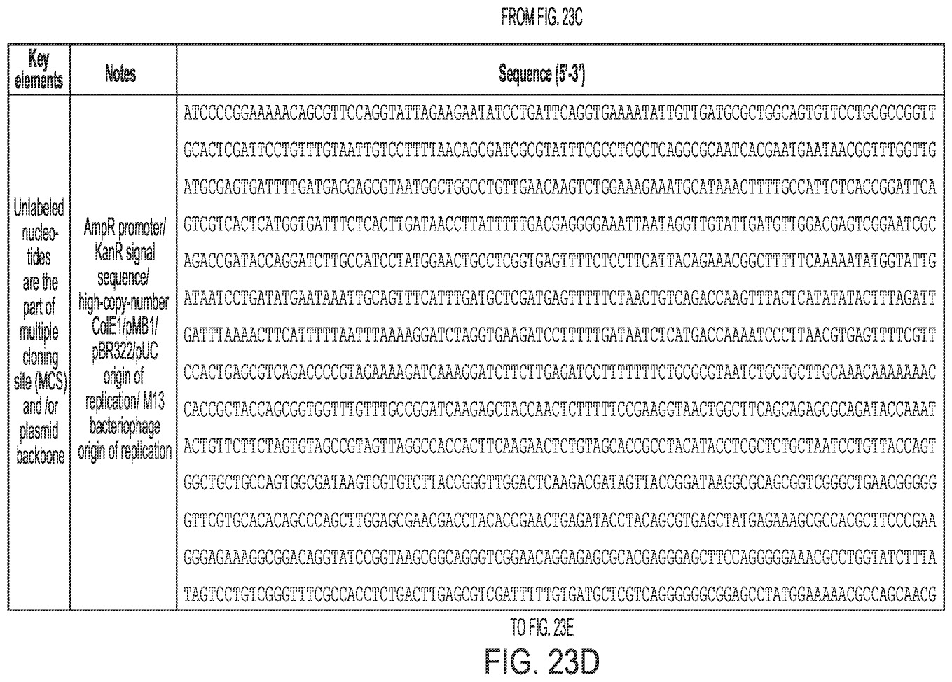

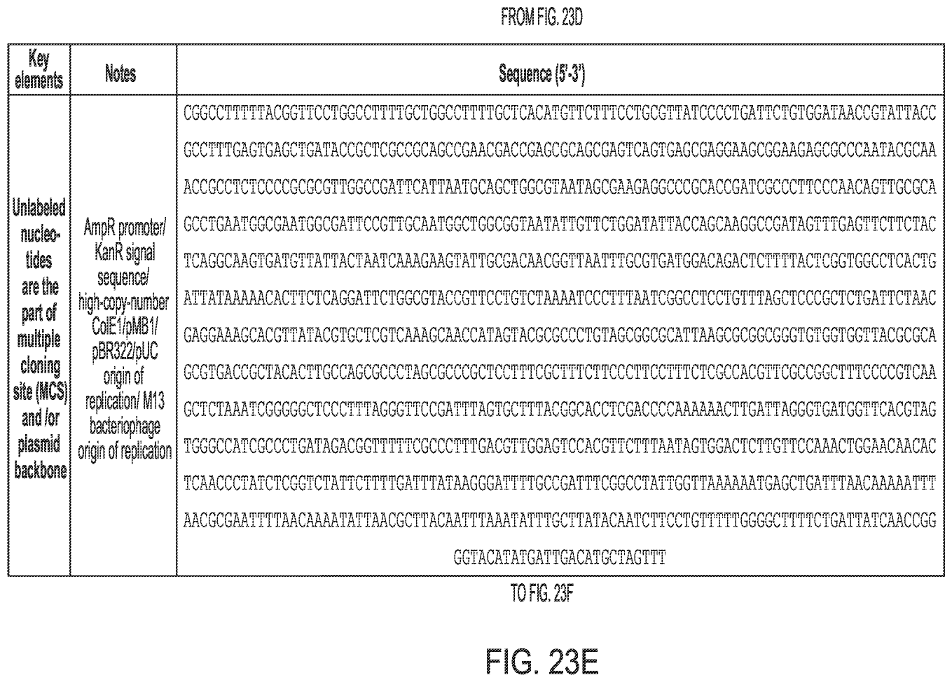

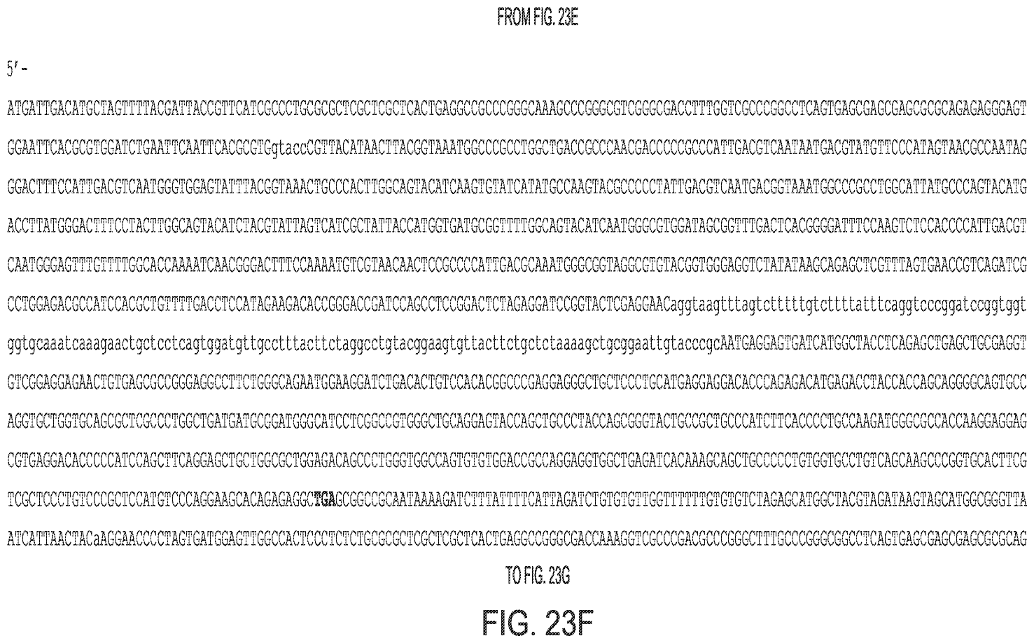

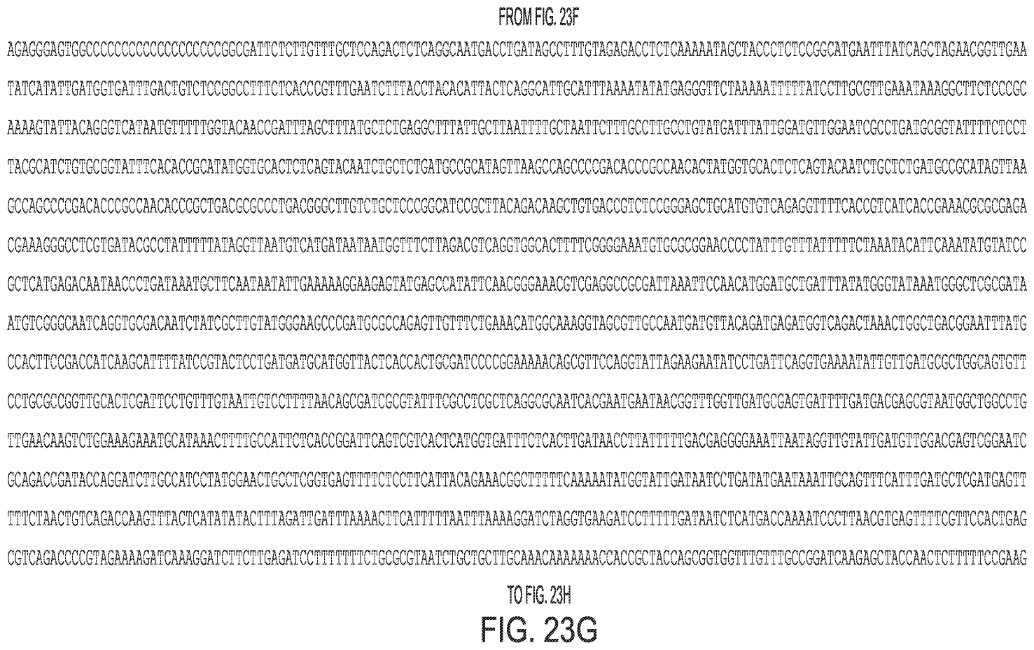

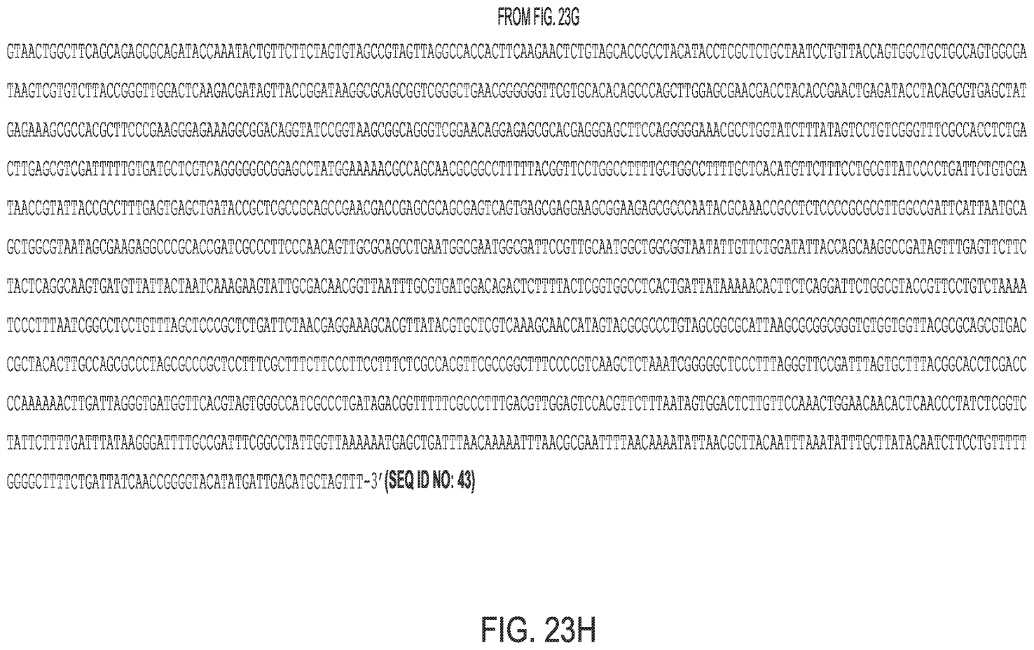

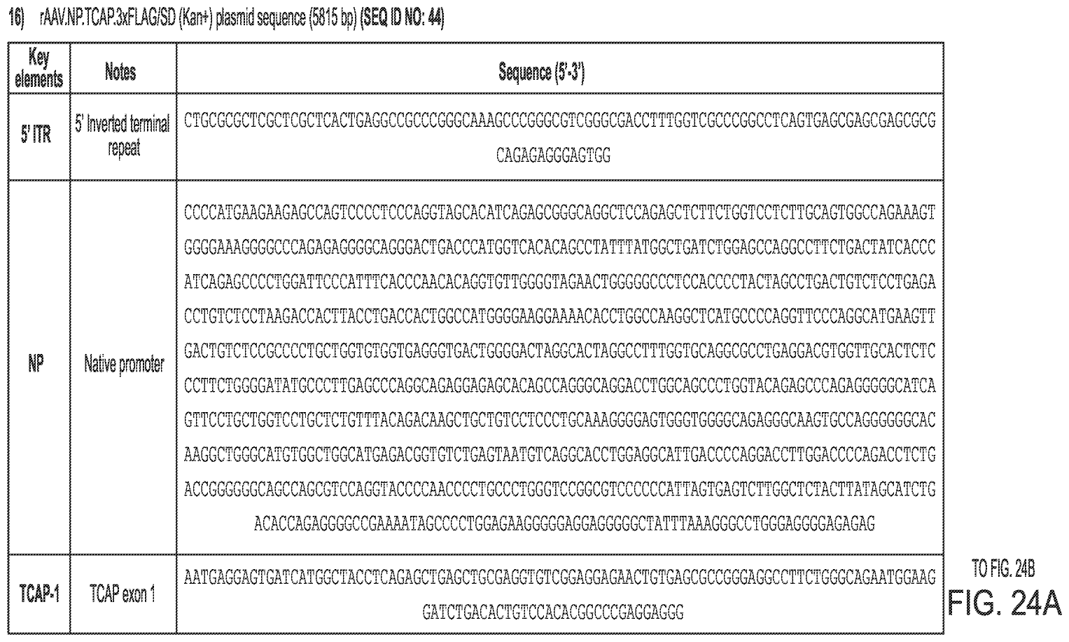

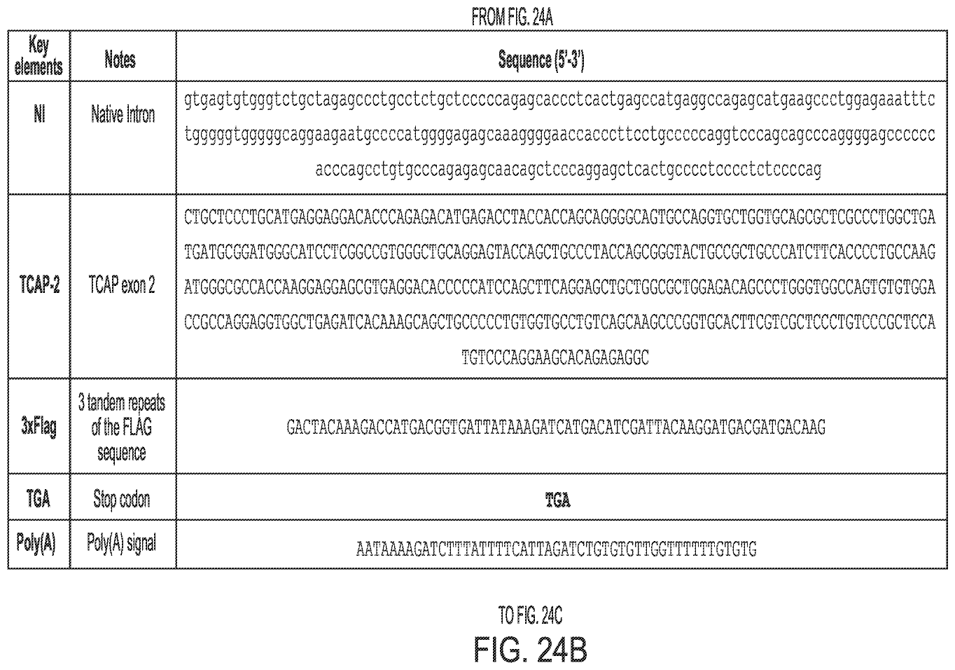

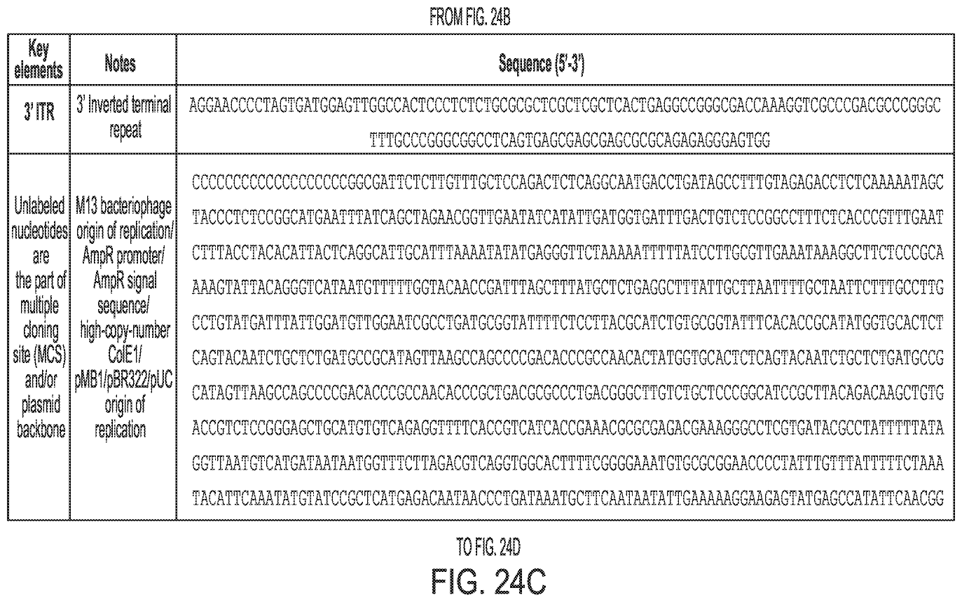

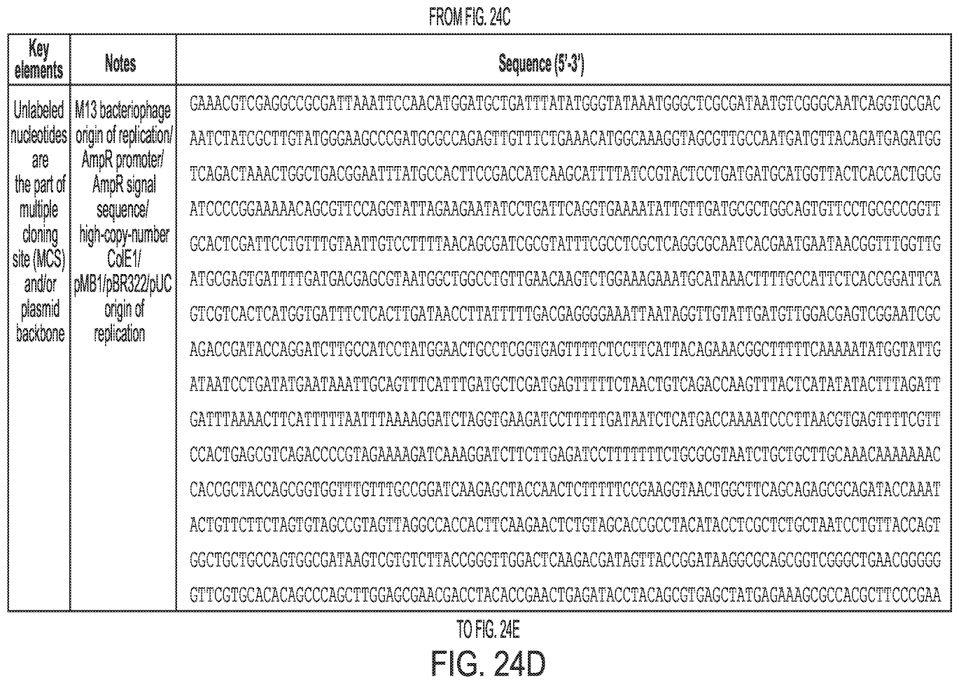

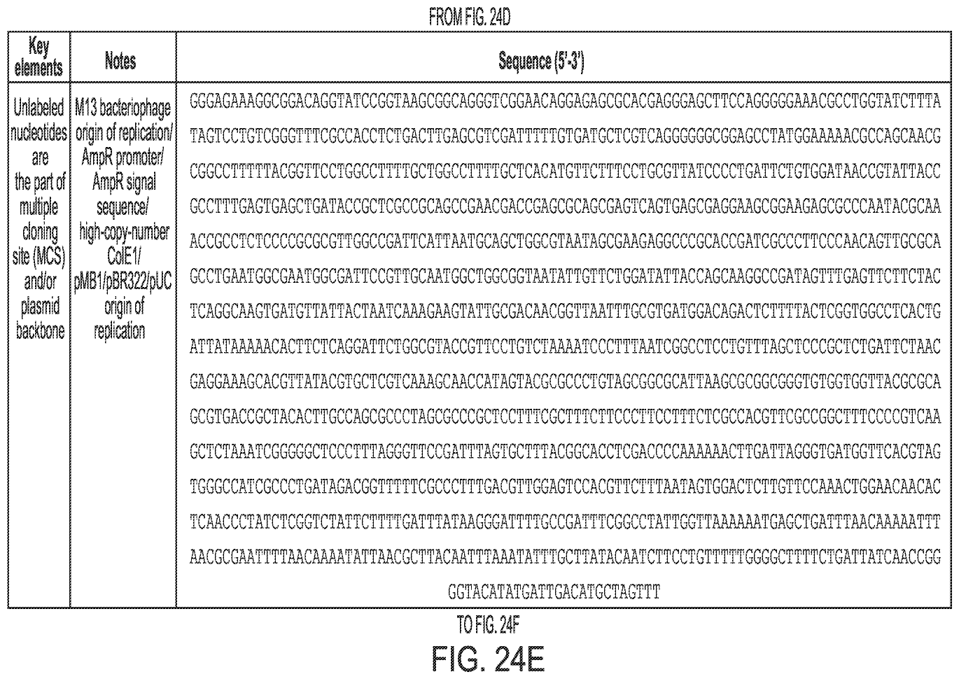

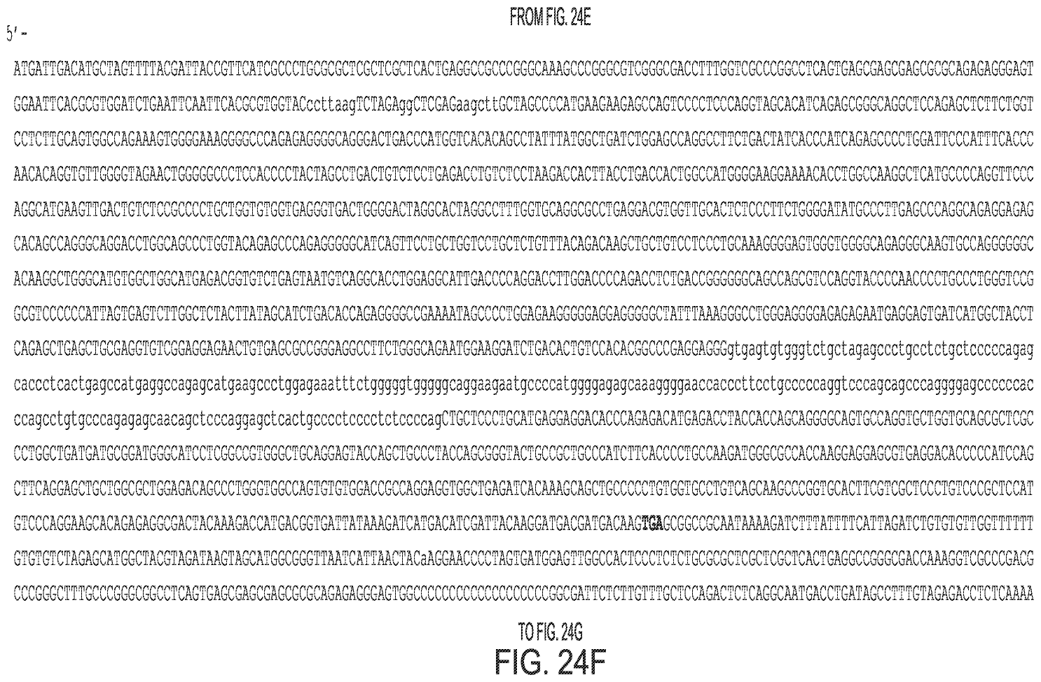

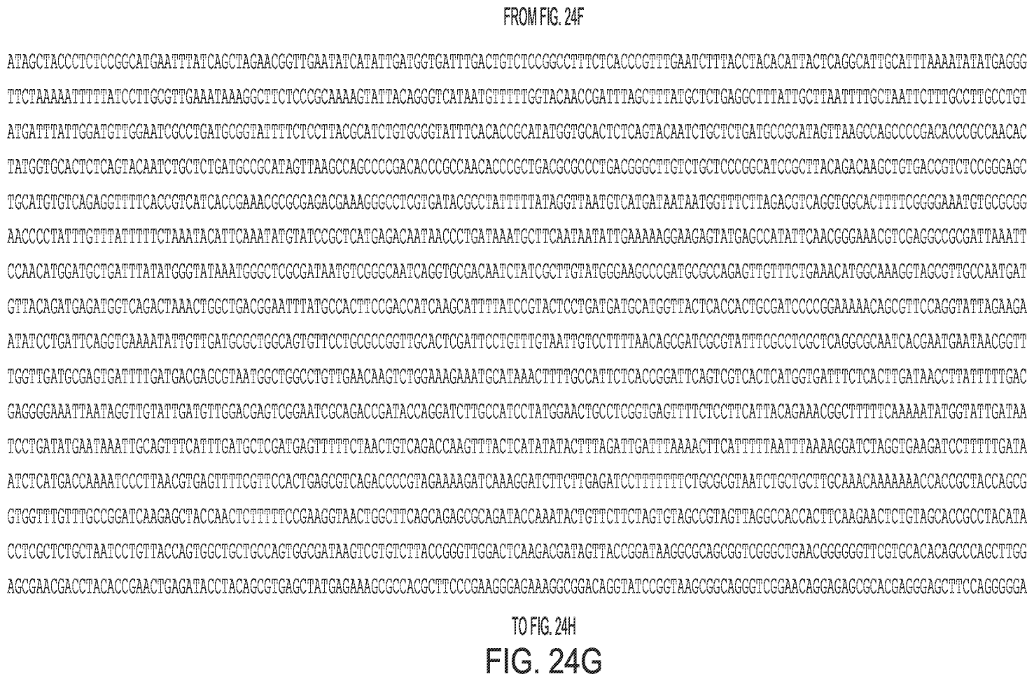

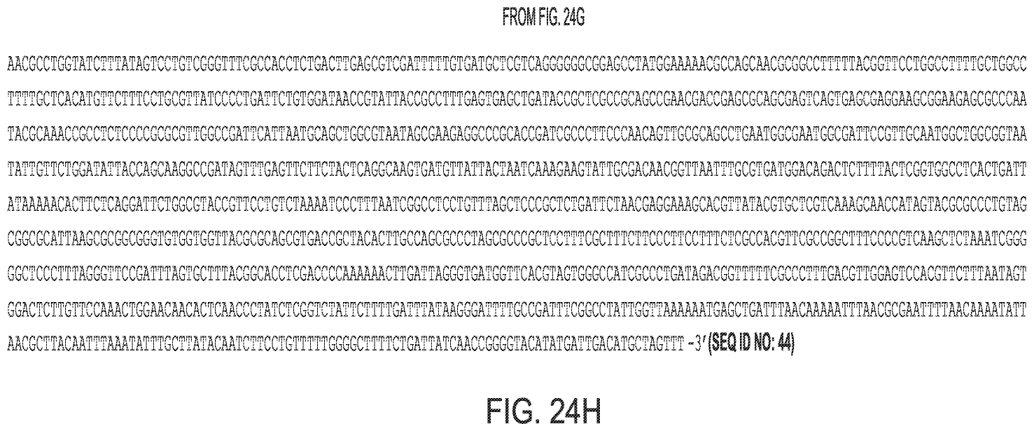

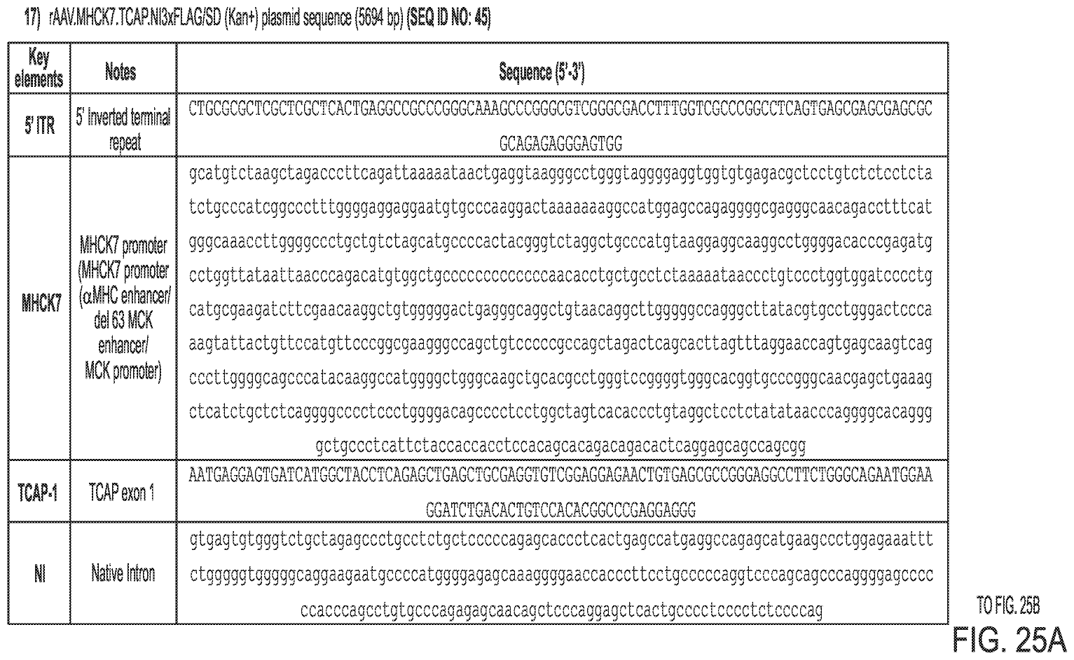

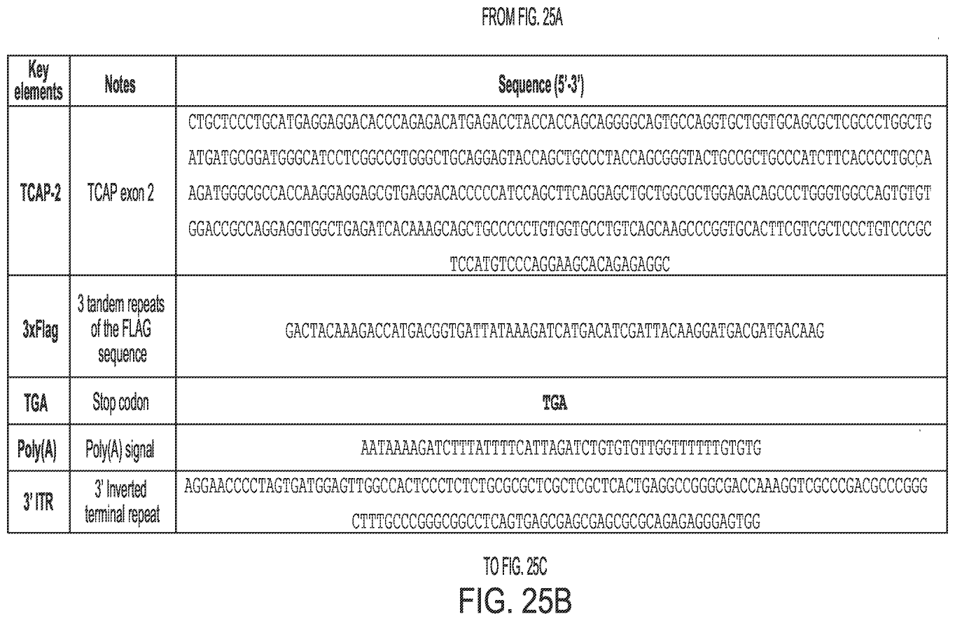

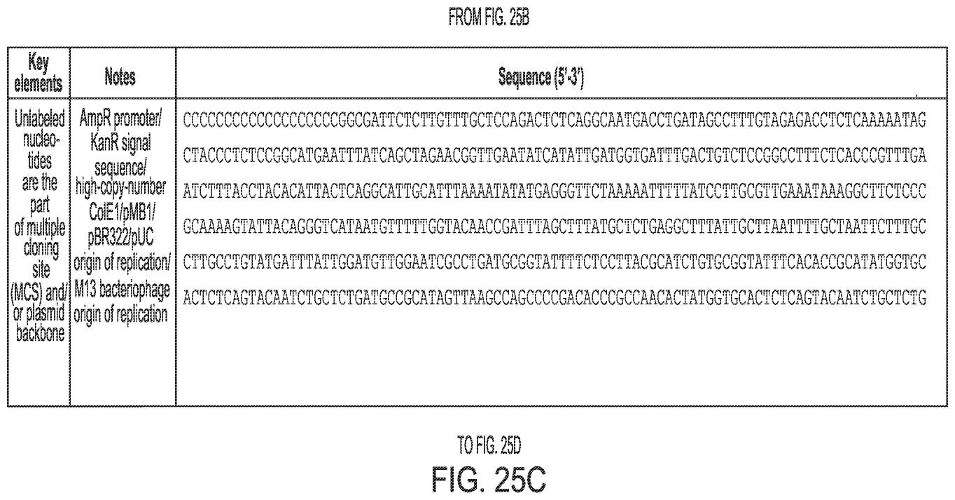

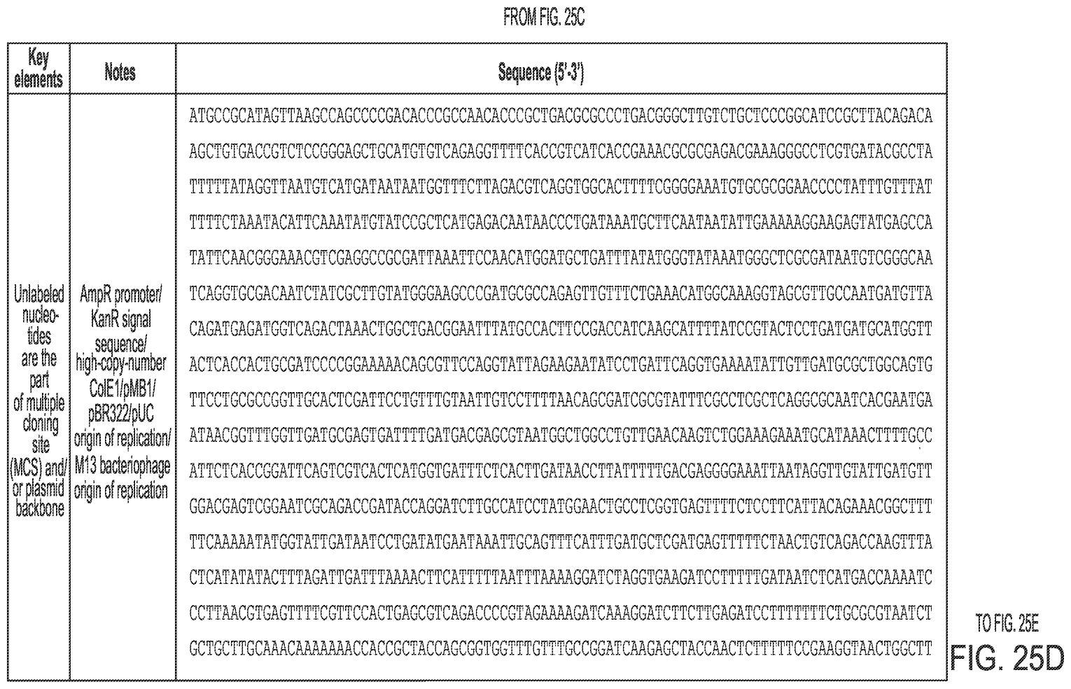

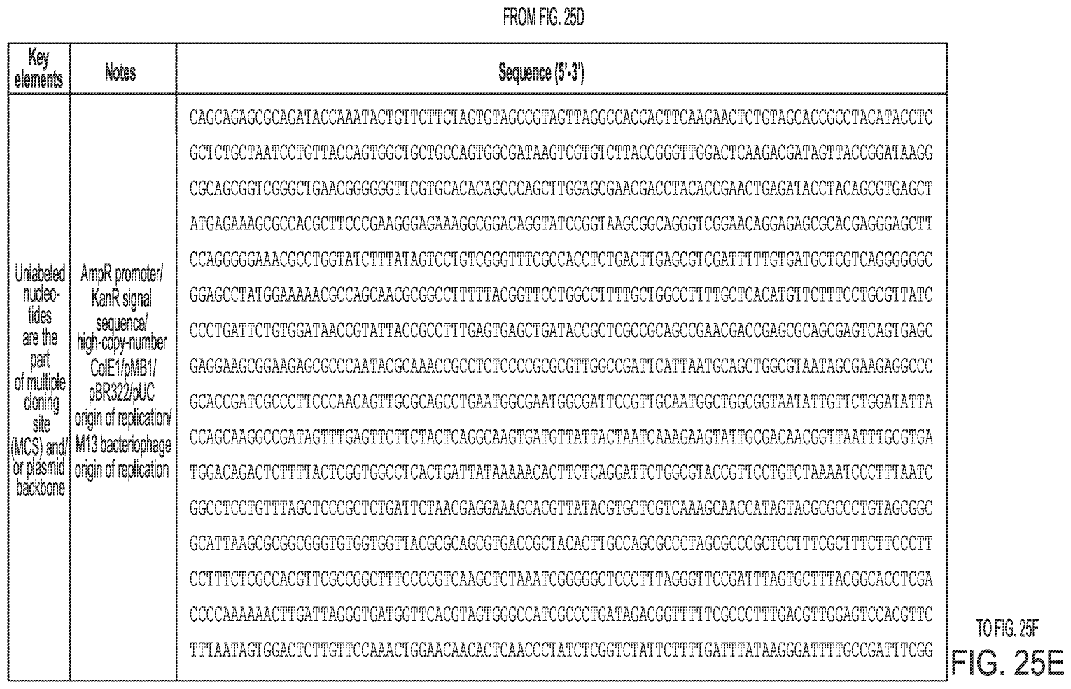

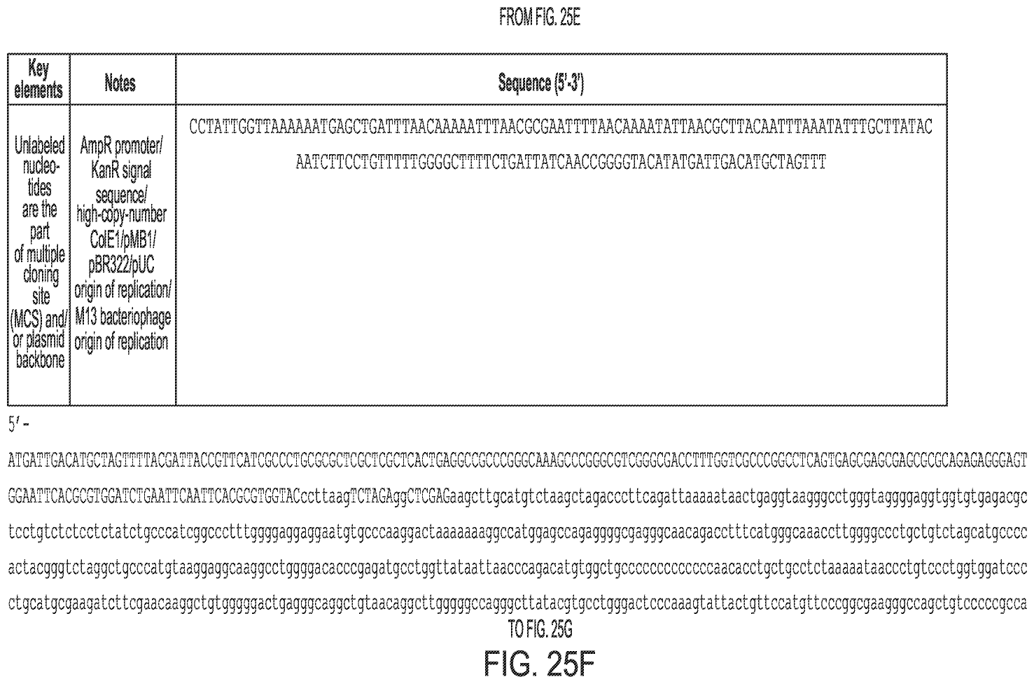

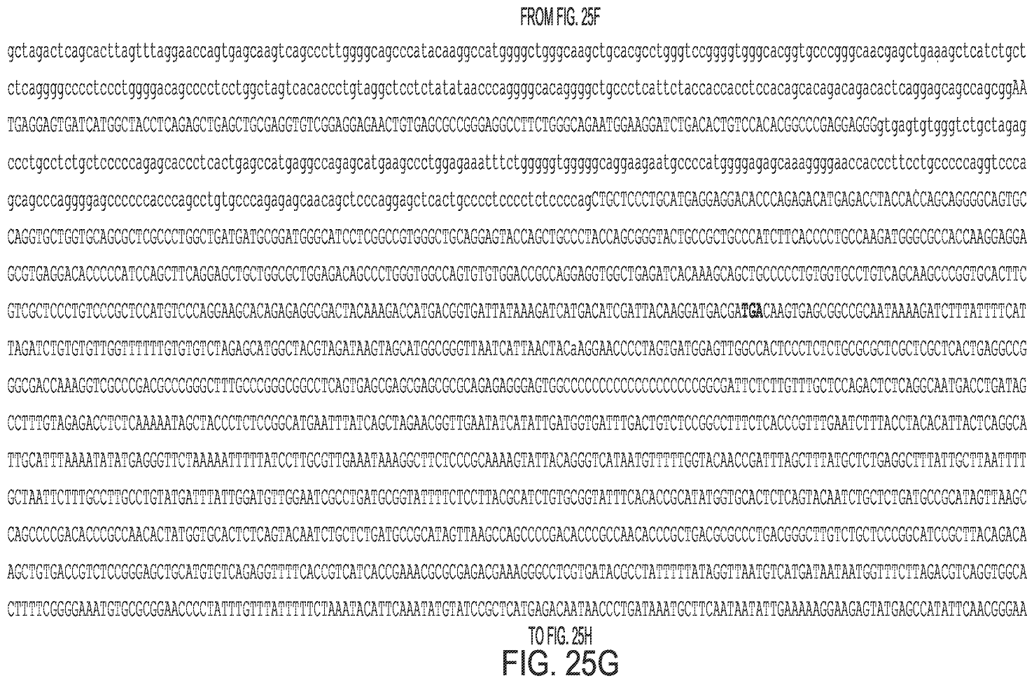

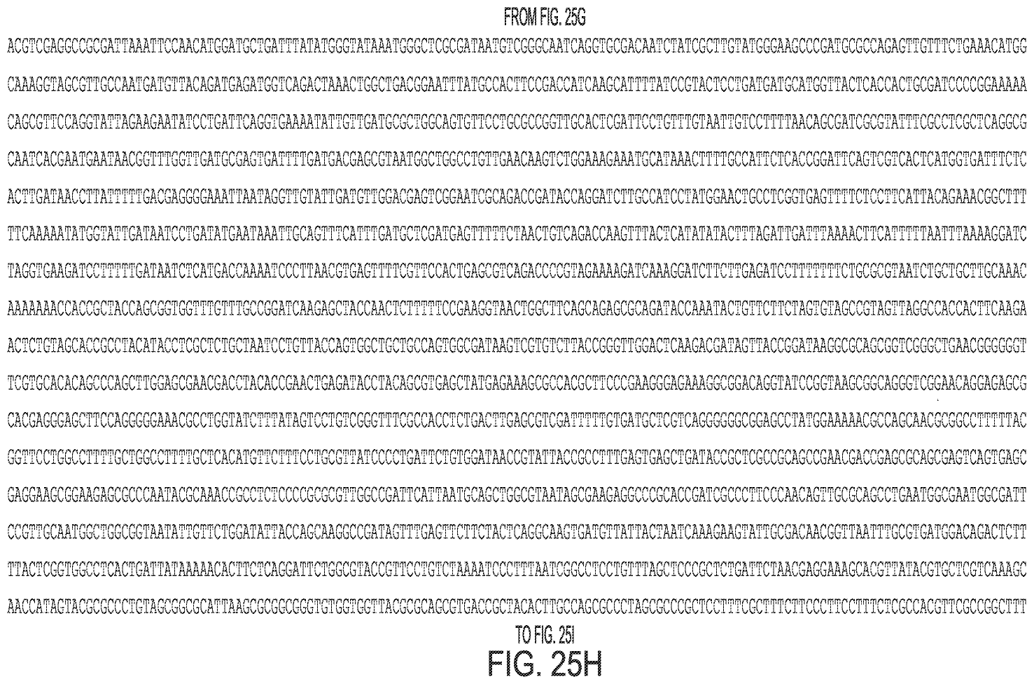

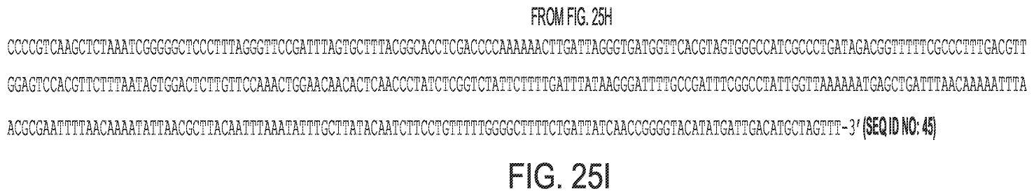

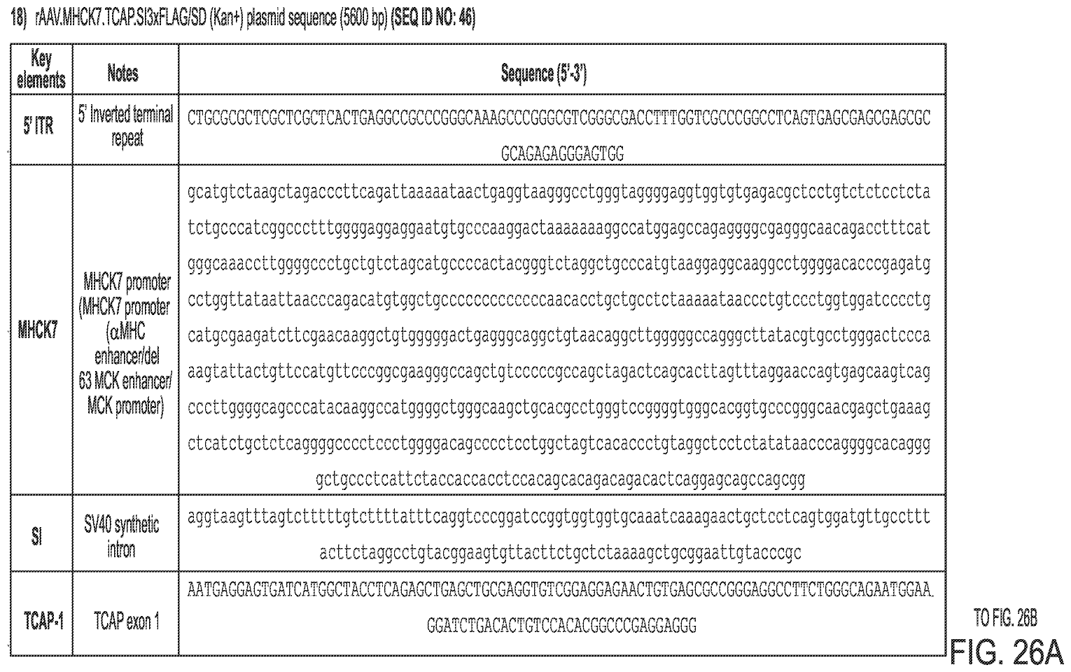

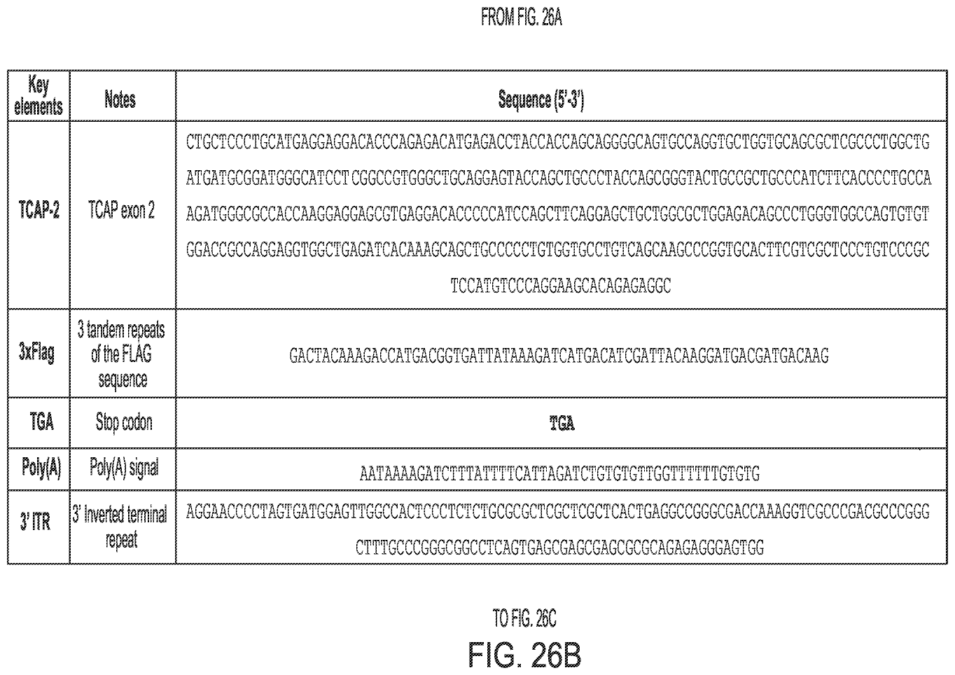

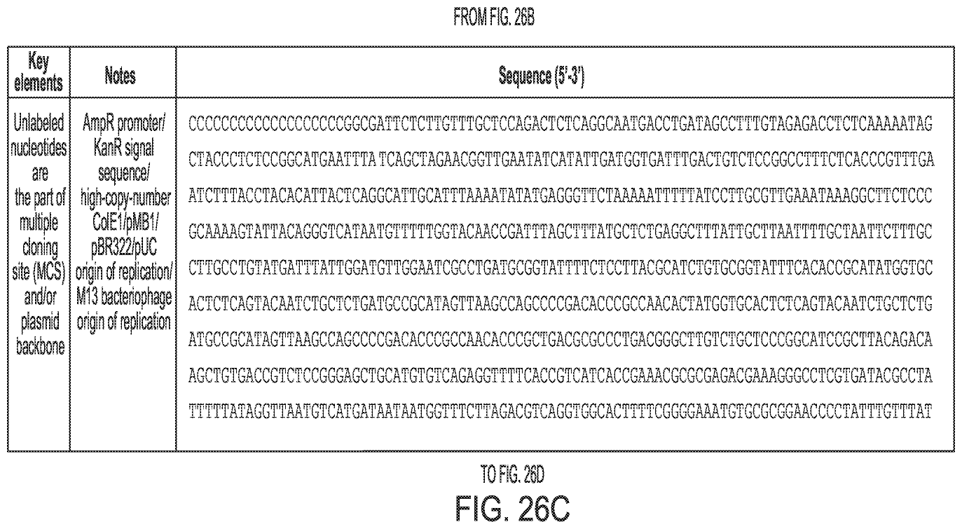

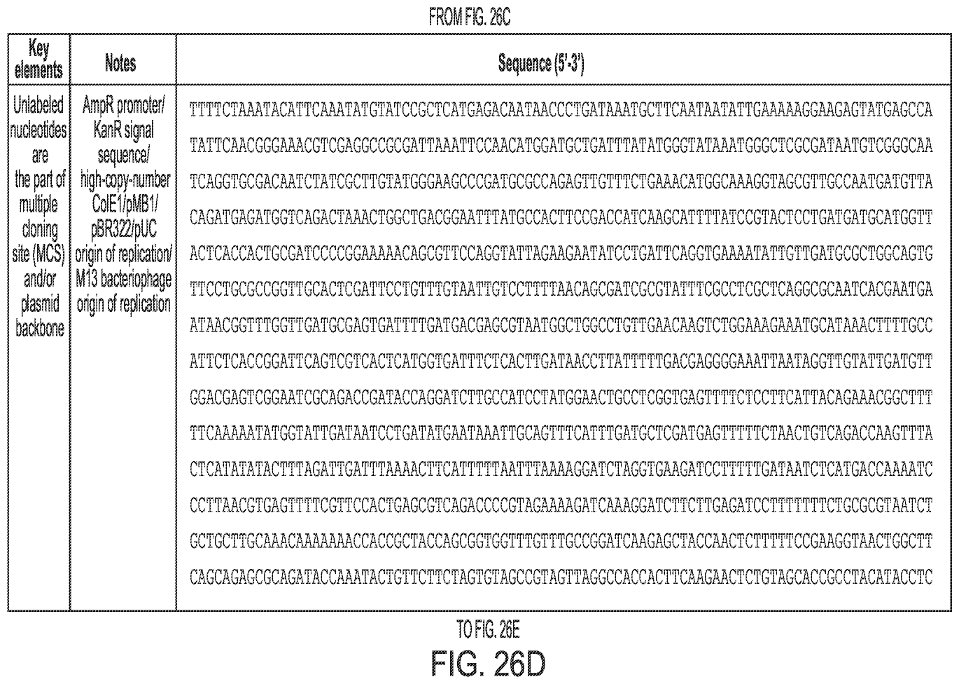

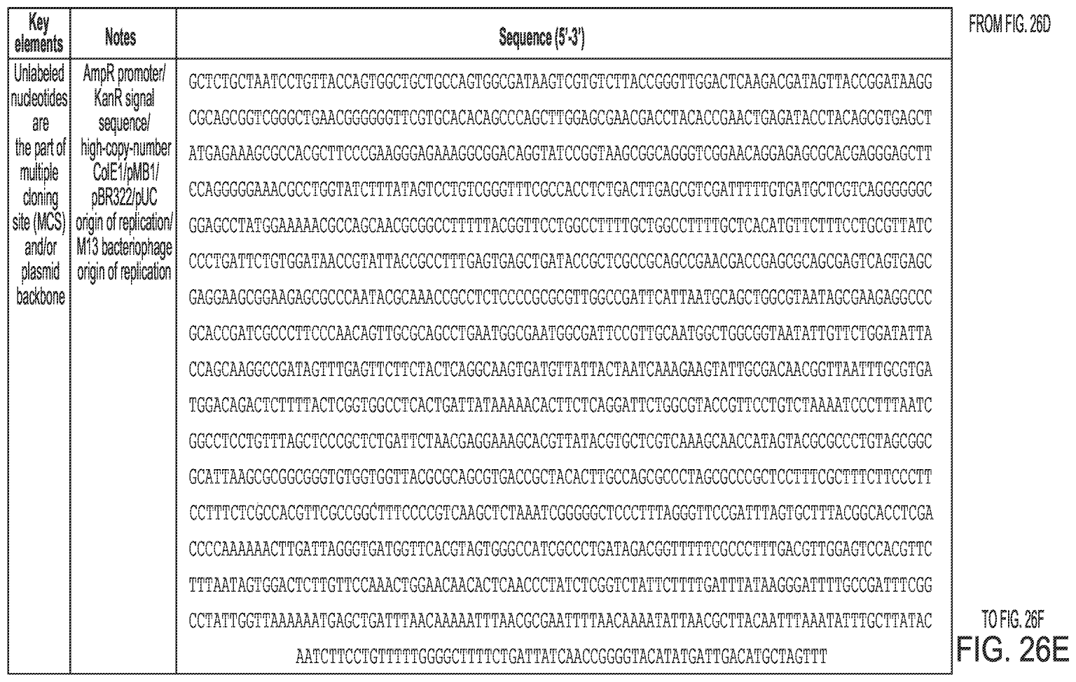

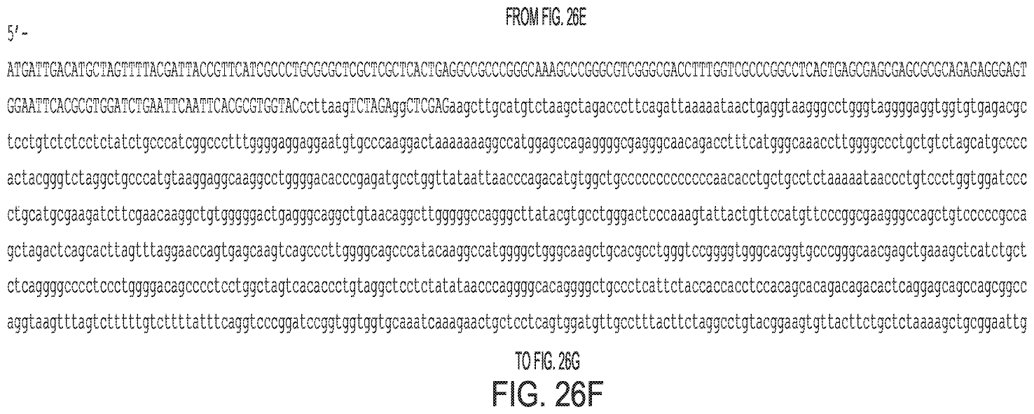

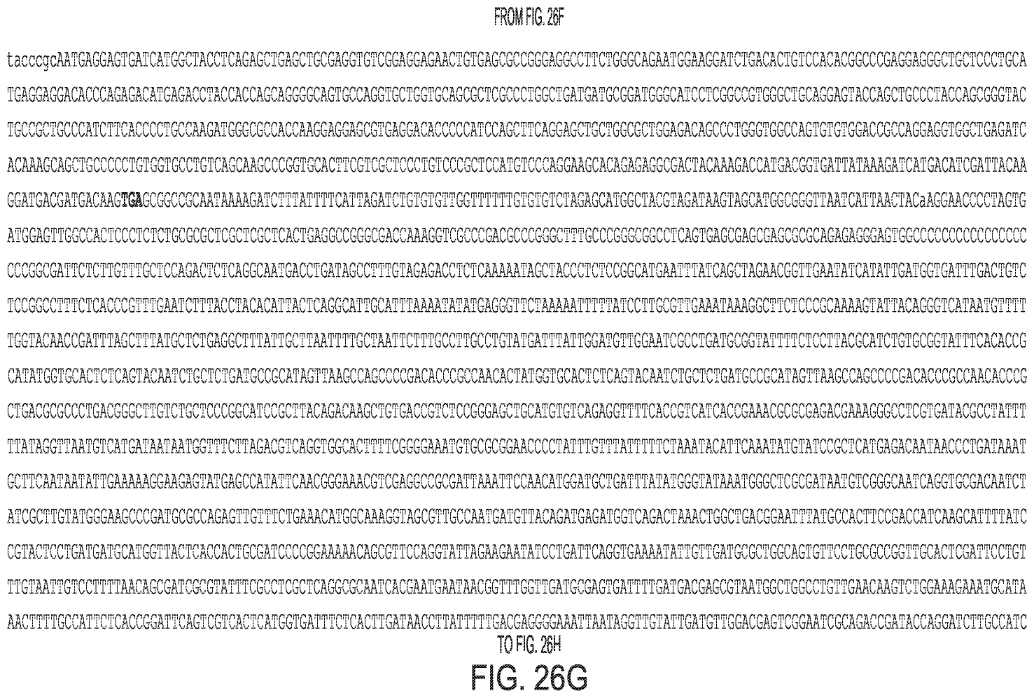

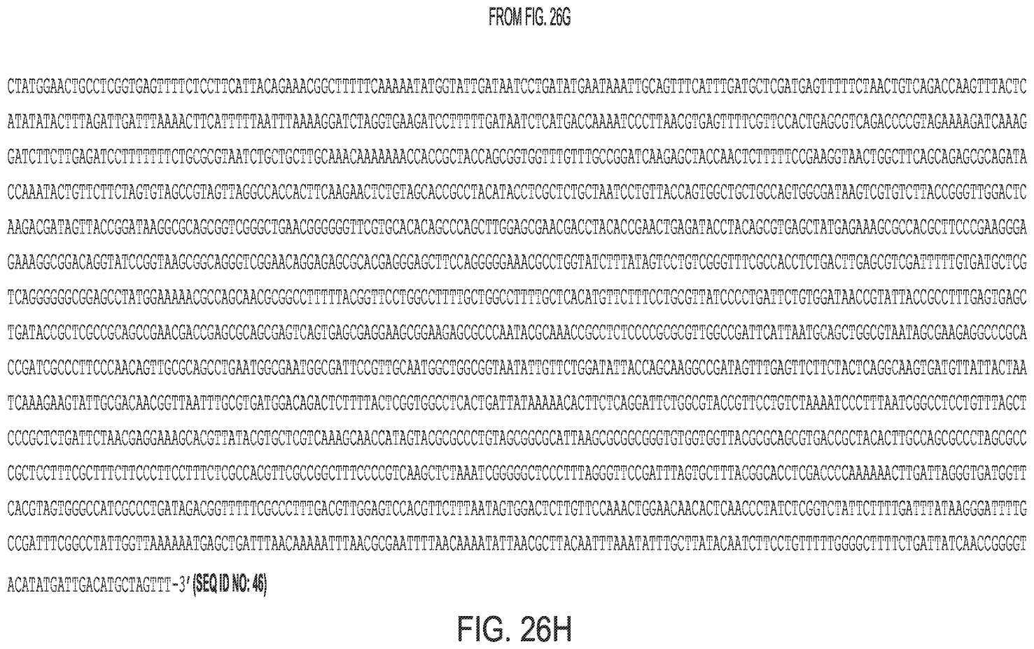

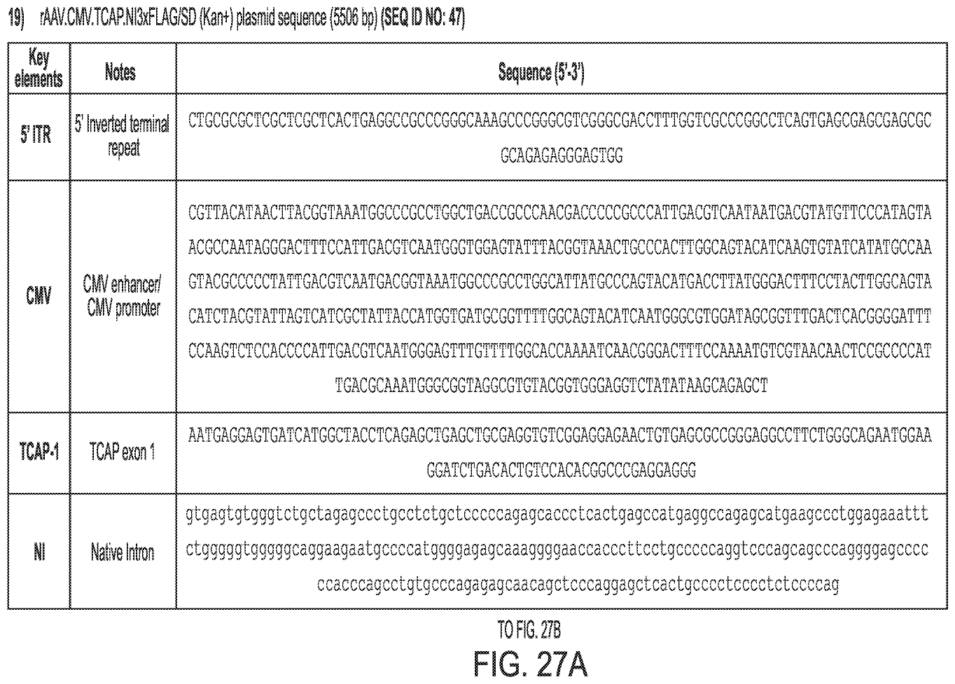

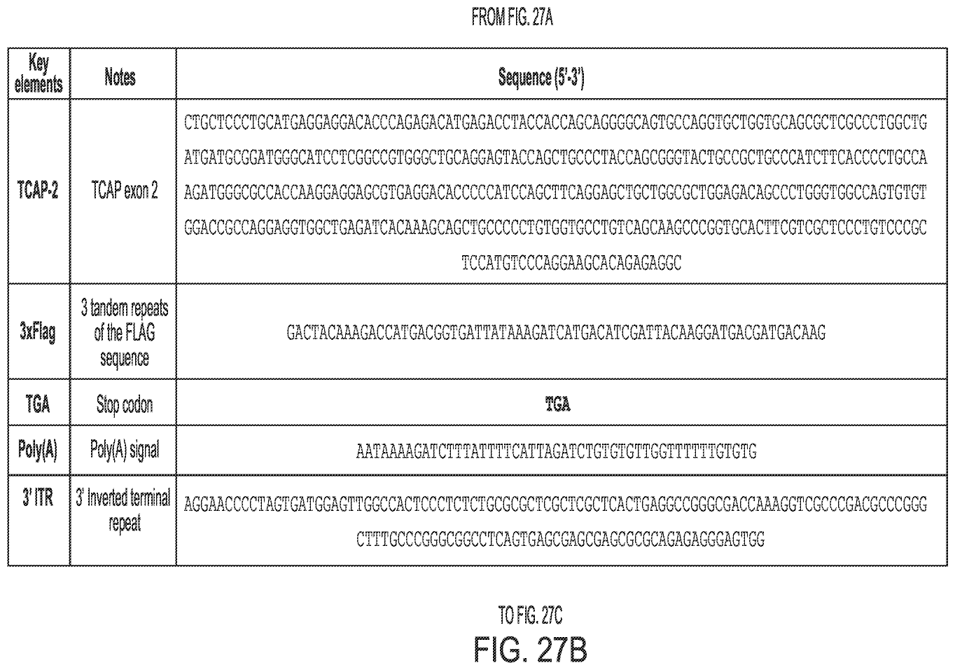

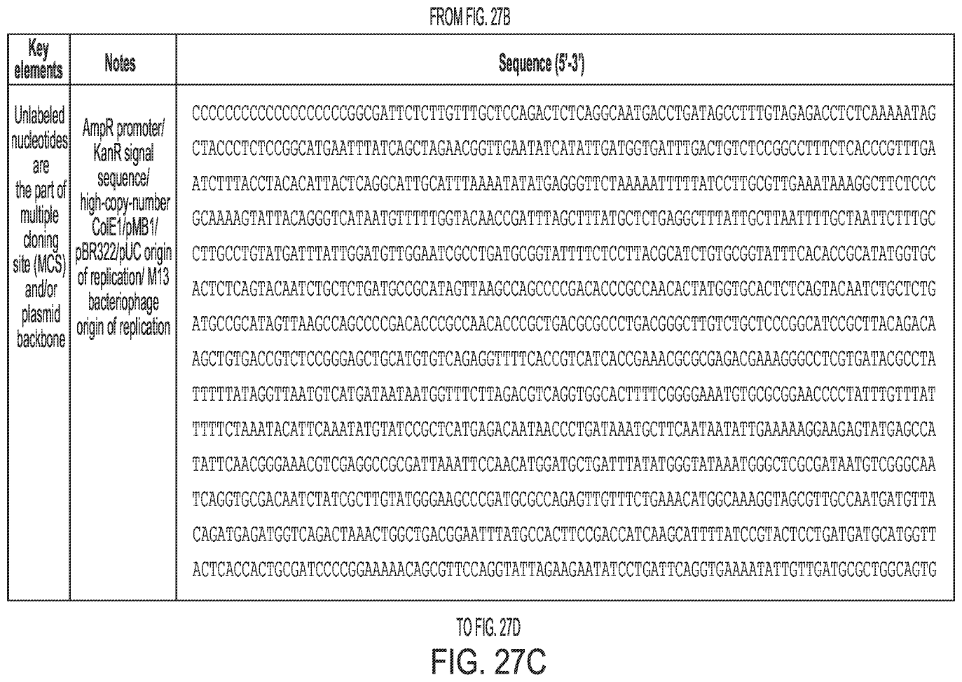

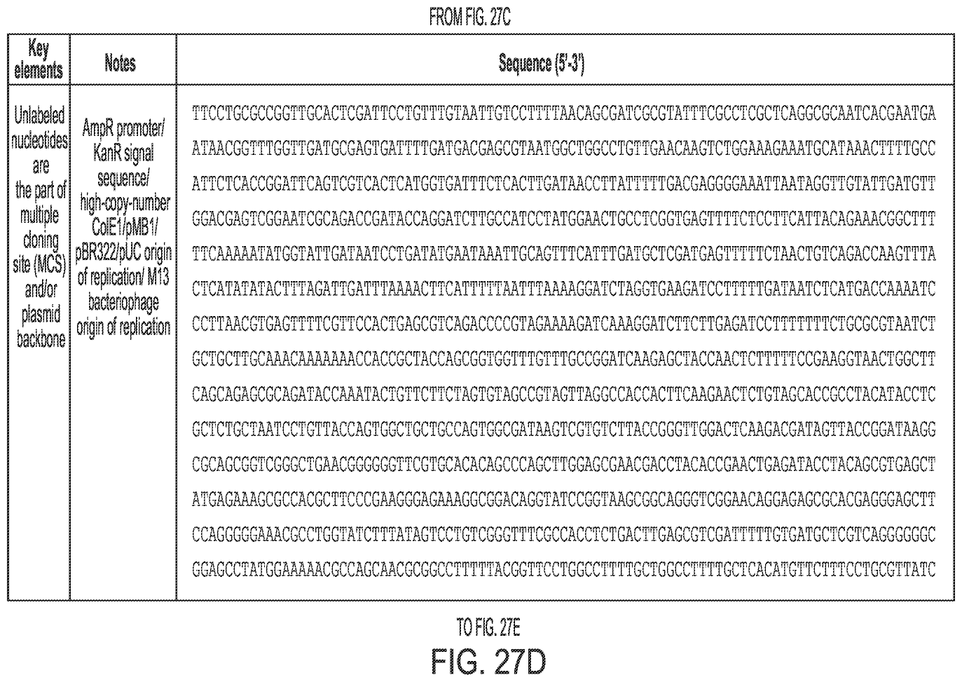

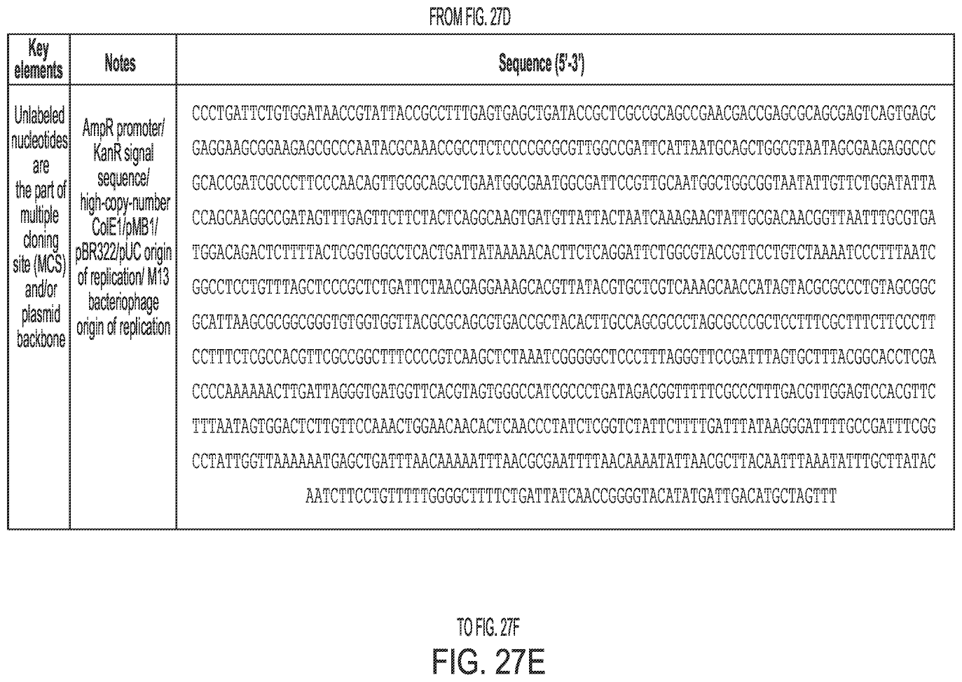

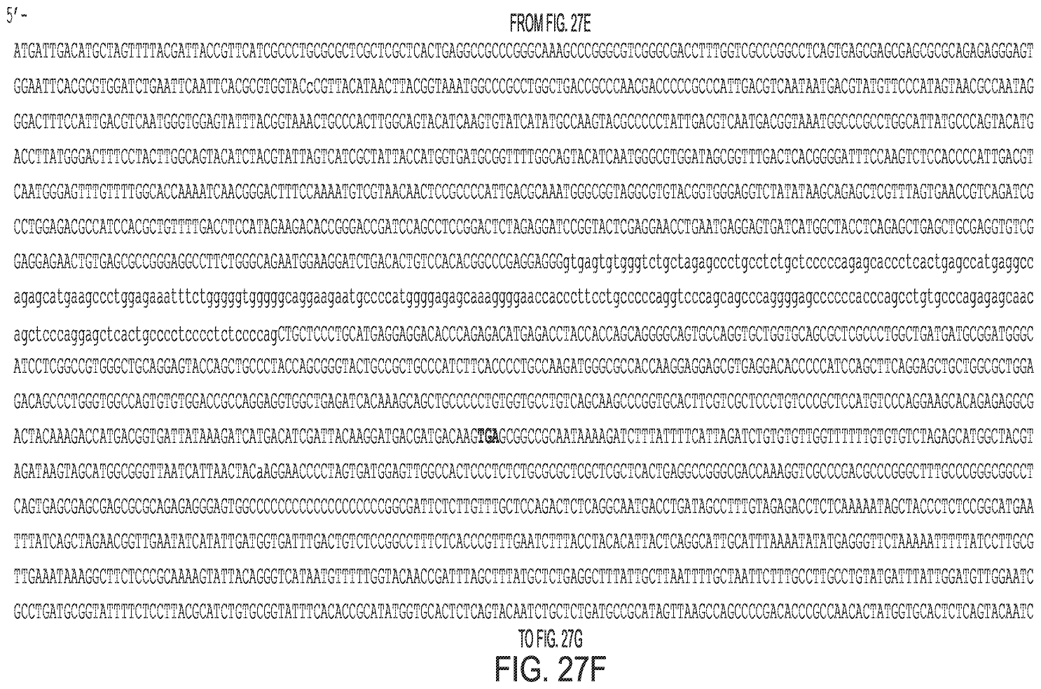

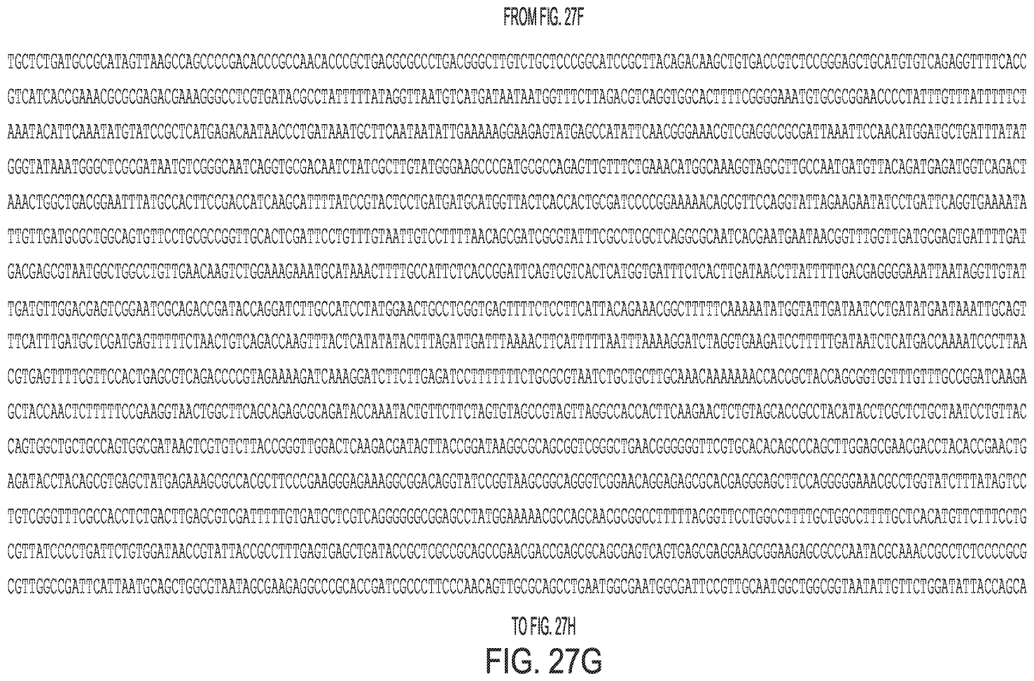

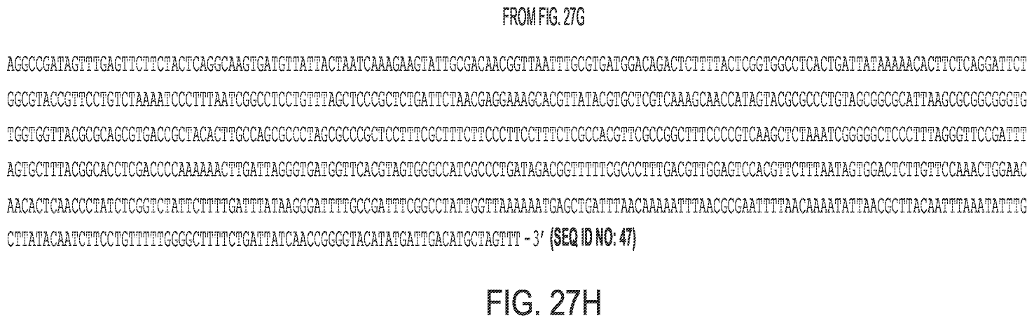

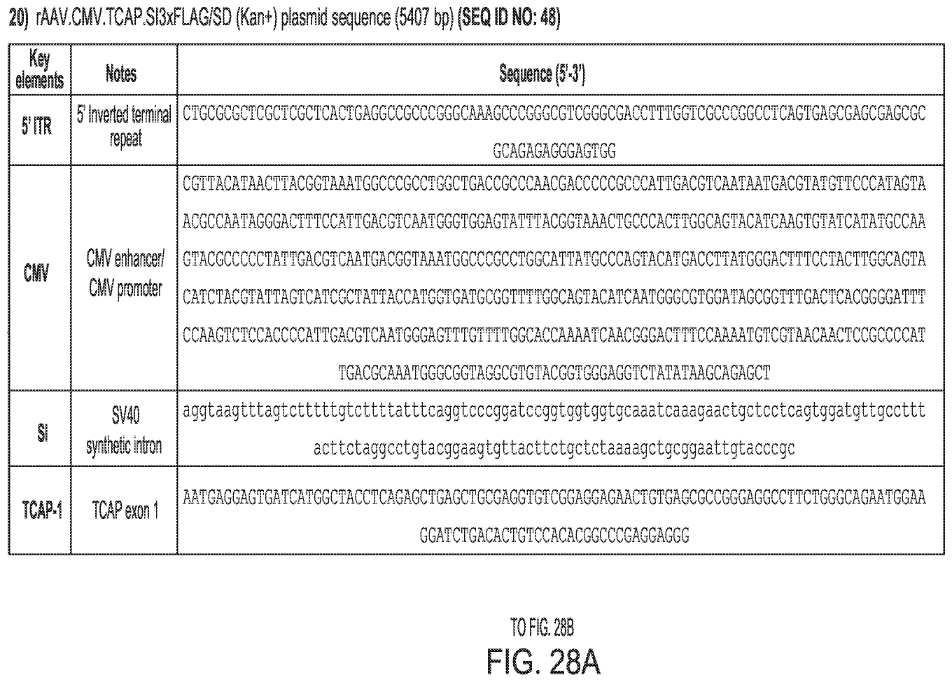

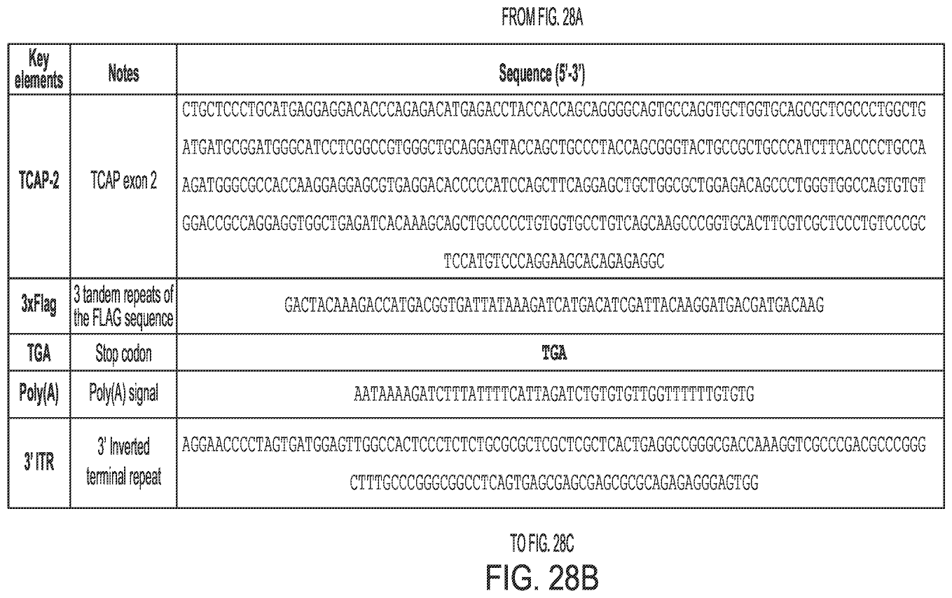

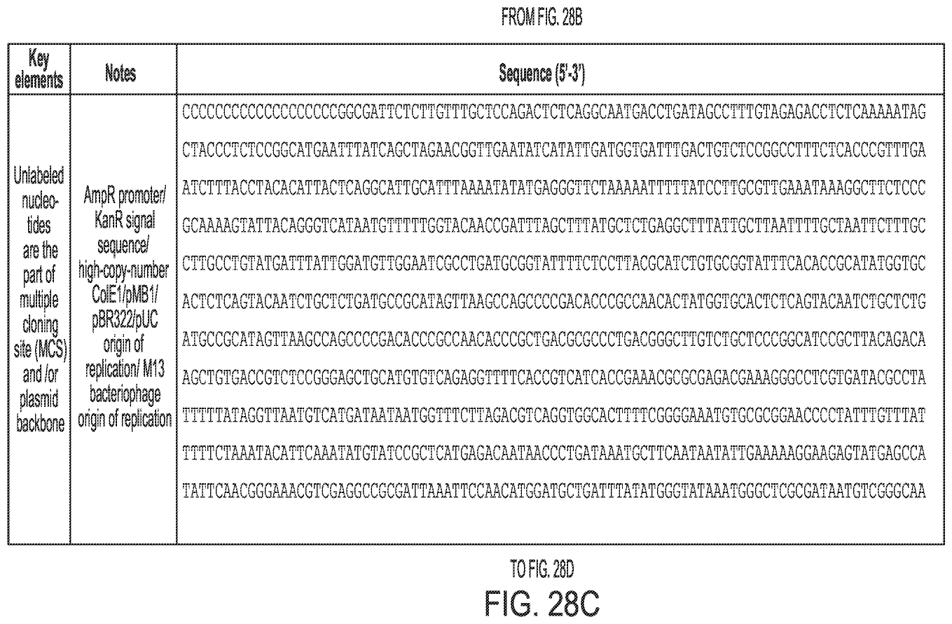

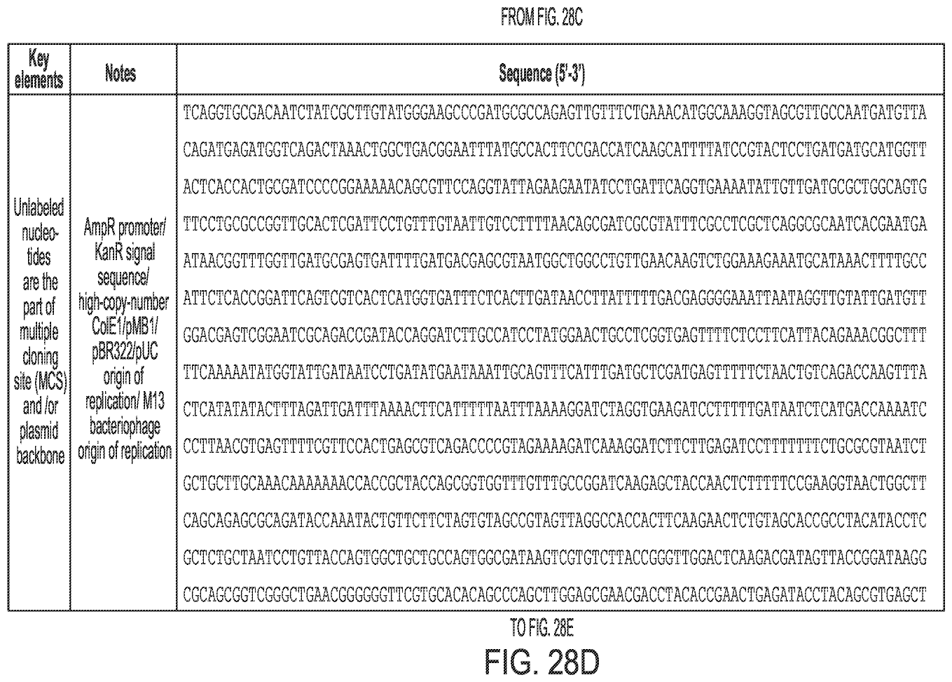

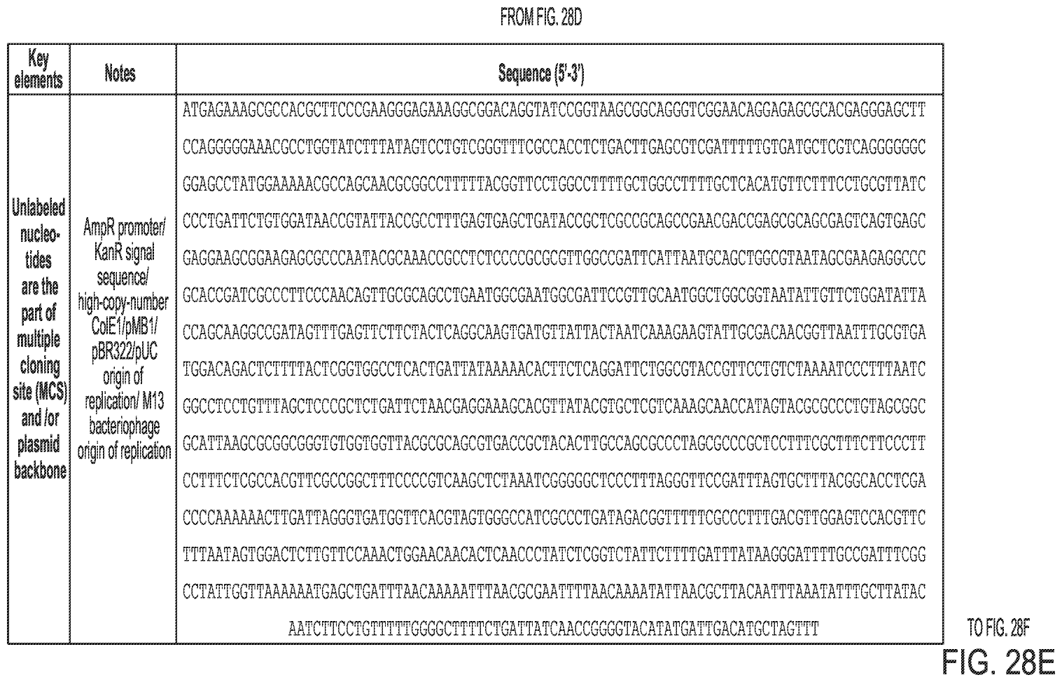

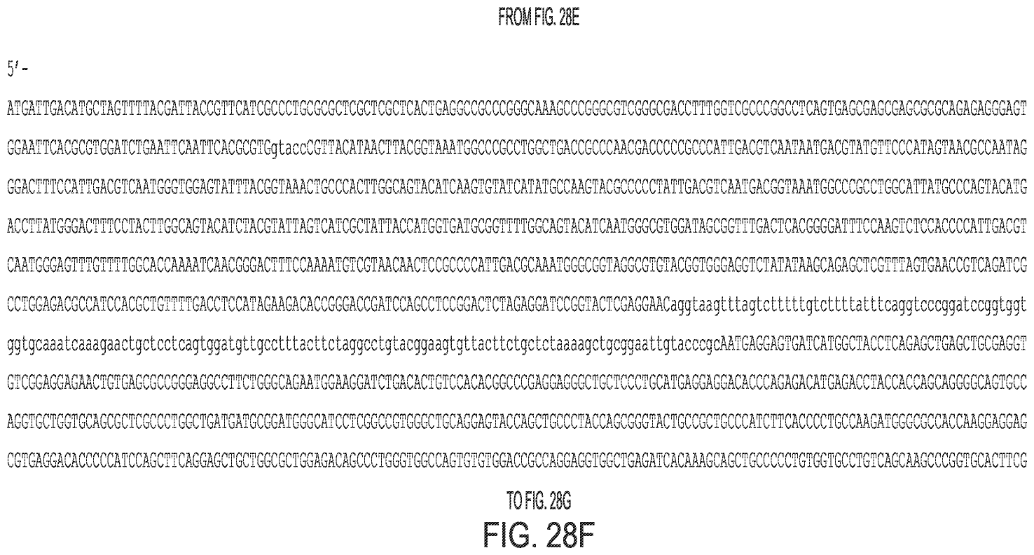

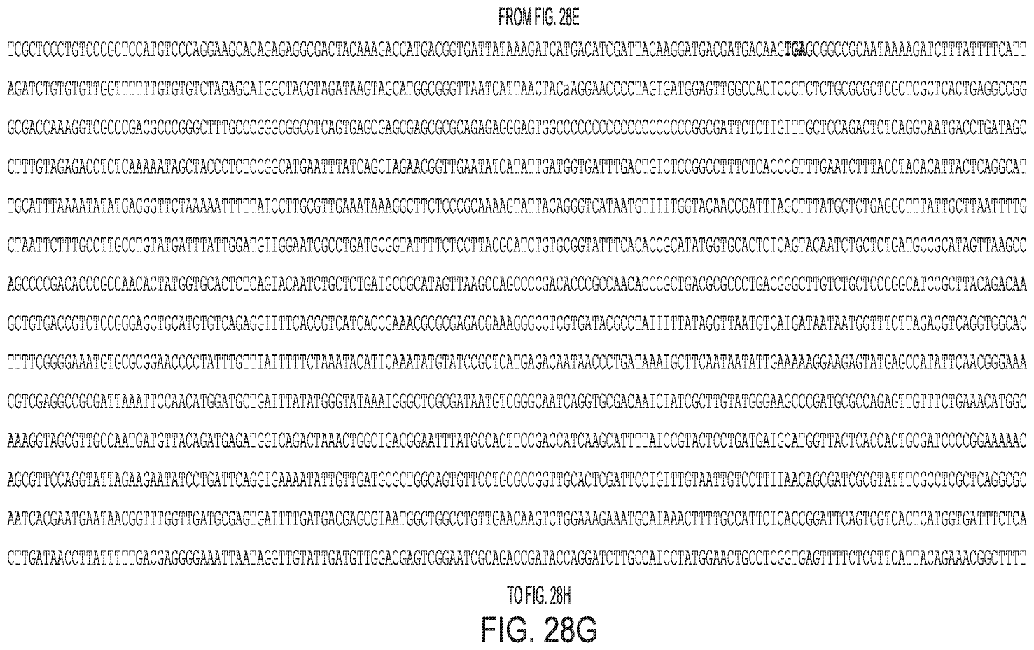

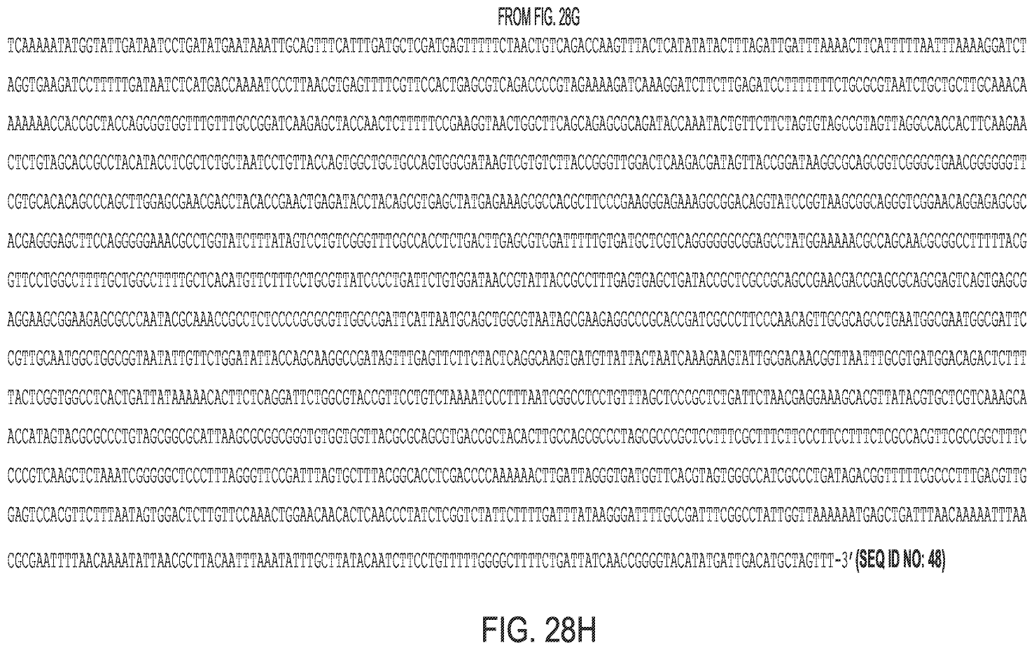

TABLE 3
Sequences of the AAV comprising the TCAP gene
including the promoter and additional elements.

| | | | Total | |
|---|---|---|---|---|
| | | Total | plasmid | |
| | | plasmid | SEQ ID | Sequence (5′-3′) |
| # | Name | size (bp) | NO: | set out in the referenced Figure |
| 1 | rAAV.NP.TCAP | 5627 | 19 | FIGS. 1 and 9 |
| 2 | rAAV.MHCK7.TCAP.NI | 5506 | 20 | FIGS. 1 and 10 |
| 3 | rAAV.MHCK7.TCAP.SI | 5412 | 21 | FIGS. 1 and 11 |
| 4 | rAAV.CMV.TCAP.NI | 5351 | 22 | FIGS. 1 and 12 |
| 5 | rAAV.CMV.TCAP.SI | 5252 | 23 | FIGS. 1 and 13 |
| 6 | rAAV.NP.TCAP.3xFLAG | 5693 | 24 | FIGS. 1 and 14 |
| 7 | rAAV.MHCK7.TCAP.NI3xFLAG | 5575^{α} | 25 | FIGS. 1-3A-B, 7, 8A-E, and 15 |
| 8 | rAAV.MHCK7.TCAP.SI3xFLAG | 5476^{β} | 26 | FIGS. 1-8A-E, and 16 |
| 9 | rAAV.CMV.TCAP.NI3xFLAG | 5417 | 27 | FIGS. 1-3A-B, 8A-E, and 17 |
| 10 | rAAV.CMV.TCAP.SI3xFLAG | 5140^{γ} | 28 | FIGS. 1-3A-B, 8A-E, and 18 |
| 11 | rAAV.NP.TCAP/SD | 5749 | 39 | FIGS. 1 and 19 |
| 12 | rAAV.MHCK7.TCAP.NI/SD | 5628 | 40 | FIGS. 1 and 20 |
| 13 | rAAV.MHCK7.TCAP.SI/SD | 5534 | 41 | FIGS. 1 and 21 |
| 14 | rAAV.CMV.TCAP.NI/SD | 5440 | 42 | FIGS. 1 and 22 |
| 15 | rAAV.CMV.TCAP.SI/SD | 5341 | 43 | FIGS. 1 and 23 |
| 16 | rAAV.NP.TCAP.3xFLAG/SD | 5815 | 44 | FIGS. 1 and 24 |
| 17 | rAAV.MHCK7.TCAP.NI3xFLAG/SD | 5694 | 45 | FIGS. 1 and 25 |
| 18 | rAAV.MHCK7.TCAP.SI3xFLAG/SD | 5600 | 46 | FIGS. 1 and 26 |
| 19 | rAAV.CMV.TCAP.NI3xFLAG/SD | 5506 | 47 | FIGS. 1 and 27 |
| 20 | rAAV.CMV.TCAP.SI3xFLAG/SD | 5407 | 48 | FIGS. 1 and 28 |

^{α}MHCK7 promoter shows a three nucleotide insertion in poly(C) region. The plasmid's expected size is 5572 bp.
^{β}MHCK7 promoter shows a two nucleotide deletion in poly(C) region. The plasmid's expected size is 5478 bp.
^{γ}5′ITR has a deletion of 72 bp. The 5′ backbone has a 106 nucleotide deletion. The plasmid's expected size is 5318 bp.

Such AAV vectors can be replicated and packaged into infectious viral particles when present in a host cell that has been transfected with a vector encoding and expressing rep and cap gene products. AAV is a single-stranded replication-deficient DNA parvovirus that grows only in cells in which certain functions are provided by a co-infecting helper virus. The genome of AAV is about 4.7 kb in length including 145 nucleotide inverted terminal repeat (ITRs). There are multiple serotypes of AAV that have been characterized. General information and reviews of AAV can be found in, for example, Carter, 1989, Handbook of Parvoviruses, Vol. 1, pp. 169-228¹⁸, and Berns, 1990, Virology, pp. 1743-1764, Raven Press, (New York)¹⁹. However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.²⁰; and Rose, Comprehensive Virology 3:1-61 (1974)²¹). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45:555-564 (1983)²² as corrected by Ruffing et al., J Gen Virol, 75:3385-3392 (1994)²³. As other examples, the complete genome of AAV-1 is provided in GenBank Accession No. NC_002077; the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78:6381-6388 (2004)²⁴; the AAV10 genome is provided in Mol. Ther., 13 (1): 67-76 (2006)²⁵; and the AAV11 genome is provided in Virology, 330 (2): 375-383 (2004)²⁶. Cloning of the AAVrh.74 serotype is described in Rodino-Klapac., et al. Journal of translational medicine 5, 45 (2007)²⁷.

Cis-acting sequences directing viral DNA replication (rep), encapsidation/packaging and host cell chromo-some integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992)²⁸.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus, making cold preservation of AAV less critical. AAV may be lyophilized and AAV-infected cells are not resistant to superinfection.

In some aspects, the AAV lacks rep and cap genes. In some aspects, the AAV is a recombinant linear AAV (rAAV), a single-stranded AAV, or a recombinant self-complementary AAV (scAAV). The self-complementary (sc) technology allows for binding of the single-stranded viral DNA genome onto itself, thereby priming second strand DNA synthesis. This sc element both quickens and strengthens gene expression relative to constructs lacking the sc element.

Advances in AAV vectors have led to safer and more efficient viral vehicles to deliver therapeutic transgenes in a single injection, and gene therapy is now a favorable therapeutic intervention for monogenic diseases. AAV vectors can provide long-term expression of gene products in post-mitotic target tissues. Thus, current AAV-based strategies may only require one-time vector administration.

Recombinant AAV genomes of the disclosure comprise one or more AAV ITRs flanking a polynucleotide encoding, for example, one or more MPZ inhibitory RNAs or MPZ miRNAs. The genomes of the rAAV provided herein either further comprise an RNAi-resistant replacement MPZ gene, or the RNAi-resistant replacement MPZ gene is present in a separate rAAV. The miRNA- and replacement MPZ-encoding polynucleotides are operatively linked to transcriptional control DNAs, for example promoter DNAs, which are functional in a target cell. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCER™ siRNA Construction Kit (Ambion Inc., Austin, TX) or psiRNA System (InvivoGen, San Diego, CA).

Provided herein are rAAV, each comprising one or more TCAP genes. An rAAV comprising one or more TCAP genes can encode one, two, three, four, five, six, seven or eight TCAP proteins.

In some aspects, therefore, the viral vector is an AAV, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAV10 (i.e., an AAV containing AAV10 ITRs and AAV10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAVanc80 (i.e., an AAV containing AAVanc80 ITRs and AAVanc80 capsid proteins), AAVrh.74 (i.e., an AAV containing AAVrh.74 ITRs and AAVrh.74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), or pseudotyped AAV, such as AAV2/1, AAV2/8, or AAV2/9, or AAVMYO, or any of their derivatives.

In various aspects, the AAV is AAV9. AAV9 has become the most widely used vector for muscular and/or neurological indications with an established safety profile in the clinic. Intrathecal administration of AAV9 permits dissemination of transgenes throughout the nervous system and is currently approved by FDA for spinal muscular atrophy (SMA, NCT03381729), and in trials for the treatment of neuronal ceroid lipofuscinosis 3 (CLN3, NCT03770572), CLN6 (NCT02725580), giant axonal neuropathy (GAN, NCT02362438), mucopolysaccharidoses types 3A (NCT02716246) and 3B (NCT03315182), and exon 2 duplications in the DMD gene (NCT04240314). Such features make AAV9 an ideal gene delivery method for treatment of genetic disorders, such as mutations in TCAP, where muscle and heart are the most affected organs. It has been shown that AAV9 can also target Schwann cells, and other peripheral neuropathies. More importantly, AAV9 was reported to transduce Schwann cells in large animals and non-human primates, indicating that it is a desirable viral vector for clinical applications requiring delivery of therapeutic genes into the human Schwann cells. Finally, data from studies in other models of muscle disease show that an AAV9 vector efficiently transfects skeletal muscle, heart, and diaphragm in mice and non-human primates.

DNA plasmids of the disclosure comprise rAAV genomes of the disclosure. In some aspects, the DNA plasmids are transferred to cells permissible for infection with a helper virus of AAV (e.g., adenovirus, E1-deleted adenovirus or herpes virus) for assembly of the rAAV genome into infectious viral particles. Thus, in some aspects, the disclosure includes AAV vectors to deliver therapeutic agents into a cell. In some aspects, the cell is a neuronal cell. In some aspects, the neuronal cell is a Schwann cell.

An “AAV virion” or “AAV viral particle” or “AAV particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVrh.74, AAVrh.8, or AAVrh.10, AAVAnc80, AAV7m8, AAV2/1, AAV2/8, AAV2/9, or AAVMYO and their derivatives. In some aspects, AAV DNA in the rAAV genomes is from any AAV serotype for which a recombinant virus can be derived including, but not limited to, AAV serotypes AAV is AAV1, AAV2, AAV3, AAV4, AAV5, AAV6, AAV7, AAV8, AAV9, AAV10, AAV11, AAV12, AAV13, AAVrh.74, AAVrh.8, or AAVrh.10, AAVAnc80, AAV7m8, AAV2/1, AAV2/8, AAV2/9, or AAVMYO, and their derivatives. Other types of rAAV variants, including those for example with capsid mutations, are also included in the disclosure. Such variants include, but are not limited to, MyoAAV or AAVMYO, and other variants as described, for example, in Marsic et al., Molecular Therapy 22 (11): 1900-1909 (2014)²⁹; Weismann, J., et al., Nat Commun 11 (1): 5432 (2020)³⁰ and Tabebordbar, M. et al., Cell 184 (19): 4919-4938 e22 (2021)³¹, which are incorporated for use herein by reference in their entirety. As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAV11, AAV12, AAV13, AAVrh.74, AAVrh.8, or AAVrh.10, AAVAnc80, AAV7m8, AAV2/1, AAV2/8, AAV2/9, or AAVMYO and their derivatives. Other types of rAAV variants, for example rAAV with capsid mutations, are also contemplated. See, for example, Marsic et al., Molecular Therapy, 22 (11): 1900-1909 (2014)²⁹. The nucleotide sequences of the genomes of various AAV serotypes are known in the art.

Multiple studies have demonstrated long-term (>1.5 years) recombinant AAV-mediated protein expression in muscle. See, Clark et al., Hum Gene Ther, 8:659-669 (1997)³²; Kessler et al., Proc Nat. Acad Sc. USA, 93:14082-14087 (1996)³³; and Xiao et al., J Virol, 70:8098-8108 (1996)³⁴. See also, Chao et al., Mol Ther, 2:619-623 (2000)³⁵ and Chao et al., Mol Ther, 4:217-222 (2001)³⁶. Further, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997)³⁷ and Murphy et al., Proc Natl Acad Sci USA, 94:13921-13926 (1997)³⁸. Moreover, Lewis et al., J Virol, 76:8769-8775 (2002)³⁹ demonstrated that skeletal myofibers possess the necessary cellular factors for correct antibody glycosylation, folding, and secretion, indicating that muscle is capable of stable expression of secreted protein therapeutics.

Recombinant AAV genomes, in various aspects, comprise nucleic acids of the disclosure and one or more AAV ITRs flanking the nucleic acid. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAV10, AAV11, AAV12, AAV13, AAVrh.74, AAVrh.8, or AAVrh.10, AAVAnc80, AAV7m8, AAV2/1, AAV2/8, AAV2/9, or AAVMYO and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014)²⁹. As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV9, AAV1, AAV2, AAV3, AAV4, AAV5, AAV6, AAV7, AAVrh.74, AAV8, AAV10, AAV11, AAV12 and AAV13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing⁴⁰, addition of synthetic linkers containing restriction endonuclease cleavage sites⁴¹ or by direct, blunt-end ligation⁴². The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539⁴³; and Muzyczka, 1992, Curr. Topics in Microbial. and Immunol., 158:97-129²⁸. Various approaches are described in Ratschin et al., Mol. Cell. Biol. 4:2072 (1984)⁴⁴; Hermonat et al., Proc. Natl. Acad. Sci. USA, 81:6466 (1984)⁴⁵; Tratschin et al., Mol. Cell. Biol. 5:3251 (1985)⁴⁶; McLaughlin et al., J. Virol., 62:1963 (1988)⁴⁷; and Lebkowski et al., Mol. Cell. Biol., October; 8 (10): 3988-96 (1988)⁵⁴; Samulski et al., J. Virol., 63:3822-3828 (1989)⁴⁸; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995)⁴⁹; Paul et al. Human Gene Therapy 4:609-615 (1993)⁵⁰; Clark et al. Gene Therapy 3:1124-1132 (1996)⁵¹; U.S. Pat. Nos. 5,786,211; 5,871,982; and 6,258,595. The foregoing documents are hereby incorporated by reference in their entirety herein, with particular emphasis on those sections of the documents relating to rAAV production. The production and use of self-complementary (sc) rAAV are specifically contemplated and exemplified.

The disclosure thus provides packaging cells that produce infectious rAAV. In one embodiment, packaging cells are stably transformed cancer cells, such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, rAAV is purified by methods standard in the art, such as by column chromatography or cesium chloride gradients. Methods for purifying rAAV vectors from helper virus are known in the art and include methods disclosed in, for example, Clark et al., Hum. Gene Ther., 10 (6): 1031-1039 (1999)⁵²; Schenpp and Clark, Methods Mol. Med., 69 427-443 (2002)⁵³; U.S. Pat. No. 6,566,118 and WO 98/09657.

Compositions comprising the nucleic acids and viral vectors of the disclosure are provided. Compositions comprising delivery vehicles (such as rAAV) described herein are provided. In various aspects, such compositions also comprise a pharmaceutically acceptable carrier. In some aspects, a pharmaceutically acceptable carrier is a diluent, excipient, or buffer. The compositions may also comprise other ingredients, such as adjuvants.

Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include, but are not limited to, a dosage of about 1×10⁷, about 1×10⁸, about 1×10⁹, about 5×10⁹, about 6×10⁹, about 7×10⁹, about 8×10⁹, about 9×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 7×10¹³, about 8×10¹³, about 9×10¹³, about 1×10¹⁴, about 2×10¹⁴, about 3×10¹⁴, about 4×10¹⁴, about 5×10¹⁴, about 1×10¹⁵, to about 1×10¹⁶, or more total viral genomes.

Dosages of about 1×10⁹ to about 1×10¹⁰, about 5×10⁹ to about 5×10¹⁰, about 1×10¹⁰ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, about 1×10¹³ to about 1×10¹⁵ vg and about 6×10¹³ to about 1.0×10¹⁴ vg are also contemplated. One dose exemplified herein is 1×10¹³ vg administered via intravenous or intraperitoneal delivery.

Dosages of rAAV to be administered also, in various aspects, are expressed in units of vg/kg. Such dosages include, but are not limited to, a dosage of about 1×10⁷ vg/kg, about 1×10⁸ vg/kg, about 1×10⁹ vg/kg, about 5×10⁹ vg/kg, about 6×10⁹ vg/kg, about 7×10⁹ vg/kg, about 8×10⁹ vg/kg, about 9×10⁹ vg/kg, about 1×10¹⁰ vg/kg, about 2×10¹⁰ vg/kg, about 3×10¹⁰ vg/kg, about 4×10¹⁰ vg/kg, about 5×10¹⁰ vg/kg, about 1×10¹¹ vg/kg, about 5×10¹¹ vg/kg, about 1×10¹² vg/kg, about 2×10¹² vg/kg, about 3×10¹² vg/kg, about 4×10¹² vg/kg, about 5×10¹² vg/kg, about 6×10¹² vg/kg, about 7×10¹² vg/kg, about 8×10¹² vg/kg, about 9×10¹² vg/kg, about 1×10¹³ vg/kg, about 1.1×10¹³ vg/kg, about 1.2×10¹³ vg/kg, about 1.3×10¹³ vg/kg, about 1.5×10¹³ vg/kg, about 2×10¹³ vg/kg, about 2.5×10¹³ vg/kg, about 3×10¹³ vg/kg, about 3.5×10¹³ vg/kg, about 4×10¹³ vg/kg, about 4.5×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹⁴ vg/kg, about 2×10¹⁴ vg/kg, about 3×10¹⁴ vg/kg, about 4×10¹⁴ vg/kg, about 5×10¹⁴ vg/kg, about 1×10¹⁵ vg/kg, or about 1×10¹⁶ vg/kg.

Dosages of about 1×10⁹ vg/kg to about 1×10¹⁰ vg/kg, about 5×10⁹ vg/kg to about 5×10¹⁰ vg/kg, about 1×10¹⁰ vg/kg to about 1×10¹¹ vg/kg, about 1×10¹¹ vg/kg to about 1×10¹⁵ vg/kg, about 1×10¹² vg/kg to about 1×10¹⁵ vg/kg, about 1×10¹² vg/kg to about 1×10¹⁴ vg/kg, about 1×10¹³ vg/kg to about 2×10¹⁴ vg/kg, about 1×10¹³ vg/kg to about 1×10¹⁵ vg/kg and about 6×10¹³ vg/kg to about 1.0×10¹⁴ vg/kg are also included in various aspects. Some doses exemplified herein are about 1.5×10¹¹ vg/kg or about 3×10¹³ vg/kg administered via intramuscular, intravenous, or intraperitoneal delivery.

Transduction or transfection of cells with rAAV of the disclosure results in sustained expression of the TCAP gene/protein. As used herein, the terms “transduction” and “transfection” are used interchangeably. The term “transduction” or “transfection” is used to refer to, as an example, the administration/delivery of the TCAP gene to a target cell either in vivo or in vitro, via a replication-deficient rAAV described herein resulting in the expression of the TCAP gene/protein by the target cell. The disclosure thus provides methods of administering/delivering rAAV which express the TCAP gene to a cell or to a subject. In some aspects, the subject is a mammal. In some aspects, the mammal is a human. These methods include transducing cells and tissues (including, but not limited to, peripheral motor neurons, sensory motor neurons, neurons, Schwann cells, and other tissues or organs, such as muscle, liver and brain) with one or more rAAV described herein. Transduction may be carried out with gene cassettes comprising cell-specific control elements.

Methods of transducing a target cell with a delivery vehicle (such as a nanoparticle, extracellular vesicle, exosome, or vector (e.g., rAAV)), in vivo or in vitro, are provided. The in vivo methods comprise the step of administering an effective dose, or effective multiple doses, of a composition comprising a delivery vehicle (such as rAAV) to an animal (including a human subject or patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Thus, methods are provided of administering an effective dose (or doses, administered essentially simultaneously or doses given at intervals) of rAAV described herein to a subject in need thereof.

Provided herein are medicaments and methods for treating, ameliorating, or preventing diseases associated with a mutant TCAP gene or aberrant TCAP gene expression. Molecular, biochemical, histological, and functional outcome measures demonstrate the therapeutic efficacy of the methods. The level of human TCAP transcript in animals in can be confirmed by RT-PCR and/or RNAseq. The TCAP protein expression level in skeletal muscles, including heart and diaphragm can be assessed using western blotting. TCAP localization and proper co-localization with its binding partners in Z-discs as well as hallmarks of the pathology of muscles and inflammation can be confirmed by immunohistochemistry. To assess efficacy of potential treatment in mice, measurements of muscle contractile function can be performed using Aurora Whole Animal Muscle Test System. In patients, a variety of functional outcome measures may be used to assess successful treatment, including: 100 meter timed test, 10 meter walk/run test, North Star Ambulatory Assessment for limb girdle type muscular dystrophies (NSAD), Performance of Upper Limb (PUL) 2.0, and myometry assessments of force (including measures of shoulder abduction, elbow flexion/extension, and knee flexion/extensionforce).

In the methods of the disclosure, expression of the TCAP protein is increased by at least 10, at least 20, at least 30, at least 40, at least 50, at least 60, at least 70, at least 80, at least 90, at least 95, at least 98 percent, at least 99 percent, or 100 percent.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins and polypeptides, and di peptide boronic acid molecules, such as Bortezomib.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, methotrexate, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include rapamycin (also known as sirolimus), everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and rapamycin (also known as sirolimus).

Purine analogs block nucleotide synthesis and include IMDH inhibitors. Exemplary purine analogs include azathioprine, mycophenolate such as mycophenolate acid or mycophenolate mofetil and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

One exemplary combination therapy is the delivery of rapamycin and rituximab prior to, or contemporaneous with, delivery of the AAV vector. Another exemplary combination therapy is the delivery of rapamycin, rituximab, and a corticosteroid, such as prednisone.

Administration of an effective dose of a nucleic acid, nanoparticle, extracellular vesicle, exosome, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the miRNAs.

In particular, actual administration of delivery vehicle (such as rAAV) may be accomplished by using any physical method that will transport the delivery vehicle (such as rAAV) into a target cell of a subject. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the methods of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, suMPZ or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides kits for use in the treatment of a disease or disorder described herein. Such kits include at least a first sterile composition comprising any of the nucleic acids described herein above or any of the viral vectors described herein above in a pharmaceutically acceptable carrier. Another component is optionally a second therapeutic agent for the treatment of the disorder along with suitable container and vehicles for administrations of the therapeutic compositions. The kits optionally comprise solutions or buffers for suspending, diluting or effecting the delivery of the first and second compositions.

In one embodiment, such a kit includes the nucleic acids or vectors in a diluent packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, stabilizers, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications, patents and patent applications cited in this specification are herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims. Thus, the following examples are provided by way of illustration and not limitation.

### EXAMPLES

### Example 1

### Gene Therapy Constructs Encoding TCAP and Materials and Methods

AAV genome vector constructs encoding TCAP were generated as set forth in FIG. 1, which depicts the rAAV9 vector design with the full-length transcript of TCAP cDNA under the control of a TCAP native promoter (SEQ ID NO: 3), an MHCK7 promoter (SEQ ID NO: 4), or a CMV promoter (SEQ ID NO: 5).

Ten rAAV vector constructs were designed as set out in FIG. 1 to test the restoration of TCAP expression. The vectors comprise the following promoters: human TCAP native promoter (NP), the mouse analog of which showed to be involved in circadian TCAP regulation in skeletal muscles (orange, constructs 1-2); MHCK7 promoter (MHCK7), which shows high-level expression in fast and slow skeletal and cardiac muscle (lavender, constructs 3-6); human cytomegalovirus (CMV) promoter, which shows high level of expression in all tissue and organs (purple, constructs 7-10). The human TCAP gene is represented as exon 1 (lighter green), intron 1 (gold, NI, native intron) and exon 2 (darker green). A chimeric simian virus 40 (SV40) intron (SI, synthetic intron) is represented in light gold.

Tables 4A-B, set out below, provides specifics regarding the transgene sequences and the sequences in AAV vectors.

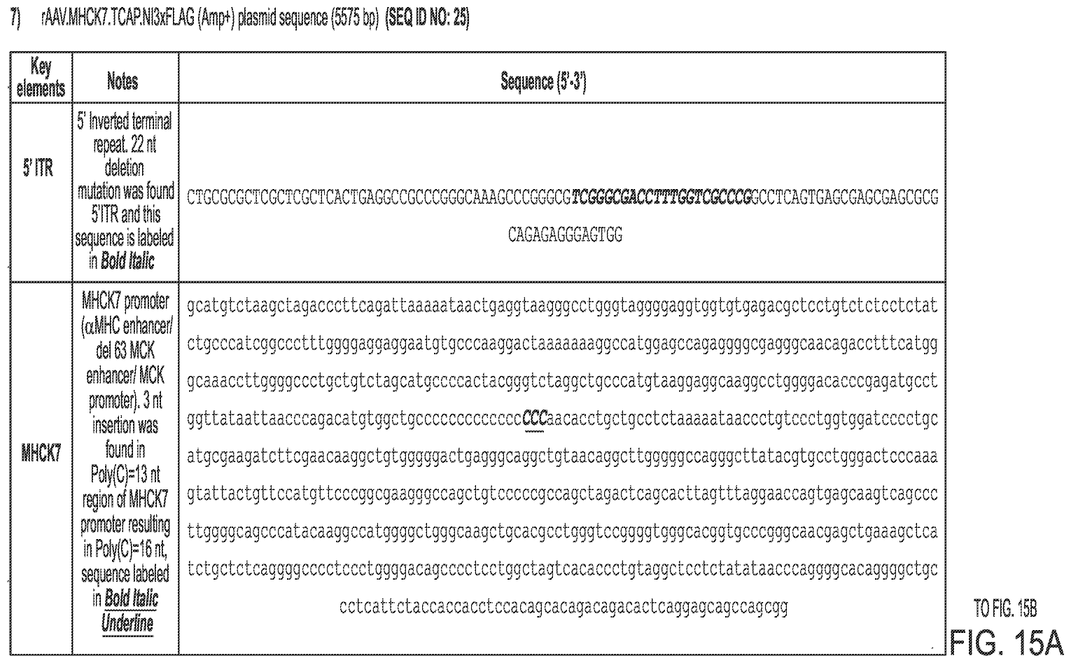

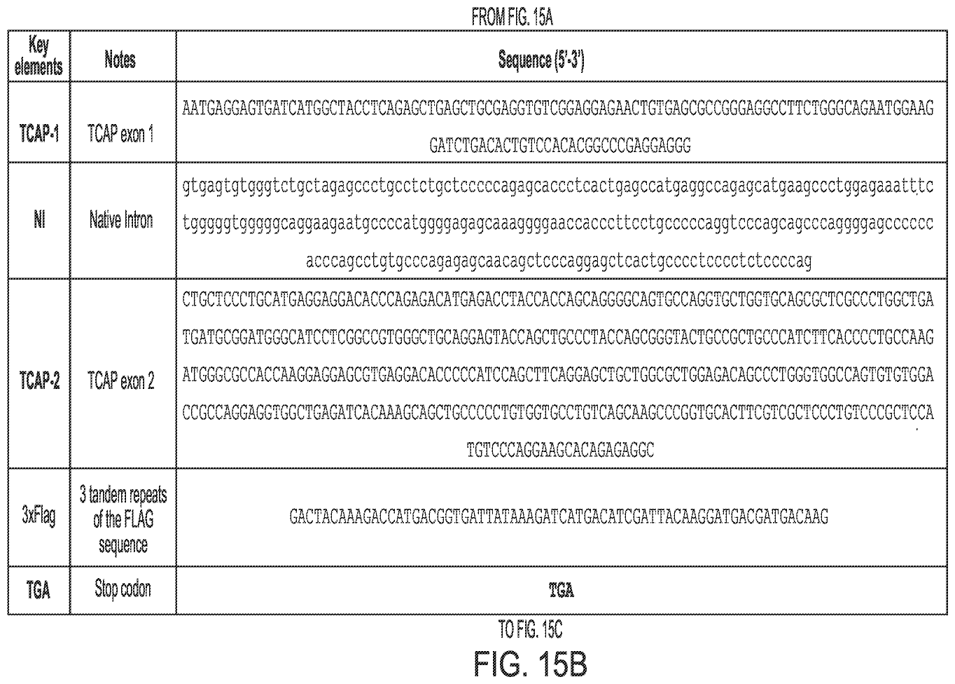

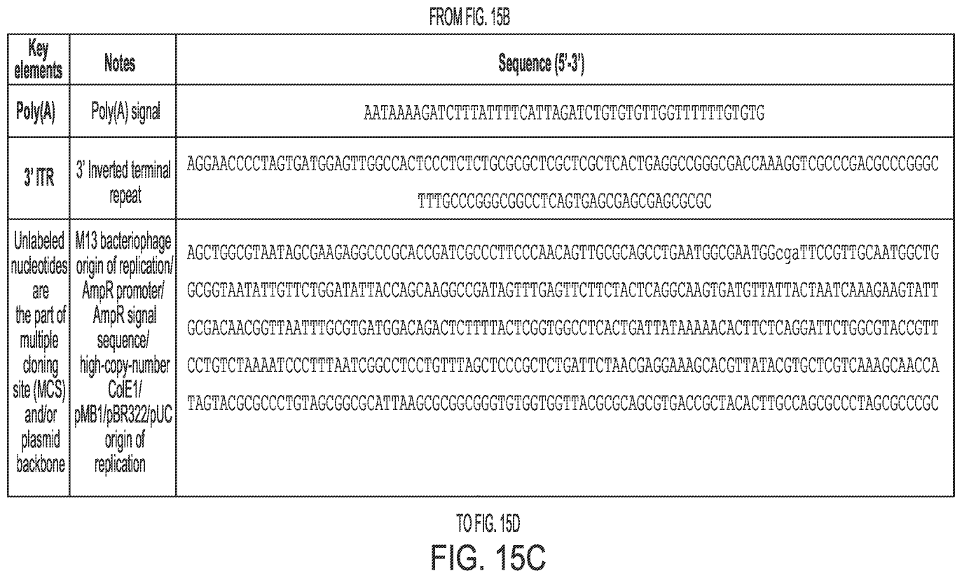

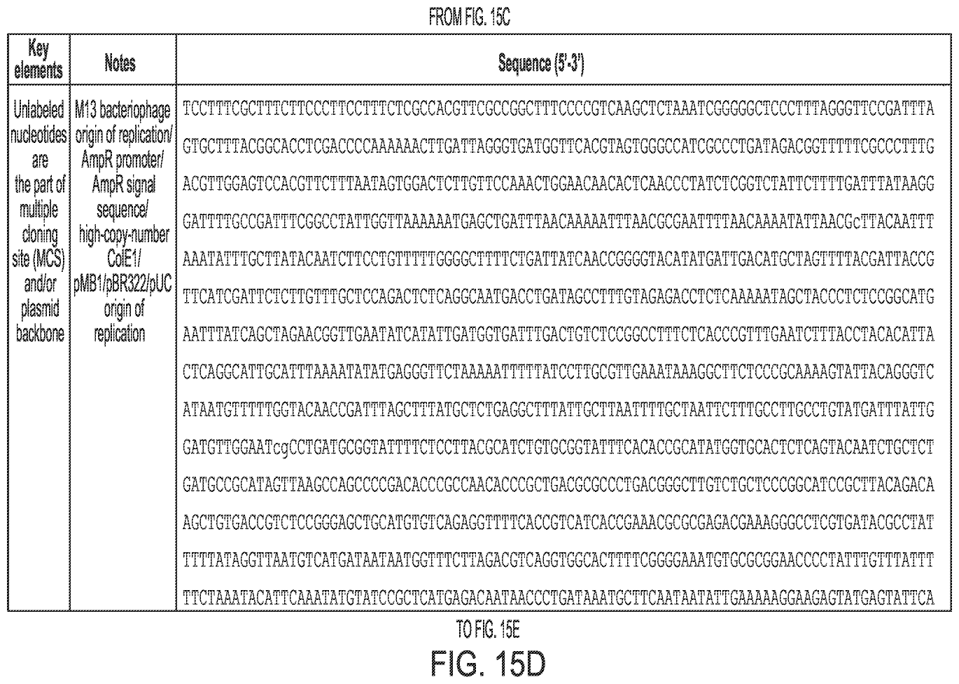

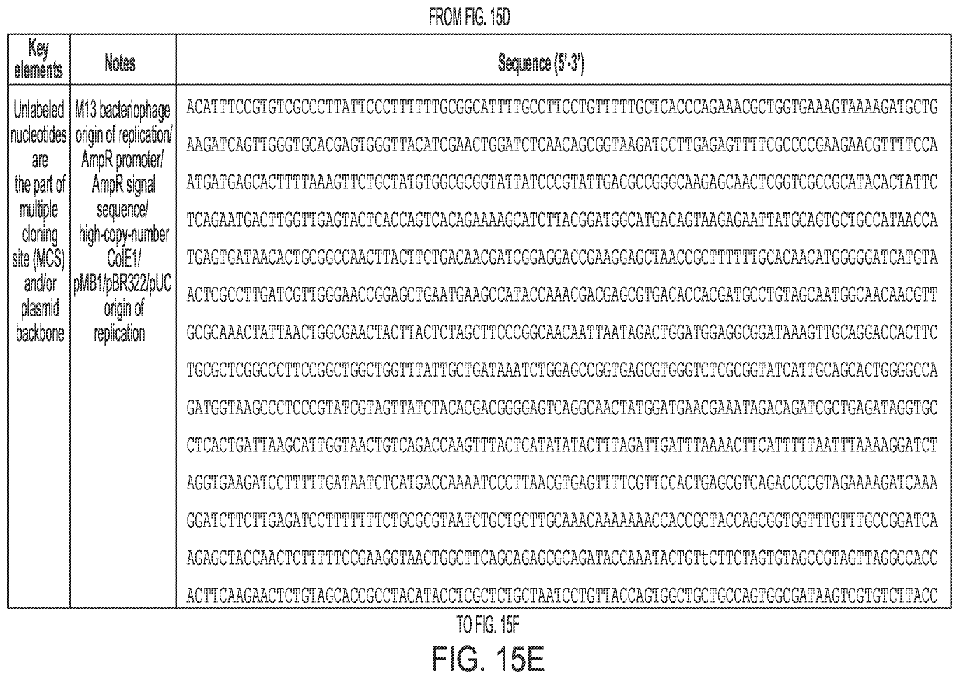

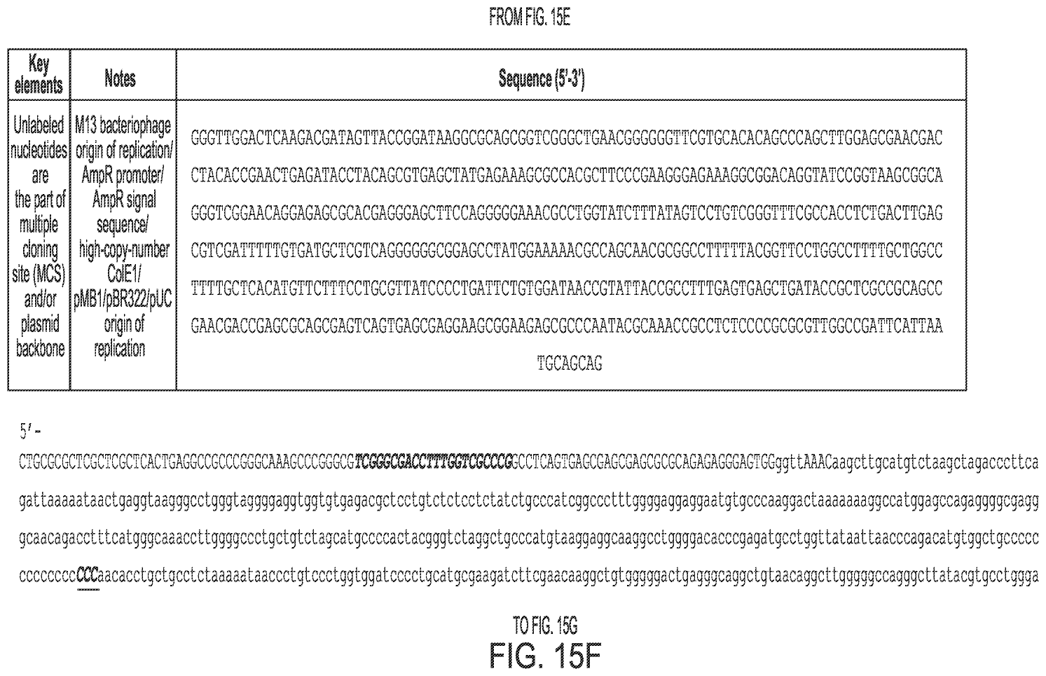

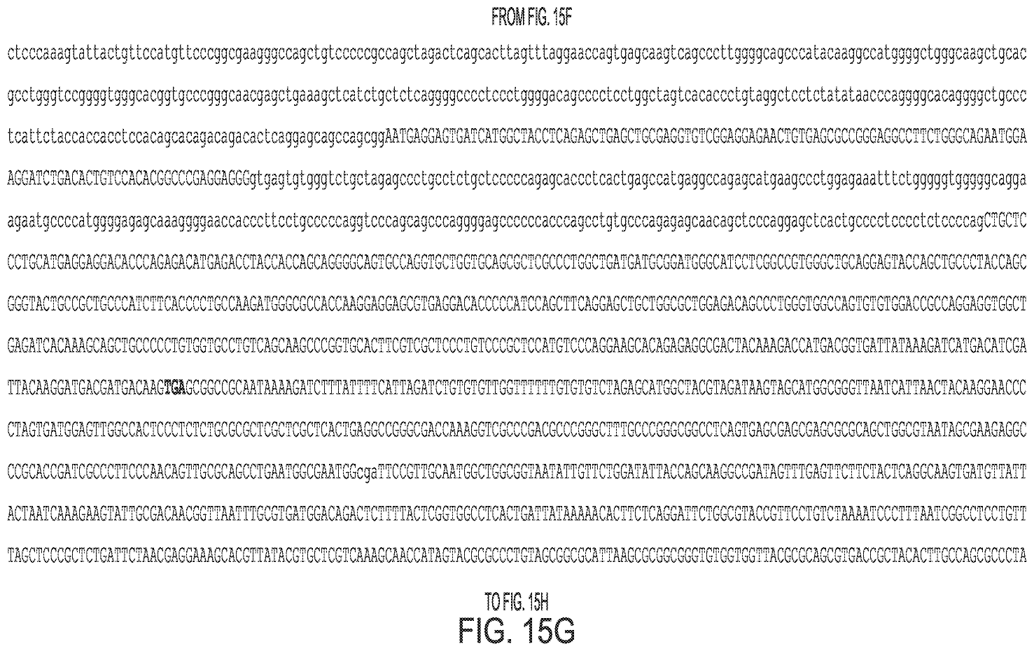

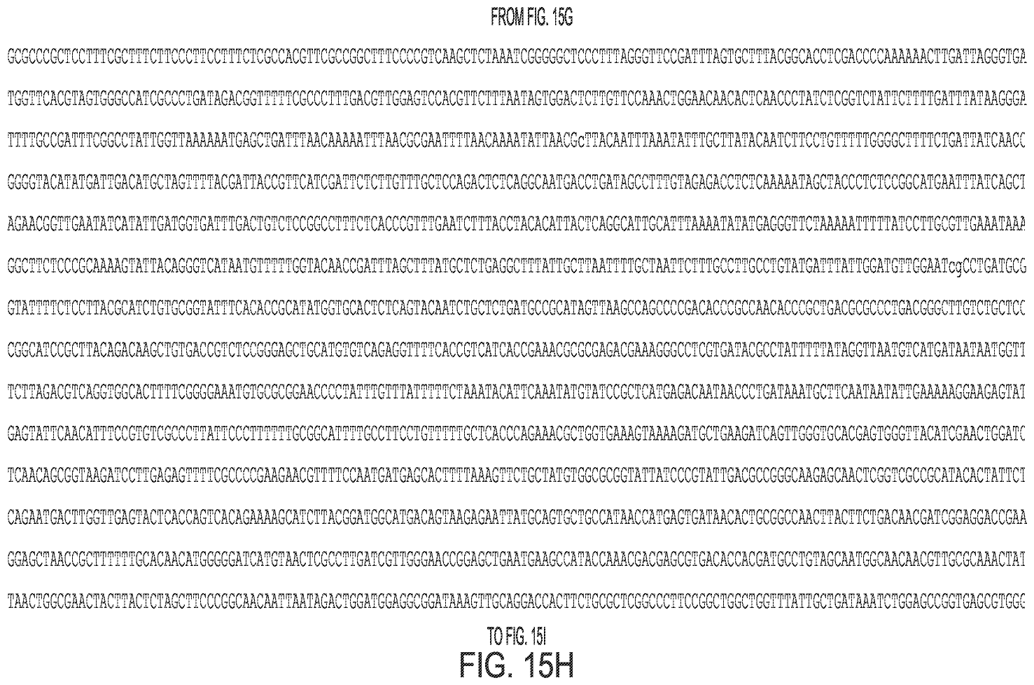

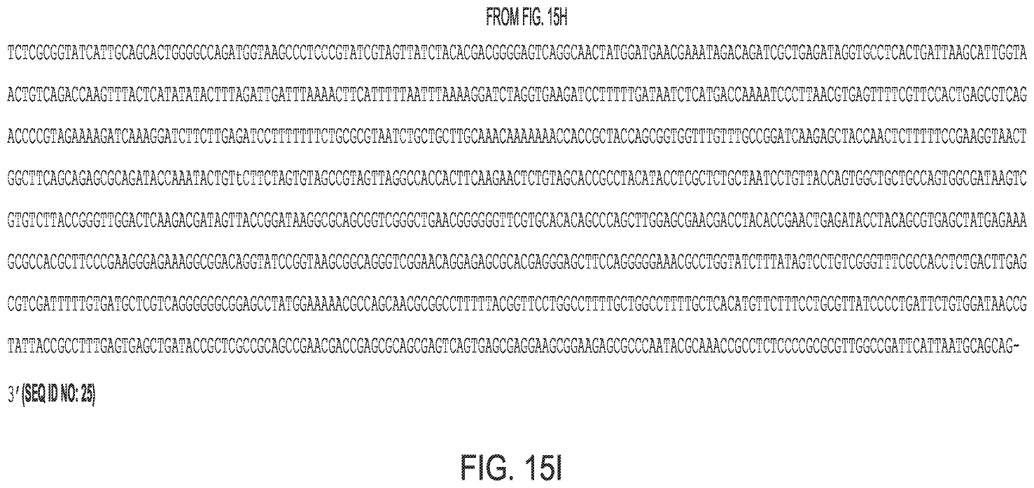

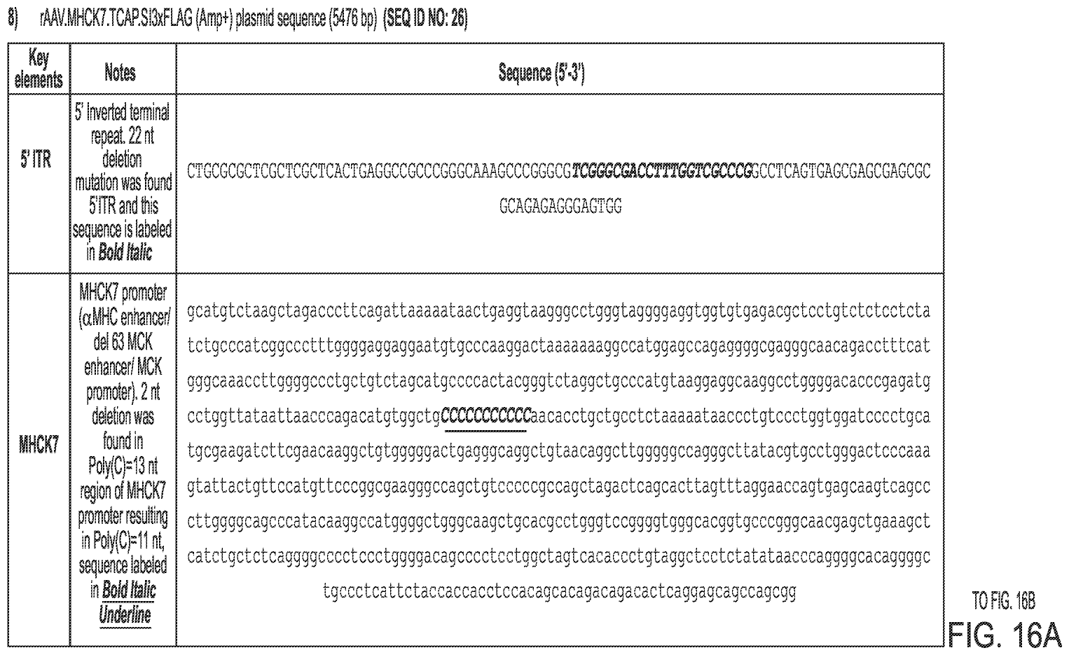

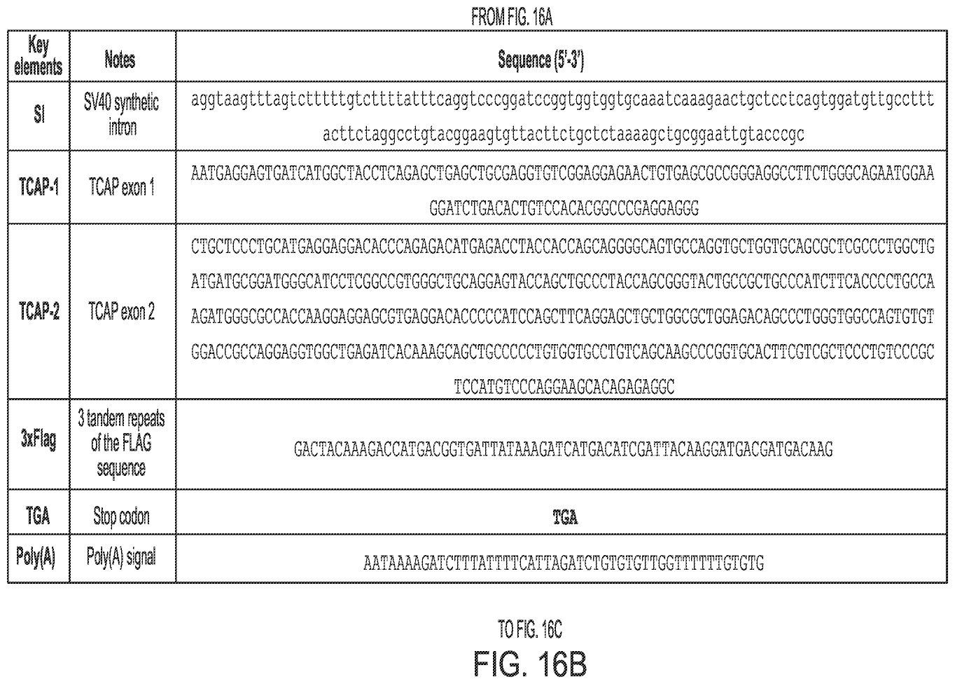

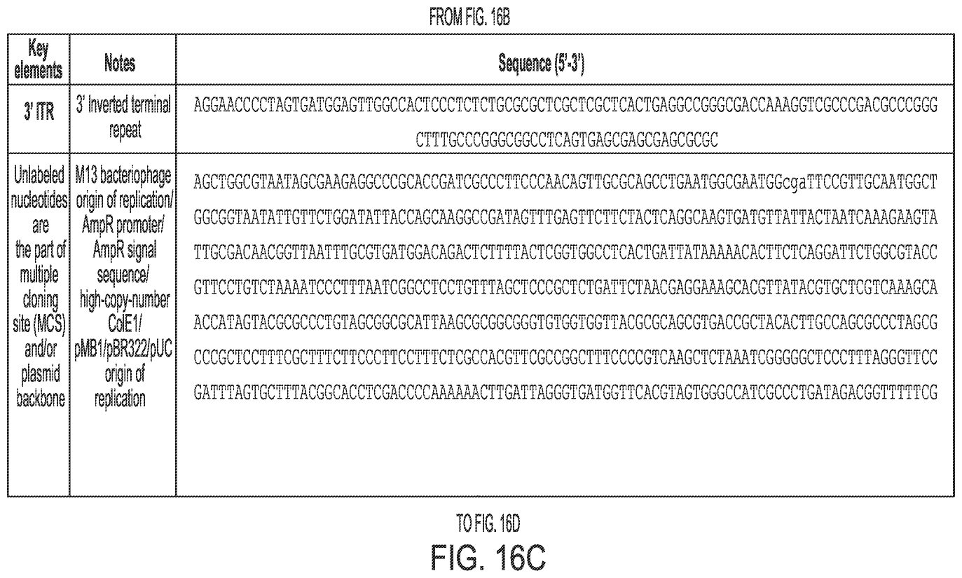

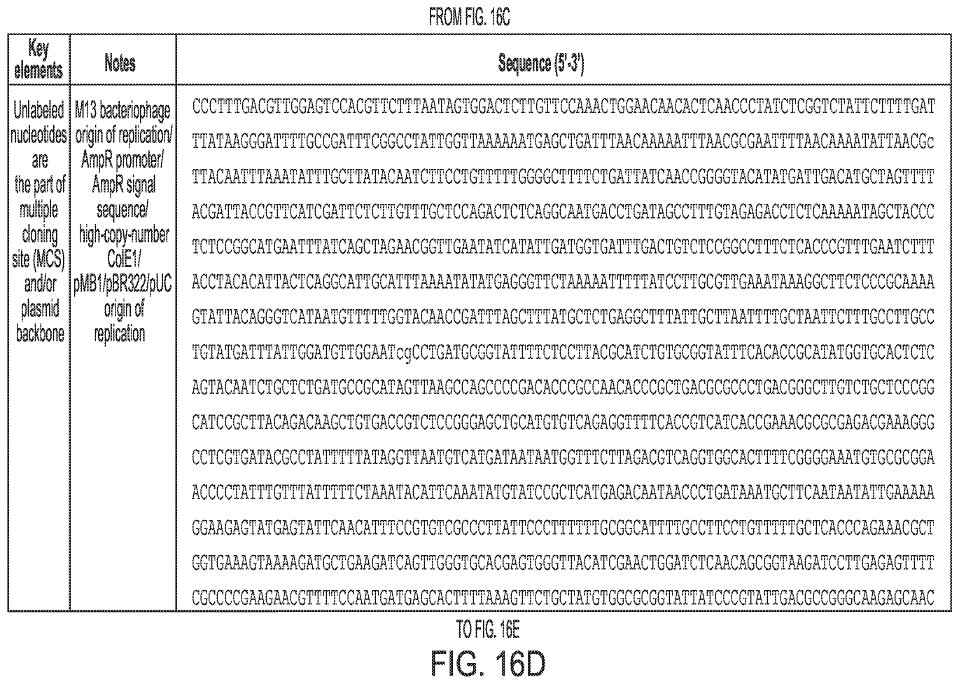

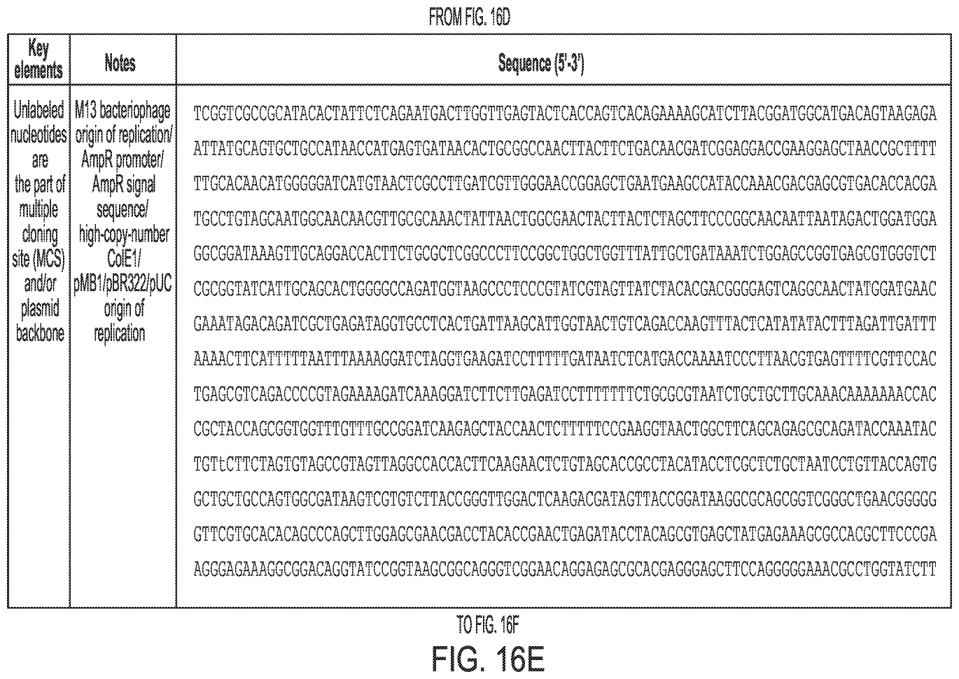

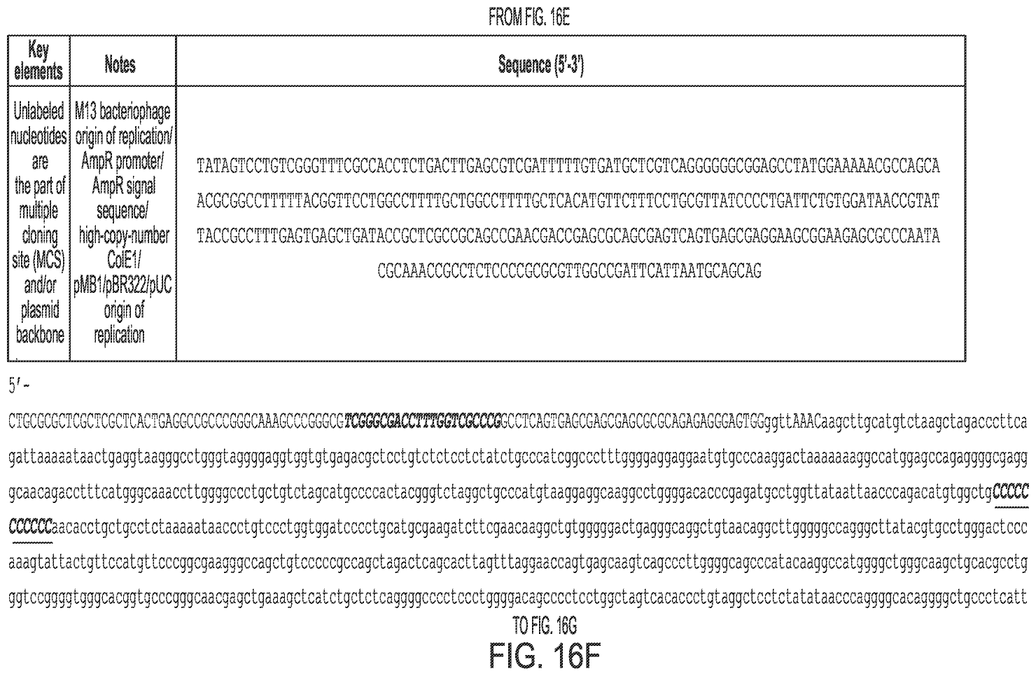

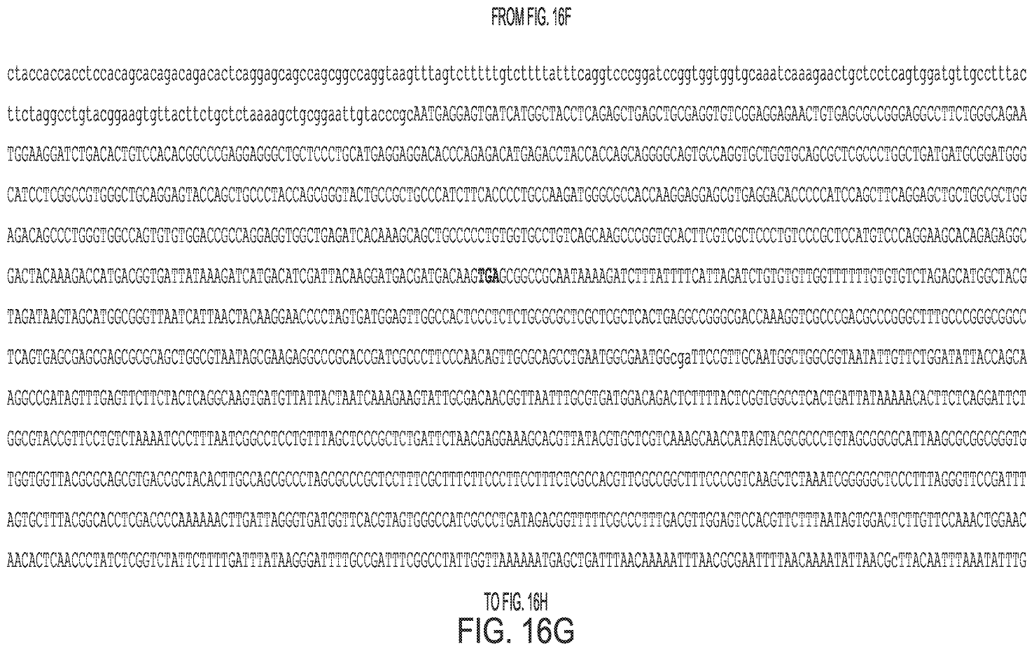

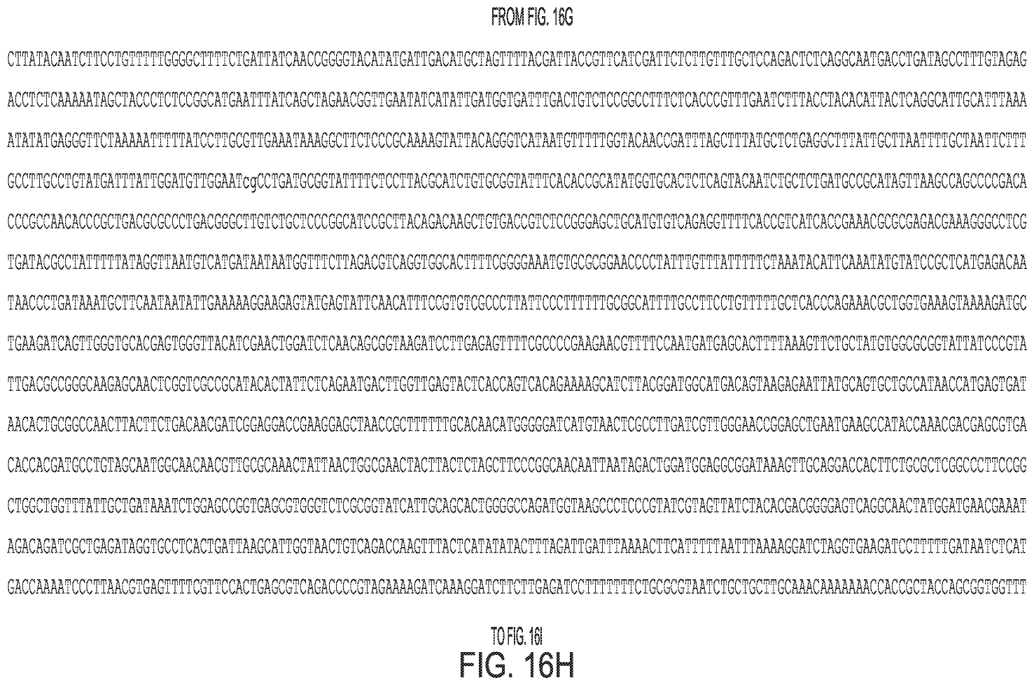

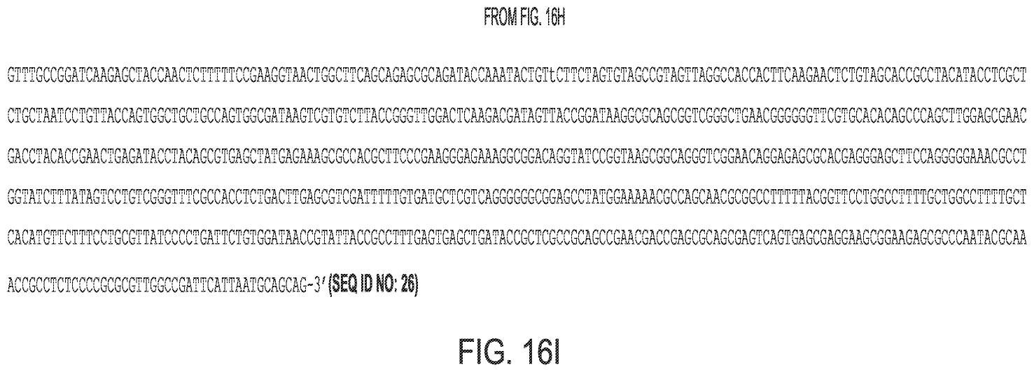

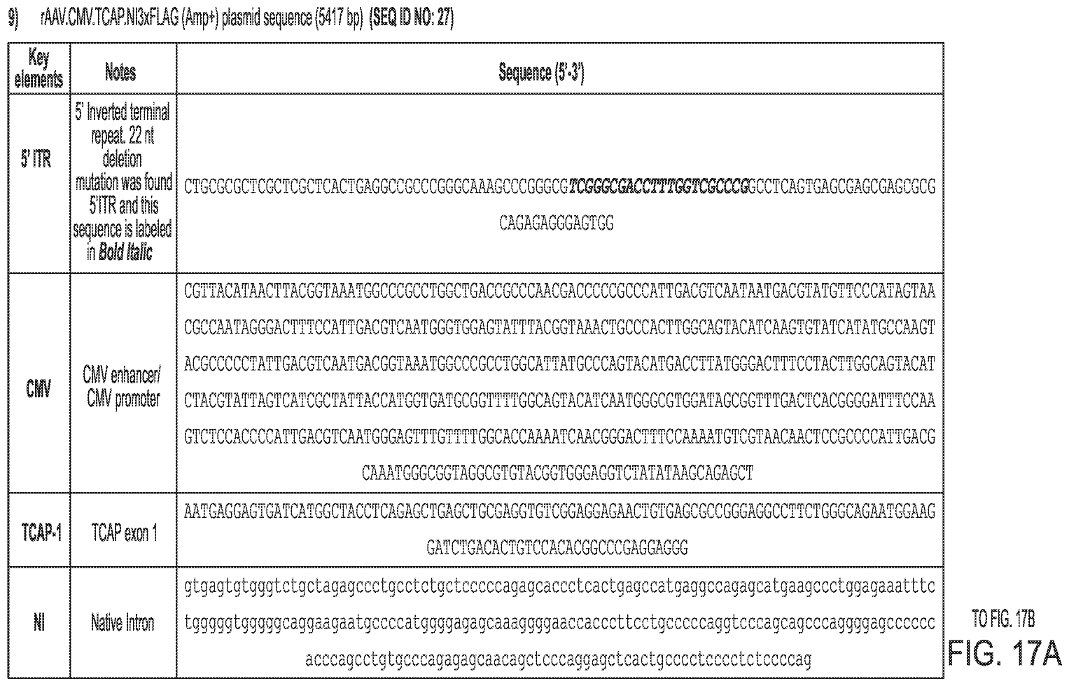

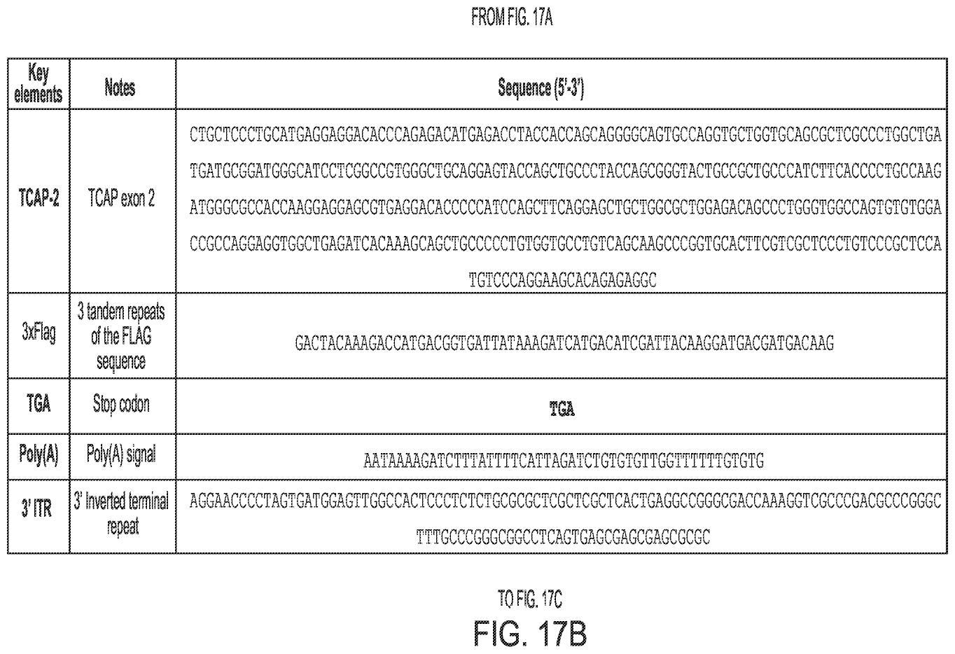

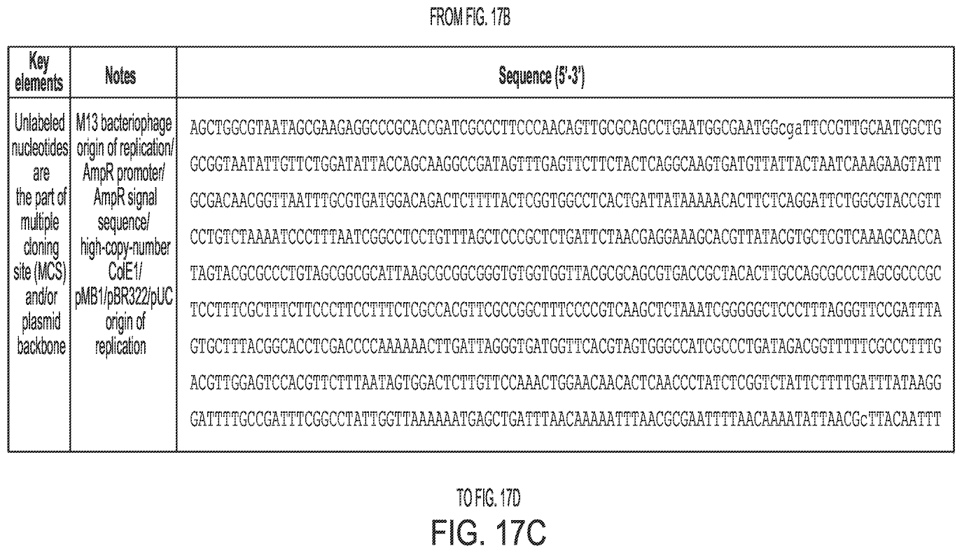

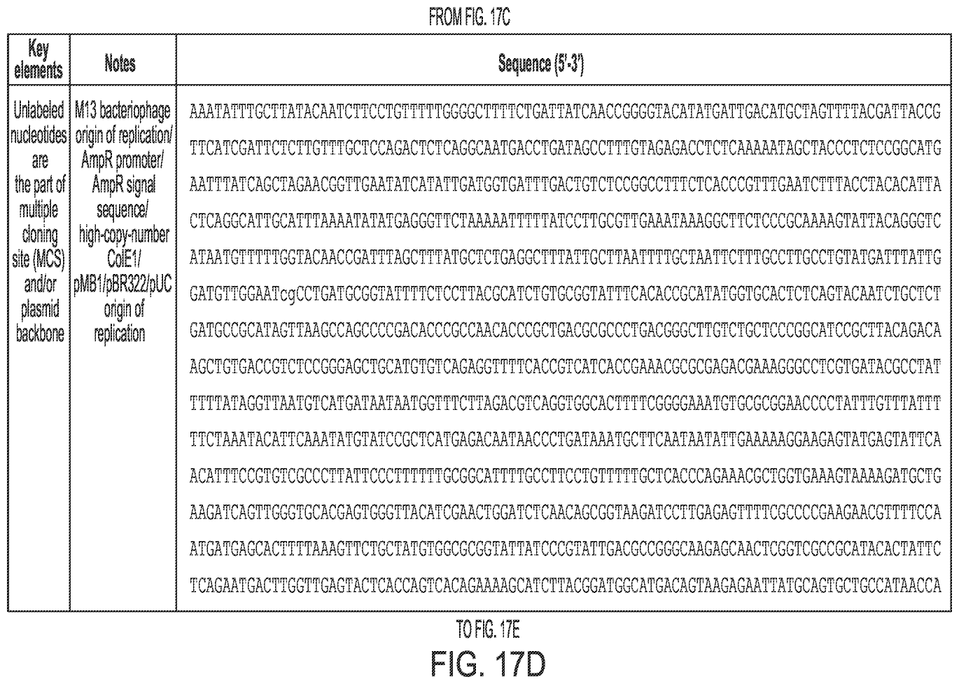

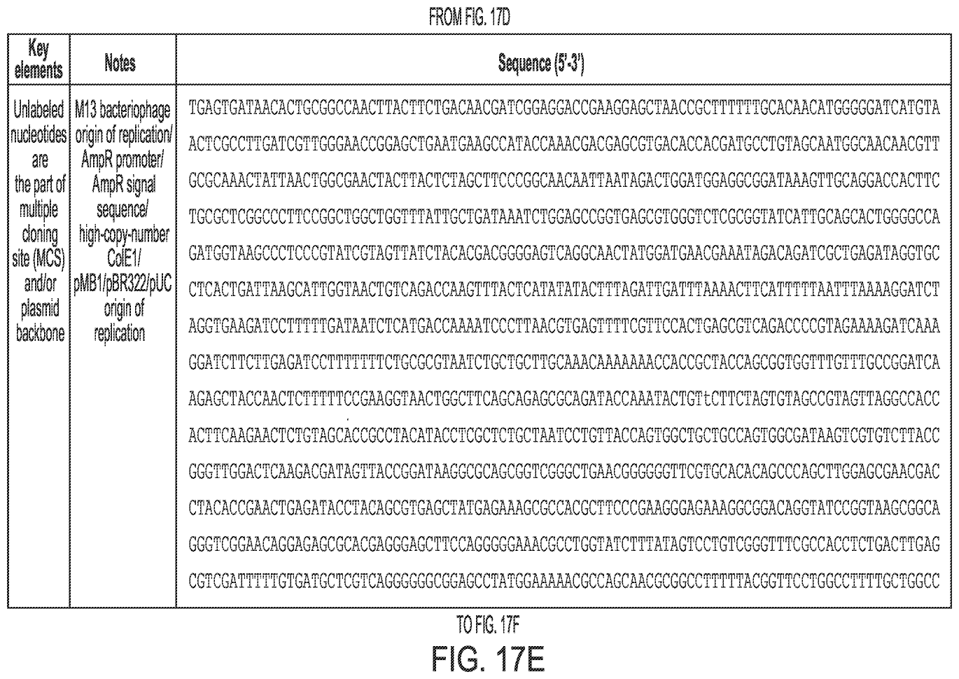

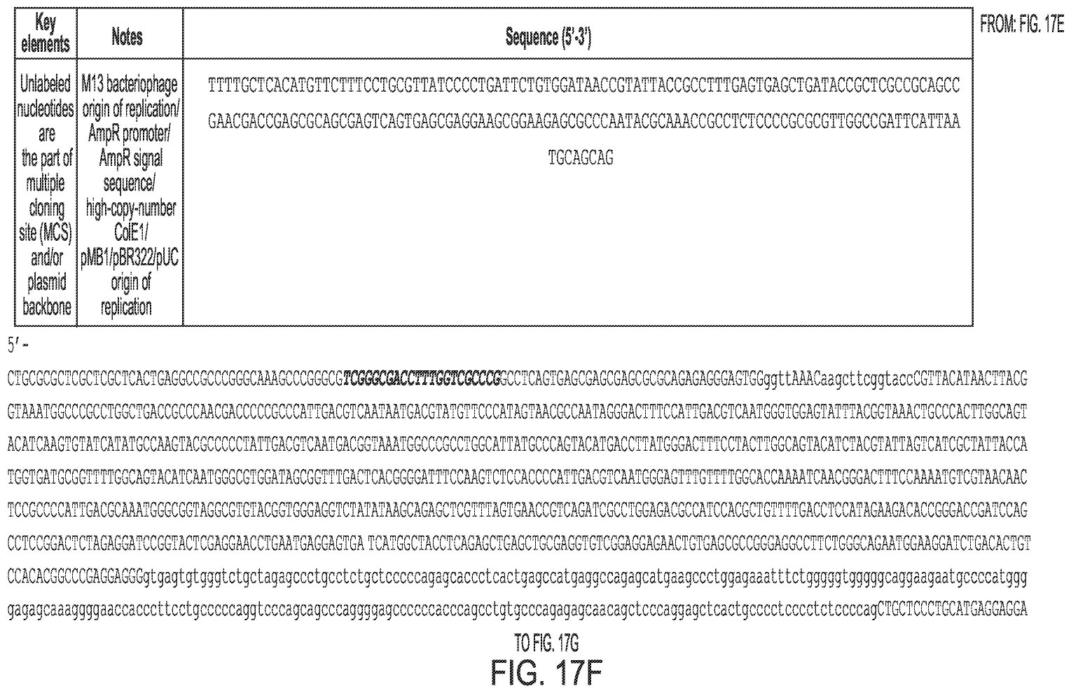

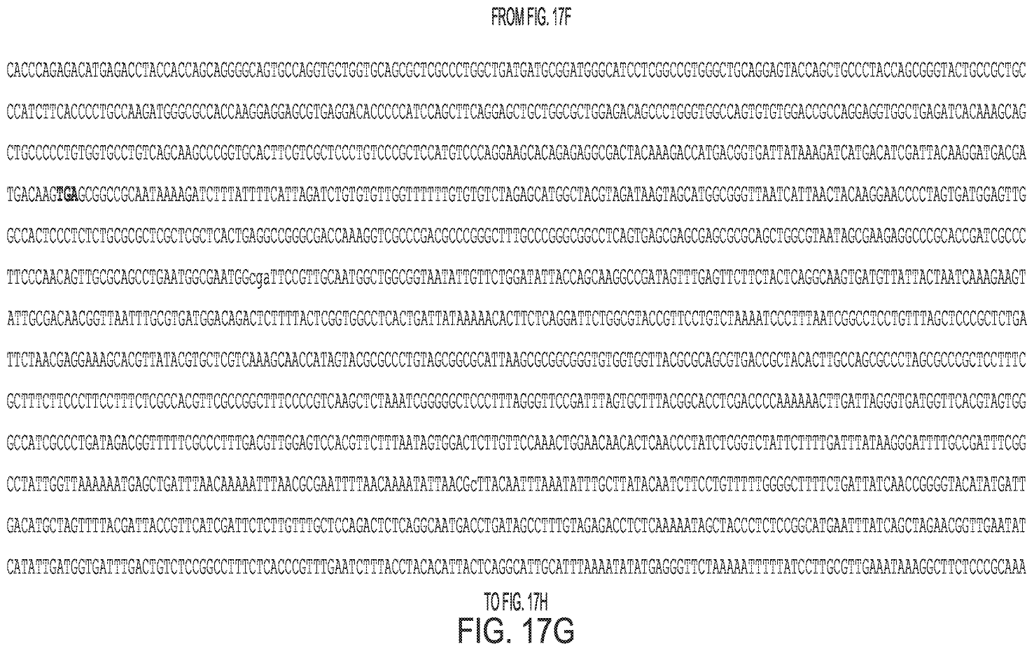

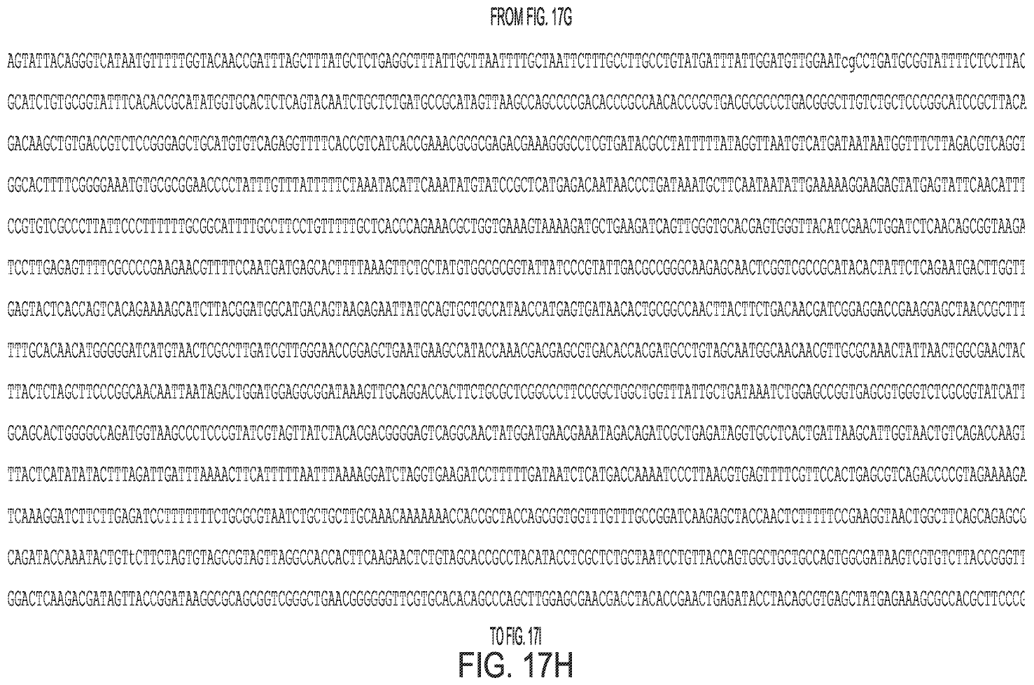

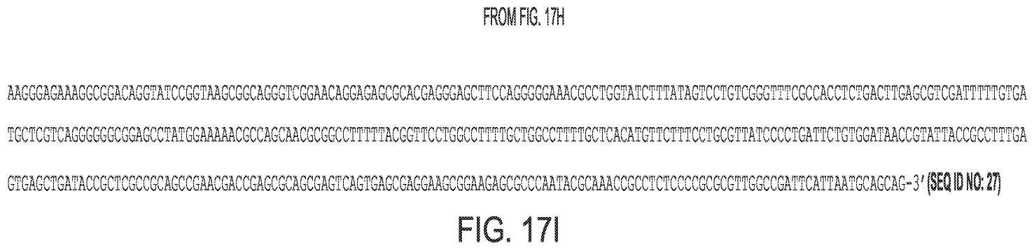

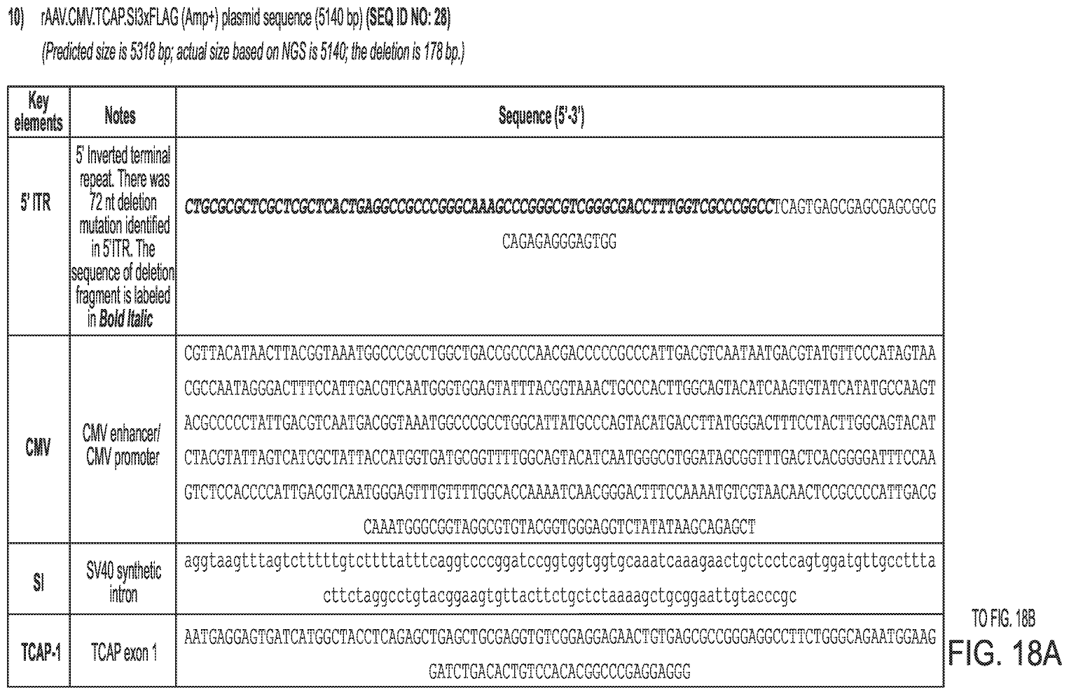

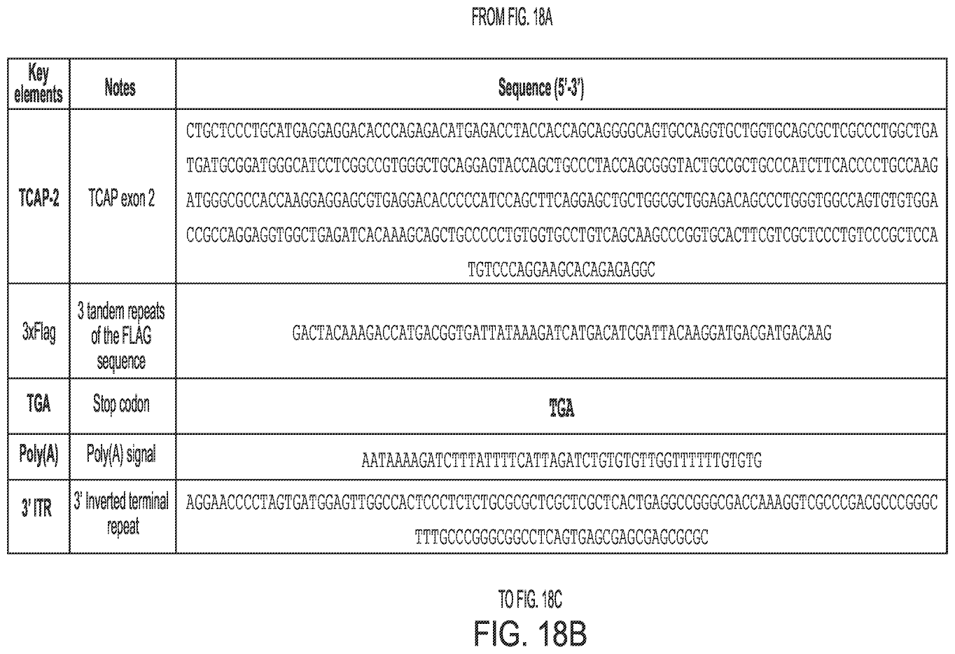

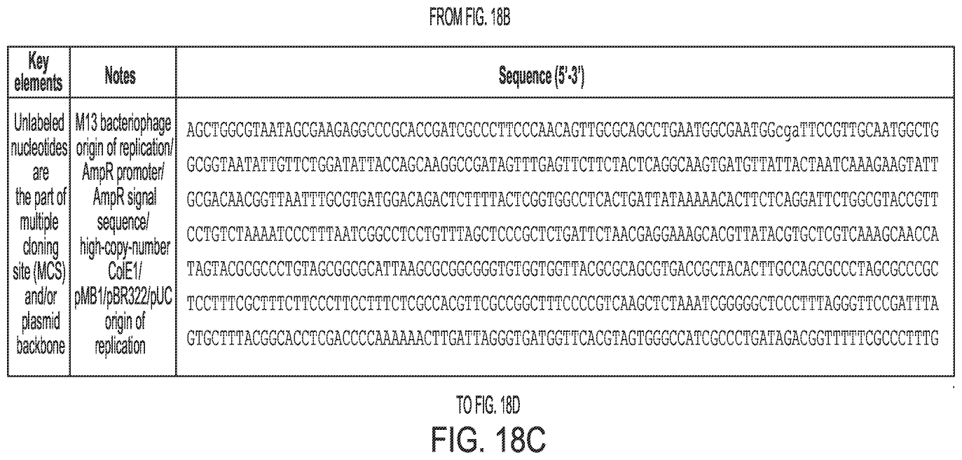

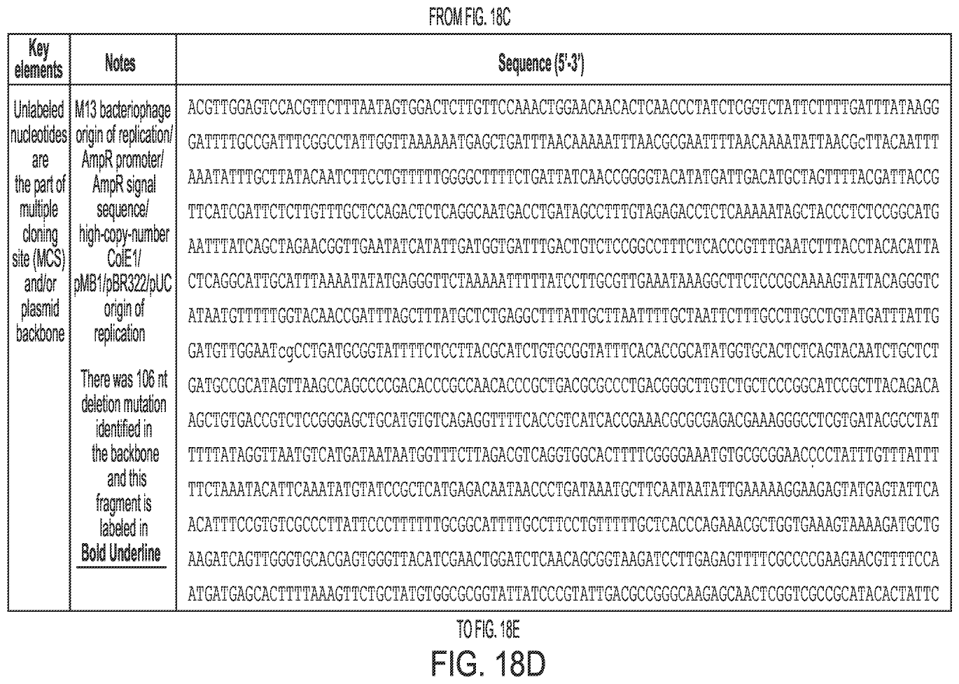

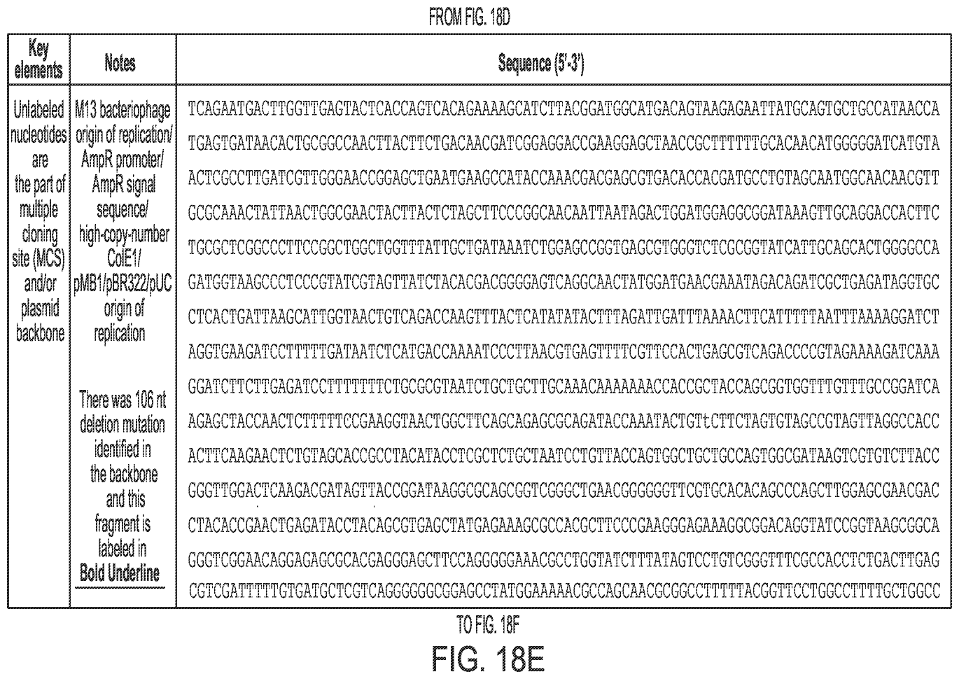

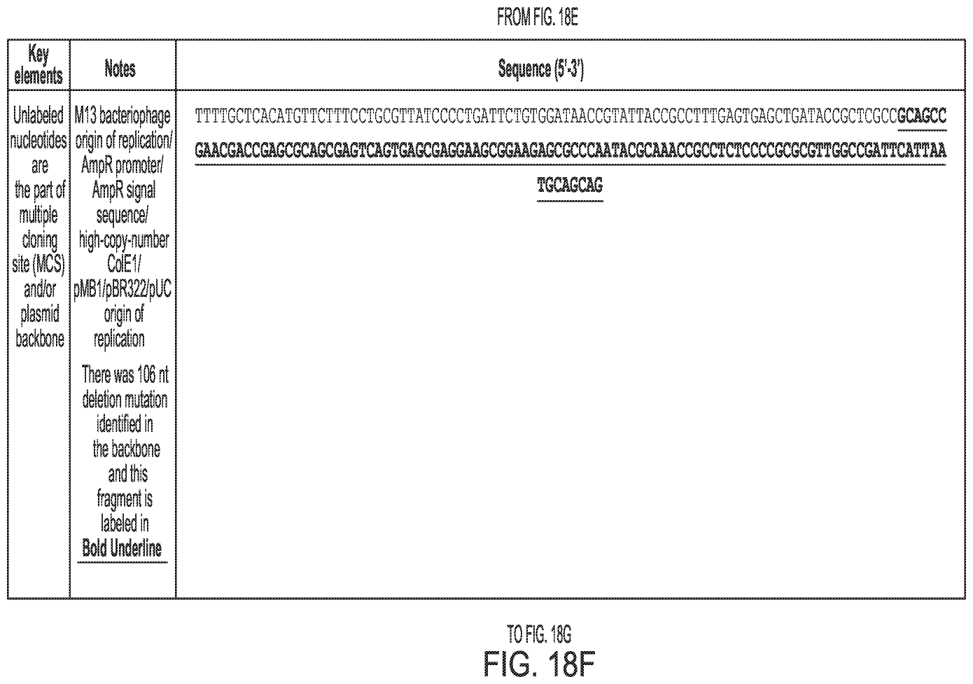

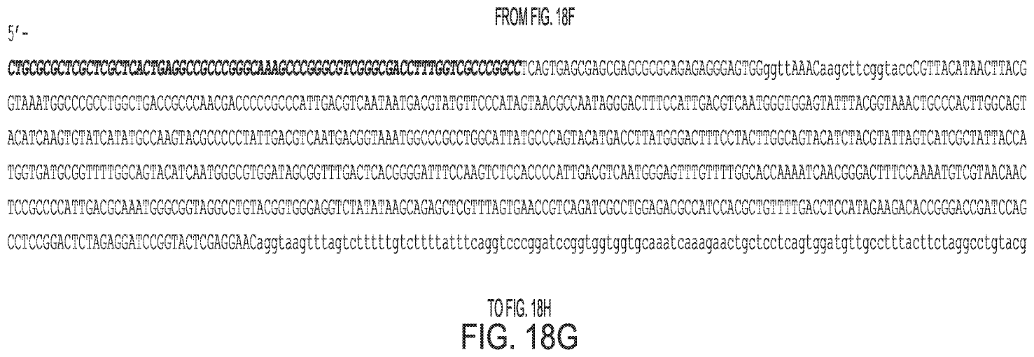

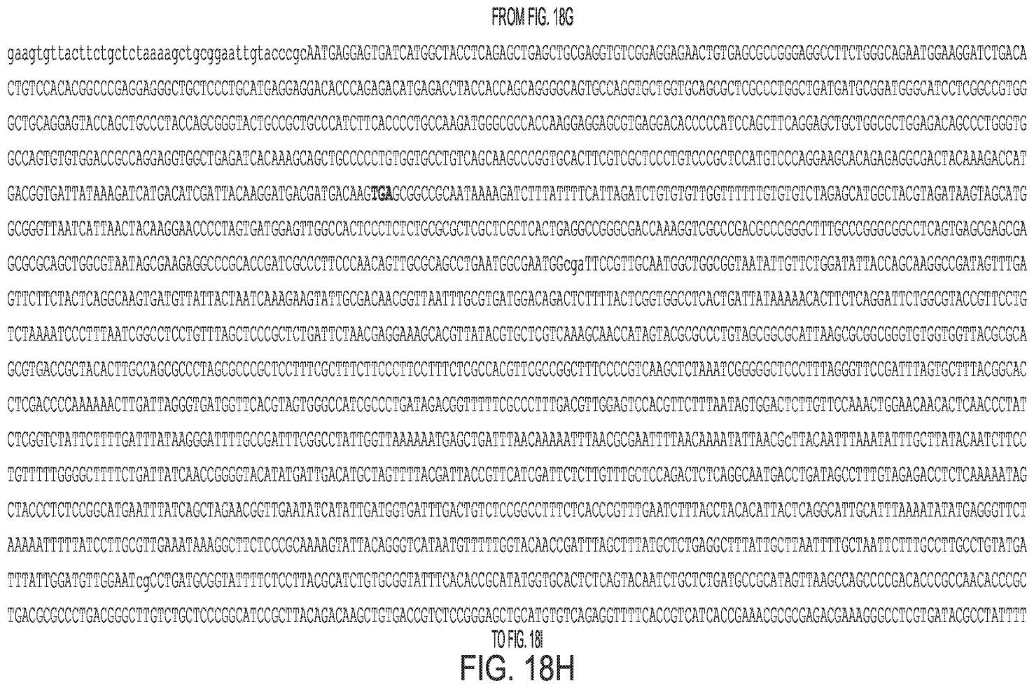

TABLE 4A
TCAP construct overview.

| | | | Total | Transgene size | | | |
|---|---|---|---|---|---|---|---|
| | | Total | plasmid | without | Transgene | | |
| | | Plasmid | SEQ ID | 5′ and 3′ ITR | SEQ ID | | Other |
| No. | Name | size (bp)** | NO: | sequences (bp) | NO: | Promoter | elements |
| 1 | rAAV.NP.TCAP (FIG. 9) | 5627 | 19 | 1725 | 9 | NP | No FLAG |
| 2 | rAAV.MHCK7.TCAP.NI (FIG. 10) | 5506 | 20 | 1609 | 10 | MHCK7 | No FLAG |
| 3 | rAAV.MHCK7.TCAP.SI (FIG. 11) | 5412 | 21 | 1515 | 11 | MHCK7 | No FLAG |
| 4 | rAAV.CMV.TCAP.NI (FIG. 12) | 5351 | 22 | 1447 | 12 | CMV | No FLAG |
| 5 | rAAV.CMV.TCAP.SI (FIG. 13) | 5252 | 23 | 1348 | 13 | CMV | No FLAG |
| 6 | rAAV.NP.TCAP.3xFLAG (FIG. 14) | 5693 | 24 | 1791 | 14 | NP | 3xFLAG |
| 7 | rAAV.MHCK7.TCAP.NI3xFLAG (FIG. 15) | 5575^{α} | 25 | 1678^{α} | 15 | MHCK7 | 3xFLAG |
| 8 | rAAV.MHCK7.TCAP.SI3xFLAG (FIG. 16) | 5476^{β} | 26 | 1579^{β} | 16 | MHCK7 | 3xFLAG |
| 9 | rAAV.CMV.TCAP.NI3xFLAG (FIG. 17) | 5417 | 27 | 1513 | 17 | CMV | 3xFLAG |
| 10 | rAAV.CMV.TCAP.SI3xFLAG (FIG. 18) | 5140^{γ} | 28 | 1414 | 18 | CMV | 3xFLAG |
| 11 | rAAV.NP.TCAP/SD (FIG. 19) | 5749 | 39 | 1725 | 29 | NP | No FLAG |
| 12 | rAAV.MHCK7.TCAP.NI/SD (FIG. 20) | 5628 | 40 | 1609 | 30 | MHCK7 | No FLAG |
| 13 | rAAV.MHCK7.TCAP.SI/SD (FIG. 21) | 5534 | 41 | 1515 | 31 | MHCK7 | No FLAG |
| 14 | rAAV.CMV.TCAP.NI/SD (FIG. 22) | 5440 | 42 | 1447 | 32 | CMV | No FLAG |
| 15 | rAAV.CMV.TCAP.SI/SD (FIG. 23) | 5341 | 43 | 1348 | 33 | CMV | No FLAG |
| 16 | rAAV.NP.TCAP.3xFLAG/SD (FIG. 24) | 5815 | 44 | 1791 | 34 | NP | 3xFLAG |
| 17 | rAAV.MHCK7.TCAP.NI3xFLAG/SD (FIG. 25) | 5694 | 45 | 1675 | 35 | MHCK7 | 3xFLAG |
| 18 | rAAV.MHCK7.TCAP.SI3xFLAG/SD | 5600 | 46 | 1581 | 36 | MHCK7 | 3xFLAG |
| 19 | rAAV.CMV.TCAP.NI3xFLAG/SD | 5506 | 47 | 1513 | 37 | CMV | 3xFLAG |
| 20 | rAAV.CMV.TCAP.SI3xFLAG/SD | 5407 | 48 | 1414 | 38 | CMV | 3xFLAG |

NP—native promoter
NI—native intron
SI—synthetic intron
3xFLAG—3 tandem repeats of the FLAG sequence
^{α}Total plasmid size and transgene size should be 5572 bp and 1675 bp, respectively. This discrepancy is due to additional 3 nt insertion found in the MHCK7 promoter (see Tables 2 and 3).
^{β}Total plasmid size and transgene size should be 5478 bp and 1581 bp, respectively. This discrepancy is due to 2 nt deletion found in the MHCK7 promoter (see Tables 2 and 3).
^{γ}Total plasmid size and transgene size should be 5318 bp. This discrepancy is due to a deletion mutation found in the 5′ITR (72 bp) and 5′ backbone (106 bp) (see Tables 2 and 3).

TABLE 4B
TCAP construct transgene sequences with ITRs (SEQ ID NOs: 49-58).

| | | Transgene | | |
|---|---|---|---|---|
| | | size with | | |
| | | 5′ and 3′ | | |
| | | ITR | SEQ | |
| | | sequences | ID | |
| No. | Name | (bp) | NO: | Sequence (5′-3′) |
| 11 | NP.TCAP/SD | 2096 | 49 | CTGCGCGCTCGCTCGCTCACTGAGGCCGCCCGGGCAAAGCCCGGG |
| | | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACccttaagTCTAGAggCTCGAGaagcttGCTAGCCC |
| | | | | CATGAAGAAGAGCCAGTCCCCTCCCAGGTAGCACATCAGAGCGGG |
| | | | | CAGGCTCCAGAGCTCTTCTGGTCCTCTTGCAGTGGCCAGAAAGTG |
| | | | | GGGAAAGGGGCCCAGAGAGGGGCAGGGACTGACCCATGGTCACAC |
| | | | | AGCCTATTTATGGCTGATCTGGAGCCAGGCCTTCTGACTATCACC |
| | | | | CATCAGAGCCCCTGGATTCCCATTTCACCCAACACAGGTGTTGGG |
| | | | | GTAGAACTGGGGGCCCTCCACCCCTACTAGCCTGACTGTCTCCTG |
| | | | | AGACCTGTCTCCTAAGACCACTTACCTGACCACTGGCCATGGGGA |
| | | | | AGGAAAACACCTGGCCAAGGCTCATGCCCCAGGTTCCCAGGCATG |
| | | | | AAGTTGACTGTCTCCGCCCCTGCTGGTGTGGTGAGGGTGACTGGG |
| | | | | GACTAGGCACTAGGCCTTTGGTGCAGGCGCCTGAGGACGTGGTTG |
| | | | | CACTCTCCCTTCTGGGGATATGCCCTTGAGCCCAGGCAGAGGAGA |
| | | | | GCACAGCCAGGGCAGGACCTGGCAGCCCTGGTACAGAGCCCAGAG |
| | | | | GGGGCATCAGTTCCTGCTGGTCCTGCTCTGTTTACAGACAAGCTG |
| | | | | CTGTCCTCCCTGCAAAGGGGAGTGGGTGGGGCAGAGGGCAAGTGC |
| | | | | CAGGGGGGCACAAGGCTGGGCATGTGGCTGGCATGAGACGGTGTC |
| | | | | TGAGTAATGTCAGGCACCTGGAGGCATTGACCCCAGGACCTTGGA |
| | | | | CCCCAGACCTCTGACCGGGGGGCAGCCAGCGTCCAGGTACCCCAA |
| | | | | CCCCTGCCCTGGGTCCGGCGTCCCCCCATTAGTGAGTCTTGGCTC |
| | | | | TACTTATAGCATCTGACACCAGAGGGGCCGAAAATAGCCCCTGGA |
| | | | | GAAGGGGGAGGAGGGGGCTATTTAAAGGGCCTGGGAGGGGAGAGA |
| | | | | GAATGAGGAGTGATCATGGCTACCTCAGAGCTGAGCTGCGAGGTG |
| | | | | TCGGAGGAGAACTGTGAGCGCCGGGAGGCCTTCTGGGCAGAATGG |
| | | | | AAGGATCTGACACTGTCCACACGGCCCGAGGAGGGgtgagtgtgg |
| | | | | gtctgctagagccctgcctctgctcccccagagcaccctcactga |
| | | | | gccatgaggccagagcatgaagccctggagaaatttctgggggtg |
| | | | | ggggcaggaagaatgccccatggggagagcaaaggggaaccaccc |
| | | | | ttcctgcccccaggtcccagcagcccaggggagccccccacccag |
| | | | | cctgtgcccagagagcaacagctcccaggagctcactgcccctcc |
| | | | | cctctccccagCTGCTCCCTGCATGAGGAGGACACCCAGAGACAT |
| | | | | GAGACCTACCACCAGCAGGGGCAGTGCCAGGTGCTGGTGCAGCGC |
| | | | | TCGCCCTGGCTGATGATGCGGATGGGCATCCTCGGCCGTGGGCTG |
| | | | | CAGGAGTACCAGCTGCCCTACCAGCGGGTACTGCCGCTGCCCATC |
| | | | | TTCACCCCTGCCAAGATGGGCGCCACCAAGGAGGAGCGTGAGGAC |
| | | | | ACCCCCATCCAGCTTCAGGAGCTGCTGGCGCTGGAGACAGCCCTG |
| | | | | GGTGGCCAGTGTGTGGACCGCCAGGAGGTGGCTGAGATCACAAAG |
| | | | | CAGCTGCCCCCTGTGGTGCCTGTCAGCAAGCCCGGTGCACTTCGT |
| | | | | CGCTCCCTGTCCCGCTCCATGTCCCAGGAAGCACAGAGAGGCTGA |
| | | | | GCGGCCGCAATAAAAGATCTTTATTTTCATTAGATCTGTGTGTTG |
| | | | | GTTTTTTGTGTGTCTAGAGCATGGCTACGTAGATAAGTAGCATGG |
| | | | | CGGGTTAATCATTAACTACaAGGAACCCCTAGTGATGGAGTTGGC |
| | | | | CACTCCCTCTCTGCGCGCTCGCTCGCTCACTGAGGCCGGGCGACC |
| | | | | AAAGGTCGCCCGACGCCCGGGCTTTGCCCGGGCGGCCTCAGTGAG |
| | | | | CGAGCGAGCGCGCAGAGAGGGAGTGG |
| 12 | MHCK7.TCAP.NI/ | 1975 | 50 | CTGCGCGCTCGCTCGCTCACTGAGGCCGCCCGGGCAAAGCCCGGG |
| | SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACccttaagTCTAGAggCTCGAGaagcttgcatgtct |
| | | | | aagctagacccttcagattaaaaataactgaggtaagggcctggg |
| | | | | taggggaggtggtgtgagacgctcctgtctctcctctatctgccc |
| | | | | atcggccctttggggaggaggaatgtgcccaaggactaaaaaaag |
| | | | | gccatggagccagaggggcgagggcaacagacctttcatgggcaa |
| | | | | accttggggccctgctgtctagcatgccccactacgggtctaggc |
| | | | | tgcccatgtaaggaggcaaggcctggggacacccgagatgcctgg |
| | | | | ttataattaacccagacatgtggctgcccccccccccccaacacc |
| | | | | tgctgcctctaaaaataaccctgtccctggtggatcccctgcatg |
| | | | | cgaagatcttcgaacaaggctgtgggggactgagggcaggctgta |
| | | | | acaggcttgggggccagggcttatacgtgcctgggactcccaaag |
| | | | | tattactgttccatgttcccggcgaagggccagctgtcccccgcc |
| | | | | agctagactcagcacttagtttaggaaccagtgagcaagtcagcc |
| | | | | cttggggcagcccatacaaggccatggggctgggcaagctgcacg |
| | | | | cctgggtccggggtgggcacggtgcccgggcaacgagctgaaagc |
| | | | | tcatctgctctcaggggcccctccctggggacagcccctcctggc |
| | | | | tagtcacaccctgtaggctcctctatataacccaggggcacaggg |
| | | | | gctgccctcattctaccaccacctccacagcacagacagacactc |
| | | | | aggagcagccagcggAATGAGGAGTGATCATGGCTACCTCAGAGC |
| | | | | TGAGCTGCGAGGTGTCGGAGGAGAACTGTGAGCGCCGGGAGGCCT |
| | | | | TCTGGGCAGAATGGAAGGATCTGACACTGTCCACACGGCCCGAGG |
| | | | | AGGGgtgagtgtgggtctgctagagccctgcctctgctcccccag |
| | | | | agcaccctcactgagccatgaggccagagcatgaagccctggaga |
| | | | | aatttctggggggggggcaggaagaatgccccatggggagagcaa |
| | | | | aggggaaccacccttcctgcccccaggtcccagcagcccagggga |
| | | | | gccccccacccagcctgtgcccagagagcaacagctcccaggagc |
| | | | | tcactgcccctcccctctccccagCTGCTCCCTGCATGAGGAGGA |
| | | | | CACCCAGAGACATGAGACCTACCACCAGCAGGGGCAGTGCCAGGT |
| | | | | GCTGGTGCAGCGCTCGCCCTGGCTGATGATGCGGATGGGCATCCT |
| | | | | CGGCCGTGGGCTGCAGGAGTACCAGCTGCCCTACCAGCGGGTACT |
| | | | | GCCGCTGCCCATCTTCACCCCTGCCAAGATGGGCGCCACCAAGGA |
| | | | | GGAGCGTGAGGACACCCCCATCCAGCTTCAGGAGCTGCTGGCGCT |
| | | | | GGAGACAGCCCTGGGTGGCCAGTGTGTGGACCGCCAGGAGGTGGC |
| | | | | TGAGATCACAAAGCAGCTGCCCCCTGTGGTGCCTGTCAGCAAGCC |
| | | | | CGGTGCACTTCGTCGCTCCCTGTCCCGCTCCATGTCCCAGGAAGC |
| | | | | ACAGAGAGGCTGAGCGGCCGCAATAAAAGATCTTTATTTTCATTA |
| | | | | GATCTGTGTGTTGGTTTTTTGTGTGTCTAGAGCATGGCTACGTAG |
| | | | | ATAAGTAGCATGGCGGGTTAATCATTAACTACaAGGAACCCCTAG |
| | | | | TGATGGAGTTGGCCACTCCCTCTCTGCGCGCTCGCTCGCTCACTG |
| | | | | AGGCCGGGCGACCAAAGGTCGCCCGACGCCCGGGCTTTGCCCGGG |
| | | | | CGGCCTCAGTGAGCGAGCGAGCGCGCAGAGAGGGAGTGG |
| 13 | MHCK7.TCAP.SI/ | 1881 | 51 | CTGCGCGCTCGCTCGCTCACTGAGGCCGCCCGGGCAAAGCCCGGG |
| | SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACccttaagTCTAGAggCTCGAGaagcttgcatgtct |
| | | | | aagctagacccttcagattaaaaataactgaggtaagggcctggg |
| | | | | taggggaggtggtgtgagacgctcctgtctctcctctatctgccc |
| | | | | atcggccctttggggaggaggaatgtgcccaaggactaaaaaaag |
| | | | | gccatggagccagaggggcgagggcaacagacctttcatgggcaa |
| | | | | accttggggccctgctgtctagcatgccccactacgggtctaggc |
| | | | | tgcccatgtaaggaggcaaggcctggggacacccgagatgcctgg |
| | | | | ttataattaacccagacatgtggctgcccccccccccccaacacc |
| | | | | tgctgcctctaaaaataaccctgtccctggtggatcccctgcatg |
| | | | | cgaagatcttcgaacaaggctgtgggggactgagggcaggctgta |
| | | | | acaggcttgggggccagggcttatacgtgcctgggactcccaaag |
| | | | | tattactgttccatgttcccggcgaagggccagctgtcccccgcc |
| | | | | agctagactcagcacttagtttaggaaccagtgagcaagtcagcc |
| | | | | cttggggcagcccatacaaggccatggggctgggcaagctgcacg |
| | | | | cctgggtccggggtgggcacggtgcccgggcaacgagctgaaagc |
| | | | | tcatctgctctcaggggcccctccctggggacagcccctcctggc |
| | | | | tagtcacaccctgtaggctcctctatataacccaggggcacaggg |
| | | | | gctgccctcattctaccaccacctccacagcacagacagacactc |
| | | | | aggagcagccagcggccaggtaagtttagtctttttgtcttttat |
| | | | | ttcaggtcccggatccggtggtggtgcaaatcaaagaactgctcc |
| | | | | tcagtggatgttgcctttacttctaggcctgtacggaagtgttac |
| | | | | ttctgctctaaaagctgcggaattgtacccgcAATGAGGAGTGAT |
| | | | | CATGGCTACCTCAGAGCTGAGCTGCGAGGTGTCGGAGGAGAACTG |
| | | | | TGAGCGCCGGGAGGCCTTCTGGGCAGAATGGAAGGATCTGACACT |
| | | | | GTCCACACGGCCCGAGGAGGGCTGCTCCCTGCATGAGGAGGACAC |
| | | | | CCAGAGACATGAGACCTACCACCAGCAGGGGCAGTGCCAGGTGCT |
| | | | | GGTGCAGCGCTCGCCCTGGCTGATGATGCGGATGGGCATCCTCGG |
| | | | | CCGTGGGCTGCAGGAGTACCAGCTGCCCTACCAGCGGGTACTGCC |
| | | | | GCTGCCCATCTTCACCCCTGCCAAGATGGGCGCCACCAAGGAGGA |
| | | | | GCGTGAGGACACCCCCATCCAGCTTCAGGAGCTGCTGGCGCTGGA |
| | | | | GACAGCCCTGGGTGGCCAGTGTGTGGACCGCCAGGAGGTGGCTGA |
| | | | | GATCACAAAGCAGCTGCCCCCTGTGGTGCCTGTCAGCAAGCCCGG |
| | | | | TGCACTTCGTCGCTCCCTGTCCCGCTCCATGTCCCAGGAAGCACA |
| | | | | GAGAGGCTGAGCGGCCGCAATAAAAGATCTTTATTTTCATTAGAT |
| | | | | CTGTGTGTTGGTTTTTTGTGTGTCTAGAGCATGGCTACGTAGATA |
| | | | | AGTAGCATGGCGGGTTAATCATTAACTACaAGGAACCCCTAGTGA |
| | | | | TGGAGTTGGCCACTCCCTCTCTGCGCGCTCGCTCGCTCACTGAGG |
| | | | | CCGGGCGACCAAAGGTCGCCCGACGCCCGGGCTTTGCCCGGGCGG |
| | | | | CCTCAGTGAGCGAGCGAGCGCGCAGAGAGGGAGTGG |
| 14 | CMV.TCAP.NI/SD | 1787 | 52 | CTGCGCGCTCGCTCGCTCACTGAGGCCGCCCGGGCAAAGCCCGGG |
| | | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACCCGTTACATAACTTACGGTAAATGGCCCGCCTGGC |
| | | | | TGACCGCCCAACGACCCCCGCCCATTGACGTCAATAATGACGTAT |
| | | | | GTTCCCATAGTAACGCCAATAGGGACTTTCCATTGACGTCAATGG |
| | | | | GTGGAGTATTTACGGTAAACTGCCCACTTGGCAGTACATCAAGTG |
| | | | | TATCATATGCCAAGTACGCCCCCTATTGACGTCAATGACGGTAAA |
| | | | | TGGCCCGCCTGGCATTATGCCCAGTACATGACCTTATGGGACTTT |
| | | | | CCTACTTGGCAGTACATCTACGTATTAGTCATCGCTATTACCATG |
| | | | | GTGATGCGGTTTTGGCAGTACATCAATGGGCGTGGATAGCGGTTT |
| | | | | GACTCACGGGGATTTCCAAGTCTCCACCCCATTGACGTCAATGGG |
| | | | | AGTTTGTTTTGGCACCAAAATCAACGGGACTTTCCAAAATGTCGT |
| | | | | AACAACTCCGCCCCATTGACGCAAATGGGCGGTAGGCGTGTACGG |
| | | | | TGGGAGGTCTATATAAGCAGAGCTCGTTTAGTGAACCGTCAGATC |
| | | | | GCCTGGAGACGCCATCCACGCTGTTTTGACCTCCATAGAAGACAC |
| | | | | CGGGACCGATCCAGCCTCCGGACTCTAGAGGATCCGGTACTCGAG |
| | | | | GAACCTGAATGAGGAGTGATCATGGCTACCTCAGAGCTGAGCTGC |
| | | | | GAGGTGTCGGAGGAGAACTGTGAGCGCCGGGAGGCCTTCTGGGCA |
| | | | | GAATGGAAGGATCTGACACTGTCCACACGGCCCGAGGAGGGgtga |
| | | | | gtgtgggtctgctagagccctgcctctgctcccccagagcaccct |
| | | | | cactgagccatgaggccagagcatgaagccctggagaaatttctg |
| | | | | ggggtgggggcaggaagaatgccccatggggagagcaaaggggaa |
| | | | | ccacccttcctgcccccaggtcccagcagcccaggggagcccccc |
| | | | | acccagcctgtgcccagagagcaacagctcccaggagctcactgc |
| | | | | ccctcccctctccccagCTGCTCCCTGCATGAGGAGGACACCCAG |
| | | | | AGACATGAGACCTACCACCAGCAGGGGCAGTGCCAGGTGCTGGTG |
| | | | | CAGCGCTCGCCCTGGCTGATGATGCGGATGGGCATCCTCGGCCGT |
| | | | | GGGCTGCAGGAGTACCAGCTGCCCTACCAGCGGGTACTGCCGCTG |
| | | | | CCCATCTTCACCCCTGCCAAGATGGGCGCCACCAAGGAGGAGCGT |
| | | | | GAGGACACCCCCATCCAGCTTCAGGAGCTGCTGGCGCTGGAGACA |
| | | | | GCCCTGGGTGGCCAGTGTGTGGACCGCCAGGAGGTGGCTGAGATC |
| | | | | ACAAAGCAGCTGCCCCCTGTGGTGCCTGTCAGCAAGCCCGGTGCA |
| | | | | CTTCGTCGCTCCCTGTCCCGCTCCATGTCCCAGGAAGCACAGAGA |
| | | | | GGCtgaGCGGCCGCAATAAAAGATCTTTATTTTCATTAGATCTGT |
| | | | | GTGTTGGTTTTTTGTGTGTCTAGAGCATGGCTACGTAGATAAGTA |
| | | | | GCATGGGGGGTTAATCATTAACTACaAGGAACCCCTAGTGATGGA |
| | | | | GTTGGCCACTCCCTCTCTGCGCGCTCGCTCGCTCACTGAGGCCGG |
| | | | | GCGACCAAAGGTCGCCCGACGCCCGGGCTTTGCCCGGGGGGCCTC |
| | | | | AGTGAGCGAGCGAGCGCGCAGAGAGGGAGTGG |
| 15 | CMV.TCAP.SI/SD | 1688 | 53 | CTGCGCGCTCGCTCGCTCACTGAGGCCGCCCGGGCAAAGCCCGGG |
| | | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGgtaccCGTTACATAACTTACGGTAAATGGCCCGCCTGGC |
| | | | | TGACCGCCCAACGACCCCCGCCCATTGACGTCAATAATGACGTAT |
| | | | | GTTCCCATAGTAACGCCAATAGGGACTTTCCATTGACGTCAATGG |
| | | | | GTGGAGTATTTACGGTAAACTGCCCACTTGGCAGTACATCAAGTG |
| | | | | TATCATATGCCAAGTACGCCCCCTATTGACGTCAATGACGGTAAA |
| | | | | TGGCCCGCCTGGCATTATGCCCAGTACATGACCTTATGGGACTTT |
| | | | | CCTACTTGGCAGTACATCTACGTATTAGTCATCGCTATTACCATG |
| | | | | GTGATGCGGTTTTGGCAGTACATCAATGGGCGTGGATAGCGGTTT |
| | | | | GACTCACGGGGATTTCCAAGTCTCCACCCCATTGACGTCAATGGG |
| | | | | AGTTTGTTTTGGCACCAAAATCAACGGGACTTTCCAAAATGTCGT |
| | | | | AACAACTCCGCCCCATTGACGCAAATGGGCGGTAGGCGTGTACGG |
| | | | | TGGGAGGTCTATATAAGCAGAGCTCGTTTAGTGAACCGTCAGATC |
| | | | | GCCTGGAGACGCCATCCACGCTGTTTTGACCTCCATAGAAGACAC |
| | | | | CGGGACCGATCCAGCCTCCGGACTCTAGAGGATCCGGTACTCGAG |
| | | | | GAACaggtaagtttagtctttttgtcttttatttcaggtcccgga |
| | | | | tccggtggtggtgcaaatcaaagaactgctcctcagtggatgttg |
| | | | | cctttacttctaggcctgtacggaagtgttacttctgctctaaaa |
| | | | | gctgcggaattgtacccgcAATGAGGAGTGATCATGGCTACCTCA |
| | | | | GAGCTGAGCTGCGAGGTGTCGGAGGAGAACTGTGAGCGCCGGGAG |
| | | | | GCCTTCTGGGCAGAATGGAAGGATCTGACACTGTCCACACGGCCC |
| | | | | GAGGAGGGCTGCTCCCTGCATGAGGAGGACACCCAGAGACATGAG |
| | | | | ACCTACCACCAGCAGGGGCAGTGCCAGGTGCTGGTGCAGCGCTCG |
| | | | | CCCTGGCTGATGATGCGGATGGGCATCCTCGGCCGTGGGCTGCAG |
| | | | | GAGTACCAGCTGCCCTACCAGCGGGTACTGCCGCTGCCCATCTTC |
| | | | | ACCCCTGCCAAGATGGGCGCCACCAAGGAGGAGCGTGAGGACACC |
| | | | | CCCATCCAGCTTCAGGAGCTGCTGGCGCTGGAGACAGCCCTGGGT |
| | | | | GGCCAGTGTGTGGACCGCCAGGAGGTGGCTGAGATCACAAAGCAG |
| | | | | CTGCCCCCTGTGGTGCCTGTCAGCAAGCCCGGTGCACTTCGTCGC |
| | | | | TCCCTGTCCCGCTCCATGTCCCAGGAAGCACAGAGAGGCTGAGCG |
| | | | | GCCGCAATAAAAGATCTTTATTTTCATTAGATCTGTGTGTTGGTT |
| | | | | TTTTGTGTGTCTAGAGCATGGCTACGTAGATAAGTAGCATGGCGG |
| | | | | GTTAATCATTAACTACaAGGAACCCCTAGTGATGGAGTTGGCCAC |
| | | | | TCCCTCTCTGCGCGCTCGCTCGCTCACTGAGGCCGGGCGACCAAA |
| | | | | GGTCGCCCGACGCCCGGGCTTTGCCCGGGGGGCCTCAGTGAGCGA |
| | | | | GCGAGCGCGCAGAGAGGGAGTGG |
| 16 | NP.TCAP.3xFLAG | 2162 | 54 | CTGCGCGCTCGCTCGCTCACTGAGGCCGCCCGGGCAAAGCCCGGG |
| | /SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACccttaagTCTAGAggCTCGAGaagcttGCTAGCCC |
| | | | | CATGAAGAAGAGCCAGTCCCCTCCCAGGTAGCACATCAGAGCGGG |
| | | | | CAGGCTCCAGAGCTCTTCTGGTCCTCTTGCAGTGGCCAGAAAGTG |
| | | | | GGGAAAGGGGCCCAGAGAGGGGCAGGGACTGACCCATGGTCACAC |
| | | | | AGCCTATTTATGGCTGATCTGGAGCCAGGCCTTCTGACTATCACC |
| | | | | CATCAGAGCCCCTGGATTCCCATTTCACCCAACACAGGTGTTGGG |
| | | | | GTAGAACTGGGGGCCCTCCACCCCTACTAGCCTGACTGTCTCCTG |
| | | | | AGACCTGTCTCCTAAGACCACTTACCTGACCACTGGCCATGGGGA |
| | | | | AGGAAAACACCTGGCCAAGGCTCATGCCCCAGGTTCCCAGGCATG |
| | | | | AAGTTGACTGTCTCCGCCCCTGCTGGTGTGGTGAGGGTGACTGGG |
| | | | | GACTAGGCACTAGGCCTTTGGTGCAGGCGCCTGAGGACGTGGTTG |
| | | | | CACTCTCCCTTCTGGGGATATGCCCTTGAGCCCAGGCAGAGGAGA |
| | | | | GCACAGCCAGGGCAGGACCTGGCAGCCCTGGTACAGAGCCCAGAG |
| | | | | GGGGCATCAGTTCCTGCTGGTCCTGCTCTGTTTACAGACAAGCTG |
| | | | | CTGTCCTCCCTGCAAAGGGGAGTGGGTGGGGCAGAGGGCAAGTGC |
| | | | | CAGGGGGGCACAAGGCTGGGCATGTGGCTGGCATGAGACGGTGTC |
| | | | | TGAGTAATGTCAGGCACCTGGAGGCATTGACCCCAGGACCTTGGA |
| | | | | CCCCAGACCTCTGACCGGGGGGCAGCCAGCGTCCAGGTACCCCAA |
| | | | | CCCCTGCCCTGGGTCCGGCGTCCCCCCATTAGTGAGTCTTGGCTC |
| | | | | TACTTATAGCATCTGACACCAGAGGGGCCGAAAATAGCCCCTGGA |
| | | | | GAAGGGGGAGGAGGGGGCTATTTAAAGGGCCTGGGAGGGGAGAGA |
| | | | | GAATGAGGAGTGATCATGGCTACCTCAGAGCTGAGCTGCGAGGTG |
| | | | | TCGGAGGAGAACTGTGAGCGCCGGGAGGCCTTCTGGGCAGAATGG |
| | | | | AAGGATCTGACACTGTCCACACGGCCCGAGGAGGGgtgagtgtgg |
| | | | | gtctgctagagccctgcctctgctcccccagagcaccctcactga |
| | | | | gccatgaggccagagcatgaagccctggagaaatttctgggggtg |
| | | | | ggggcaggaagaatgccccatggggagagcaaaggggaaccaccc |
| | | | | ttcctgcccccaggtcccagcagcccaggggagccccccacccag |
| | | | | cctgtgcccagagagcaacagctcccaggagctcactgcccctcc |
| | | | | cctctccccagCTGCTCCCTGCATGAGGAGGACACCCAGAGACAT |
| | | | | GAGACCTACCACCAGCAGGGGCAGTGCCAGGTGCTGGTGCAGCGC |
| | | | | TCGCCCTGGCTGATGATGCGGATGGGCATCCTCGGCCGTGGGCTG |
| | | | | CAGGAGTACCAGCTGCCCTACCAGCGGGTACTGCCGCTGCCCATC |
| | | | | TTCACCCCTGCCAAGATGGGCGCCACCAAGGAGGAGCGTGAGGAC |
| | | | | ACCCCCATCCAGCTTCAGGAGCTGCTGGCGCTGGAGACAGCCCTG |
| | | | | GGTGGCCAGTGTGTGGACCGCCAGGAGGTGGCTGAGATCACAAAG |
| | | | | CAGCTGCCCCCTGTGGTGCCTGTCAGCAAGCCCGGTGCACTTCGT |
| | | | | CGCTCCCTGTCCCGCTCCATGTCCCAGGAAGCACAGAGAGGCGAC |
| | | | | TACAAAGACCATGACGGTGATTATAAAGATCATGACATCGATTAC |
| | | | | AAGGATGACGATGACAAGTGAGCGGCCGCAATAAAAGATCTTTAT |
| | | | | TTTCATTAGATCTGTGTGTTGGTTTTTTGTGTGTCTAGAGCATGG |
| | | | | CTACGTAGATAAGTAGCATGGGGGGTTAATCATTAACTACaAGGA |
| | | | | ACCCCTAGTGATGGAGTTGGCCACTCCCTCTCTGCGCGCTCGCTC |
| | | | | GCTCACTGAGGCCGGGCGACCAAAGGTCGCCCGACGCCCGGGCTT |
| | | | | TGCCCGGGCGGCCTCAGTGAGCGAGCGAGCGCGCAGAGAGGGAGT |
| | | | | GG |
| 17 | MHCK7.TCAP.NI3 | 2041 | 55 | CTGCGCGCTCGCTCGCTCACTGAGGCCGCCCGGGCAAAGCCCGGG |
| | xFLAG/SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACccttaagTCTAGAggCTCGAGaagcttgcatgtct |
| | | | | aagctagacccttcagattaaaaataactgaggtaagggcctggg |
| | | | | taggggaggtggtgtgagacgctcctgtctctcctctatctgccc |
| | | | | atcggccctttggggaggaggaatgtgcccaaggactaaaaaaag |
| | | | | gccatggagccagaggggcgagggcaacagacctttcatgggcaa |
| | | | | accttggggccctgctgtctagcatgccccactacgggtctaggc |
| | | | | tgcccatgtaaggaggcaaggcctggggacacccgagatgcctgg |
| | | | | ttataattaacccagacatgtggctgcccccccccccccaacacc |
| | | | | tgctgcctctaaaaataaccctgtccctggtggatcccctgcatg |
| | | | | cgaagatcttcgaacaaggctgtgggggactgagggcaggctgta |
| | | | | acaggcttgggggccagggcttatacgtgcctgggactcccaaag |
| | | | | tattactgttccatgttcccggcgaagggccagctgtcccccgcc |
| | | | | agctagactcagcacttagtttaggaaccagtgagcaagtcagcc |
| | | | | cttggggcagcccatacaaggccatggggctgggcaagctgcacg |
| | | | | cctgggtccgggggggcacggtgcccgggcaacgagctgaaagct |
| | | | | catctgctctcaggggcccctccctggggacagcccctcctggct |
| | | | | agtcacaccctgtaggctcctctatataacccaggggcacagggg |
| | | | | ctgccctcattctaccaccacctccacagcacagacagacactca |
| | | | | ggagcagccagcggAATGAGGAGTGATCATGGCTACCTCAGAGCT |
| | | | | GAGCTGCGAGGTGTCGGAGGAGAACTGTGAGCGCCGGGAGGCCTT |
| | | | | CTGGGCAGAATGGAAGGATCTGACACTGTCCACACGGCCCGAGGA |
| | | | | GGGgtgagtgtgggtctgctagagccctgcctctgctcccccaga |
| | | | | gcaccctcactgagccatgaggccagagcatgaagccctggagaa |
| | | | | atttctgggggtgggggcaggaagaatgccccatggggagagcaa |
| | | | | aggggaaccacccttcctgcccccaggtcccagcagcccagggga |
| | | | | gccccccacccagcctgtgcccagagagcaacagetoccaggage |
| | | | | tcactgcccctcccctctccccagCTGCTCCCTGCATGAGGAGGA |
| | | | | CACCCAGAGACATGAGACCTACCACCAGCAGGGGCAGTGCCAGGT |
| | | | | GCTGGTGCAGCGCTCGCCCTGGCTGATGATGCGGATGGGCATCCT |
| | | | | CGGCCGTGGGCTGCAGGAGTACCAGCTGCCCTACCAGCGGGTACT |
| | | | | GCCGCTGCCCATCTTCACCCCTGCCAAGATGGGCGCCACCAAGGA |
| | | | | GGAGCGTGAGGACACCCCCATCCAGCTTCAGGAGCTGCTGGCGCT |
| | | | | GGAGACAGCCCTGGGTGGCCAGTGTGTGGACCGCCAGGAGGTGGC |
| | | | | TGAGATCACAAAGCAGCTGCCCCCTGTGGTGCCTGTCAGCAAGCC |
| | | | | CGGTGCACTTCGTCGCTCCCTGTCCCGCTCCATGTCCCAGGAAGC |
| | | | | ACAGAGAGGCGACTACAAAGACCATGACGGTGATTATAAAGATCA |
| | | | | TGACATCGATTACAAGGATGACGATGACAAGTGAGCGGCCGCAAT |
| | | | | AAAAGATCTTTATTTTCATTAGATCTGTGTGTTGGTTTTTTGTGT |
| | | | | GTCTAGAGCATGGCTACGTAGATAAGTAGCATGGCGGGTTAATCA |
| | | | | TTAACTACaAGGAACCCCTAGTGATGGAGTTGGCCACTCCCTCTC |
| | | | | TGCGCGCTCGCTCGCTCACTGAGGCCGGGCGACCAAAGGTCGCCC |
| | | | | GACGCCCGGGCTTTGCCCGGGCGGCCTCAGTGAGCGAGCGAGCGC |
| | | | | GCAGAGAGGGAGTGG |
| 18 | MHCK7.TCAP.SI3 | 1947 | 56 | CTGCGCGCTCGCTCGCTCACTGAGGCCGCCCGGGCAAAGCCCGGG |
| | xFLAG/SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACccttaagTCTAGAggCTCGAGaagcttgcatgtct |
| | | | | aagctagacccttcagattaaaaataactgaggtaagggcctggg |
| | | | | taggggaggtggtgtgagacgctcctgtctctcctctatctgccc |
| | | | | atcggccctttggggaggaggaatgtgcccaaggactaaaaaaag |
| | | | | gccatggagccagaggggcgagggcaacagacctttcatgggcaa |
| | | | | accttggggccctgctgtctagcatgccccactacgggtctaggc |
| | | | | tgcccatgtaaggaggcaaggcctggggacacccgagatgcctgg |
| | | | | ttataattaacccagacatgtggctgcccccccccccccaacacc |
| | | | | tgctgcctctaaaaataaccctgtccctggtggatcccctgcatg |
| | | | | cgaagatcttcgaacaaggctgtgggggactgagggcaggctgta |
| | | | | acaggcttgggggccagggcttatacgtgcctgggactcccaaag |
| | | | | tattactgttccatgttcccggcgaagggccagctgtcccccgcc |
| | | | | agctagactcagcacttagtttaggaaccagtgagcaagtcagcc |
| | | | | cttggggcagcccatacaaggccatggggctgggcaagctgcacg |
| | | | | cctgggtccggggtgggcacggtgcccgggcaacgagctgaaagc |
| | | | | tcatctgctctcaggggcccctccctggggacagcccctcctggc |
| | | | | tagtcacaccctgtaggctcctctatataacccaggggcacaggg |
| | | | | gctgccctcattctaccaccacctccacagcacagacagacactc |
| | | | | aggagcagccagcggccaggtaagtttagtctttttgtcttttat |
| | | | | ttcaggtcccggatccggtggtggtgcaaatcaaagaactgctcc |
| | | | | tcagtggatgttgcctttacttctaggcctgtacggaagtgttac |
| | | | | ttctgctctaaaagctgcggaattgtacccgcAATGAGGAGTGAT |
| | | | | CATGGCTACCTCAGAGCTGAGCTGCGAGGTGTCGGAGGAGAACTG |
| | | | | TGAGCGCCGGGAGGCCTTCTGGGCAGAATGGAAGGATCTGACACT |
| | | | | GTCCACACGGCCCGAGGAGGGCTGCTCCCTGCATGAGGAGGACAC |
| | | | | CCAGAGACATGAGACCTACCACCAGCAGGGGCAGTGCCAGGTGCT |
| | | | | GGTGCAGCGCTCGCCCTGGCTGATGATGCGGATGGGCATCCTCGG |
| | | | | CCGTGGGCTGCAGGAGTACCAGCTGCCCTACCAGCGGGTACTGCC |
| | | | | GCTGCCCATCTTCACCCCTGCCAAGATGGGCGCCACCAAGGAGGA |
| | | | | GCGTGAGGACACCCCCATCCAGCTTCAGGAGCTGCTGGCGCTGGA |
| | | | | GACAGCCCTGGGTGGCCAGTGTGTGGACCGCCAGGAGGTGGCTGA |
| | | | | GATCACAAAGCAGCTGCCCCCTGTGGTGCCTGTCAGCAAGCCCGG |
| | | | | TGCACTTCGTCGCTCCCTGTCCCGCTCCATGTCCCAGGAAGCACA |
| | | | | GAGAGGCGACTACAAAGACCATGACGGTGATTATAAAGATCATGA |
| | | | | CATCGATTACAAGGATGACGATGACAAGTGAGCGGCCGCAATAAA |
| | | | | AGATCTTTATTTTCATTAGATCTGTGTGTTGGTTTTTTGTGTGTC |
| | | | | TAGAGCATGGCTACGTAGATAAGTAGCATGGGGGGTTAATCATTA |
| | | | | ACTACaAGGAACCCCTAGTGATGGAGTTGGCCACTCCCTCTCTGC |
| | | | | GCGCTCGCTCGCTCACTGAGGCCGGGCGACCAAAGGTCGCCCGAC |
| | | | | GCCCGGGCTTTGCCCGGGCGGCCTCAGTGAGCGAGCGAGCGCGCA |
| | | | | GAGAGGGAGTGG |
| 19 | CMV.TCAP.NI3xF | 1853 | 57 | CTGCGCGCTCGCTCGCTCACTGAGGCCGCCCGGGCAAAGCCCGGG |
| | LAG/SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGGTACCCGTTACATAACTTACGGTAAATGGCCCGCCTGGC |
| | | | | TGACCGCCCAACGACCCCCGCCCATTGACGTCAATAATGACGTAT |
| | | | | GTTCCCATAGTAACGCCAATAGGGACTTTCCATTGACGTCAATGG |
| | | | | GTGGAGTATTTACGGTAAACTGCCCACTTGGCAGTACATCAAGTG |
| | | | | TATCATATGCCAAGTACGCCCCCTATTGACGTCAATGACGGTAAA |
| | | | | TGGCCCGCCTGGCATTATGCCCAGTACATGACCTTATGGGACTTT |
| | | | | CCTACTTGGCAGTACATCTACGTATTAGTCATCGCTATTACCATG |
| | | | | GTGATGCGGTTTTGGCAGTACATCAATGGGCGTGGATAGCGGTTT |
| | | | | GACTCACGGGGATTTCCAAGTCTCCACCCCATTGACGTCAATGGG |
| | | | | AGTTTGTTTTGGCACCAAAATCAACGGGACTTTCCAAAATGTCGT |
| | | | | AACAACTCCGCCCCATTGACGCAAATGGGCGGTAGGCGTGTACGG |
| | | | | TGGGAGGTCTATATAAGCAGAGCTCGTTTAGTGAACCGTCAGATC |
| | | | | GCCTGGAGACGCCATCCACGCTGTTTTGACCTCCATAGAAGACAC |
| | | | | CGGGACCGATCCAGCCTCCGGACTCTAGAGGATCCGGTACTCGAG |
| | | | | GAACCTGAATGAGGAGTGATCATGGCTACCTCAGAGCTGAGCTGC |
| | | | | GAGGTGTCGGAGGAGAACTGTGAGCGCCGGGAGGCCTTCTGGGCA |
| | | | | GAATGGAAGGATCTGACACTGTCCACACGGCCCGAGGAGGGgtga |
| | | | | gtgtgggtctgctagagccctgcctctgctcccccagagcaccct |
| | | | | cactgagccatgaggccagagcatgaagccctggagaaatttctg |
| | | | | ggggtgggggcaggaagaatgccccatggggagagcaaaggggaa |
| | | | | ccacccttcctgcccccaggtcccagcagcccaggggagcccccc |
| | | | | acccagcctgtgcccagagagcaacagctcccaggagctcactgc |
| | | | | ccctcccctctccccagCTGCTCCCTGCATGAGGAGGACACCCAG |
| | | | | AGACATGAGACCTACCACCAGCAGGGGCAGTGCCAGGTGCTGGTG |
| | | | | CAGCGCTCGCCCTGGCTGATGATGCGGATGGGCATCCTCGGCCGT |
| | | | | GGGCTGCAGGAGTACCAGCTGCCCTACCAGCGGGTACTGCCGCTG |
| | | | | CCCATCTTCACCCCTGCCAAGATGGGCGCCACCAAGGAGGAGCGT |
| | | | | GAGGACACCCCCATCCAGCTTCAGGAGCTGCTGGCGCTGGAGACA |
| | | | | GCCCTGGGTGGCCAGTGTGTGGACCGCCAGGAGGTGGCTGAGATC |
| | | | | ACAAAGCAGCTGCCCCCTGTGGTGCCTGTCAGCAAGCCCGGTGCA |
| | | | | CTTCGTCGCTCCCTGTCCCGCTCCATGTCCCAGGAAGCACAGAGA |
| | | | | GGCGACTACAAAGACCATGACGGTGATTATAAAGATCATGACATC |
| | | | | GATTACAAGGATGACGATGACAAGtgaGCGGCCGCAATAAAAGAT |
| | | | | CTTTATTTTCATTAGATCTGTGTGTTGGTTTTTTGTGTGTCTAGA |
| | | | | GCATGGCTACGTAGATAAGTAGCATGGGGGTTAATCATTAACTAC |
| | | | | aAGGAACCCCTAGTGATGGAGTTGGCCACTCCCTCTCTGCGCGCT |
| | | | | CGCTCGCTCACTGAGGCCGGGCGACCAAAGGTCGCCCGACGCCCG |
| | | | | GGCTTTGCCCGGGCGGCCTCAGTGAGCGAGCGAGCGCGCAGAGAG |
| | | | | GGAGTGG |
| 20 | CMV.TCAP.SI3xF | 1754 | 58 | CTGCGCGCTCGCTCGCTCACTGAGGCCGCCCGGGCAAAGCCCGGG |
| | LAG/SD | | | CGTCGGGCGACCTTTGGTCGCCCGGCCTCAGTGAGCGAGCGAGCG |
| | | | | CGCAGAGAGGGAGTGGAATTCACGCGTGGATCTGAATTCAATTCA |
| | | | | CGCGTGgtaccCGTTACATAACTTACGGTAAATGGCCCGCCTGGC |
| | | | | TGACCGCCCAACGACCCCCGCCCATTGACGTCAATAATGACGTAT |
| | | | | GTTCCCATAGTAACGCCAATAGGGACTTTCCATTGACGTCAATGG |
| | | | | GTGGAGTATTTACGGTAAACTGCCCACTTGGCAGTACATCAAGTG |
| | | | | TATCATATGCCAAGTACGCCCCCTATTGACGTCAATGACGGTAAA |
| | | | | TGGCCCGCCTGGCATTATGCCCAGTACATGACCTTATGGGACTTT |
| | | | | CCTACTTGGCAGTACATCTACGTATTAGTCATCGCTATTACCATG |
| | | | | GTGATGCGGTTTTGGCAGTACATCAATGGGCGTGGATAGCGGTTT |
| | | | | GACTCACGGGGATTTCCAAGTCTCCACCCCATTGACGTCAATGGG |
| | | | | AGTTTGTTTTGGCACCAAAATCAACGGGACTTTCCAAAATGTCGT |
| | | | | AACAACTCCGCCCCATTGACGCAAATGGGCGGTAGGCGTGTACGG |
| | | | | TGGGAGGTCTATATAAGCAGAGCTCGTTTAGTGAACCGTCAGATC |
| | | | | GCCTGGAGACGCCATCCACGCTGTTTTGACCTCCATAGAAGACAC |
| | | | | CGGGACCGATCCAGCCTCCGGACTCTAGAGGATCCGGTACTCGAG |
| | | | | GAACaggtaagtttagtctttttgtcttttatttcaggtcccgga |
| | | | | tccggtggtggtgcaaatcaaagaactgctcctcagtggatgttg |
| | | | | cctttacttctaggcctgtacggaagtgttacttctgctctaaaa |
| | | | | getgeggaattgtacccgcAATGAGGAGTGATCATGGCTACCTCA |
| | | | | GAGCTGAGCTGCGAGGTGTCGGAGGAGAACTGTGAGCGCCGGGAG |
| | | | | GCCTTCTGGGCAGAATGGAAGGATCTGACACTGTCCACACGGCCC |
| | | | | GAGGAGGGCTGCTCCCTGCATGAGGAGGACACCCAGAGACATGAG |
| | | | | ACCTACCACCAGCAGGGGCAGTGCCAGGTGCTGGTGCAGCGCTCG |
| | | | | CCCTGGCTGATGATGCGGATGGGCATCCTCGGCCGTGGGCTGCAG |
| | | | | GAGTACCAGCTGCCCTACCAGCGGGTACTGCCGCTGCCCATCTTC |
| | | | | ACCCCTGCCAAGATGGGCGCCACCAAGGAGGAGCGTGAGGACACC |
| | | | | CCCATCCAGCTTCAGGAGCTGCTGGCGCTGGAGACAGCCCTGGGT |
| | | | | GGCCAGTGTGTGGACCGCCAGGAGGTGGCTGAGATCACAAAGCAG |
| | | | | CTGCCCCCTGTGGTGCCTGTCAGCAAGCCCGGTGCACTTCGTCGC |
| | | | | TCCCTGTCCCGCTCCATGTCCCAGGAAGCACAGAGAGGCGACTAC |
| | | | | AAAGACCATGACGGTGATTATAAAGATCATGACATCGATTACAAG |
| | | | | GATGACGATGACAAGTGAGCGGCCGCAATAAAAGATCTTTATTTT |
| | | | | CATTAGATCTGTGTGTTGGTTTTTTGTGTGTCTAGAGCATGGCTA |
| | | | | CGTAGATAAGTAGCATGGCGGGTTAATCATTAACTACaAGGAACC |
| | | | | CCTAGTGATGGAGTTGGCCACTCCCTCTCTGCGCGCTCGCTCGCT |
| | | | | CACTGAGGCCGGGCGACCAAAGGTCGCCCGACGCCCGGGCTTTGC |
| | | | | CCGGGGGGCCTCAGTGAGCGAGCGAGCGCGCAGAGAGGGAGTGG |

Ten rAAV vector constructs were designed as set out in FIG. 1 to test the restoration of TCAP expression. The vectors comprise the following promoters: human TCAP native promoter (NP) (orange), the mouse analog of which showed to be involved in circadian TCAP regulation in skeletal muscles (constructs 1 and 2); MHCK7 promoter (MHCK7) (lavender), which shows high-level expression in fast and slow skeletal and cardiac muscle; human cytomegalovirus (CMV) promoter (purple), which shows high level of expression in all tissue and organs. The human TCAP gene is represented as exon 1 (light green), intron 1 (gold, NI, native intron) and exon 2 (dark green). A chimeric simian virus 40 (SV40) intron (SI, synthetic intron) is represented in light gold.

The specific sequences for each of the ten rAAV vector constructs are provided in FIGS. 9-28 (i.e., FIGS. 9-18 are the first generation of the ten rAAV constructs; FIGS. 19-28 are second generation of the ten rAAV constructs).

### Materials and Methods

### Vector Production.

All vectors were produced by the Vector core at Nationwide Children's Hospital (now known as Andelyn Biosciences). Physical titer determination was based on degradation of non-encapsidated DNA following digestion of viral capsids. Released encapsidated DNA was quantified by qPCR to determine the DNase Resistant Particle (DRP) titer (DRP/ml or vg/ml), utilizing a unique probe directed toward the MHCK7 or CMV promoters and a linearized plasmid standard. The vectors were diluted in TMN200P buffer, containing 20 mM Tris-HCl (pH 8.0), 200 mM NaCl, and 1 mM MgCl₂, 0.001% (w/w) Poloxamer 188, was provided by the VVC at NCH and utilized as a diluent for vector preparation. Poloxamer 188 (P188) is a nonionic linear copolymer having an average molecular weight of 8400 Daltons and is also referred to as PLURONIC F68, FLOCOR and RheothRx.

### Cell Lines.

The C2C12 cell line, which is the immortalized line of mouse skeletal myoblasts, was purchased from ATCC. FM=human FibroMyoD derived control cell lines; FM193ML TCAP-null=FibroMyoD patient-derived cell line from a TCAP mutated patient, and FM000KF, FM190KL or FM021PK controls=FibroMyoD patient-derived cell line from normal male or female patients were provided by the Cell Banking Core at Nationwide Children's Hospital.

### Cell Culture.

C2C12 cells were cultured in DMEM medium (Dulbecco's modified Eagle's medium, Corning, cat #10013CV or alternative) supplemented with 10% (v/v) FBS (R&D Systems, cat #S11550 or alternative) in the presence of antibiotics (Gibco, cat #15240062 or alternative) and were maintained at 37° C. in 5% CO₂. Human FibroMyoD derived control cell lines were cultured in DMEM medium supplemented with 10% (v/v) FBS in the presence of antibiotics and were maintained at 37° C. in 5% CO₂. To convert cells to myoblasts, cells were cultured in Skeletal Muscle Cell Growth Medium (Ready-to-use) (Promocell, cat #c-23060 or alternative) in the presence of doxycycline hydrochloride (Fisher BioReagents, cat #BP2653-5 or alternative) at the concentration of 8 μg/mL for three days. Then, untreated cells or AAV-transduced cells were cultured in Skeletal Muscle Cell Differentiation Medium (Ready-to-use) (Promocell, cat #C-23061 or alternative) for up to 5-15 days as described in individual experiments.

### Mice and Source of Mice.

C57Bl/6 (Bl6 or C57) mice were purchased from Jackson Laboratories (stock #000664) and have been maintained by in-house breeding. hTCAPKI mouse model was kindly provided by Drs. Charles Emerson (University of California, San Diego), Greg Cox (The Jackson Laboratory), and Scot Wolfe (University of Massachusetts Medical School) and transferred from the Jackson Laboratory (MTA [2020-0448]). Animals have been maintained by in-house breeding. The mouse model was created by insertion of an 8 bp fragment into the mouse TCAP gene and modifying the flanking codons to match the human TCAP gene. The novel mouse model, in various aspects, is used for developing new translational approaches, including gene therapy.

### C2C12 Cells Electroporation and Transfection.

C2C12 cells were electroporated using NHDF Nucleofector Kit (Lonza, Amaxa, cat #VPD-1001 or alternative) and rAAV.TCAP.3×FLAG (Amp+) plasmids following manufacturer's instruction and plated in 6-w plates with coverslips. In 24 hours, the myoblast medium was switched to differentiation medium, containing DMED supplemented with 2% horse serum (Sigma, cat #H1270-100ML or alternative) in the presence of antibiotics and cells were incubated in it for 8 days. At day 8 of differentiation, C2C12 myotubes (D08) were transfected by Lipofectamine 3000 (Life Technologies, cat #L3000-015 or alternative) using the same set of constructs following manufacturer's instruction. Cells were incubated for one more day and at day 9 of differentiation, coverslips were collected, washed in PBS and fixed in 4% paraformaldehyde (PFA) solution for 20 minutes, then washed in PBS and used for IF staining.

### Human FibroMyoD Cell Line Infection.

Cells were transduced on the Myoblast stage Day 3 using SCAAV1.MHCK7.TCAP.SI3×FLAG vector at the dose of 0.4-1.1E11 vg per 6-w plate, and then they were cultured as described in the cell culture section. Cells were either fixed in 4% PFA for Immunofluorescent (IF) staining or cell pellets were collected for Western blotting (WB) experiments.

### Cell Immunofluorescent (IF) Staining.

C2C12 cells: After fixation, cells were permeabilized in PBS solution, containing 0.1% Triton X-100 for 5 minutes and then incubated in PBS, containing 1% Bovine Serum Albumin (BSA) (Fisher BioReagents, cat #BP9706100 or alternative) for 30-60 minutes at room temperature. Cells were incubated in PBS with 0.5% BSA and 0.1% Triton X-100 containing following primary antibodies: anti-TCAP antibody (Abcam, cat #ab133646, anti-rabbit, dilution 1:100) or anti-Desmin antibody (Fisher, cat #MA5-13259, clone D33; anti-mouse, dilution 1:100) for 2 hours at room temperature. Then cells were washed in PBS with 0.1% Triton X-100 three times and incubated in 0.5% BSA with 0.1% Triton X-100 and 5% normal goat serum (Invitrogen, cat #10000C or alternative), containing secondary antibodies: goat anti rabbit, Alexa 568 (Invitrogen, cat #A-21069, dilution 1:500) or goat anti mouse, Alexa 488 (Invitrogen, cat #A-11017, dilution 1:500) for one hour in the dark at room temperature. Then, cells were washed in PBS with 0.2% Triton X-100 three times, washed one time in PBS and then mounted in the medium with DAPI (Vector laboratories, cat #H-1500 or alternative) to stain and visualize all cell nuclei. The fixed human FibroMyoD cells were permeabilized in PBS solution, containing 0.1% Triton X-100 for 5 minutes and then incubated in PBS, containing 1% Bovine Serum Albumin (BSA) (Fisher BioReagents, cat #BP9706100 or alternative) for 30-60 minutes at room temperature. Cells were incubated in PBS with 0.5% BSA and 0.1% Triton X-100 containing the following primary antibodies anti-TCAP antibody (MyBioSource, cat #MBS9126105, anti-rabbit polyclonal, dilution 1:100) and anti-FLAG antibody (Sigma-Aldrich, cat #SAB4200071-200UL, anti-rat monoclonal, dilution 1:200) for 2 hours at room temperature. Then cells were washed in PBS with 0.1% Triton X-100 three times and incubated in 0.5% BSA with 0.1% Triton X-100 and 5% normal goat serum (Invitrogen, cat #10000C), containing the following secondary antibodies goat anti rabbit, Alexa 647 (Invitrogen, cat #A21244, dilution 1:500) and donkey anti-rat, Alexa 488 (Jackson Labs, cat #712-546-153, dilution 1:500) for one hour in the dark at room temperature. Then, cells were washed in PBS with 0.2% Triton X-100 three times, washed one time in PBS and then mounted in the medium with DAPI (Vector laboratories, cat #H-1500 or alternative) to stain and visualize all cell nuclei.

### Cell Imaging and Fluorescent Microscopy.

C2C12 cells were imaged at 20× using an Olympus BX61 fluorescence microscope. Human FibroMyoD cells were imaged on Nikon Ti2E fluorescence microscope with a 100× oil objective.

### Skeletal Muscle Immunofluorescent (IF) Staining.

Frozen TA muscles were cut at 10 microns, air-dried, permeabilized in 0.1% Triton X-100 PBS, and blocked in 1×PBS with 10% normal goat serum (NGS, Invitrogen, cat #10000C or alternative) and 0.1% Tween 20 for 1 hour at room temperature (RT). Then, samples were blocked in goat-anti-mouse IgG (H+L) unconjugated fab fragment antibody (Jackson, cat #115-007-003 or alternative) for 2 hours at RT. Following blocking, sections were co-stained in 1:100 rabbit polyclonal anti-TCAP antibody (Mybiosource, cat #MBS9126105) and 1:20 mouse monoclonal anti-Titin antibody (DSHB, cat #9D10) for 2 hours at RT. Slides were washed 4×5 min, incubated in the appropriate Alexa Fluor647 (Invitrogen, cat #A21244, goat anti-rabbit) and Alexa Fluor 488 (Invitrogen, ca t #A11017, goat anti-mouse) conjugated secondary antibodies (1:500 dilution) for 1 hour, and washed again 3×5 minutes. Tissues were affixed with ProLong Gold Antifade mounting medium with DAPI (Invitrogen, cat #P36934 or alternative).

### Skeletal Muscle Imaging and Fluorescent Microscopy.

TA muscle images were collected on Nikon Ti2E fluorescence microscope with a100× oil objective.

### Laser-Induced Cell Damage.

The membrane injury assay was performed on untreated and AAV1-treated human FibroMyoD cells, which were plated on 35-mm dishes and differentiated for 5 days. Cells were incubated in Tyrode's solution, 140 mM NaCl, 5 mM KCl, 2 mM MgCl₂, and 10 mM HEPES, pH 7.2 with (2 mM Ca²⁺) or without (0 mM Ca²⁺) calcium ions. Cell membrane damage was induced in the presence of 2.5 μM FM4-64 amphiphilic fluorescent styryl pyridinium dye (Invitrogen, cat #T13320 or alternative) with or without 2.0 mM Ca²⁺, using the Olympus FV1000 multi-photon laser scanning confocal system. Cells were irradiated at 12.5-24% of laser power for 3 sec. Pre- and post-damage images were captured every 3 sec, continuing for 53 sec. The extent of membrane damage was analyzed using ImageJ software (NIH, version 1.53c), by measuring the fluorescence intensity encompassing the site of damage.

### Protein Expression and Western Blotting Analysis.

Protein extraction and Western blotting analysis were carried out after C57Bl/6 mice were treated intramuscularly (IM) with AAV9.TCAP vectors. Mouse tissue lysates were prepared by mechanically disrupting tissue in a Tissuelyser II (Qiagen, USA) in lysis buffer, containing 50 mM Tris-HCl (pH 7.4), 150 mM NaCl, 1% NP-40, digitonin (Sigma, cat #D141-500 MG or alternative) and protease inhibitor cocktail (Complete mini, EDTA-free protease inhibitor cocktail tablet; cat #NC0962311, Sigma Aldrich, USA). The lysate was centrifuged at 14,000 g for 20 min at 4° C. and supernatants were collected for analysis. Total protein was quantified by using BCA protein assay kit (Pierce, cat #PI23227, Thermo Scientific, USA) following manufacturer's protocol. The supernatant was mixed with a 4× Laemmli sample buffer and boiled for 5 min at 90-100° C., and 35 μg of total protein was run on a precast 4-12% Bis-Tris Protein Gels (Invitrogen, cat #NP0335BOX or alternative) for 15 minutes at 80 V and then 2 hours at 120 V. A pool of 3 samples from Bl6 mice was run alongside with C57Bl/6 AAV-treated samples as a control for TCAP and Flag quantification. Protein was transferred from gels to a 0.2 μm PVDF membrane using Trans-Blot Turbo system (Bio-Rad, cat #1704156 or alternative) at a constant 1.3-2.5 mA for 7-10 minutes. Precision plus protein dual color standards (BioRad, cat #1610394 or alternative) were used to determine the size of proteins of interest during gel running and protein transferring. To detect the expression of mouse TCAP (endogenous) and human TCAP (exogeneous), a cocktails of anti-FLAG antibody (Sigma-Aldrich, cat #SAB4200071-200UL, anti-rat monoclonal, dilution 1:1000) and anti-TCAP antibody (MyBioSource, cat #MBS9126105, anti-rabbit polyclonal, dilution 1:1000) was used in Odyssey Buffer (LI-COR, cat #927-40000) with 0.1% Tween-20. After overnight incubation at 4° C., membrane was washed (4×5 min with 0.1% Tween in PBS) and exposed to the secondary antibody cocktails, containing goat anti-rabbit IgG IRDye 800CW conjugate (LI-COR, cat #926-32211) and goat anti-rat IgG IRDye 680RD (LI-COR, cat #926-68076) for 60 min at RT at 1:5000 dilution in Odyssey blocking buffer. Blots were washed 5×5 min with 0.1% Tween in PBS, followed by 1×5 min wash in PBS. Blot was immediately immersed in ddH2O. Blots were scanned on LI-COR Odyssey CLx to detect the level of mouse and human TCAP expression in treated tissues.

Protein Extraction and Western Blotting Analysis from Human FibroMyoD Cells.

Cell lysates were prepared using manual homogenizing method in lysis buffer, containing 50 mM Tris-HCl (pH 7.4), 150 mM NaCl, 1% NP-40, digitonin (Sigma, cat #D141-500 MG or alternative) and protease inhibitor cocktail (Complete mini, EDTA-free protease inhibitor cocktail tablet; cat #NC0962311, Sigma Aldrich, USA). The lysate was centrifuged at 14,000 g for 20 min at 4° C. and supernatants were collected for analysis. Total protein was quantified by using BCA protein assay kit (Pierce, cat #PI23227, Thermo Scientific, USA) following manufacturer's protocol. The supernatant was mixed with a 4× Laemmli sample buffer and boiled for 5 min at 90-100° C., and 25 μg of total protein was run on a precast 12% Tris-Glycine Protein Gels (Invitrogen, cat #XP00120BOX) for 15 minutes at 80 V then 2 hours at 120 V. Proteins were transferred from gels to a 0.2 μm PVDF membrane using Trans-Blot Turbo system (Bio-Rad, cat #1704156) at a constant 1.3-2.5 mA for 7-10 minutes. Precision plus protein dual color standards (BioRad, cat #1610394 or alternative) were used to determine the size of proteins of interest during gel running and protein transferring. To detect total protein levels, blots were incubated in REVERT™ Total Protein Stain (LI-COR, cat #92611021, LI-COR, USA) and scanned in the 700 channel on an Odyssey CLx Imaging System (LI-COR). To detect the expression of human TCAP.FLAG, a cocktails of anti-FLAG antibody (Sigma-Aldrich, cat #SAB4200071-200UL, anti-rat monoclonal, dilution 1:1000) and anti-TCAP antibody (MyBioSource, cat #MBS9126105, anti-rabbit polyclonal, dilution 1:1000) was used in Odyssey Buffer (LI-COR, cat #927-40000) with 0.1% Tween-20 for overnight incubation at 4° C. Then, membrane was washed (4×5 min with 0.1% Tween in PBS) and exposed to the secondary antibody cocktails, containing goat anti-rabbit IgG IRDye 800CW conjugate (LI-COR, cat #926-32211) and goat anti-rat IgG IRDye 680RD (LI-COR, cat #926-68076) for 60 min at RT at 1:5000 dilution in Odyssey blocking buffer. Blots were washed 5×5 min with 0.1% Tween in PBS, followed by 1×5 min wash in PBS. Blot was immediately immersed in ddH2O. Blots were scanned on LI-COR Odyssey CLx to detect the level of TCAP expression in cells.

Protein Extraction and Western Blotting Analysis of hTCAPKI Mice after Intravenous (IV) Treatment with AAV9.TCAP Vectors.

Triceps were extracted using the RIPA buffer (Cell signaling, cat #9806S or alternative), containing PhosSTOP inhibitors (Sigma, cat #4906845001 or alternative), and Protein Inhibitor cocktail (Sigma Aldrich Fine Chemicals Biosciences, cat #11836170001 or alternative). Samples were homogenized as described for TA muscles. Novex 12% Tris-Glycine Protein Gels (Invitrogen, cat #XP00120BOX) were used for protein separation. Trans-Blot Turbo Mini PVDF Transfer Packs, 0.2 μm (Bio-Rad, cat #1704156 or alternative) were utilized to transfer proteins to the membrane. Membranes were blocked in 5% non-dry fat milk (NDFM) in TBST buffer and then probed with primary antibody cocktail containing, anti-TCAP antibody (MyBioSource, cat #MBS9126105, anti-rabbit polyclonal (1:1000) and anti-GAPDH antibody (Protein Tech, cat #60004-1-Ig, anti-mouse monoclonal, (1:50,000)) overnight at 4° C. Then, membrane was washed (4×5 min with 0.1% Tween TBS (TBST) and exposed to the secondary antibody cocktails, containing goat anti-rabbit IgG (HRP) (abcam, cat #ab6721) and goat anti-mouse IgG (HRP) (abcam, cat #ab6789) for 60 min at RT at 1:5000 dilution in 5% NDFM in TBST buffer. Then, blots were washed 5×5 min in TBST, followed by 1×5 min wash in TBS. Membranes were incubated with ECL reagent prior to visualization on Chemidoc MP Imaging System (Biorad, USA). TCAP signal in treated and control tissues was quantified using Image Lab software (Bio Rad, version 6.0.0 build 25) by normalizing the TCAP bands to the GAPDH band intensity of the respective sample. Next, the normalized TCAP signal from Bl6 control pool lanes was averaged and test samples were normalized to this average to give final TCAP quantification (%).

### Example 2

### TCAP Gene Replacement in C2C12 Myotubes

The following experiments were carried out to determine the expression of rAAV.TCAP.3×FLAG (Amp+) plasmids in C2C12 mouse myotubes after 8-9 days of differentiation by IF staining.

Cell culture: C2C12 myoblasts were electroporated using rAAV.TCAP.3×FLAG (Amp+) plasmids. 24 hours later, the medium was switched to differentiation medium, and cells were incubated in it for 8 days. C2C12 myotubes (D08) were transfected by Lipofectamine 3000 using the same sets of constructs. C2C12 myotubes (D09) on coverslips were washed in PBS and fixed in 4% PFA for IF staining.

Plasmids for electroporation/Lipofectamine 3000 transfection were as follows:
rAAV.MHCK7.TCAP.NI3×FLAG (Amp+);
rAAV.MHCK7.TCAP.SI3×FLAG (Amp+);
rAAV.CMV.TCAP.NI3×FLAG (Amp+); and
rAAV.CMV.TCAP.SI3×FLAG (Amp+).

### Cell Immunofluorescent (IF) Staining:

The IF protocol for cell staining was optimized and is described in the Materials and Methods, as described in Example 1. Briefly, cells were stained using two primary antibodies: TCAP AB (Abcam, cat #ab133646, anti-rabbit, dilution 1:100) and Desmin AB (Fisher, cat #MA5-13259, clone D33; anti-mouse, dilution 1:100). The cells were then stained using two secondary antibodies: goat anti rabbit, Alexa 568 (Invitrogen, cat #A-21069, dilution 1:500) and goat anti mouse, Alexa 488 (Invitrogen, cat #A-11017, dilution 1:500). The mounting medium included DAPI to stain and visualize all cell nuclei.

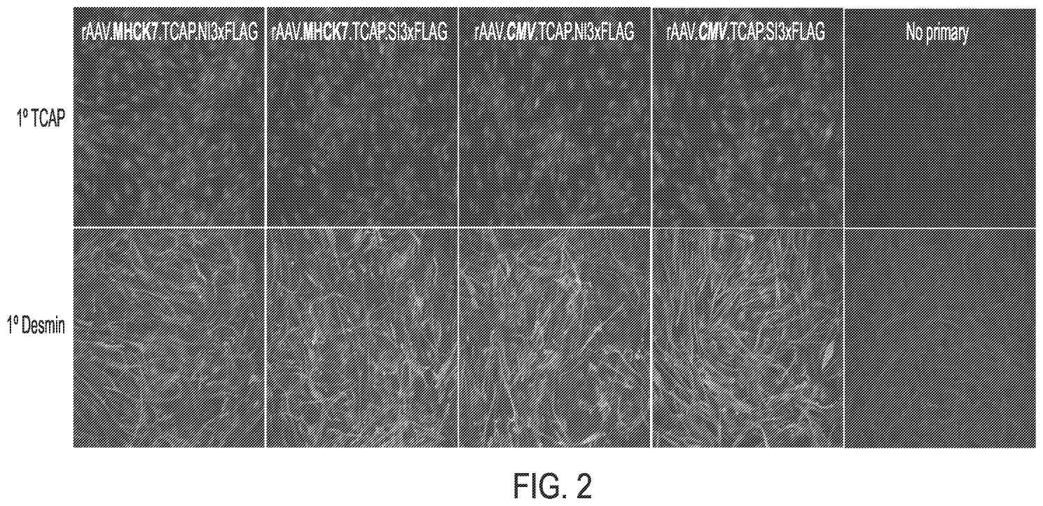

Immunofluorescent staining showed TCAP protein expression in differentiated C2C12 myotubes (Day 9) electroporated/transfected with rAAV.TCAP.3×FLAG (Amp+) plasmids (FIG. 2). C2C12 cells showed TCAP expression in rAAV.TCAP.3×FLAG (Amp+) transfected cells after nine days of differentiation.

### Example 3

### TCAP Gene Replacement in Mice

The following study was carried out to establish whether the expression of scAAV9.TCAP.FLAG (Amp+) vectors in skeletal muscles of C57Bl/6 mice could be detected. A single intramuscular injection of 1.5e11 vg per leg was delivered to the tibialis anterior (TA) muscle using each of the vectors listed below in Table 5. The TMN200P diluent was utilized as a vehicle control.

TABLE 5
rAAV.TCAP.3xFLAG (Amp+) vectors.

| | VVC Order | Titer |
|---|---|---|
| Full vector name | ID | (DRP/mL) |
| scAAV9.MHCK7.TCAP.NI3xFLAG (Amp+) | 600-1.1 | 3.0E12 |
| scAAV9.MHCK7.TCAP.SI3xFLAG (Amp+) | 600-2.1 | 4.3E12 |
| scAAV9.CMV.TCAP.NI3xFLAG (Amp+) | 602-1.1 | 4.1E12 |
| scAAV9.CMV.TCAP.SI3xFLAG (Amp+) | 602-2.1 | 6.9E12 |

Samples were collected 4 weeks post vector administration, and TCAP.3×FLAG transgene expression was determined by Western Blot using both TCAP and Flag antibodies. The diluent treated C57Bl/6 mice were utilized as controls.

All vectors were produced by the Vector core at Nationwide Children's Hospital (now known as Andelyn Biosciences). Physical titer determination was based on degradation of non-encapsidated DNA following digestion of viral capsids. Released encapsidated DNA was quantified by qPCR to determine the DNase Resistant Particle (DRP) titer (DRP/ml or vg/ml), utilizing a unique probe directed toward the MHCK7 or CMV promoters and a linearized plasmid standard. The vectors were diluted in TMN200P buffer, containing 20 mM Tris-HCl (pH 8.0), 200 mM NaCl, and 1 mM MgCl₂, 0.001% (w/w) Poloxamer 188, was provided by the VVC at NCH and utilized as a diluent for vector preparation. Poloxamer 188 (P188) is a nonionic linear copolymer having an average molecular weight of 8400 Daltons and is also referred to as PLURONIC F68, FLOCOR and RheothRx.

The research grade of TMN200P buffer, containing 20 mM Tris-HCl (pH 8.0), 200 mM NaCl, and 1 mM MgCl₂, 0.001% (w/w) Poloxamer 188, was provided by the VVC at NCH and utilized as a diluent for vector preparation. Animals were injected as provided in Table 6 set out below.

TABLE 6
C57BI/6 Animal Injection Log (Intramuscular Injections).

| | | | | Virus injection | | | | | | | |
|---|---|---|---|---|---|---|---|---|---|---|---|
| | | | | | Dose | | Age at | | | Age at | |
| Mouse | Mouse | | | | (vg per | Injection | Injection | Diluent | Necropsy | Necropsy | |
| Model | ID | Sex | DOB | Lot # | leg) | Date | (wks) | Lot # | Date | (wks) | Observations |
| | C57B1/6 - Diluent (Group 5) | | | | | | | | | | |
| C57B1/6 | 7301 | M | Jan. | N/A | N/A | Apr. | 12.4 | 030719AJS-01 | May | 16.4 | No comments |
| | | | 25, 2019 | | | 22, 2019 | | | 20, 2019 | | |
| C57B1/6 | 7302 | M | Jan. | N/A | N/A | Apr. | 12.4 | 030719AJS-01 | May | 16.4 | No comments |
| | | | 25, 2019 | | | 22, 2019 | | | 20, 2019 | | |
| C57B1/6 | 7326 | M | Jan. | N/A | N/A | Apr. | 11.7 | 030719AJS-01 | May | 15.7 | No comments |
| | | | 30, 2019 | | | 22, 2019 | | | 20, 2019 | | |
| C57B1/6 | 7327 | M | Jan. | N/A | N/A | Apr. | 11.7 | 030719AJS-01 | May | 15.7 | No comments |
| | | | 30, 2019 | | | 22, 2019 | | | 20, 2019 | | |
| C57B1/6 | 7328 | M | Jan. | N/A | N/A | Apr. | 11.7 | 030719AJS-01 | May | 15.7 | No comments |
| | | | 30, 2019 | | | 22, 2019 | | | 20, 2019 | | |
| | C57B1/6 - scAAV9.MHCK7.TCAP.NI3xFLAG (Amp+), Dose = 1.5E+11 vg/leg (Group 1) | | | | | | | | | | |
| C57B1/6 | 7149 | M | Dec. | 600-1.1 | 1.5E+11 | Mar. | 11.9 | 030719AJS-01 | Apr. | 15.9 | No comments |
| | | | 19, 2018 | | | 12, 2019 | | | 9, 2019 | | |
| C57B1/6 | 7150 | M | Dec. | 600-1.1 | 1.5E+11 | Mar. | 11.9 | 030719AJS-01 | Apr. | 15.9 | No comments |
| | | | 19, 2018 | | | 12, 2019 | | | 9, 2019 | | |
| C57B1/6 | 7151 | M | Dec. | 600-1.1 | 1.5E+11 | Mar. | 11.9 | 030719AJS-01 | Apr. | 15.9 | No comments |
| | | | 19, 2018 | | | 12, 2019 | | | 9, 2019 | | |
| C57B1/6 | 7152 | M | Dec. | 600-1.1 | 1.5E+11 | Mar. | 11.9 | 030719AJS-01 | Apr. | 15.9 | No comments |
| | | | 19, 2018 | | | 12, 2019 | | | 9, 2019 | | |
| | C57B1/6 - scAAV9.MHCK7.TCAP.SI3xFLAG (Amp+), Dose = 1.5E+11 vg/leg (Group 2) | | | | | | | | | | |
| C57B1/6 | 7153 | M | Dec. | 600-2.1 | 1.5E+11 | Mar. | 11.6 | 030719AJS-01 | Apr. | 15.6 | No comments |
| | | | 21, 2018 | | | 12, 2019 | | | 9, 2019 | | |
| C57B1/6 | 7154 | M | Dec. | 600-2.1 | 1.5E+11 | Mar. | 11.6 | 030719AJS-01 | Apr. | 15.6 | No comments |
| | | | 21, 2018 | | | 12, 2019 | | | 9, 2019 | | |
| C57B1/6 | 7155 | M | Dec. | 600-2.1 | 1.5E+11 | Mar. | 11.6 | 030719AJS-01 | Apr. | 15.6 | No comments |
| | | | 21, 2018 | | | 12, 2019 | | | 9, 2019 | | |
| C57B1/6 | 7213 | M | Jan. | 600-2.1 | 1.5E+11 | Apr. | 12.1 | 030719AJS-01 | May | 16.1 | No comments |
| | | | 8, 2019 | | | 3, 2019 | | | 1, 2019 | | |
| | C57B1/6 - scAAV9.CMV.TCAP.NI3xFLAG (Amp+), Dose = 1.5E+11 vg/leg (Group 3) | | | | | | | | | | |
| C57B1/6 | 7214 | M | Jan. | 602-1.1 | 1.5E+11 | Apr. | 12.1 | 030719AJS-01 | May | 16.1 | No comments |
| | | | 8, 2019 | | | 3, 2019 | | | 1, 2019 | | |
| C57B1/6 | 7215 | M | Jan. | 602-1.1 | 1.5E+11 | Apr. | 12.1 | 030719AJS-01 | May | 16.1 | No comments |
| | | | 8, 2019 | | | 3, 2019 | | | 1, 2019 | | |
| C57B1/6 | 7216 | M | Jan. | 602-1.1 | 1.5E+11 | Apr. | 12.1 | 030719AJS-01 | May | 16.1 | No comments |
| | | | 8, 2019 | | | 3, 2019 | | | 1, 2019 | | |
| C57B1/6 | 7217 | M | Jan. | 602-1.1 | 1.5E+11 | Apr. | 12.1 | 030719AJS-01 | May | 16.1 | No comments |
| | | | 8, 2019 | | | 3, 2019 | | | 1, 2019 | | |
| | C57B1/6 - scAAV9.CMV.TCAP.SI3xFLAG (Amp+), Dose = 1.5E+11 vg/leg (Group 4) | | | | | | | | | | |
| C57B1/6 | 7265 | M | Jan. | 602-2.1 | 1.5E+11 | Apr. | 12.0 | 030719AJS-01 | May | 16.0 | No comments |
| | | | 18, 2019 | | | 12, 2019 | | | 10, 2019 | | |
| C57B1/6 | 7266 | M | Jan. | 602-2.1 | 1.5E+11 | Apr. | 12.0 | 030719AJS-01 | May | 16.0 | No comments |
| | | | 18, 2019 | | | 12, 2019 | | | 10, 2019 | | |
| C57B1/6 | 7267 | M | Jan. | 602-2.1 | 1.5E+11 | Apr. | 12.0 | 030719AJS-01 | May | 16.0 | No comments |
| | | | 18, 2019 | | | 12, 2019 | | | 10, 2019 | | |
| C57B1/6 | 7268 | M | Jan. | 602-2.1 | 1.5E+11 | Apr. | 12.0 | 030719AJS-01 | May | 16.0 | No comments |
| | | | 18, 2019 | | | 12, 2019 | | | 10, 2019 | | |

Western Blot analysis was carried out. Mouse tissue lysates were prepared by mechanically disrupting tissue in a Tissuelyser II (Qiagen, USA) in lysis buffer, containing 50 mM Tris-HCl (pH 7.4), 150 mM NaCl, 1% NP-40, digitonin (Sigma, cat #D141-500 MG or alternative) and protease inhibitor cocktail (Complete mini, EDTA-free protease inhibitor cocktail tablet; cat #NC0962311, Sigma Aldrich, USA). The lysate was centrifuged at 14,000 g for 20 min at 4° C. and supernatants were collected for analysis. Total protein was quantified by using BCA protein assay kit (Pierce, cat #PI23227, Thermo Scientific, USA) following manufacturer's protocol. The supernatant was mixed with a 4× Laemmli sample buffer and boiled for 5 min at 90-100° C., and 35 μg of total protein was run on a precast 4-12% Bis-Tris Protein Gels (Invitrogen, cat #NP0335BOX) for 15 minutes at 80 V and then 2 hours at 120 V. A pool of 3 samples from Bl6 mice was run alongside with C57Bl/6 AAV-treated samples as a control for TCAP and Flag quantification. Protein was transferred from gels to a 0.2 μm PVDF membrane using Trans-Blot Turbo system (Bio-Rad, cat #1704156 or alternative) at a constant 1.3-2.5 mA for 7-10 minutes. Precision plus protein dual color standards (BioRad, cat #1610394 or alternative) were used to determine the size of proteins of interest during gel running and protein transferring. To detect the expression of mouse TCAP (endogenous) and human TCAP (exogeneous), a cocktails of anti-FLAG antibody (Sigma-Aldrich, cat #SAB4200071-200UL, anti-rat monoclonal, dilution 1:1000) and anti-TCAP antibody (MyBioSource, cat #MBS9126105, anti-rabbit polyclonal, dilution 1:1000) was used in Odyssey Buffer (LI-COR, cat #927-40000 or alternative) with 0.1% Tween-20. After overnight incubation at 4° C., membrane was washed (4×5 min with 0.1% Tween in PBS) and exposed to the secondary antibody cocktails, containing goat anti-rabbit IgG IRDye 800CW conjugate (LI-COR, cat #926-32211) and goat anti-rat IgG IRDye 680RD (LI-COR, cat #926-68076) for 60 min at RT at 1:5000 dilution in Odyssey blocking buffer. Blots were washed 5×5 min with 0.1% Tween in PBS, followed by 1×5 min wash in PBS. Blot was immediately immersed in ddH2O. Blots were scanned on LI-COR Odyssey CLx to detect the level of mouse and human TCAP expression in treated tissues.

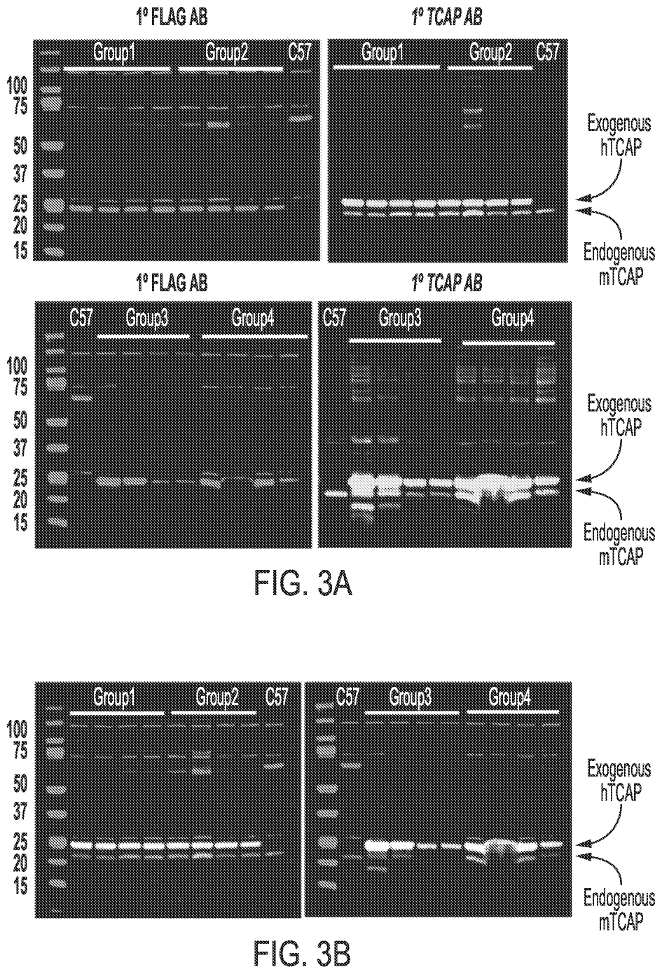

The co-expression of endogenous mouse TCAP (mTCAP) and exogenous human TCAP.3×FLAG (hTCAP) proteins in C57Bl/6 (C57) mice intramuscularly (IM) injected with scAAV9.TCAP.FLAG (Amp+) vectors is shown in FIG. 3A-B. FIG. 3A shows individual channels showing FLAG (red) and TCAP (green) signals in the animals injected with Diluent (C57, Group 5) and scAAV9.MHCK7.TCAP.NI3×FLAG (Amp+) (Group 1), scAAV9.MHCK7.TCAP.SI3×FLAG (Amp+) (Group 2), scAAV9.CMV.TCAP.NI3×FLAG (Amp+) (Group 3), and scAAV9.CMV.TCAP.SI3×FLAG (Amp+) (Group 4). FIG. 3B shows the merged images showing the co-expression (yellow) of both proteins in each of Groups 1˜4 in TA muscles.

This experiment shows that all scAAV9.TCAP.FLAG (Amp+) test articles resulted in human TCAP protein overexpression in tibialis anterior muscles of C57Bl/6 mice 4 weeks post vector administration at the dose of 1.5E11 vg per muscle.

### Example 4

### TCAP Gene Expression in TCAP-Null Cells

The following study was carried out to establish whether TCAP gene expression could be carried out in TCAP-null cells. TCAP-null cells (see MTA #[2019-0217]) were used to determine the efficacy of the rAAV vectors in vitro using both Western Blot analysis and immunofluorescent (IF) staining. The functional activity of the expressed TCAP protein was determined using infrared multi-photon laser microscopy.

Human immortalized tet-inducible-MyoD fibroblast TCAP-null cells were transfected with scAAV1.MHCK7.TCAP.SI3×FLAG (Amp+) vector as set out in Table 7 below. The following human immortalized tet-inducible-MyoD cells were used in the experiments: FM193ML (TCAP-null cells); FM021PK (a normal female cell line); and FM190KL (a normal male cell line).

TABLE 7
rAAV.TCAP.3xFLAG vector information.

| | VVC Order | Titer |
|---|---|---|
| Full vector name | ID | (DRP/mL) |
| scAAV1.MHCK7.TCAP.SI3xFLAG | TT657-1 | 6.0E12 |
| (Amp+) | | |

Western Blot analysis was carried out. Cell lysates were prepared using manual homogenizing method in lysis buffer, containing 50 mM Tris-HCl (pH 7.4), 150 mM NaCl, 1% NP-40, digitonin (Sigma, cat #D141-500 MG or alternative) and protease inhibitor cocktail (Complete mini, EDTA-free protease inhibitor cocktail tablet; cat #NC0962311, Sigma Aldrich, USA). The lysate was centrifuged at 14,000 g for 20 min at 4° C. and supernatants were collected for analysis. Total protein was quantified by using BCA protein assay kit (Pierce, cat #PI23227, Thermo Scientific, USA) following manufacturer's protocol. The supernatant was mixed with a 4× Laemmli sample buffer and boiled for 5 min at 90-100° C., and 25 μg of total protein was run on a precast 12% Tris-Glycine Protein Gels (Invitrogen, cat #XP00120BOX) for 15 minutes at 80 V and then 2 hours at 120 V. Proteins were transferred from gels to a 0.2 μm PVDF membrane using Trans-Blot Turbo system (Bio-Rad, cat #1704156 or alternative) at a constant 1.3-2.5 mA for 7-10 minutes. Precision plus protein dual color standards (BioRad, cat #1610394 or alternative) were used to determine the size of proteins of interest during gel running and protein transferring. To detect total protein levels, blots were incubated in REVERT™ Total Protein Stain (LI-COR, cat #92611021, LI-COR, USA or alternative) and scanned in the 700 channel on an Odyssey CLx Imaging System (LI-COR). To detect the expression of human TCAP.FLAG, a cocktails of anti-FLAG antibody (Sigma-Aldrich, cat #SAB4200071-200UL, anti-rat monoclonal, dilution 1:1000) and anti-TCAP antibody (MyBioSource, cat #MBS9126105, anti-rabbit polyclonal, dilution 1:1000) was used in Odyssey Buffer (LI-COR, cat #927-40000 or alternative) with 0.1% Tween-20 for overnight incubation at 4° C. Then, membrane was washed (4×5 min with 0.1% Tween in PBS) and exposed to the secondary antibody cocktails, containing goat anti-rabbit IgG IRDye 800CW conjugate (LI-COR, cat #926-32211) and goat anti-rat IgG IRDye 680RD (LI-COR, cat #926-68076) for 60 min at RT at 1:5000 dilution in Odyssey blocking buffer. Blots were washed 5×5 min with 0.1% Tween in PBS, followed by 1×5 min wash in PBS. The blots were immediately immersed in ddH2O. Blots were scanned on LI-COR Odyssey CLx to detect the level of TCAP expression in cells.

The expected bands were (1) endogenous human TCAP in WT controls cells (about 19 kDa), and (2) exogeneous human TCAP in FM193ML (TCAP-null) cells treated with SCAAV1.MHCK7.TCAP.SI3×FLAG (Amp+) about 21-22 kDa.

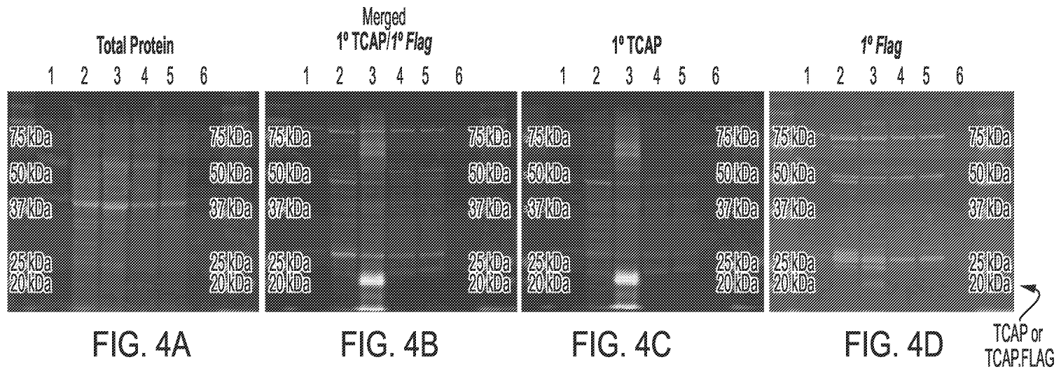

Representative images from the Western Blot analysis showed restoration of the TCAP protein in TCAP-null cells treated with scAAV1.MHCK7.TCAP.SI3×FLAG (Amp+) (FIG. 4A-D). Lanes 1-6 of the Western Blots (FIG. 4A-D) contained protein lysates as follows: 1) FM193ML Fibroblasts; 2) FM193ML Myoblasts, Day 3; 3) FM193ML Myotubes treated with scAAV1.MHCK7.TCAP.SI3×FLAG (Amp+) vector, Day 14 differentiation; 4) FM193ML Myotubes untreated, Day 14 differentiation; 5) FM021PK Myotubes, Day 14 differentiation; and 6) FM190KL Myotubes, Day 15 differentiation. FIG. 4A shows total protein level detected by using Total Protein Stain (REVERT) from LiCOR. FIG. 4B-D provides merged images and individual images, respectively, with membrane that was probed with anti-TCAP (FIG. 4C) and anti-FLAG (FIG. 4D) antibodies followed by secondary IRDye 800 or IRDye 680 RD conjugated antibody.

This study showed that treatment with an AAV1 vector comprising the TCAP gene (i.e., scAAV1.MHCK7.TCAP.SI3×FLAG (Amp+)) was efficient to express the TCAP protein detected by both anti-TCAP and anti-FLAG antibodies in human TCAP-null cells after 14 days of differentiation. Unfortunately, the human control cell lines (both male and female) did not show any visible TCAP protein expression at similar conditions. Additional experiments are being carried out using a new male cell line, FM000KF, to determine the endogenous TCAP level in controls and to quantify the TCAP.3×FLAG level restored in treated FM193ML cells.

Immunofluorescent (IF) staining was carried out. The following human immortalized tet-inducible-MyoD cells were used in the experiment: FM193ML (TCAP-null untreated cells) and FM193ML (TCAP-null cells treated with scAAV1.MHCK7.TCAP.SI3×FLAG (Amp+)) test articles. Both untreated and treated cells were differentiated for 14 days and fixed in 4% PFA. The fixed human FibroMyoD cells were permeabilized in PBS solution, containing 0.1% Triton X-100 for 5 minutes and then incubated in PBS, containing 1% Bovine Serum Albumin (BSA) (Fisher BioReagents, cat #BP9706100 or alternative) for 30-60 minutes at RT. Cells were incubated in PBS with 0.5% BSA and 0.1% Triton X-100 containing the following primary antibodies anti-TCAP antibody (MyBioSource, cat #MBS9126105, anti-rabbit polyclonal, dilution 1:100) and anti-FLAG antibody (Sigma-Aldrich, cat #SAB4200071-200UL, anti-rat monoclonal, dilution 1:200) for 2 hours at room temperature. Then cells were washed in PBS with 0.1% Triton X-100 three times and incubated in 0.5% BSA with 0.1% Triton X-100 and 5% normal goat serum (Invitrogen, cat #10000C or alternative), containing the following secondary antibodies goat anti rabbit, Alexa 647 (Invitrogen, cat #A21244, dilution 1:500) and donkey anti-rat, Alexa 488 (Jackson Labs, cat #712-546-153, dilution 1:500) for one hour in the dark at room temperature. Then, cells were washed in PBS with 0.2% Triton X-100 three times, washed one time in PBS and then mounted in the medium with DAPI (Vector laboratories, cat #H-1500 or alternative) to stain and visualize all cell nuclei.

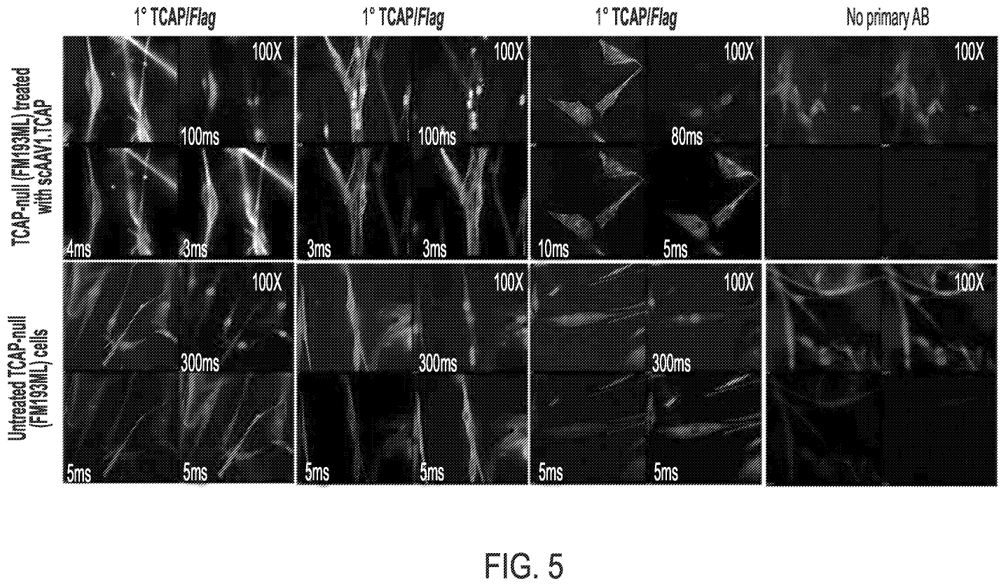

Human FibroMyoD cells were imaged on Nikon Ti2E fluorescence microscope with a 100× oil objective. Representative IF images showing TCAP (Alexa 647, magenta channel) and FLAG (Alexa 488, green channel) in TCAP-null cells (FM193ML) treated with SCAAV1.MHCK7.TCAP.SI3×FLAG (Amp+) vector (FIG. 5; top panel) and untreated (FIG. 5; bottom panel) after 14 days of differentiation are shown. All images contain 4 small squares, each of them represents either merged channels (left top corner) or DAPI (nuclei staining, right top corner), FLAG (left bottom corner) and TCAP (right bottom corner). The images were taken on a Nikon Ti2E microscope with a 100× oil objective. Tissue sections stained with secondary antibody only (no primary antibody) were used as a negative control to determine non-specific background of images.

The fluorescent microscopy revealed the expression of TCAP protein by detecting both TCAP and FLAG signals in TCAP-null cells treated with test articles and confirmed their colocalization in the cytoplasm with more pronounced signal located on the plasma membrane. Some non-specific signal was evident in untreated FM193ML cells, as determined for both TCAP and FLAG antibodies. Although non-specific signal was detected, the brightness of these non-specific signals was reduced, even though the exposure time was almost twice the exposure time used for scAAV1 treated cells.

Laser-Induced Cell Damage. The membrane injury assay was performed on untreated and AAV1-treated human FibroMyoD cells, which were plated on 35-mm dishes and differentiated for 5 days. Cells were incubated in Tyrode's solution, 140 mM NaCl, 5 mM KCl, 2 mM MgCl₂, and 10 mM HEPES, pH 7.2 with (2 mM Ca²⁺) or without (0 mM Ca²⁺) calcium ions. Cell membrane damage was induced in the presence of 2.5 M FM4-64 amphiphilic fluorescent styryl pyridinium dye (Invitrogen, cat #T13320 or alternative) with or without 2.0 mM Ca²⁺, using the Olympus FV1000 multi-photon laser scanning confocal system. Cells were irradiated at 12.5-24% of laser power for 3 sec. Pre- and post-damage images were captured every 3 sec, continuing for 53 sec. The extent of membrane damage was analyzed using ImageJ software (NIH, version 1.53c), by measuring the fluorescence intensity encompassing the site of damage.

To confirm the functional activity of TCAP in TCAP-null cells, laser injury was carried out on the cells. Pre- and post-damage images were captured every 3 sec, continuing for 53 sec. The Olympus FV1000 multi-photon laser scanning confocal system was utilized.

The following human immortalized tet-inducible-MyoD cells were used in the experiment: FM193ML-TCAP-null untreated cells; FM193ML-TCAP-null cells treated scAAV1.MHCK7.TCAP.SI3×FLAG (Amp+) test articles; and FM000KF-normal male cell line. All cell lines were differentiated for 5 days. 35-mm dishes with coverslips coated with fibronectin solution were prepared for plating the cells.

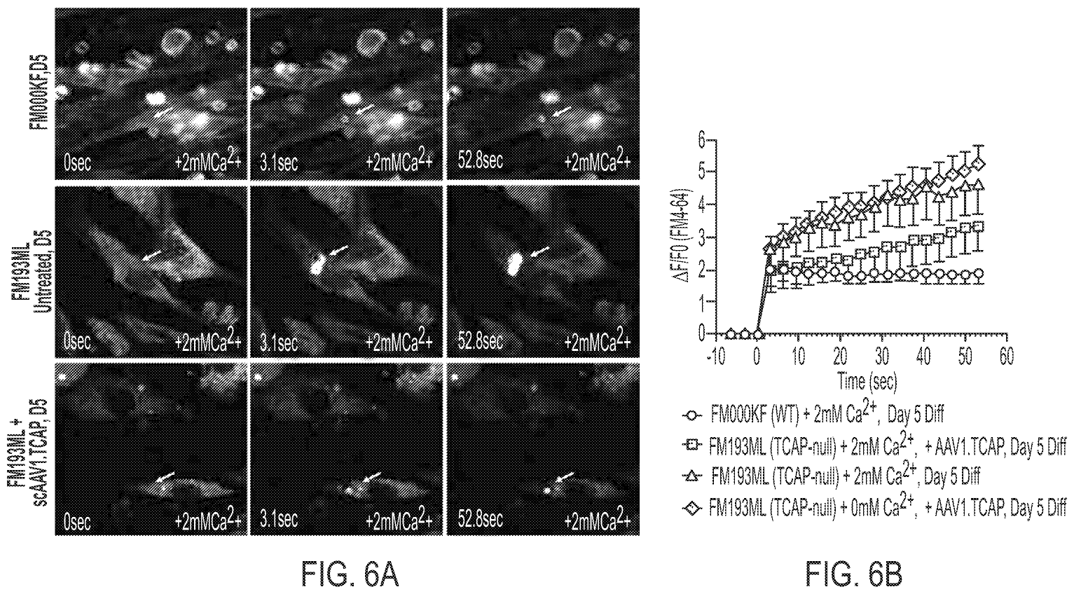

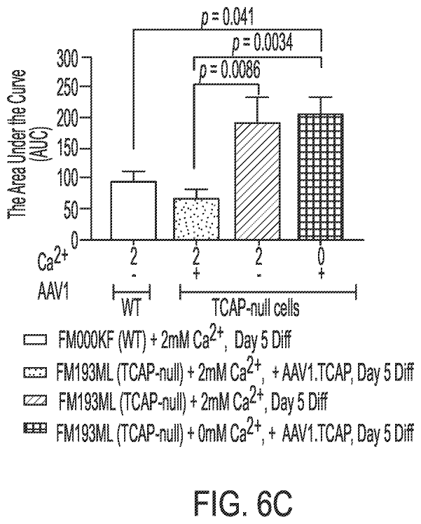

The extent of membrane damage was analyzed using ImageJ software, by measuring the fluorescence intensity encompassing the site of damage. The background was subtracted for each measurement. FIG. 6A provides representative images of laser injury in cells after 5 days of differentiation (Day 5 Diff). White arrows indicate the wounding site at 0 sec, 3.1 sec and 52.8 sec after laser injury in the presence of calcium ions (2 mM Ca²⁺). FIG. 6B shows time-dependent accumulation of FM4-64 dye in FM000KF (normal male cells, WT cells) and FM193ML (TCAP-null) untreated cells and treated with scAAV1.MHCK7.TCAP.SI3×FLAG (Amp+) test articles, the analysis of which is represented as the Area Under the Curve (AUC). AUC is the definitive integral of a dye accumulation curve between 0 sec and 53.04 sec. FIG. 6C shows AUC of FM4-64 fluorescence traces displays the membrane-resealing process in normal human myotubes (FM000KF, Day 5 Diff), TCAP-deficient (FM193ML, Day 5 Diff) or TCAP-treated (FM193ML treated with scAAV1.MHCK7.TCAP.SI3×FLAG (Amp+), Day 5 Diff) cells. The effect of the absence of calcium ions (0 mM Ca²⁺) is recorded for scAAV1 treated FM193ML cells. Data shown are represented as means±SEM (n=9-12). The Tukey-ANOVA was utilized for statistical analysis.

Human myotubes deficient in the TCAP protein displayed compromised membrane repair. Restoration of TCAP function, i.e., the transduction of TCAP cDNA into the myotubes, led to an increase in the membrane repair capacity up to the level detected in normal human myotubes. Results also show that calcium ions play an essential role in this process, because the absence of extracellular Ca²⁺ resulted in significant dye entry following laser injury despite TCAP expression in transduced/treated cells suggesting that calcium is important in membrane repair.

### Example 5

TCAP.FLAG Expression in Humanized TCAP KnockIn (hTCAPKI) Mice Treated Intramuscularly (IM) with scAAV9.TCAP.FLAG (Amp+) Vectors

The following study was carried out to determine if human TCAP protein expression could be achieved in humanized TCAP KnockIn (hTCAPKI) mice.

The hTCAPKI mouse model was kindly provided by Drs. Charles Emerson (University of California, San Diego), Greg Cox (The Jackson Laboratory), and Scot Wolfe (University of Massachusetts Medical School) and transferred from the Jackson Laboratory (MTA #[2020-0448]). Animals have been maintained by in-house breeding. The mouse model was created by insertion of an 8 bp fragment into the mouse TCAP gene and modifying the flanking codons to match the human TCAP gene. The novel mouse model can be used for developing new translational approaches, including gene therapy. Due to the limited number of animals available, only one to two mice were tested in this experiment (see Table 8, set out below, for animal injection log).

TABLE 8
hTCAPKI Animal Injection Log (Intramuscular Injections).

| | | | | Virus/Diluent injection | | | | | | | |
|---|---|---|---|---|---|---|---|---|---|---|---|
| | | | | | Dose | | Age at | | | Age at | |
| Mouse | Mouse | | | Vector | (vg per | Injection | Injection | Diluent | Necropsy | Necropsy | |
| Model | ID | Sex | DOB | Lot # | leg) | Date | (wks) | Lot # | Date | (wks) | Observations |
| | C57B1/6 - untreated (Group 1) | | | | | | | | | | |
| C57B1/6 | A0199 | M | Dec. | N/A | N/A | N/A | 11.0 | n/a | Mar. | 15.0 | No comments |
| | | | 4, 2020 | | | | | | 19, 2021 | | |
| | hTCAPKI - Diluent treated (Group 2) | | | | | | | | | | |
| hTCAPKI | A0307 | M | Dec. | N/A | N/A | Feb. | 9.57 | TMNP0619-B | Mar. | 13.6 | No comments |
| | | | 14, 2020 | | | 19, 2021 | | | 19, 2021 | | |
| | hTCAPKI - scAAV9.MHCK7.TCAP.NI3xFLAG (Amp+), Dose = 1.5E+11 vg/leg (Group 3) | | | | | | | | | | |
| hTCAPKI | A0305 | M | Dec. | 600-1.1 | 1.5E+11 | Feb. | 9.57 | TMNP0619-B | Mar. | 13.6 | No comments |
| | | | 14, 2020 | | | 19, 2021 | | | 19, 2021 | | |
| hTCAPKI | A0306 | M | Dec. | 600-1.1 | 1.5E+11 | Feb. | 9.57 | TMNP0619-B | Mar. | 13.6 | No comments |
| | | | 14, 2020 | | | 19, 2021 | | | 19, 2021 | | |
| | hTCAPKI - scAAV9.MHCK7.TCAP.SI3xFLAG (Amp+), Dose = 1.5E+11 vg/leg (Group 4) | | | | | | | | | | |
| hTCAPKI | A0308 | M | Dec. | 600-2.1 | 1.5E+11 | Feb. | 9.57 | TMNP0619-B | Mar. | 13.6 | No comments |
| | | | 14, 2020 | | | 19, 2021 | | | 19, 2021 | | |
| hTCAPKI | A0309 | M | Dec. | 600-2.1 | 1.5E+11 | Feb. | 9.57 | TMNP0619-B | Mar. | 13.6 | No comments |
| | | | 14, 2020 | | | 19, 2021 | | | 19, 2021 | | |

### Experimental Protocol:

hTCAPKI mice were injected in the tibialis anterior (TA) muscle with one of two constructs:
a) scAAV9.TCAP.MHCK7.NI3×Flag (Amp+), and
b) scAAV9.TCAP.MHCK7.SI3×Flag (Amp+).
   Control Mice were Used as Follows:
1) C57Bl/6 (Bl6): one B16 mouse was utilized as a control and was not injected with any vehicles; and
2) hTCAPKI: one hTCAPKI mouse was injected intramuscularly with vehicle (Diluent) into each TA muscle at 10 weeks of age.

hTCAPKI mice (two mice per group, each at 10-week old) were intramuscularly injected with 1.5E11 vg of test articles into the TA muscles. TCAP vectors for this study were produced in the Viral Vector Core (VVC) Laboratory at the Abigail Wexner Research Institute, Nationwide Children's Hospital (Columbus, OH), under research-grade conditions. The qPCR Physical Titers listed in Table 2 were generated according to a Linear plasmid standard. The vectors were diluted in 20 mM Tris-HCl (pH 8.0), 200 mM NaCl, and 1 mM MgCl₂, 0.001% Pluronic F68. The research grade of TMN200P buffer, containing 20 mM Tris-HCl (pH 8.0), 200 mM NaCl, and 1 mM MgCl₂, 0.001% (w/w) Poloxamer 188, was provided by the VVC Laboratory and utilized as a diluent for vector preparation.

All mice were euthanized 4-week post vector administration. TA muscles were collected 4 weeks post vector administration, and TCAP.3×FLAG (Amp+) transgene expression was determined by IF using TCAP and Titin antibodies. Tissues from untreated and diluent-treated C57Bl/6 and hTCAPKI-null mice were utilized as positive and negative controls.

Immunofluorescent staining. Frozen TA muscles were cut at 10 microns, air-dried, permeabilized in 0.1% Triton X-100 PBS, and blocked in 1×PBS with 10% normal goat serum (NGS, Invitrogen, cat #10000C or alternative) and 0.1% Tween 20 for 1 hour at RT. Then, samples were blocked in goat-anti-mouse IgG (H+L) unconjugated fab fragment antibody (Jackson, cat #115-007-003 or alternative) for 2 hours at RT. Following blocking, sections were co-stained in 1:100 rabbit polyclonal anti-TCAP antibody (Mybiosource, cat #MBS9126105) and 1:20 mouse monoclonal anti-Titin antibody (DSHB, cat #9D10) for 2 hours at RT. Slides were washed 4×5 min, incubated in the appropriate Alexa Fluor647 (Invitrogen, cat #A21244, goat anti-rabbit) and Alexa Fluor 488 (Invitrogen, cat #A11017, goat anti-mouse) conjugated secondary antibodies (1:500 dilution) for 1 hour, and washed again 3×5 minutes. Tissues were affixed with ProLong Gold Antifade mounting medium with DAPI (Invitrogen, cat #P36934 or alternative). TA muscle images were collected on Nikon Ti2E fluorescence microscope with a 100× oil objective.

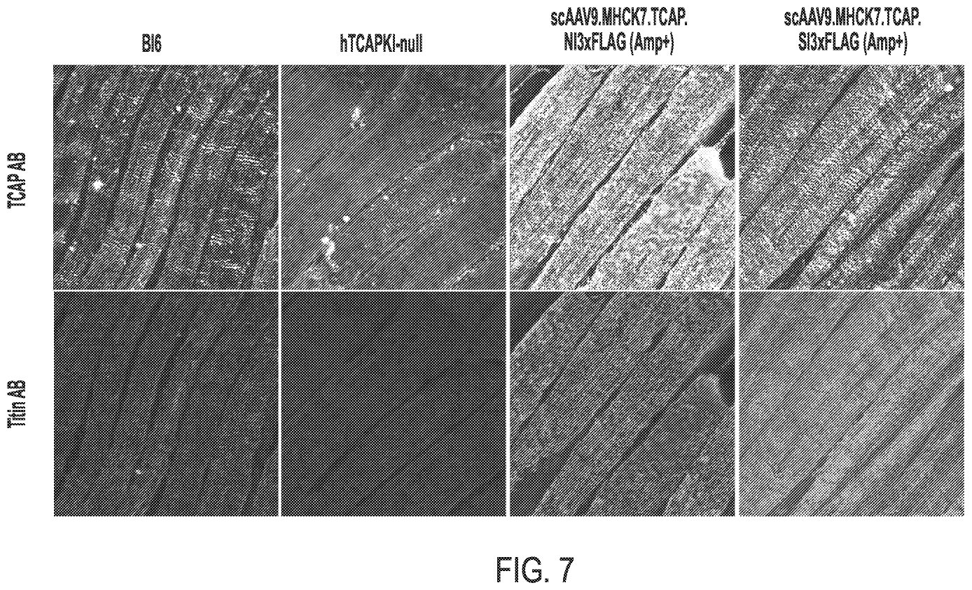

FIG. 7 shows representative IF images showing TCAP (Alexa 647, magenta channel) and Titin (Alexa 488, green channel) co-localization. 10-week-old hTCAPKI males were intramuscularly injected with scAAV9.MHCK7.TCAP.NI3×FLAG (Amp+) or scAAV9.MHCK7.TCAP.SI3×FLAG (Amp+) vectors and TA muscles were collected 4 weeks post-vector administration. The images were taken on a Nikon Ti2E microscope with a 100× oil objective. Scale bar is 10 μm. T Tissue sections stained with secondary antibody only (no primary antibody) were used as a negative control to determine non-specific background of images. Scale bar=10 μm.

The fluorescent microscopy revealed the significant restoration of human TCAP protein in TA muscles from hTCAPKI-null males 4 weeks post scAAV9.TCAP.FLAG (Amp+) vector administration. Titin protein expression, as well as its proper co-localization with TCAP, was observed. A significant increase in titin protein expression was observed in hTCAPKI treated mice. hTCAPKI controls stained with TCAP antibody showed some propensity for nonspecific binding leading to a background signal seen in hTCAPKI diluent group, which is being optimized in ongoing IF experiments.

### Example 6

TCAP.FLAG Expression in Humanized TCAP KnockIn (hTCAPKI) Mice Intravenously (IV) Treated with scAAV9.TCAP.FLAG (Amp+) Vectors

The following study was carried out to determine lead candidates using four vectors for human TCAP protein expression in humanized TCAP KnockIn (hTCAPKI) mice. To determine the lead candidate, four TCAP.FLAG (Amp+) vectors were selected for systemic delivery in hTCAPKI males:
a) scAAV9.TCAP.MHCK7.NI3×Flag (Amp+);
b) scAAV9.TCAP.MHCK7.SI3×Flag (Amp+);
c) scAAV9.TCAP.CMV.NI3×Flag (Amp+); and
d) SCAAV9.TCAP.CMV.SI3×Flag (Amp+).

Due to the limited number of animals available, only three to four mice per group were tested (see Table 9, set out below, for animal injection log).

TABLE 9
hTCAPKI Animal Injection Log (Intravenous Injections).

| | | | | Virus injection | | | | | | | |
|---|---|---|---|---|---|---|---|---|---|---|---|
| | | | | | | | Age at | | | Age at | |
| Mouse | Mouse | | | Vector | Dose | Injection | Injection | Diluent | Necropsy | Necropsy | |
| Model | ID | Sex | DOB | Lot # | (vg/kg) | Date | (wks) | Lot # | Date | (wks) | Observations |
| | C57B1/6 - untreated (Group 1) | | | | | | | | | | |
| C57B1/6 | A0564 | M | Feb. | N/A | N/A | N/A | N/A | N/A | Apr. | 10.4 | No comments |
| | | | 14, 2021 | | | | | | 28, 2021 | | |
| C57B1/6 | A0565 | M | Feb. | N/A | N/A | N/A | N/A | N/A | Apr. | 10.4 | No comments |
| | | | 14, 2021 | | | | | | 28, 2021 | | |
| C57B1/6 | A0566 | M | Feb. | N/A | N/A | N/A | N/A | N/A | Apr. | 10.4 | No comments |
| | | | 14, 2021 | | | | | | 28, 2021 | | |
| C57B1/6 | A0567 | M | Feb. | N/A | N/A | N/A | N/A | N/A | Apr. | 10.4 | No comments |
| | | | 14, 2021 | | | | | | 28, 2021 | | |
| | hTCAPKI - Diluent treated (Group 2) | | | | | | | | | | |
| hTCAPKI | A0556 | M | Feb. | N/A | N/A | Apr. | 7.0 | TMNP0619-B | Apr. | 10.9 | No comments |
| | | | 12, 2021 | | | 2, 2021 | | | 29, 2021 | | |
| hTCAPKI | A0557 | M | Feb. | N/A | N/A | Apr. | 7.0 | TMNP0619-B | Apr. | 10.9 | No comments |
| | | | 12, 2021 | | | 2, 2021 | | | 29, 2021 | | |
| hTCAPKI | A0558 | M | Feb. | N/A | N/A | Apr. | 7.0 | TMNP0619-B | Apr. | 10.9 | No comments |
| | | | 12, 2021 | | | 2, 2021 | | | 29, 2021 | | |
| hTCAPKI | A0561 | M | Feb. | N/A | N/A | Apr. | 7.0 | TMNP0619-B | Apr. | 10.9 | No comments |
| | | | 12, 2021 | | | 2, 2021 | | | 29, 2021 | | |
| | hTCAPKI - scAAV9.MHCK7.TCAP.NI3xFLAG (Amp+), Dose = 3.0E13 vg/kg (Group 3) | | | | | | | | | | |
| hTCAPKI | A0559 | M | Feb. | 600-1.1 | 3.0E+13 | Apr. | 7.0 | TMNP0619-B | Apr. | 11.0 | No comments |
| | | | 12, 2021 | | | 2, 2021 | | | 30, 2021 | | |
| hTCAPKI | A0560 | M | Feb. | 600-1.1 | 3.0E+13 | Apr. | 7.0 | TMNP0619-B | Apr. | 11.0 | No comments |
| | | | 12, 2021 | | | 2, 2021 | | | 30, 2021 | | |
| hTCAPKI | A0562 | M | Feb. | 600-1.1 | 3.0E+13 | Apr. | 7.0 | TMNP0619-B | Apr. | 11.0 | No comments |
| | | | 12, 2021 | | | 2, 2021 | | | 30, 2021 | | |
| | hTCAPKI - scAAV9.MHCK7.TCAP.SI3xFLAG (Amp+), Dose = 3.0E13 vg/kg (Group 4) | | | | | | | | | | |
| hTCAPKI | A0602 | M | Feb. | 600-2.1 | 3.0E+13 | Apr. | 6.0 | TMNP0619-B | May | 10.1 | No comments |
| | | | 22, 2021 | | | 5, 2021 | | | 4, 2021 | | |
| hTCAPKI | A0603 | M | Feb. | 600-2.1 | 3.0E+13 | Apr. | 6.0 | TMNP0619-B | May | 10.1 | No comments |
| | | | 22, 2021 | | | 5, 2021 | | | 4, 2021 | | |
| hTCAPKI | A0604 | M | Feb. | 600-2.1 | 3.0E+13 | Apr. | 6.0 | TMNP0619-B | May | 10.1 | No comments |
| | | | 22, 2021 | | | 5, 2021 | | | 4, 2021 | | |
| hTCAPKI | A0605 | M | Feb. | 600-2.1 | 3.0E+13 | Apr. | 6.0 | TMNP0619-B | May | 10.1 | No comments |
| | | | 22, 2021 | | | 5, 2021 | | | 4, 2021 | | |
| | hTCAPKI - scAAV9.CMV.TCAP.NI3xFLAG (Amp+), Dose = 3.0E13 vg/kg (Group 5) | | | | | | | | | | |
| hTCAPKI | A0615 | M | Feb. | 602-1.1 | 3.0E+13 | Apr. | 5.57 | TMNP0619-B | Apr. | 9.6 | No comments |
| | | | 22, 2021 | | | 2, 2021 | | | 30, 2021 | | |
| hTCAPKI | A0609 | M | Feb. | 602-1.1 | 3.0E+13 | Apr. | 5.71 | TMNP0619-B | Apr. | 9.7 | No comments |
| | | | 21, 2021 | | | 2, 2021 | | | 30, 2021 | | |
| hTCAPKI | A0610 | M | Feb. | 602-1.1 | 3.0E+13 | Apr. | 5.71 | TMNP0619-B | Apr. | 9.7 | No comments |
| | | | 21, 2021 | | | 2, 2021 | | | 30, 2021 | | |
| | hTCAPKI - scAAV9.CMV.TCAP.SI3xFLAG (Amp+), Dose = 3.0E13 vg/kg (Group 6) | | | | | | | | | | |
| hTCAPKI | A0593 | M | Feb. | 602-2.1 | 3.0E+13 | Apr. | 6.0 | TMNP0619-B | May | 10.3 | No comments |
| | | | 22, 2021 | | | 5, 2021 | | | 5, 2021 | | |
| hTCAPKI | A0594 | M | Feb. | 602-2.1 | 3.0E+13 | Apr. | 6.0 | TMNP0619-B | May | 10.3 | No comments |
| | | | 22, 2021 | | | 5, 2021 | | | 5, 2021 | | |
| hTCAPKI | A0595 | M | Feb. | 602-2.1 | 3.0E+13 | Apr. | 6.0 | TMNP0619-B | May | 10.3 | No comments |
| | | | 22, 2021 | | | 5, 2021 | | | 5, 2021 | | |
| hTCAPKI | A0598 | M | Feb. | 602-2.1 | 3.0E+13 | Apr. | 6.0 | TMNP0619-B | May | 10.3 | No comments |
| | | | 22, 2021 | | | 5, 2021 | | | 5, 2021 | | |

Control mice: C57Bl/6 (Bl6): four Bl6 mice were utilized as a control group and were not injected with any vehicles. hTCAPKI: four hTCAPKI mice were intravenously injected with vehicle (Diluent) at 7 weeks of age.

Treated mice: three to four 6-7 week-old hTCAPKI mice from each group were intravenously injected with 3.0E13 vg/kg. All mice were euthanized 4-week post-vector administration.

Skeletal muscles and other organs and tissues were collected 4 weeks post-vector administration. TCAP.3×FLAG transgene expression was determined by Western blot analysis using TCAP and GAPDH antibodies. Tissues from untreated and diluent-treated C57Bl/6 and hTCAPKI-null mice were utilized as positive and negative controls.

All TCAP vectors were produced by the Vector core at Nationwide Children's Hospital (now known as Andelyn Biosciences). Physical titer determination was based on degradation of non-encapsidated DNA following digestion of viral capsids. Released encapsidated DNA was quantified by qPCR to determine the DNase Resistant Particle (DRP) titer (DRP/ml or vg/ml), utilizing a unique probe directed toward the MHCK7 or CMV promoters and a linearized plasmid standard. The vectors were diluted in TMN200P buffer, containing 20 mM Tris-HCl (pH 8.0), 200 mM NaCl, and 1 mM MgCl₂, 0.001% (w/w) Poloxamer 188, was provided by the VVC at NCH and utilized as a diluent for vector preparation. Poloxamer 188 (P188) is a nonionic linear copolymer having an average molecular weight of 8400 Daltons and is also referred to as PLURONIC F68, FLOCOR and RheothRx.

Western Blot analysis. Triceps were extracted using the RIPA buffer (Cell signaling, Cat #9806S), containing PhosSTOP inhibitors (Sigma, cat #4906845001 or alternative), and Protein Inhibitor cocktail (Sigma Aldrich Fine Chemicals Biosciences, cat #11836170001 or alternative). Samples were homogenized as described for TA muscles. Novex 12% Tris-Glycine Protein Gels (Invitrogen, cat #XP00120BOX or alternative) were used for protein separation. Trans-Blot Turbo Mini PVDF Transfer Packs, 0.2 μm (Bio-Rad, cat #1704156) were utilized to transfer proteins to the membrane. Membranes were blocked in 5% non-dry fat milk (NDFM) in TBST buffer and then probed with primary antibody cocktail containing, anti-TCAP antibody (MyBioSource, cat #MBS9126105, anti-rabbit polyclonal (1:1000) and anti-GAPDH antibody (Protein Tech, cat #60004-1-Ig, anti-mouse monoclonal, (1:50,000)) overnight at 4° C. Then, membrane was washed (4×5 min with 0.1% Tween TBS (TBST)) and exposed to the secondary antibody cocktails, containing goat anti-rabbit IgG (HRP) (abcam, cat #ab6721) and goat anti-mouse IgG (HRP) (abcam, cat #ab6789 or alternative) for 60 min at RT at 1:5000 dilution in 5% NDFM in TBST buffer. Then, blots were washed 5×5 min in TBST, followed by 1×5 min wash in TBS. Membranes were incubated with ECL reagent prior to visualization on Chemidoc MP Imaging System (Biorad, USA). TCAP signal in treated and control tissues was quantified using Image Lab software (Bio Rad, version 6.0.0 build 25) by normalizing the TCAP bands to the GAPDH band intensity of the respective sample. Next, the normalized TCAP signal from Bl6 control pool lanes was averaged and test samples were normalized to this average to give final TCAP quantification (%).

Expected bands: No bands were expected for lanes comprising hTCAPKI mice samples (negative control). Endogenous human TCAP in Bl6 mice ˜19 kDa (positive control). Exogenous human TCAP in hTCAPKI mice treated with scAAV9.TCAP.FLAG vectors ˜21-22 kDa.

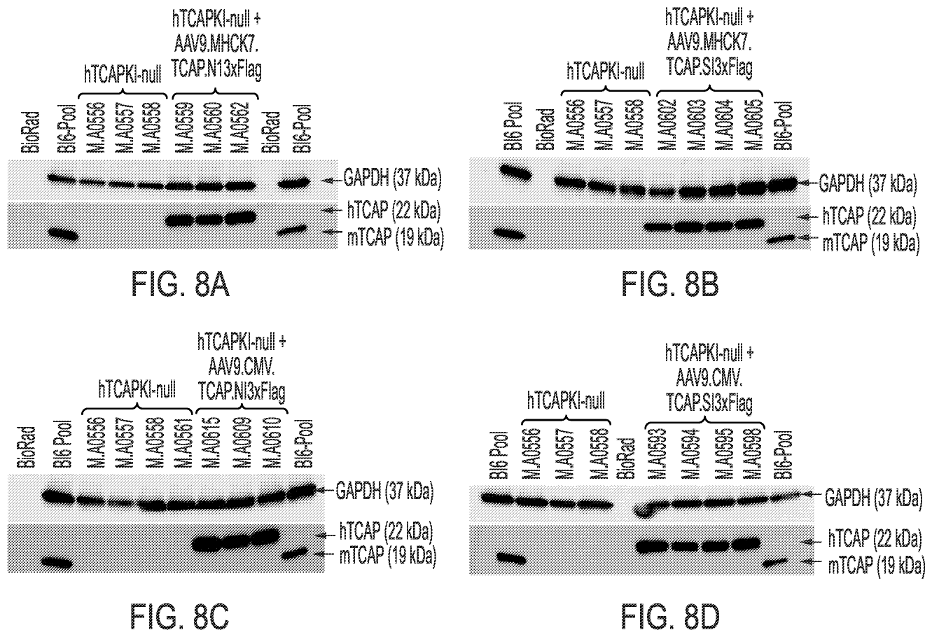

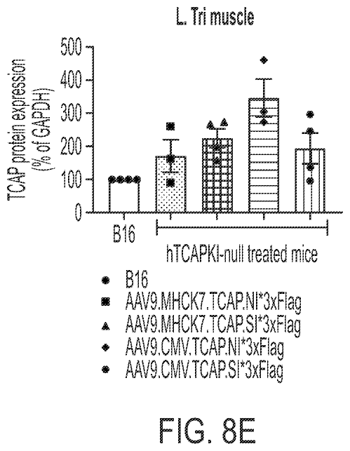

FIG. 8A-E show the results obtained after systemic delivery of scAV9.TCAP.FLAG. There was significant TCAP protein expression in triceps muscle 4 weeks post-injection. Representative images of immunoblots show increases in TCAP expression in triceps post-administration with (FIG. 8A) scAAV9.TCAP.MHCK7.NI3×Flag, (FIG. 8B) scAAV9.TCAP.MHCK7.SI3×Flag, (FIG. 8C) scAAV9.TCAP.CMV.NI3×Flag and (FIG. 8D) scAAV9.TCAP.CMV.SI3×Flag. (FIG. 8E) Densitometric analysis of the Western blots presented in FIG. 8A-D was carried out using Image Lab software (Bio Rad, version 6.0.0 build 25). The intensity of TCAP bands in treated groups was normalized to the GAPDH band intensity of the respective sample. Data are means±SEM (n=3-4 for each group).

All systemically delivered scAAV9.TCAP.FLAG vectors showed a significant level of TCAP protein expression in triceps muscles. Samples from C57Bl6 (Bl6) animals revealed that the endogenously expressed mouse TCAP (mTCAP) protein is smaller in size than exogenously expressed human TCAP (hTCAP) protein, as expected due to a 3×Flag sequence extension.

### Example 7

### Second Generation of Self-Complementary TCAP Constructs

The following second generation of self-complementary recombinant AAV vectors comprising either the NP, the MHCK, or the CMV promoter were designed and constructed (Tables 10A-C, FIGS. 19-28). Tables 10A-B and FIGS. 19-28 provide specifics regarding the total plasmid size, transgene size (with and without ITR sequences), promoter, and other elements.

TABLE 10A
Second generation of self-complementary TCAP
construct (TCAP Nos. 11-20) overview. *

| | | | Transgene | | |
|---|---|---|---|---|---|
| | | | size | | |
| | | | without | | |
| | | | 5′ and 3′ | | |
| | | Total | ITR | | |
| | | Plasmid | sequences | | Other |
| NO. | Name | size (bp) | (bp) | Promoter | elements |
| 11 | rAAV.NP.TCAP/SD | 5749 | 1725 | NP | No FLAG |
| 12 | rAAV.MHCK7.TCAP.NI/SD | 5628 | 1609 | MHCK7 | No FLAG |
| 13 | rAAV.MHCK7.TCAP.SI/SD | 5534 | 1515 | MHCK7 | No FLAG |
| 14 | rAAV.CMV.TCAP.NI/SD | 5440 | 1447 | CMV | No FLAG |
| 15 | rAAV.CMV.TCAP.SI/SD | 5341 | 1348 | CMV | No FLAG |
| 16 | rAAV.NP.TCAP.3xFLAG/SD | 5815 | 1791 | NP | 3xFLAG |
| 17 | rAAV.MHCK7.TCAP.NI3xFLAG/SD | 5694 | 1675 | MHCK7 | 3xFLAG |
| 18 | rAAV.MHCK7.TCAP.SI3xFLAG/SD | 5600 | 1581 | MHCK7 | 3xFLAG |
| 19 | rAAV.CMV.TCAP.NI3xFLAG/SD | 5506 | 1513 | CMV | 3xFLAG |
| 20 | rAAV.CMV.TCAP.SI3xFLAG/SD | 5407 | 1414 | CMV | 3xFLAG |

NP—native promoter
NI—native intron
SI—synthetic intron
3xFLAG—3 tandem repeats of the FLAG sequence
SD—indicates the backbone that was used for generation of the plasmids.

TABLE 10B
TCAP construct (TCAP Nos. 11-20) transgene sequences.

| | | Transgene | | Transgene | Transgene |
|---|---|---|---|---|---|
| | | size (bp) | | size (bp) | w/5′ and 3′ |
| | | without | | with | ITR |
| | | 5′ and 3′ | Transgene | 5′ and 3′ | sequences |
| | | ITR | SEQ ID | ITR | SEQ ID |
| No. | Name | sequences | NO: | sequences | NO: |
| 11 | rAAV.NP.TCAP/SD | 1725 | 29 | 2096 | 49 |
| 12 | rAAV.MHCK7.TCAP.NI/SD | 1609 | 30 | 1975 | 50 |
| 13 | rAAV.MHCK7.TCAP.SI/SD | 1515 | 31 | 1881 | 51 |
| 14 | rAAV.CMV.TCAP.NI/SD | 1447 | 32 | 1787 | 52 |
| 15 | rAAV.CMV.TCAP.SI/SD | 1348 | 33 | 1688 | 53 |
| 16 | rAAV.NP.TCAP.3xFLAG/SD | 1791 | 34 | 2162 | 54 |
| 17 | rAAV.MHCK7.TCAP.NI3xFLAG/ | 1675 | 35 | 2041 | 55 |
| | SD | | | | |
| 18 | rAAV.MHCK7.TCAP.SI3xFLAG/ | 1581 | 36 | 1947 | 56 |
| | SD | | | | |
| 19 | rAAV.CMV.TCAP.NI3xFLAG/SD | 1513 | 37 | 1853 | 57 |
| 20 | rAAV.CMV.TCAP.SI3xFLAG/SD | 1414 | 38 | 1754 | 58 |

SEQ ID NOs: 29-38 = transgene; 49-58 = transgene w/ITR; and 39-48 = rAAV TCAP construct.

### Example 8

### Testing Second Generation of Self-Complementary TCAP Constructs

Recombinant AAV vectors comprising either the NP, the MHCK, or the CMV promoter are tested in humanized TCAPKI (hTCAPKI) mice for further use in human clinical trials. The minimum efficacious dose (MED) is established by comparing several doses of the lead construct driven by a promoter selected based upon previous experiments. The mouse and human TCAP transcripts are determined using RT-PCR analysis. TCAP protein expression is determined by Western Blot (WB) and/or immunofluorescent (IF) analyses. The vector(s) biodistribution in skeletal muscles and organs is determined by qPCR and/or ddPCR analysis. In addition, muscle function is assessed by rotarod, hindlimb and forelimb grip strength, as well as in vitro and in vivo muscle physiology measurements. Histological (H&E, Masson's trichrome) examination of skeletal muscle and heart muscle is used to assess tissue pathology. Echocardiograms are carried out to evaluate cardiac function. Measurements and responses are compared for normal and age-matched wild-type C57Bl/6 (Bl6) mice and in untreated and/or treated with vehicle control hTCAPKI mice. The vector(s) biodistribution is determined by qPCR and/or ddPCR analysis.

### Example 9

### TCAP Expression/Restoration in Muscle

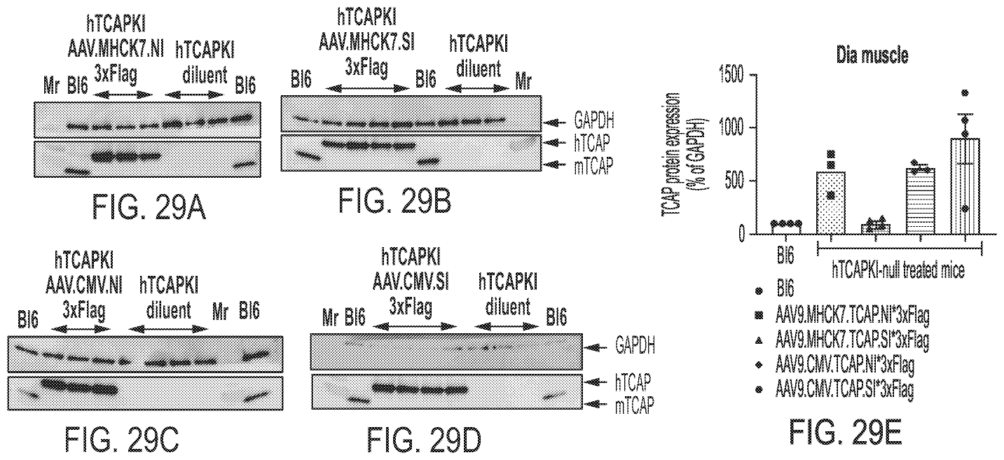

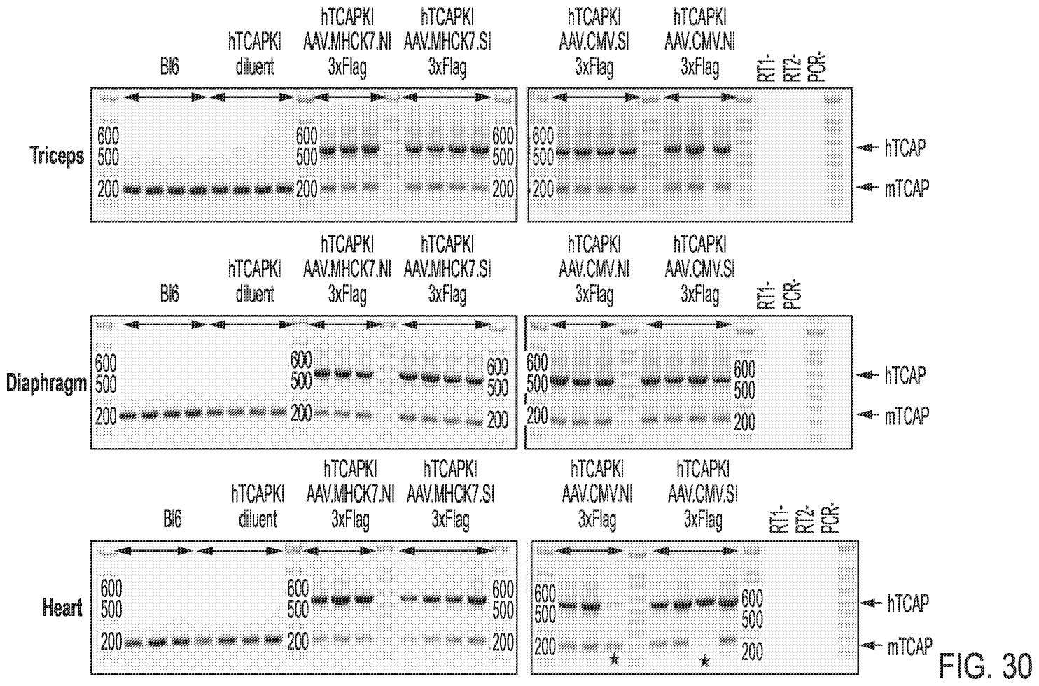

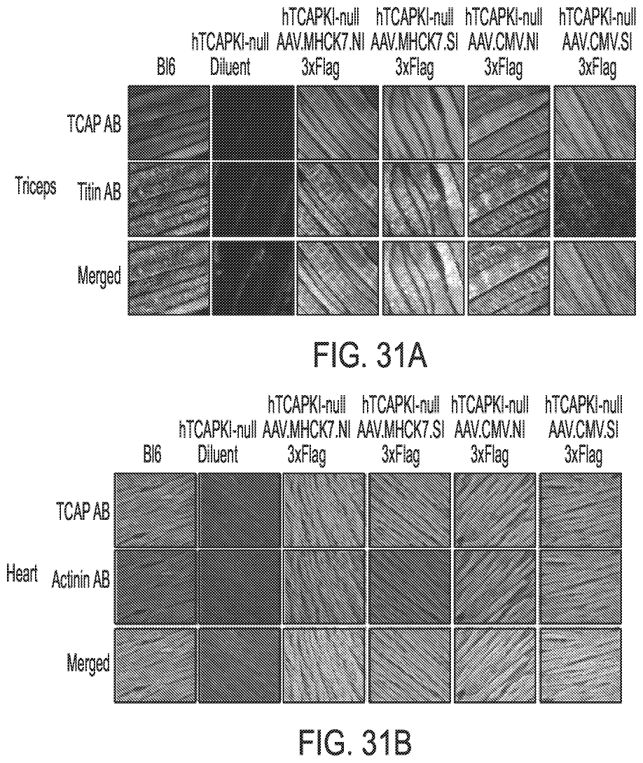

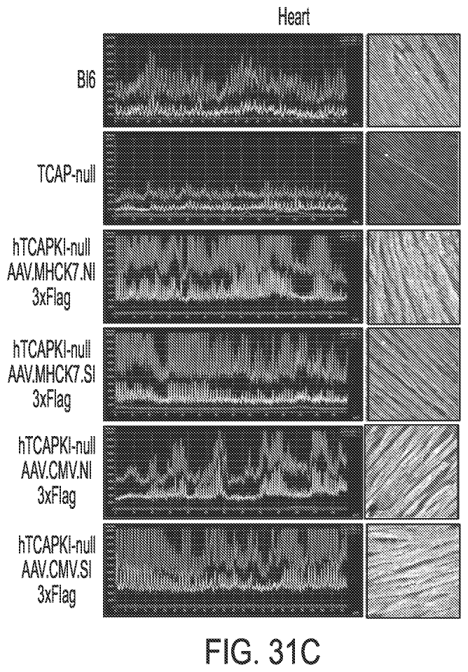

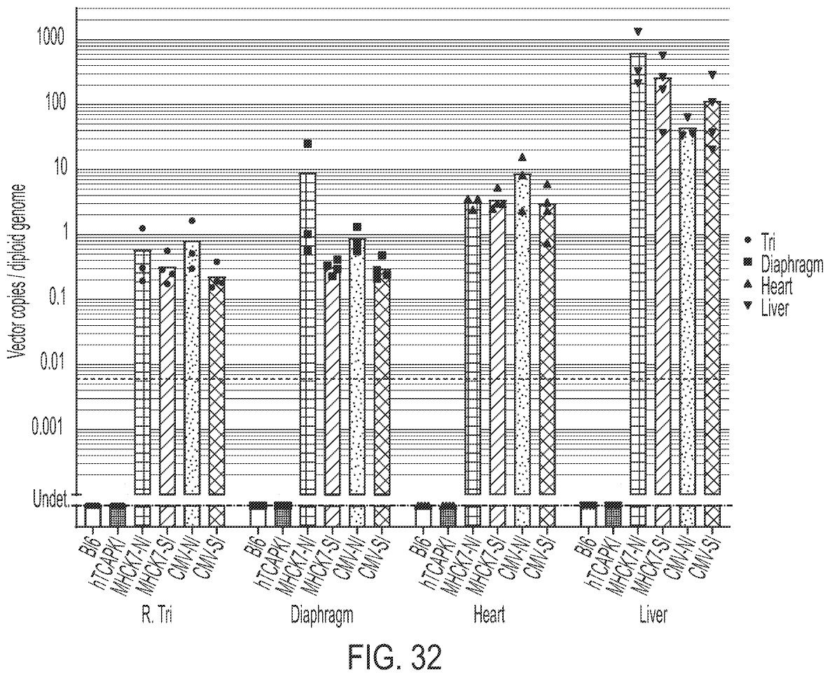

This example provides supplementary data from experiments reported in Example 6. Recombinant AAV.TCAP vectors were tested in humanized TCAPKI (hTCAPKI) mice to determine expression and effect of TCAP protein. hTCAPKI mice (n=3-4) were treated intravenously for 4 weeks with rAAV.TCAP vectors (scAAV9.TCAP.MHCK7.NI3×FLAG, scAAV9.TCAP.MHCK7.SI3×FLAG, scAAV9.TCAP.CMV.NI3×FLAG, and scAAV9.TCAP.CMV.SI3×FLAG) at a dose of 3E13 vg/kg or with saline as a control. Age-matched untreated C57Bl6 (Bl6) and diluent treated hTCAPKI-null (hTCAPKI) mice were used as controls. Animals were sacrificed 4 weeks post-vector administration and various muscles or organs were subject to various tests as described below and as shown in FIGS. 29A-E, 30, 31A-C, and 32.

### Materials and Methods

### Western Blotting

Diaphragms were extracted using the RIPA buffer (Cell signaling, cat #9806S or alternative), containing PhosSTOP inhibitors (Sigma, cat #4906845001), and Protein Inhibitor cocktail (Sigma Aldrich Fine Chemicals Biosciences, cat #11836170001). Samples were homogenized as described for TA muscles. Novex 12% Tris-Glycine Protein Gels (Invitrogen, cat #XP00120BOX) were used for protein separation. Trans-Blot Turbo Mini PVDF Transfer Packs, 0.2 μm (Bio-Rad, cat #1704156) were utilized to transfer proteins to the membrane. Membranes were blocked in 5% non-dry fat milk (NDFM) in TBST buffer and then probed with primary antibody cocktail containing, anti-TCAP antibody (MyBioSource, cat #MBS9126105, anti-rabbit polyclonal (1:1000) and anti-GAPDH antibody (Protein Tech, cat #60004-1-Ig, anti-mouse monoclonal, (1:50,000)) overnight at 4° C. Membrane was washed (4×5 min with 0.1% Tween TBS (TBST) and exposed to the secondary antibody cocktails, containing goat anti-rabbit IgG (HRP) (abcam, cat #ab6721) and goat anti-mouse IgG (HRP) (abcam, cat #ab6789) for 60 min at RT at 1:5000 dilution in 5% NDFM in TBST buffer. Blots then were washed 5×5 min in TBST, followed by 1×5 min wash in TBS. Membranes were incubated with ECL reagent prior to visualization on Chemidoc MP Imaging System (Biorad, USA). TCAP signal in treated and control tissues was quantified using Image Lab software (Bio Rad, version 6.0.0 build 25) by normalizing the TCAP bands to the GAPDH band intensity of the respective sample. The normalized TCAP signal from Bl6 control pool lanes was averaged and test samples were normalized to this average to give final TCAP quantification (%).

### RNA Extraction and RT-PCR Analysis

Total RNA was extracted from frozen skeletal muscles and hearts through standard TRIzol/chloroform extraction (Life Technologies, cat #15596018, Carlsbad, CA; Fisher Bioreagents, cat #C297-4, Hampton, NH) and then purified using RNA Clean & Concentrator-25 according to the manufacturer's instruction (Zymo Research, cat #R1018, Tustin, CA). Reverse transcription (RT) was performed on 500-1000 ng of total RNA with RevertAid RT Kit and random hexamer primers according to the manufacturer's protocol (Thermo Scientific, cat #K1691, Waltham, MA). cDNA was amplified via PCR (Thermo Fisher, cat #K0171, Waltham, MA) using primers specific to the mouse and human TCAP sequences. The gel images were taken using ChemiDoc Imaging System (Bio-Rad). Primer sequences were used as follows:

### Human Transcript:

forward:
5′-AATGAGGAGTGATCATGGCTACCTC-3′,
reverse:
5′-TCACTTGTCATCGTCATCCTTGTAATCG-3′;
Mouse transcript:
forward:
5′-CATCTTCACGCCCACCAAG-3′,
reverse:
5′-ACATGGATCGAGACAGGGTA-3′.

### IF Staining

Triceps brachii and heart tissues were stained using the following method. 10 μm-thick longitudinally oriented sections of cryopreserved tissues were air-dried for 15 min before staining. Following rehydration in PBS for 15 min, sections were incubated for 1 h with blocking solution containing 10% normal goat serum (NGS, Invitrogen, cat #10000C or alternative) in PBS followed by a 2 h incubation with a goat-anti-mouse Fab IgG (Jackson, cat #115-007-003 or alternative; dilution 1:10) in PBS-0.1% Tween-20 (PBST) at room temperature (RT). Sections were incubated with primary antibodies for 2 hours at RT or overnight at 4° C., and then washed with 1×PBST 4 times for 5 min at RT. Sections were incubated in the appropriate Alexa Fluor 647 (Invitrogen, cat #A21244, goat anti-rabbit) and Alexa Fluor 488 (Invitrogen, cat #A11017, goat anti-mouse) conjugated secondary antibodies (1:500 dilution) for 1 hour and washed again with 1×PBST 3 times for 5 min and last time with PBS for 5 min. Tissues were then affixed with ProLong Gold Antifade mounting medium with DAPI (Invitrogen, cat #P36934 or alternative). The following primary antibodies were used: rabbit polyclonal anti-TCAP antibody (1:100; Mybiosource, cat #MBS9126105); mouse monoclonal anti-Titin antibody (1:20; DSHB, cat #9D10), mouse monoclonal anti-sarcomeric alpha-actinin antibody (1:200; Invitrogen, cat #MA1-22863, clone EA-53).

### Microscope Imaging

Triceps brachii and heart muscle images were taken on a Nikon Ti2-E inverted widefield fluorescent microscope with a 100× oil objective using NIS-Elements (version 5.21.00, build 1483). To analyze sarcomere assembly in hTCAPKI-treated cardiomyocytes, TCAP and alpha-actinin signal intensity plots were taken for multiple sarcomeres in a single representative cardiomyocyte, which is shown by a yellow line on the merged images.

TCAP qPCR Biodistribution

Vector genomes were quantified in cardiac, skeletal muscles (triceps brachii and diaphragm) and liver. Total DNA was extracted from tissue samples using DNeasy Blood & Tissue Kit (Qiagen, catalog #69506, Germantown, MD, USA), and vector genomes quantified by quantitative PCR (qPCR) using a linearized AAV.TCAP plasmid to generate a standard curve for each transgene individually allowing absolute quantification. The DNA samples were analyzed using TaqMan Universal PCR Master Mix (Applied Biosystems, catalog #4304437) and Quantstudio6 Flex (Applied Biosystems, Foster City, CA USA), following the procedures recommended by the manufacturer and a set of primers unique to the transgene. Primer and probe sequences used were as follows:

qPCR.hTCAP-forward:
CATGTCCCAGGAAGCACAG
qPCR.hTCAP-reverse:
TCGTCATCCTTGTAATCGATGTC
qPCR.hTCAP-probe:
CGTCATGGTCTTTGTAGTCGCCTCT
(/56-FAM/CG TCA TGG T/ZEN/C
TTT GTA GTC GCC TCT /3IABKFQ/)

Genomic DNA from non-injected C57Bl6 (Bl6) and diluent treated hTCAPKI mouse tissues served as experimental control samples. Each sample was run in triplicate.

### Results

The western blot results revealed that all systemically delivered scAAV9.TCAP.FLAG vectors showed a significant level of TCAP protein expression in diaphragm muscles. Samples from C57Bl6 (Bl6) animals revealed that endogenously expressed mouse TCAP (mTCAP) protein is smaller in size than exogenously expressed human TCAP (hTCAP) protein, as expected due to the 3×Flag sequence extension.

The RT-PCR data showed significant amplification of mouse TCAP (mTCAP) mRNA transcripts in all untreated and rAAV.TCAP treated mice. The human TCAP (hTCAP) mRNA transcripts were detected exceptionally in hTCAPKI mice treated with rAAV.TCAP vectors.

Biodistribution of human TCAP transgene was assessed by quantitative real-time PCR (qPCR) using DNA extracted from four tissues: triceps brachiim, diaphragm, heart and liver. As expected, the highest values for vector copies per diploid genome (vc/dg) were found in liver ranging from 44.2-620.9 vc/dg. Skeletal muscles and heart contained 0.58-8.9 vc/dg one month post vectors administration.

### Example 10

Restoration of TCAP Expression in Skeletal Muscles of hTCAPKI-Null Mice Systemically Injected with AAV.TCAP Vectors

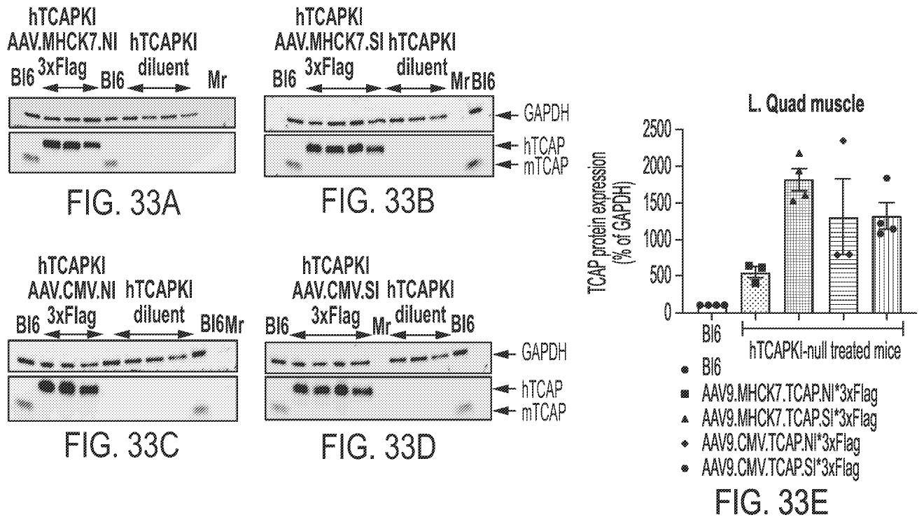

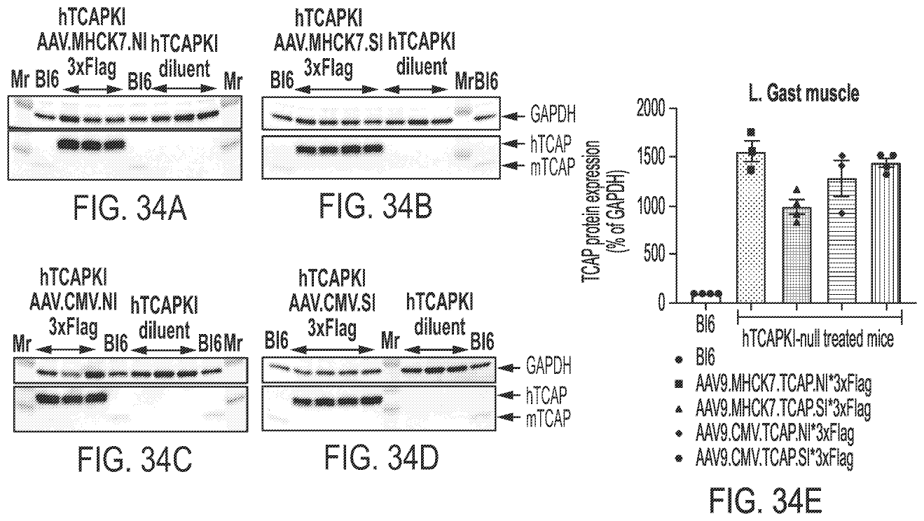

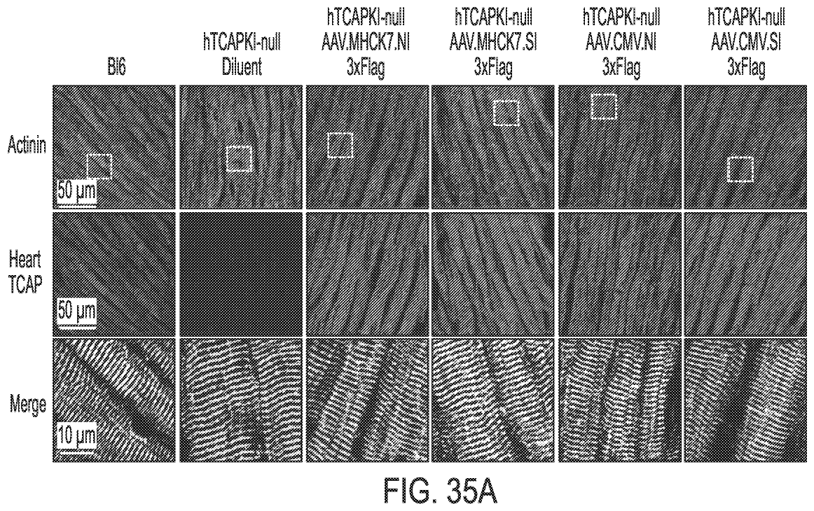

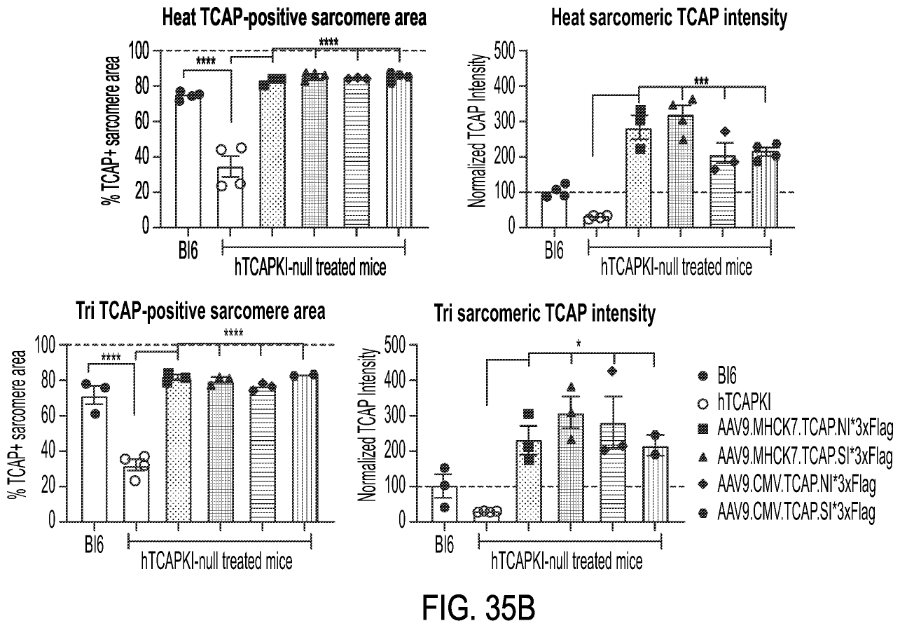

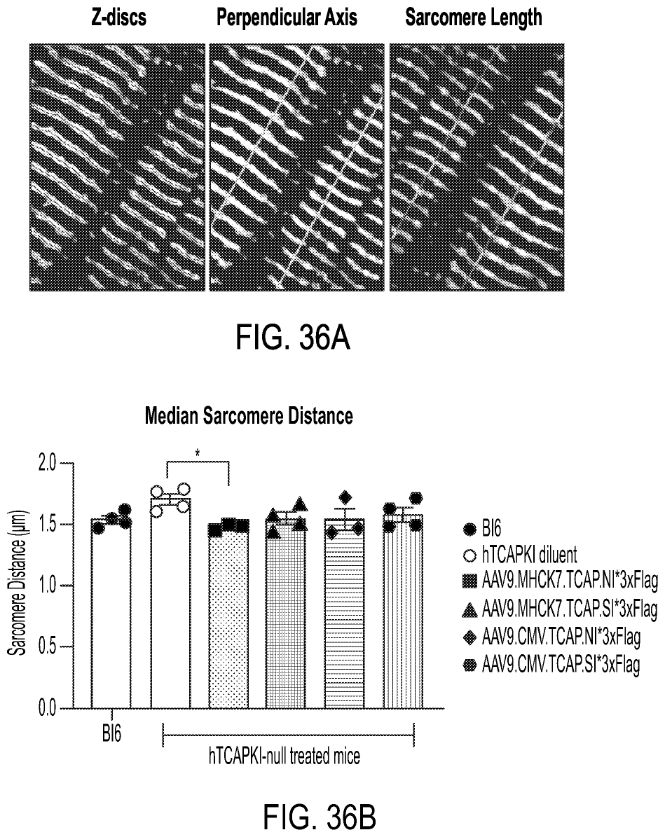

This example provides supplementary data from experiments reported in Example 6. Recombinant AAV.TCAP vectors were tested in humanized TCAPKI (hTCAPKI) mice to determine expression and effect of TCAP protein. hTCAPKI mice (n=3-4) were treated intravenously for 4 weeks with rAAV.TCAP vectors (scAAV9.TCAP.MHCK7.NI3×FLAG, scAAV9.TCAP.MHCK7.SI3×FLAG, scAAV9.TCAP.CMV.NI3×FLAG, and scAAV9.TCAP.CMV.SI3×FLAG) at a dose of 3E13 vg/kg. Age-matched untreated C57Bl6 (Bl6) and diluent treated hTCAPKI-null (hTCAPKI) mice were used as controls (Table 9). Animals were sacrificed 4 weeks post-vector administration and skeletal muscles were subject to various tests as described below and as shown in FIGS. 33A-E, 34A-E, 35A-B, and 36A-B.

### Materials and Methods

### Protein Extraction and Western Blotting Analysis

Mouse tissue lysates were prepared by mechanically disrupting left sides of quadriceps (L. Quad) and gastrocnemius (L. Gast) tissues in a TissueLyser II (Qiagen, USA) in RIPA buffer (Cell signaling, cat #9806S or alternative), containing PhosSTOP inhibitors (Sigma, cat #4906845001), and Protein Inhibitor cocktails (Sigma Aldrich Fine Chemicals Biosciences, cat #11836170001 and cat #05892970001). The lysate was centrifuged at 14,000 g for 20 min at 4° C. and supernatants were collected for analysis. Total protein was quantified by using the BCA protein assay kit (Pierce, cat #PI23227, Thermo Scientific, USA) following manufacturer's protocol. The supernatant was mixed with a 4× Laemmli sample buffer and boiled for 10 min at 90-100° C., and 25 μg of total protein was run on a precast Novex 12% Tris-Glycine Protein Gels (Invitrogen, cat #XP00120BOX). Trans-Blot Turbo Mini PVDF Transfer Packs, 0.2 μm (Bio-Rad, cat #1704156) were utilized to transfer proteins to the membrane. Membranes were blocked in 5% non-dry fat milk (NDFM) in TBST buffer and then probed with primary antibody cocktail containing, anti-TCAP antibody (MyBioSource, cat #MBS9126105, anti-rabbit polyclonal (1:1000)) and anti-GAPDH antibody (Protein Tech, cat #60004-1-Ig, anti-mouse monoclonal, (1:50,000)) overnight at 4° C. Membrane was washed (4×5 min with 0.1% Tween TBS (TBST)) and exposed to the secondary antibody cocktails, containing goat anti-rabbit IgG (HRP) (abcam, cat #ab6721) and goat anti-mouse IgG (HRP) (abcam, cat #ab6789) for 60 min at RT at 1:5000 dilution in 1-5% NDFM in TBST buffer. Blots then were washed 5×5 min in TBST, followed by 1×5 min wash in TBS. Membranes were incubated with ECL reagent prior to visualization on Chemidoc MP Imaging System (Bio Rad, USA). TCAP signal in treated and control tissues was quantified using Image Lab software (Bio Rad, version 6.0.0 build 25) by normalizing the TCAP bands to the GAPDH band intensity of the respective sample. The normalized TCAP signal from Bl6 control pool lanes was averaged and test samples were normalized to this average to give final TCAP quantification (%).

### Immunofluorescent (IF) Staining

Triceps brachii (Tri) and heart tissues were stained using the following method. 7 μm-thick longitudinally oriented sections of cryopreserved tissues were air-dried for 15 min before staining. Following rehydration in TBS for 15 min, sections were incubated for 1 hour with blocking solution containing 10% normal goat serum (NGS, Invitrogen, cat #10000C or alternative) and 1% bovine serum albumin (BSA, Sigma cat #A3294 or alternative) in TBST buffer (TBS-0.1% Tween 20) followed by 2 hours incubation with a goat-anti-mouse Fab IgG (Jackson, cat #115-007-003 or alternative; dilution 1:10) in TBST at room temperature (RT). Sections were incubated with primary antibodies for 2 hours at RT or overnight at 4° C., and then washed with 1×TBST 4 times for 5 min at RT. Sections were incubated in the appropriate Alexa Fluor 647 (Invitrogen, cat #A21244, goat anti-rabbit) and Alexa Fluor 488 (Invitrogen, cat #A11017, goat anti-mouse) conjugated secondary antibodies (1:500 dilution) for 1 hour and washed again with 1×TBST 3 times for 5 min and last time with TBS for 5 min. Tissues were then affixed with ProLong Gold Antifade mounting medium with DAPI (Invitrogen, cat #P36934 or alternative). The following primary antibodies were used: rabbit polyclonal anti-TCAP antibody (1:200; Mybiosource, cat #MBS9126105) and mouse monoclonal anti-sarcomeric alpha-actinin antibody (1:100; Invitrogen, cat #MA1-22863, clone EA-53).

### Microscope Imaging and Analysis of Sarcomere Distance in the Heart

Triceps brachii (Tri) and heart muscle images were taken on a Nikon Ti2-E inverted widefield fluorescent microscope. Four ROI mages per heart were captured as Z-stacks at 90× magnification and a final resolution of 27 nm/pixel, and the Z-stacks were reduced to a single 2D image per ROI. Thresholds for positive actinin and TCAP signal were calculated as 50% of the mean whole-image intensity for each channel in the Bl6 control images and were applied identically and automatically to all images. The proportion of actinin-positive pixels also having TCAP-positive signal, and the mean TCAP signal intensity of all actinin-positive regions, were then measured to determine TCAP positivity and intensity. The same approach was used on the Triceps.

In the heart, sarcomere distance in reduced Z-stacks was measured by manually drawing straight lines along the axis perpendicular to sarcomere direction, followed by automated unbiased detection of skeletonized sarcomere Z-discs. The perpendicular axis line was broken at each Z-disc intersection, and the resulting fragment lengths were measured to calculate sarcomere distance.

### Results

The western blot results revealed that all systemically delivered SCAAV9.TCAP.FLAG vectors showed a significant level of TCAP protein expression in both quadriceps (L. Quad) and gastrocnemius (L. Gast) muscles. Samples from C57Bl6 (Bl6) animals confirmed a previously observed result showing that the endogenously expressed mouse TCAP (mTCAP) protein is smaller (19 kDa) than the exogenously expressed human TCAP (hTCAP) protein (˜22 kDa), as expected, due to the extension of the 3×Flag sequence.

Immunofluorescent (IF) staining revealed significant restoration of TCAP-positive sarcomeric signal and TCAP intensity in both triceps brachii (Tri) and heart muscles from hTCAPKI-null males 4 weeks post scAAV9.TCAP.FLAG (Amp+) vectors administration. The staining also revealed the proper localization of restored TCAP protein in skeletal muscles.

Quantification analysis of IF images also confirmed that the distance between individual sarcomeres in the heart of hTCAPKI-null mice is higher than in control Bl6 mice and significantly reduced after restoration of TCAP expression in scAAV9.TCAP.FLAG-treated mice eaching the normal level seen in controls.

TABLE 11
Sequence Index Table.

| SEQ | |
|---|---|
| ID NO: | Sequence Descriptor |
| 1 | Human TCAP gene |
| 2 | Human TCAP gene codon-optimized |
| 3 | Human TCAP gene codon-optimized |
| 4 | Human TCAP gene codon-optimized |
| 5 | Human TCAP polypeptide |
| 6 | CMV promoter |
| 7 | MHCK7 promoter |
| 8 | Native TCP promoter |
| 9 | NP.TCAP (1^{st} gen) |
| 10 | MHCK7.TCAP.NI (1^{st} gen) |
| 11 | MHCK7.TCAP.SI (1^{st} gen) |
| 12 | CMV.TCAP.NI (1^{st} gen) |
| 13 | CMV.TCAP.SI (1^{st} gen) |
| 14 | NP.TCAP.3xFLAG (1^{st} gen) |
| 15 | MHCK7.TCAP.NI3xFLAG (1^{st} gen) |
| 16 | MHCK7.TCAP.SI3xFLAG (1^{st} gen) |
| 17 | CMV.TCAP.NI3xFLAG (1^{st} gen) |
| 18 | CMV.TCAP.SI3xFLAG (1^{st} gen) |
| 19 | rAAV.NP.TCAP (FIG. 9) (1^{st} gen) |
| 20 | rAAV.MHCK7.TCAP.NI (FIG. 10) (1^{st} gen) |
| 21 | rAAV.MHCK7.TCAP.SI (FIG. 11) (1^{st} gen) |
| 22 | rAAV.CMV.TCAP.NI (FIG. 12) (1^{st} gen) |
| 23 | rAAV.CMV.TCAP.SI (FIG. 13) (1^{st} gen) |
| 24 | rAAV.NP.TCAP.3xFLAG (FIG. 14) (1^{st} gen) |
| 25 | rAAV.MHCK7.TCAP.NI3xFLAG (FIG. 15) (1^{st} gen) |
| 26 | rAAV.MHCK7.TCAP.SI3xFLAG (FIG. 16) (1^{st} gen) |
| 27 | rAAV.CMV.TCAP.NI3xFLAG (FIG. 17) (1^{st} gen) |
| 28 | rAAV.CMV.TCAP.SI3xFLAG (FIG. 18) (1^{st} gen) |
| 29 | NP.TCAP/SD (2^{nd} gen.) |
| 30 | MHCK7.TCAP.NI/SD (2^{nd} gen.) |
| 31 | MHCK7.TCAP.SI/SD (2^{nd} gen.) |
| 32 | CMV.TCAP.NI/SD (2^{nd} gen.) |
| 33 | CMV.TCAP.SI/SD (2^{nd} gen.) |
| 34 | NP.TCAP.3xFLAG/SD (2^{nd} gen.) |
| 35 | MHCK7.TCAP.NI3xFLAG/SD (2^{nd} gen.) |
| 36 | MHCK7.TCAP.SI3xFLAG/SD (2^{nd} gen.) |
| 37 | CMV.TCAP.NI3xFLAG/SD (2^{nd} gen.) |
| 38 | CMV.TCAP.SI3xFLAG/SD (2^{nd} gen.) |
| 39 | rAAV.NP.TCAP/SD (FIG. 19) (2^{nd} gen.) |
| 40 | rAAV.MHCK7.TCAP.NI/SD (FIG. 20) (2^{nd} gen.) |
| 41 | rAAV.MHCK7.TCAP.SI/SD (FIG. 21) (2^{nd} gen.) |
| 42 | rAAV.CMV.TCAP.NI/SD (FIG. 22) (2^{nd} gen.) |
| 43 | rAAV.CMV.TCAP.SI/SD (FIG. 23) (2^{nd} gen.) |
| 44 | rAAV.NP.TCAP.3xFLAG/SD (FIG. 24) (2^{nd} gen.) |
| 45 | rAAV.MHCK7.TCAP.NI3xFLAG/SD (FIG. 25) (2^{nd} gen.) |
| 46 | rAAV.MHCK7.TCAP.SI3xFLAG/SD (FIG. 26) (2^{nd} gen.) |
| 47 | rAAV.CMV.TCAP.NI3xFLAG/SD (FIG. 27) (2^{nd} gen.) |
| 48 | rAAV.CMV.TCAP.SI3xFLAG/SD (FIG. 28) (2^{nd} gen.) |
| 49 | NP.TCAP/SD (2^{nd} gen.) transgene with 5′ and 3′ ITR sequences |
| 50 | MHCK7.TCAP.NI/SD (2^{nd} gen.) transgene with 5′ and 3′ ITR sequences |
| 51 | MHCK7.TCAP.SI/SD (2^{nd} gen.) transgene with 5′ and 3′ ITR sequences |
| 52 | CMV.TCAP.NI/SD (2^{nd} gen.) transgene with 5′ and 3′ ITR sequences |
| 53 | CMV.TCAP.SI/SD (2^{nd} gen.) transgene with 5′ and 3′ ITR sequences |
| 54 | NP.TCAP.3xFLAG/SD (2^{nd} gen.) transgene with 5′ and 3′ ITR sequences |
| 55 | MHCK7.TCAP.NI3xFLAG/SD (2^{nd} gen.) transgene with 5′ and 3′ ITR |
| | sequences |
| 56 | MHCK7.TCAP.SI3xFLAG/SD (2^{nd} gen.) transgene with 5′ and 3′ ITR |
| | sequences |
| 57 | CMV.TCAP.NI3xFLAG/SD (2^{nd} gen.) transgene with 5′ and 3′ ITR |
| | sequences |
| 58 | CMV.TCAP.SI3xFLAG/SD (2^{nd} gen.) transgene with 5′ and 3′ ITR |
| | sequences |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control. References referred to herein with numbering are provided with the full citation as shown herein below.

### REFERENCES

1. Barthelemy, F., Blouin, C., Wein, N., Mouly, V., Courrier, S., Dionnet, E., Kergourlay, V., Mathieu, Y., Garcia, L., Butler-Browne, G., Lamaze, C., et al. (2015). Exon 32 Skipping of Dysferlin Rescues Membrane Repair in Patients' Cells. J Neuromuscul Dis 2, 281-290. 10.3233/JND-150109.
2. Malcher, J., Heidt, L., Goyenvalle, A., Escobar, H., Marg, A., Beley, C., Benchaouir, R., Bader, M., Spuler, S., Garcia, L., and Schowel, V. (2018). Exon Skipping in a Dysf-Missense Mutant Mouse Model. Mol Ther Nucleic Acids 13, 198-207. 10.1016/j.omtn.2018.08.013.
3. Verwey, N., Gazzoli, I., Krause, S., Mamchaoui, K., Mouly, V., and Aartsma-Rus, A. (2020). Antisense-Mediated Skipping of Dysferlin Exons in Control and Dysferlinopathy Patient-Derived Cells. Nucleic Acid Ther 30, 71-79. 10.1089/nat.2019.0788.
4. Wein, N., Vulin, A., Falzarano, M. S., Szigyarto, C. A., Maiti, B., Findlay, A., Heller, K. N., Uhlen, M., Bakthavachalu, B., Messina, S., Vita, G., et al. (2014). Translation from a DMD exon 5 IRES results in a functional dystrophin isoform that attenuates dystrophinopathy in humans and mice. Nat Med 20, 992-1000. 10.1038/nm.3628.
5. Charleston, J. S., Schnell, F. J., Dworzak, J., Donoghue, C., Lewis, S., Chen, L., Young, G. D., Milici, A. J., Voss, J., DeAlwis, U., Wentworth, B., et al. (2018). Eteplirsen treatment for Duchenne muscular dystrophy: Exon skipping and dystrophin production. Neurology 90, e2146-e2154. 10.1212/WNL.0000000000005680.
6. Lee, J. J. A., Saito, T., Duddy, W., Takeda, S., and Yokota, T. (2018). Direct Reprogramming of Human DMD Fibroblasts into Myotubes for In Vitro Evaluation of Antisense-Mediated Exon Skipping and Exons 45-55 Skipping Accompanied by Rescue of Dystrophin Expression. Methods Mol Biol 1828, 141-150. 10.1007/978-1-4939-8651-4_8.
7. Echevarria, L., Aupy, P., and Goyenvalle, A. (2018). Exon-skipping advances for Duchenne muscular dystrophy. Hum Mol Genet 27, R163-R172. 10.1093/hmg/ddyl71.
8. Lee, E. H., Gao, M., Pinotsis, N., Wilmanns, M., and Schulten, K. (2006). Mechanical strength of the titin Z1Z2-telethonin complex. Structure 14, 497-509. 10.1016/j.str.2005.12.005.
9. Zou, P., Pinotsis, N., Lange, S., Song, Y. H., Popov, A., Mavridis, I., Mayans, O. M., Gautel, M., and Wilmanns, M. (2006). Palindromic assembly of the giant muscle protein titin in the sarcomeric Z-disk. Nature 439, 229-233. 10.1038/nature04343.
10. Zhang, R., Yang, J., Zhu, J., and Xu, X. (2009). Depletion of zebrafish Tcap leads to muscular dystrophy via disrupting sarcomere-membrane interaction, not sarcomere assembly. Hum Mol Genet 18, 4130-4140. 10.1093/hmg/ddp362.
11. Amato, A. A. (2011). Other limb-girdle muscular dystrophies. Handb Clin Neurol 101, 119-124. 10.1016/B978-0-08-045031-5.00008-6.
12. Podobed, P. S., Alibhai, F. J., Chow, C. W., and Martino, T. A. (2014). Circadian regulation of myocardial sarcomeric Titin-cap (Tcap, telethonin): identification of cardiac clock-controlled genes using open access bioinformatics data. PLOS One 9, e104907. 10.1371/journal.pone.0104907.
13. Wirianto, M., Yang, J., Kim, E., Gao, S., Paudel, K. R., Choi, J. M., Choe, J., Gloston, G. F., Ademoji, P., Parakramaweera, R., Jin, J., et al. (2020). The GSK-3beta-FBXL21 Axis Contributes to Circadian TCAP Degradation and Skeletal Muscle Function. Cell Rep 32, 108140. 10.1016/j.celrep.2020.108140.
14. Valle, G., Faulkner, G., De Antoni, A., Pacchioni, B., Pallavicini, A., Pandolfo, D., Tiso, N., Toppo, S., Trevisan, S., and Lanfranchi, G. (1997). Telethonin, a novel sarcomeric protein of heart and skeletal muscle. FEBS Lett 415, 163-168. 10.1016/s0014-5793 (97) 01108-3.
15. Gregorio, C. C., Trombitas, K., Centner, T., Kolmerer, B., Stier, G., Kunke, K., Suzuki, K., Obermayr, F., Herrmann, B., Granzier, H., Sorimachi, H., et al. (1998). The NH2 terminus of titin spans the Z-disc: its interaction with a novel 19-kD ligand (T-cap) is required for sarcomeric integrity. J Cell Biol 143, 1013-1027. 10.1083/jcb.143.4.1013.
16. Grieger, J. C., and Samulski, R. J. (2005). Adeno-associated virus as a gene therapy vector: vector development, production and clinical applications. Adv Biochem Eng Biotechnol 99, 119-145.
17. Xiao, X., Li, J., and Samulski, R. J. (1998). Production of high-titer recombinant adeno-associated virus vectors in the absence of helper adenovirus. J Virol 72, 2224-2232. 10.1128/JVI.72.3.2224-2232.1998.
18. Tijssen, P. (1990). CRC handbook of parvoviruses (Boca Raton, Fla.: CRC Press).
19. Berns, K. I. (1990). Parvoviridae and their replication (New York: Raven Press).
20. Pattison, J. R. (1988). Parvoviruses and human disease (Boca Raton, Fla.: CRC Press).
21. Fraenkel-Conrat, H., and Wagner, R. R. (1974). Comprehensive virology (New York: Plenum Press).
22. Srivastava, A., Lusby, E. W., and Berns, K. I. (1983). Nucleotide sequence and organization of the adeno-associated virus 2 genome. J Virol 45, 555-564. 10.1128/JVI.45.2.555-564.1983.
23. Ruffing, M., Heid, H., and Kleinschmidt, J. A. (1994). Mutations in the carboxy terminus of adeno-associated virus 2 capsid proteins affect viral infectivity: lack of an RGD integrin-binding motif. J Gen Virol 75 (Pt 12), 3385-3392. 10.1099/0022-1317-75-12-3385.
24. Gao, G., Vandenberghe, L. H., Alvira, M. R., Lu, Y., Calcedo, R., Zhou, X., and Wilson, J. M. (2004). Clades of Adeno-associated viruses are widely disseminated in human tissues. J Virol 78, 6381-6388. 10.1128/JVI.78.12.6381-6388.2004.
25. De, B. P., Heguy, A., Hackett, N. R., Ferris, B., Leopold, P. L., Lee, J., Pierre, L., Gao, G., Wilson, J. M., and Crystal, R. G. (2006). High levels of persistent expression of alpha1-antitrypsin mediated by the nonhuman primate serotype rh.10 adeno-associated virus despite preexisting immunity to common human adeno-associated viruses. Mol Ther 13, 67-76. 10.1016/j.ymthe.2005.09.003.
26. Mori, S., Wang, L., Takeuchi, T., and Kanda, T. (2004). Two novel adeno-associated viruses from cynomolgus monkey: pseudotyping characterization of capsid protein. Virology 330, 375-383. 10.1016/j.virol.2004.10.012.
27. Rodino-Klapac, L. R., Janssen, P. M., Montgomery, C. L., Coley, B. D., Chicoine, L. G., Clark, K. R., and Mendell, J. R. (2007). A translational approach for limb vascular delivery of the micro-dystrophin gene without high volume or high pressure for treatment of Duchenne muscular dystrophy. J Transl Med 5, 45. 10.1186/1479-5876-5-45.
28. Muzyczka, N. (1992). Use of adeno-associated virus as a general transduction vector for mammalian cells. Curr Top Microbiol Immunol 158, 97-129. 10.1007/978-3-642-75608-5_5.
29. Marsic, D., Govindasamy, L., Currlin, S., Markusic, D. M., Tseng, Y. S., Herzog, R. W., Agbandje-McKenna, M., and Zolotukhin, S. (2014). Vector design Tour de Force: integrating combinatorial and rational approaches to derive novel adeno-associated virus variants. Mol Ther 22, 1900-1909. 10.1038/mt.2014.139.
30. Weinmann, J., Weis, S., Sippel, J., Tulalamba, W., Remes, A., El Andari, J., Herrmann, A. K., Pham, Q. H., Borowski, C., Hille, S., Schonberger, T., et al. (2020). Identification of a myotropic AAV by massively parallel in vivo evaluation of barcoded capsid variants. Nat Commun 11, 5432. 10.1038/s41467-020-19230-w.
31. Tabebordbar, M., Lagerborg, K. A., Stanton, A., King, E. M., Ye, S., Tellez, L., Krunnfusz, A., Tavakoli, S., Widrick, J. J., Messemer, K. A., Troiano, E. C., et al. (2021). Directed evolution of a family of AAV capsid variants enabling potent muscle-directed gene delivery across species. Cell 184, 4919-4938 e4922. 10.1016/j.cell.2021.08.028.
32. Clark, K. R., Sferra, T. J., and Johnson, P. R. (1997). Recombinant adeno-associated viral vectors mediate long-term transgene expression in muscle. Hum Gene Ther 8, 659-669. 10.1089/hum.1997.8.6-659.
33. Kessler, P. D., Podsakoff, G. M., Chen, X., McQuiston, S. A., Colosi, P. C., Matelis, L. A., Kurtzman, G. J., and Byrne, B. J. (1996). Gene delivery to skeletal muscle results in sustained expression and systemic delivery of a therapeutic protein. Proc Natl Acad Sci USA 93, 14082-14087. 10.1073/pnas.93.24.14082.
34. Xiao, X., Li, J., and Samulski, R. J. (1996). Efficient long-term gene transfer into muscle tissue of immunocompetent mice by adeno-associated virus vector. J Virol 70, 8098-8108. 10.1128/JVI.70.11.8098-8108.1996.
35. Chao, H., Liu, Y., Rabinowitz, J., Li, C., Samulski, R. J., and Walsh, C. E. (2000). Several log increase in therapeutic transgene delivery by distinct adeno-associated viral serotype vectors. Mol Ther 2, 619-623. 10.1006/mthe.2000.0219.
36. Chao, H., Monahan, P. E., Liu, Y., Samulski, R. J., and Walsh, C. E. (2001). Sustained and complete phenotype correction of hemophilia B mice following intramuscular injection of AAV1 serotype vectors. Mol Ther 4, 217-222. 10.1006/mthe.2001.0449.
37. Herzog, R. W., Hagstrom, J. N., Kung, S. H., Tai, S. J., Wilson, J. M., Fisher, K. J., and High, K. A. (1997). Stable gene transfer and expression of human blood coagulation factor IX after intramuscular injection of recombinant adeno-associated virus. Proc Natl Acad Sci USA 94, 5804-5809. 10.1073/pnas.94.11.5804.
38 Murphy, J. E., Zhou, S., Giese, K., Williams, L. T., Escobedo, J. A., and Dwarki, V. J. (1997). Long-term correction of obesity and diabetes in genetically obese mice by a single intramuscular injection of recombinant adeno-associated virus encoding mouse leptin. Proc Natl Acad Sci USA 94, 13921-13926. 10.1073/pnas.94.25.13921.
39. Lewis, A. D., Chen, R., Montefiori, D. C., Johnson, P. R., and Clark, K. R. (2002). Generation of neutralizing activity against human immunodeficiency virus type 1 in serum by antibody gene transfer. J Virol 76, 8769-8775. 10.1128/jvi.76.17.8769-8775.2002.
40. Samulski, R. J., Berns, K. I., Tan, M., and Muzyczka, N. (1982). Cloning of adeno-associated virus into pBR322: rescue of intact virus from the recombinant plasmid in human cells. Proc Natl Acad Sci USA 79, 2077-2081. 10.1073/pnas.79.6.2077.
41. Laughlin, C. A., Tratschin, J. D., Coon, H., and Carter, B. J. (1983). Cloning of infectious adeno-associated virus genomes in bacterial plasmids. Gene 23, 65-73. 10.1016/0378-1119 (83) 90217-2.
42. Senapathy, P., and Carter, B. J. (1984). Molecular cloning of adeno-associated virus variant genomes and generation of infectious virus by recombination in mammalian cells. J Biol Chem 259, 4661-4666.
43. Carter, B. J. (1992). Adeno-associated virus vectors. Curr Opin Biotechnol 3, 533-539. 10.1016/0958-1669 (92) 90082-t.
44 Tratschin, J. D., West, M. H., Sandbank, T., and Carter, B. J. (1984). A human parvovirus, adeno-associated virus, as a eucaryotic vector: transient expression and encapsidation of the procaryotic gene for chloramphenicol acetyltransferase. Mol Cell Biol 4, 2072-2081. 10.1128/mcb.4.10.2072-2081.1984.
45. Hermonat, P. L., and Muzyczka, N. (1984). Use of adeno-associated virus as a mammalian DNA cloning vector: transduction of neomycin resistance into mammalian tissue culture cells. Proc Natl Acad Sci USA 81, 6466-6470. 10.1073/pnas.81.20.6466.
46. Tratschin, J. D., Miller, I. L., Smith, M. G., and Carter, B. J. (1985). Adeno-associated virus vector for high-frequency integration, expression, and rescue of genes in mammalian cells. Mol Cell Biol 5, 3251-3260. 10.1128/mcb.5.11.3251-3260.1985.
47. Mclaughlin, S. K., Collis, P., Hermonat, P. L., and Muzyczka, N. (1988). Adeno-associated virus general transduction vectors: analysis of proviral structures. J Virol 62, 1963-1973. 10.1128/JVI.62.6.1963-1973.1988.
48. Samulski, R. J., Chang, L. S., and Shenk, T. (1989). Helper-free stocks of recombinant adeno-associated viruses: normal integration does not require viral gene expression. J Virol 63, 3822-3828. 10.1128/JVI.63.9.3822-3828.1989.
49. Perrin, P., Madhusudana, S., Gontier-Jallet, C., Petres, S., Tordo, N., and Merten, O. W. (1995). An experimental rabies vaccine produced with a new BHK-21 suspension cell culture process: use of serum-free medium and perfusion-reactor system. Vaccine 13, 1244-1250. 10.1016/0264-410x(94)00022-f.
50. Paul, R. W., Morris, D., Hess, B. W., Dunn, J., and Overell, R. W. (1993). Increased viral titer through concentration of viral harvests from retroviral packaging lines. Hum Gene Ther 4, 609-615. 10.1089/hum. 1993.4.5-609.
51. Clark, K. R., Voulgaropoulou, F., and Johnson, P. R. (1996). A stable cell line carrying adenovirus-inducible rep and cap genes allows for infectivity titration of adeno-associated virus vectors. Gene Ther 3, 1124-1132.
52. Clark, K. R., Liu, X., McGrath, J. P., and Johnson, P. R. (1999). Highly purified recombinant adeno-associated virus vectors are biologically active and free of detectable helper and wild-type viruses. Hum Gene Ther 10, 1031-1039. 10.1089/10430349950018427.
53. Schnepp, B., and Clark, K. R. (2002). Highly purified recombinant adeno-associated virus vectors. Preparation and quantitation. Methods Mol Med 69, 427-443. 10.1385/1-59259-141-8:427.
54. Lebkowski J S, McNally M M, Okarma T B, Lerch L B. (1988) Adeno-associated virus: a vector system for efficient introduction and integration of DNA into a variety of mammalian cell types. Mol Cell Biol October; 8 (10): 3988-96. doi: 10.1128/mcb.8.10.3988-3996.
55. Mazzone A et al. (2008) A mutation in telethonin alters Nav1.5 function. J Biol Chem June 13; 283 (24): 16537-44. doi: 10.1074/jbc.M801744200.

1. A nucleic acid comprising a polynucleotide comprising
(a) one or more regulatory control element(s); and
(b) a titin-cap protein (TCAP) cDNA sequence.

2. The nucleic acid of claim 1, wherein the TCAP cDNA comprises
(a) a nucleotide sequence having at least 90% sequence identity to any one of SEQ ID NOs: 1-4;
(b) the nucleotide sequence set forth in any one of SEQ ID NOs: 1-4; or
(c) a nucleotide sequence encoding TCAP comprising the amino acid sequence set forth in SEQ ID NO: 5.

3. The nucleic acid of claim 1, wherein the one or more regulatory control element(s) is an H1 promoter, an EF1-alpha promoter, a minimal EF1-alpha promoter, an unc45b promoter, a CK1 promoter, a CK6 promoter, a CK7 promoter, a CK8e promoter, a cardiac troponin C (cTnC) promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a desmin promoter, the chicken β actin promoter (CBA), the P546 promoter, the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter, or a native TCAP promoter.

4. The nucleic acid of claim 1, wherein the one or more regulatory control element(s) is a CMV promoter, an MHCK7 promoter, a native TCAP promoter, or a fragment of any of the CMV promoter, the MHCK7 promoter, or the native TCAP promoter.

5. The nucleic acid of claim 1, wherein the regulatory control element comprises
(a) a nucleotide sequence having at least 90% sequence identity to SEQ ID NO: 6, 7 or 8; or
(b) the nucleotide sequence set forth in SEQ ID NO: 6, 7, or 8.

6. The nucleic acid of claim 1 comprising
(a) a nucleotide sequence having at least 90% sequence identity to any one of SEQ ID NOs: 29-38; or
(b) the nucleotide sequence set forth in any one of SEQ ID NOs: 29-38.

7. The nucleic acid of claim 1 further comprising an inverted terminal repeat sequence.

8. The nucleic acid of claim 7 comprising
(a) a nucleotide sequence having at least 90% sequence identity to any one of SEQ ID NOs: 49-58; or
(b) the nucleotide sequence set forth in any one of SEQ ID NOs: 49-58.

9. The nucleic acid of claim 1 comprising
(a) a nucleotide sequence having at least 90% sequence identity to any one of SEQ ID NOs: 39-48; or
(b) the nucleotide sequence set forth in any one of SEQ ID NOs: 39-48.

10. A nanoparticle, extracellular vesicle, exosome, or vector comprising the nucleic acid of claim 1 or a combination of any one or more thereof.

11. The vector of claim 10, wherein the vector is a viral vector.

12. The vector of claim 11, wherein the viral vector is an adeno-associated virus (AAV), adenovirus, lentivirus, retrovirus, poxvirus, baculovirus, herpes simplex virus, vaccinia virus, or a synthetic virus.

13. The vector of claim 12, wherein the vector is an AAV vector.

14. The vector of claim 13, wherein the AAV vector lacks rep and cap genes.

15. The vector of claim 14, wherein the AAV is a recombinant AAV (rAAV), a self-complementary recombinant AAV (scAAV), or a single-stranded recombinant AAV (ssAAV).

16. The vector of claim 15, wherein the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAVMYO, or any of their derivatives.

17. The vector of claim 16, wherein the AAV is AAV9.

18. An rAAV particle comprising the vector of claim 12.

19. A composition comprising
the nucleic acid of claim 1;
and
a pharmaceutically acceptable carrier.

20. The composition of claim 19, wherein the composition is formulated for intramuscular or intravenous delivery.

21. A method of increasing the expression of a TCAP gene or TCAP protein in a cell comprising contacting the cell with
the nucleic acid of claim 1.

22. The method of claim 21, wherein the cell is a muscle cell.

23. The method of claim 21, wherein the cell is a human cell.

24. The method of claim 22, wherein the cell is in a human subject.

25. A method of treating a subject comprising a TCAP gene mutation or a telethoninopathy comprising administering to the subject an effective amount of
the nucleic acid of claim 1.

26. The method of claim 25, wherein the subject is a human subject.

27. The method of claim 25, wherein the telethoninopathy is autosomal recessive limb girdle muscular dystrophy type 2G (LGMD2G), autosomal dominant dilated cardiomyopathy (DCM), hypertrophic cardiomyopathy (HCM) or idiopathic cardiomyopathy (ICM).

28. The method of claim 25, further comprising administering any one or more of a corticosteroid, rituximab, and rapamycin to the subject.

29. The method of claim 25, wherein the nucleic acid, nanoparticle, extracellular vesicle, exosome, vector, rAAV particle, or composition is administered by intravenous or intramuscular delivery.

30-41. (canceled)


SEQUENCE LISTING (58 sequences)

SEQ ID NO: 1 - DNA, 504 bp, synthetic construct
  misc_feature 1..504: note = Synthetic
  source 1..504: mol_type = other DNA
atggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctga

SEQ ID NO: 2 - DNA, 504 bp, synthetic construct
  misc_feature 1..504: note = Synthetic
  source 1..504: mol_type = other DNA
atggcgacctccgaactgagctgcgaagtgtctgaagaaaactgcgaacgccgtgaagcgttttgggcggaatggaaggatctgaccctgagcacgcgcccggaagagggttgctctctgcacgaagaggatacccagcgtcatgaaacctatcaccagcagggtcagtgccaggttctggtgcagcgttccccgtggctgatgatgcgtatgggtattctcggtcgcgggctgcaggaataccagctgccgtatcagcgtgttctcccgctgccgatttttaccccggcgaaaatgggcgcaaccaaagaagaacgcgaagataccccgatccagctgcaggaactgctggcgctggagactgcgctgggcggtcagtgcgtggaccgtcaggaggtggcggagattaccaaacagctgccgccggtggtgccggtgagcaagccgggcgcactgcgccgctccctgagccgcagcatgtcccaggaggcgcagcgtggctga

SEQ ID NO: 3 - DNA, 504 bp, synthetic construct
  misc_feature 1..504: note = Synthetic
  source 1..504: mol_type = other DNA
atggccacctctgagctgagctgcgaggtttccgaggagaactgtgagcgtcgcgaggccttctgggccgaatggaaagatctgacgctgtccacccgtcccgaggagggctgctccctgcatgaggaagacacccagcgccatgagacctaccaccagcagggccagtgccaggtcctggtgcagcgctctccctggctgatgatgcgcatgggcatcctcggccgtggcctgcaggagtatcagctgccctaccagcgcgtgctcccgctgcccatcttcacccctgccaagatgggtgccaccaaggaagaacgtgaggacacccccatccagctgcaggagctgctggcgctggaaaccgccctgggtggccagtgtgtggaccgccaggaagttgccgaaatcactaagcagctgccccctgttgttcctgttagcaagcccggtgcacttcgtcgtagcctgtcccgttccatgagccaggaagcacagcgcggctga

SEQ ID NO: 4 - DNA, 504 bp, synthetic construct
  misc_feature 1..504: note = Synthetic
  source 1..504: mol_type = other DNA
atggctacctctgaactgagctgcgaagtgtctgaggaaaactgtgaacgccgggaagcgttctgggccgaatggaaagatctgaccctgtccactcgtccagaagaaggctgctccctgcatgaagaagatacccagagacatgaaacctatcaccagcagggtcagtgccaggttctggttcagcgttcgccctggctgatgatgcgtatgggcattctgggccgtggtctgcaggaataccagctgccctaccagcgtgtgctgccgctgcccattttcaccccggcgaaaatgggtgcgaccaaagaagagcgtgaagataccccgattcagctgcaggaactgctggcgctggaaaccgccctgggtggtcagtgcgtggaccgccaggaggtggcggaaattacaaaacagctgccccctgtggttccggttagcaaaccgggcgcactgcgtcgtagcctgtcccgtagcatgtcccaggaagcgcagagaggttga

SEQ ID NO: 5 - protein, 167 aa, synthetic construct
  REGION 1..167: note = Synthetic
MATSELSCEVSEENCERREAFWAEWKDLTLSTRPEEGCSLHEEDTQRHETYHQQGQCQVLVQRSPWLMMRMGILGRGLQEYQLPYQRVLPLPIFTPAKMGATKEEREDTPIQLQELLALETALGGQCVDRQEVAEITKQLPPVVPVSKPGALRRSLSRSMSQEAQRG

SEQ ID NO: 6 - DNA, 508 bp, synthetic construct
  misc_feature 1..508: note = Synthetic
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - DNA, 788 bp, synthetic construct
  misc_feature 1..788: note = Synthetic
  source 1..788: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcgg

SEQ ID NO: 8 - DNA, 904 bp, synthetic construct
  misc_feature 1..904: note = Synthetic
  source 1..904: mol_type = other DNA
ccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagag

SEQ ID NO: 9 - DNA, 1725 bp, synthetic construct
  misc_feature 1..1725: note = Synthetic
  source 1..1725: mol_type = other DNA
ccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtg

SEQ ID NO: 10 - DNA, 1609 bp, synthetic construct
  misc_feature 1..1609: note = Synthetic
  source 1..1609: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtg

SEQ ID NO: 11 - DNA, 1515 bp, synthetic construct
  misc_feature 1..1515: note = Synthetic
  source 1..1515: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtg

SEQ ID NO: 12 - DNA, 1447 bp, synthetic construct
  misc_feature 1..1447: note = Synthetic
  source 1..1447: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccaggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtg

SEQ ID NO: 13 - DNA, 1348 bp, synthetic construct
  misc_feature 1..1348: note = Synthetic
  source 1..1348: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtg

SEQ ID NO: 14 - DNA, 1791 bp, synthetic construct
  misc_feature 1..1791: note = Synthetic
  source 1..1791: mol_type = other DNA
ccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 15 - DNA, 1678 bp, synthetic construct
  misc_feature 1..1678: note = Synthetic
  source 1..1678: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgccccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 16 - DNA, 1579 bp, synthetic construct
  misc_feature 1..1579: note = Synthetic
  source 1..1579: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 17 - DNA, 1513 bp, synthetic construct
  misc_feature 1..1513: note = Synthetic
  source 1..1513: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 18 - DNA, 1414 bp, synthetic construct
  misc_feature 1..1414: note = Synthetic
  source 1..1414: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 19 - DNA, 5627 bp, synthetic construct
  misc_feature 1..5627: note = Synthetic
  source 1..5627: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttgctagccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 20 - DNA, 5506 bp, synthetic construct
  misc_feature 1..5506: note = Synthetic
  source 1..5506: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 21 - DNA, 5412 bp, synthetic construct
  misc_feature 1..5412: note = Synthetic
  source 1..5412: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 22 - DNA, 5351 bp, synthetic construct
  misc_feature 1..5351: note = Synthetic
  source 1..5351: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccaggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 23 - DNA, 5252 bp, synthetic construct
  misc_feature 1..5252: note = Synthetic
  source 1..5252: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 24 - DNA, 5693 bp, synthetic construct
  misc_feature 1..5693: note = Synthetic
  source 1..5693: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttgctagccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 25 - DNA, 5575 bp, synthetic construct
  misc_feature 1..5575: note = Synthetic
  source 1..5575: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgccccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 26 - DNA, 5476 bp, synthetic construct
  misc_feature 1..5476: note = Synthetic
  source 1..5476: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 27 - DNA, 5417 bp, synthetic construct
  misc_feature 1..5417: note = Synthetic
  source 1..5417: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 28 - DNA, 5318 bp, synthetic construct
  misc_feature 1..5318: note = Synthetic
  source 1..5318: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacaagcttcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag

SEQ ID NO: 29 - DNA, 1725 bp, synthetic construct; identical to SEQ ID NO: 9
  misc_feature 1..1725: note = Synthetic
  source 1..1725: mol_type = other DNA

SEQ ID NO: 30 - DNA, 1609 bp, synthetic construct; identical to SEQ ID NO: 10
  misc_feature 1..1609: note = Synthetic
  source 1..1609: mol_type = other DNA

SEQ ID NO: 31 - DNA, 1515 bp, synthetic construct; identical to SEQ ID NO: 11
  misc_feature 1..1515: note = Synthetic
  source 1..1515: mol_type = other DNA

SEQ ID NO: 32 - DNA, 1447 bp, synthetic construct
  misc_feature 1..1447: note = Synthetic
  source 1..1447: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtg

SEQ ID NO: 33 - DNA, 1348 bp, synthetic construct; identical to SEQ ID NO: 13
  misc_feature 1..1348: note = Synthetic
  source 1..1348: mol_type = other DNA

SEQ ID NO: 34 - DNA, 1791 bp, synthetic construct; identical to SEQ ID NO: 14
  misc_feature 1..1791: note = Synthetic
  source 1..1791: mol_type = other DNA

SEQ ID NO: 35 - DNA, 1675 bp, synthetic construct
  misc_feature 1..1675: note = Synthetic
  source 1..1675: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 36 - DNA, 1581 bp, synthetic construct
  misc_feature 1..1581: note = Synthetic
  source 1..1581: mol_type = other DNA
gcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtg

SEQ ID NO: 37 - DNA, 1513 bp, synthetic construct; identical to SEQ ID NO: 17
  misc_feature 1..1513: note = Synthetic
  source 1..1513: mol_type = other DNA

SEQ ID NO: 38 - DNA, 1414 bp, synthetic construct; identical to SEQ ID NO: 18
  misc_feature 1..1414: note = Synthetic
  source 1..1414: mol_type = other DNA

SEQ ID NO: 39 - DNA, 5749 bp, synthetic construct
  misc_feature 1..5749: note = Synthetic
  source 1..5749: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgctagccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 40 - DNA, 5628 bp, synthetic construct
  misc_feature 1..5628: note = Synthetic
  source 1..5628: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 41 - DNA, 5534 bp, synthetic construct
  misc_feature 1..5534: note = Synthetic
  source 1..5534: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 42 - DNA, 5440 bp, synthetic construct
  misc_feature 1..5440: note = Synthetic
  source 1..5440: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 43 - DNA, 5341 bp, synthetic construct
  misc_feature 1..5341: note = Synthetic
  source 1..5341: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 44 - DNA, 5815 bp, synthetic construct
  misc_feature 1..5815: note = Synthetic
  source 1..5815: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgctagccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 45 - DNA, 5694 bp, synthetic construct
  misc_feature 1..5694: note = Synthetic
  source 1..5694: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 46 - DNA, 5600 bp, synthetic construct
  misc_feature 1..5600: note = Synthetic
  source 1..5600: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 47 - DNA, 5506 bp, synthetic construct
  misc_feature 1..5506: note = Synthetic
  source 1..5506: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 48 - DNA, 5407 bp, synthetic construct
  misc_feature 1..5407: note = Synthetic
  source 1..5407: mol_type = other DNA
atgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttt

SEQ ID NO: 49 - DNA, 2096 bp, synthetic construct
  misc_feature 1..2096: note = Synthetic
  source 1..2096: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgctagccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 50 - DNA, 1975 bp, synthetic construct
  misc_feature 1..1975: note = Synthetic
  source 1..1975: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 51 - DNA, 1881 bp, synthetic construct
  misc_feature 1..1881: note = Synthetic
  source 1..1881: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 52 - DNA, 1787 bp, synthetic construct
  misc_feature 1..1787: note = Synthetic
  source 1..1787: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 53 - DNA, 1688 bp, synthetic construct
  misc_feature 1..1688: note = Synthetic
  source 1..1688: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggctgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 54 - DNA, 2162 bp, synthetic construct
  misc_feature 1..2162: note = Synthetic
  source 1..2162: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgctagccccatgaagaagagccagtcccctcccaggtagcacatcagagcgggcaggctccagagctcttctggtcctcttgcagtggccagaaagtggggaaaggggcccagagaggggcagggactgacccatggtcacacagcctatttatggctgatctggagccaggccttctgactatcacccatcagagcccctggattcccatttcacccaacacaggtgttggggtagaactgggggccctccacccctactagcctgactgtctcctgagacctgtctcctaagaccacttacctgaccactggccatggggaaggaaaacacctggccaaggctcatgccccaggttcccaggcatgaagttgactgtctccgcccctgctggtgtggtgagggtgactggggactaggcactaggcctttggtgcaggcgcctgaggacgtggttgcactctcccttctggggatatgcccttgagcccaggcagaggagagcacagccagggcaggacctggcagccctggtacagagcccagagggggcatcagttcctgctggtcctgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggaggggagagagaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 55 - DNA, 2041 bp, synthetic construct
  misc_feature 1..2041: note = Synthetic
  source 1..2041: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 56 - DNA, 1947 bp, synthetic construct
  misc_feature 1..1947: note = Synthetic
  source 1..1947: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccttaagtctagaggctcgagaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 57 - DNA, 1853 bp, synthetic construct
  misc_feature 1..1853: note = Synthetic
  source 1..1853: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacctgaatgaggagtgatcatggctacctcagagctgagctgcgaggtgtcggaggagaactgtgagcgccgggaggccttctgggcagaatggaaggatctgacactgtccacacggcccgaggaggggtgagtgtgggtctgctagagccctgcctctgctcccccagagcaccctcactgagccatgaggccagagcatgaagccctggagaaatttctgggggtgggggcaggaagaatgccccatggggagagcaaaggggaaccacccttcctgcccccaggtcccagcagcccaggggagccccccacccagcctgtgcccagagagcaacagctcccaggagctcactgcccctcccctctccccagctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 58 - DNA, 1754 bp, synthetic construct
  misc_feature 1..1754: note = Synthetic
  source 1..1754: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcaatgaggagtgatcatggctacctcagagctgagctgcgaggtgtcggaggagaactgtgagcgccgggaggccttctgggcagaatggaaggatctgacactgtccacacggcccgaggagggctgctccctgcatgaggaggacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacccctgccaagatgggcgccaccaaggaggagcgtgaggacacccccatccagcttcaggagctgctggcgctggagacagccctgggtggccagtgtgtggaccgccaggaggtggctgagatcacaaagcagctgccccctgtggtgcctgtcagcaagcccggtgcacttcgtcgctccctgtcccgctccatgtcccaggaagcacagagaggcgactacaaagaccatgacggtgattataaagatcatgacatcgattacaaggatgacgatgacaagtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg